(19) United States
(12) Patent Application Publication
(10) Pub. No.: US 2026/0166160 A1
(43) Pub. Date: Jun. 18, 2026

(54) PROTACS TARGETING VIRAL ENZYMES FOR PRECISE TREATMENT OF COVID-19

(71) Applicant: Ohio University, Athens, OH (US)

(72) Inventors: Yong Han, Athens, OH (US); Shiyong Wu, Athens, OH (US)

(73) Assignee: Ohio University, Athens, OH (US)

(21) Appl. No.: 19/122,118

(22) PCT Filed: Oct. 17, 2023
(86) PCT No.: PCT/US2023/035271

§ 371 Date: Apr. 17, 2025

Related U.S. Application Data
(60) Provisional application No. 63/416,696, filed on Oct. 17, 2022.

(51) Int. Cl. A61K 47/55; A61K 47/54; A61K 47/64; A61P 31/14

(52) U.S. Cl. CPC A61K 47/55; A61K 47/545; A61K 47/64; A61P 31/14

(57) ABSTRACT

Described are proteolysis targeting chimeras (PROTACs) for use in managing and treating infectious disease. Described compositions can be used to inhibit viral replication associated with coronavirus, including severe acute respiratory syndrome coronavirus 2 (SARS-CoV-2), the virus that causes Coronavirus disease 2019 (COVID-19). Methods of synthesizing PROTACs are described. Example PROTAC compositions are provided, including dual targeting compounds having a main protease (Mpro) ligand attached to a linker, an E3 ligase ligand attached to the linker, and a papain-like protease (PLpro) inhibitor attached to the linker. PROTAC compounds useful for degrading Mpro activity and/or PLpro activity, and useful for treating coronavirus infections such as COVID-19, are described.

### RELATED APPLICATIONS

This application claims priority to U.S. Provisional Application No. 63/416,696 filed under 35 U.S.C. § 111(b) on Oct. 17, 2022, the disclosure of which is incorporated herein by reference in its entirety.

### STATEMENT REGARDING FEDERALLY SPONSORED RESEARCH

This invention was made with no government support. The government has no rights in this invention.

### BACKGROUND

The SARS-CoV-2 pandemic has created an urgent need for drugs to combat infection and reduce the number of deaths from the virus. Dealing with the unforeseen challenges caused by the COVID-19 pandemic calls for the engagement of specific sectors of health and pharma in critical research, including diagnostic tests, research in public health and prevention, and the development of new drugs and vaccines. There is a need for new approaches and technology to fight the virus.

### SUMMARY

Provided is a composition comprising an E3 ligase ligand attached to a linker; and a Mpro ligand attached to the linker. Methods of synthesizing the compositions are provided.

Provided are dual targeting PROTAC compounds comprising an E3 ligase ligand, a PLpro inhibitor, a Mpro ligand, and at least one linker connecting the E3 ligase ligand, the PLpro inhibitor, and the Mpro ligand. Methods of synthesizing the dual targeting PROTAC compounds are provided.

In certain embodiments, the Mpro ligand comprises any of ML-1, ML-2, ML-3, ML-4, ML-4, ML-5, ML-6, ML-7, ML-8, ML-9, ML-10, ML-11, ML-12, ML-13, ML-14, ML-15, ML-16, ML-17, ML-18, ML-19, or ML-20:

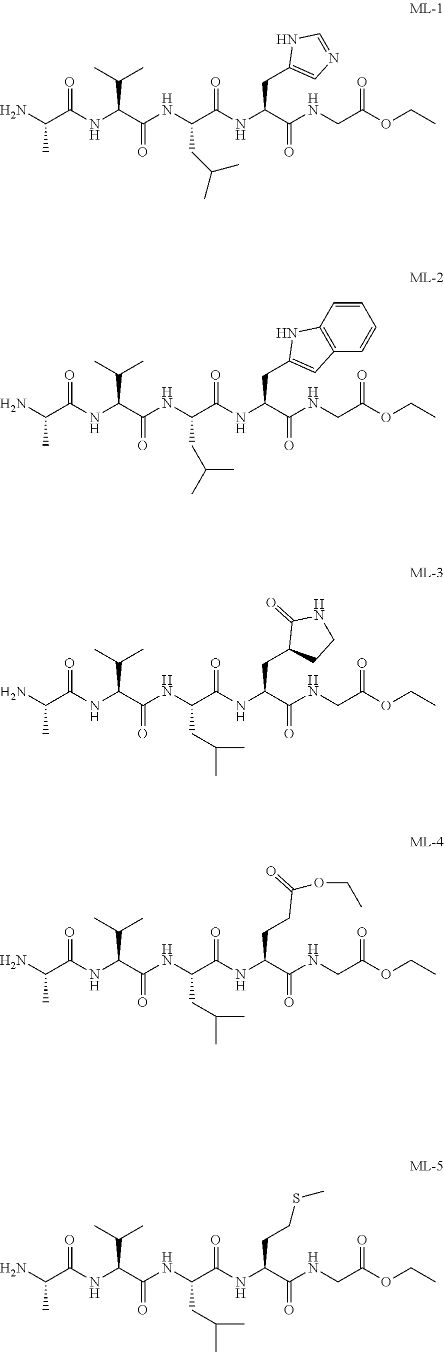 [SMILES: CCOC(=O)CNC(=O)[C@H](Cc1cnc[nH]1)NC(=O)[C@H](CC(C)C)NC(=O)[C@@H](NC(=O)[C@H](C)N)C(C)C ; CCOC(=O)CNC(=O)[C@H](Cc1cc2ccccc2[nH]1)NC(=O)[C@H](CC(C)C)NC(=O)[C@@H](NC(=O)[C@H](C)N)C(C)C ; CCOC(=O)CNC(=O)[C@H](C[C@@H]1CCNC1=O)NC(=O)[C@H](CC(C)C)NC(=O)[C@@H](NC(=O)[C@H](C)N)C(C)C ; CCOC(=O)CC[C@H](NC(=O)[C@H](CC(C)C)NC(=O)[C@@H](NC(=O)[C@H](C)N)C(C)C)C(=O)NCC(=O)OCC ; CCOC(=O)CNC(=O)[C@H](CCSC)NC(=O)[C@H](CC(C)C)NC(=O)[C@@H](NC(=O)[C@H](C)N)C(C)C]

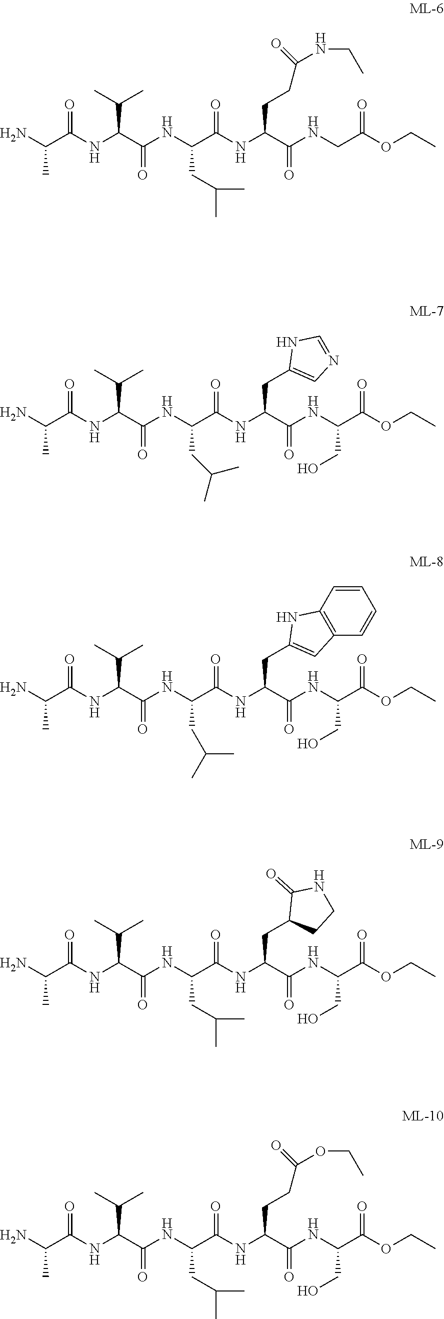 [SMILES: CCNC(=O)CC[C@H](NC(=O)[C@H](CC(C)C)NC(=O)[C@@H](NC(=O)[C@H](C)N)C(C)C)C(=O)NCC(=O)OCC ; CCOC(=O)[C@H](CO)NC(=O)[C@H](Cc1cnc[nH]1)NC(=O)[C@H](CC(C)C)NC(=O)[C@@H](NC(=O)[C@H](C)N)C(C)C ; CCOC(=O)[C@H](CO)NC(=O)[C@H](Cc1cc2ccccc2[nH]1)NC(=O)[C@H](CC(C)C)NC(=O)[C@@H](NC(=O)[C@H](C)N)C(C)C ; CCOC(=O)[C@H](CO)NC(=O)[C@H](C[C@@H]1CCNC1=O)NC(=O)[C@H](CC(C)C)NC(=O)[C@@H](NC(=O)[C@H](C)N)C(C)C ; CCOC(=O)CC[C@H](NC(=O)[C@H](CC(C)C)NC(=O)[C@@H](NC(=O)[C@H](C)N)C(C)C)C(=O)N[C@@H](CO)C(=O)OCC]

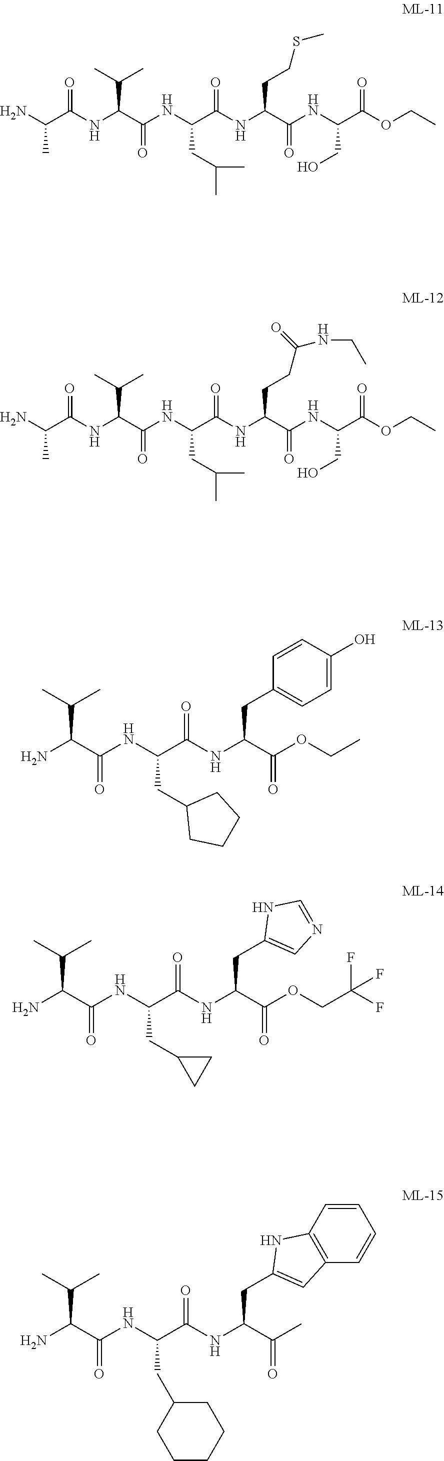 [SMILES: CCOC(=O)[C@H](CO)NC(=O)[C@H](CCSC)NC(=O)[C@H](CC(C)C)NC(=O)[C@@H](NC(=O)[C@H](C)N)C(C)C ; CCNC(=O)CC[C@H](NC(=O)[C@H](CC(C)C)NC(=O)[C@@H](NC(=O)[C@H](C)N)C(C)C)C(=O)N[C@@H](CO)C(=O)OCC ; CCOC(=O)[C@H](Cc1ccc(O)cc1)NC(=O)[C@H](CC1CCCC1)NC(=O)[C@@H](N)C(C)C ; CC(C)[C@H](N)C(=O)N[C@@H](CC1CC1)C(=O)N[C@@H](Cc1cnc[nH]1)C(=O)OCC(F)(F)F ; CC(=O)[C@H](Cc1cc2ccccc2[nH]1)NC(=O)[C@H](CC1CCCCC1)NC(=O)[C@@H](N)C(C)C]

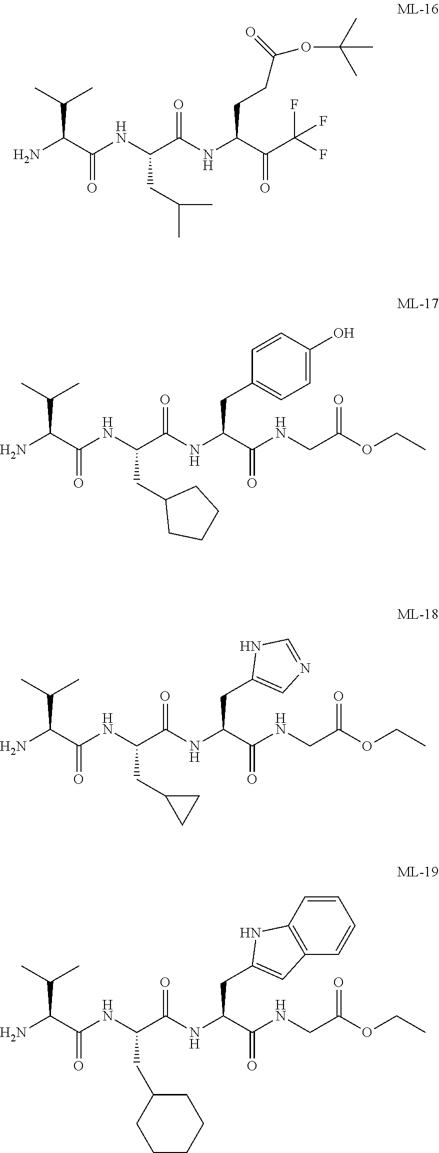 [SMILES: CC(C)C[C@H](NC(=O)[C@@H](N)C(C)C)C(=O)N[C@@H](CCC(=O)OC(C)(C)C)C(=O)C(F)(F)F ; CCOC(=O)CNC(=O)[C@H](Cc1ccc(O)cc1)NC(=O)[C@H](CC1CCCC1)NC(=O)[C@@H](N)C(C)C ; CCOC(=O)CNC(=O)[C@H](Cc1cnc[nH]1)NC(=O)[C@H](CC1CC1)NC(=O)[C@@H](N)C(C)C ; CCOC(=O)CNC(=O)[C@H](Cc1cc2ccccc2[nH]1)NC(=O)[C@H](CC1CCCCC1)NC(=O)[C@@H](N)C(C)C]

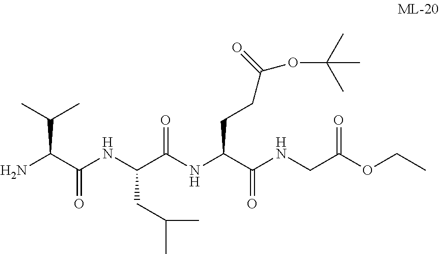 [SMILES: CCOC(=O)CNC(=O)[C@H](CCC(=O)OC(C)(C)C)NC(=O)[C@H](CC(C)C)NC(=O)[C@@H](N)C(C)C]

In certain embodiments, the E3 ligase ligand comprises pomalidomide or the von Hippel-Lindau (VHL) ligand.

In certain embodiments, the E3 ligase ligand comprises pomalidomide or a derivative thereof. In certain embodiments, the E3 ligase ligand comprises pomalidomide, an acetylated pomalidomide, an N-alkylated pomalidomide, a pomalidomide derivative with a PEG segment, 4-hydroxythalidomide, an alkyl-connected thalidomide derivative, lenalidomide, a 5-aminothalidomide derivative, thalidomide, lenalidomide, methylbestatin, nutilin-3, 4-hydroxythalidomide, or an alkyl-connected thalidomide derivatives.

In certain embodiments, the E3 ligase ligand comprises AHPC-PEGₙ-butyl COOH wherein n is 2-10; pomalidomide-PEGₙ-COOH, wherein n is 2-10; or pomalidomide-Cₙ—COOH wherein n is 3-10.

In certain embodiments, the E3 ligase ligand comprises an alkyl group, a PEG chain, an extended glycol chain, an alkyl group containing a PEG segment, an alkyelene group, a heterocyclic group, a VHL ligand, a primary amine, an alkyne, a triazole, a saturated heterocycle, or a combination thereof.

In certain embodiments, the E3 ligase ligand comprises pomalidomide and the linker comprises an alkyl group having from 1 to 6 carbons.

In certain embodiments, the E3 ligase ligand comprises pomalidomide and the linker comprises a polyethylene glycol (PEG) chain or segment.

In certain embodiments, the E3 ligase ligand comprises the von Hippel-Lindau (VHL) ligand and the linker comprises a polyethylene glycol (PEG) chain or segment.

In certain embodiments, the linker comprises a PEG chain.

In certain embodiments, the composition comprises Formula I:

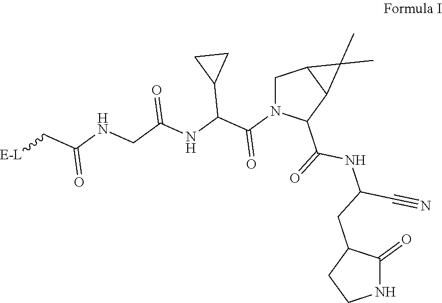

wherein E is the E3 ligase ligand, and L is the linker.

In certain embodiments, the composition comprises Formula II:

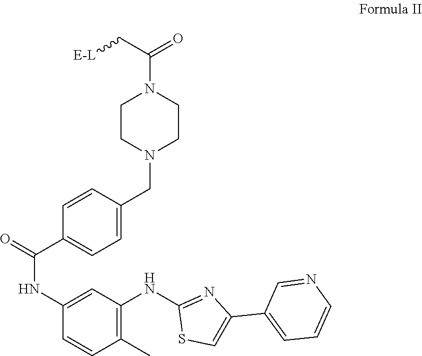

wherein E is the E3 ligase ligand, and L is the linker.

In certain embodiments, the composition comprises Formula III:

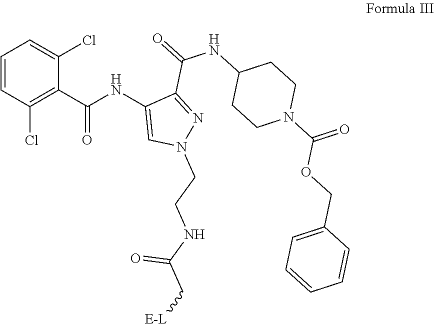

wherein E is the E3 ligase ligand, and L is the linker.

In certain embodiments, the composition comprises MP-18:

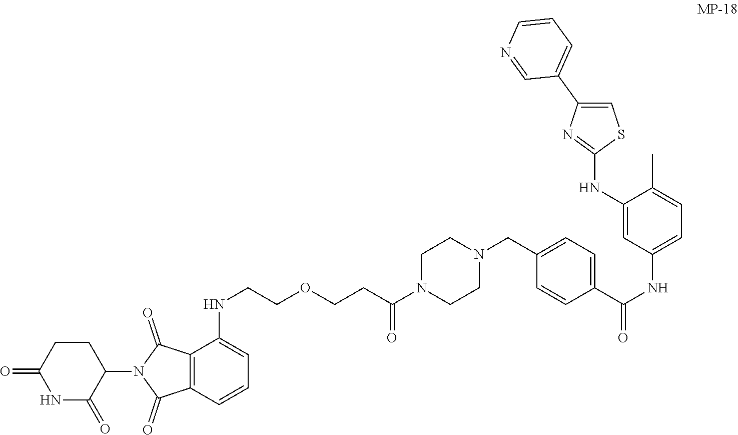 [SMILES: Cc1ccc(NC(=O)c2ccc(CN3CCN(C(=O)CCOCCNc4cccc5c4C(=O)N(C4CCC(=O)NC4=O)C5=O)CC3)cc2)cc1Nc1nc(-c2cccnc2)cs1]

In certain embodiments, the composition comprises MP-19:

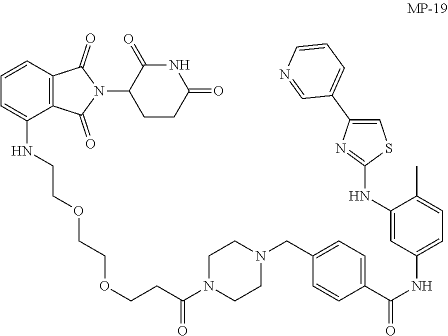 [SMILES: Cc1ccc(NC(=O)c2ccc(CN3CCN(C(=O)CCOCCOCCNc4cccc5c4C(=O)N(C4CCC(=O)NC4=O)C5=O)CC3)cc2)cc1Nc1nc(-c2cccnc2)cs1]

In certain embodiments, the composition comprises MP-20:

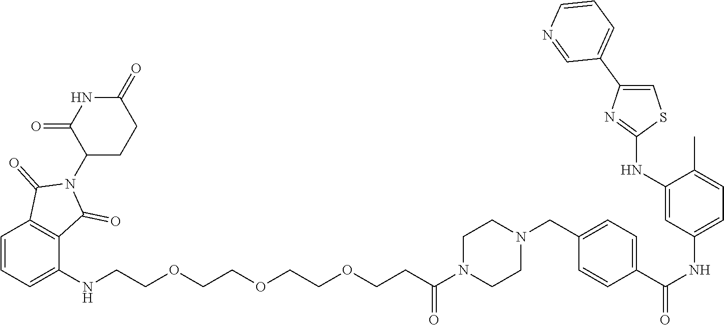 [SMILES: Cc1ccc(NC(=O)c2ccc(CN3CCN(C(=O)CCOCCOCCOCCNc4cccc5c4C(=O)N(C4CCC(=O)NC4=O)C5=O)CC3)cc2)cc1Nc1nc(-c2cccnc2)cs1]

In certain embodiments, the composition comprises MP-21:

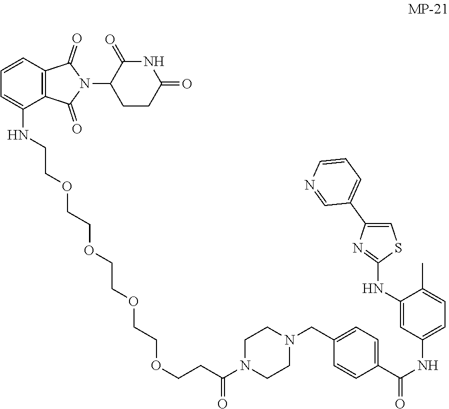 [SMILES: Cc1ccc(NC(=O)c2ccc(CN3CCN(C(=O)CCOCCOCCOCCOCCNc4cccc5c4C(=O)N(C4CCC(=O)NC4=O)C5=O)CC3)cc2)cc1Nc1nc(-c2cccnc2)cs1]

In certain embodiments, the composition comprises MP-22:

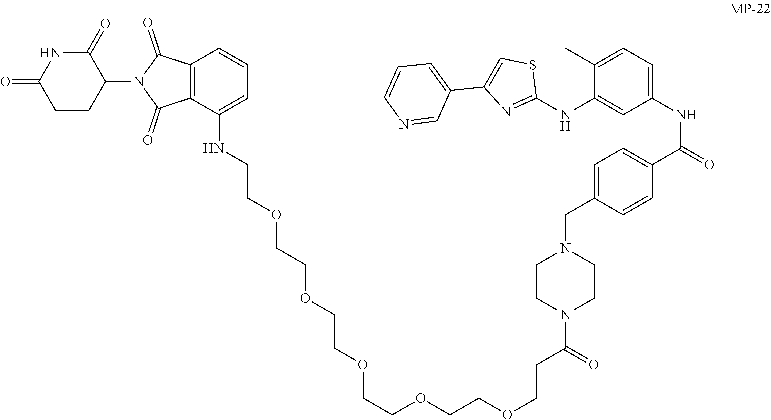 [SMILES: Cc1ccc(NC(=O)c2ccc(CN3CCN(C(=O)CCOCCOCCOCCOCCOCCNc4cccc5c4C(=O)N(C4CCC(=O)NC4=O)C5=O)CC3)cc2)cc1Nc1nc(-c2cccnc2)cs1]

In certain embodiments, the composition comprises MP-28:

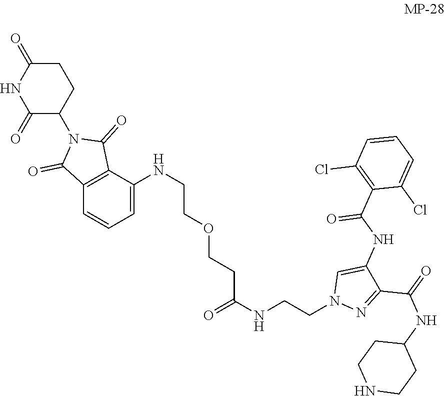 [SMILES: O=C(CCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

In certain embodiments, the composition comprises MP-29:

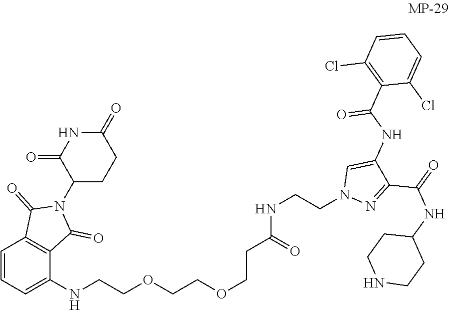 [SMILES: O=C(CCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

In certain embodiments, the composition comprises MP-30:

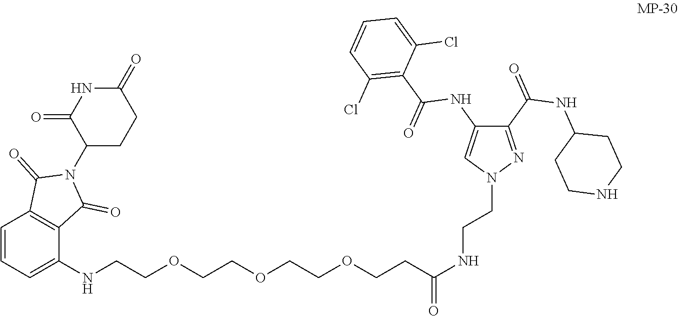 [SMILES: O=C(CCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

In certain embodiments, the composition comprises MP-31:

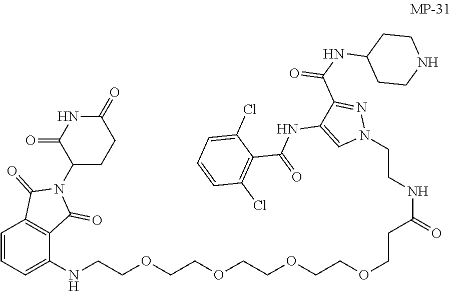 [SMILES: O=C(CCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

In certain embodiments, the composition comprises MP-32:

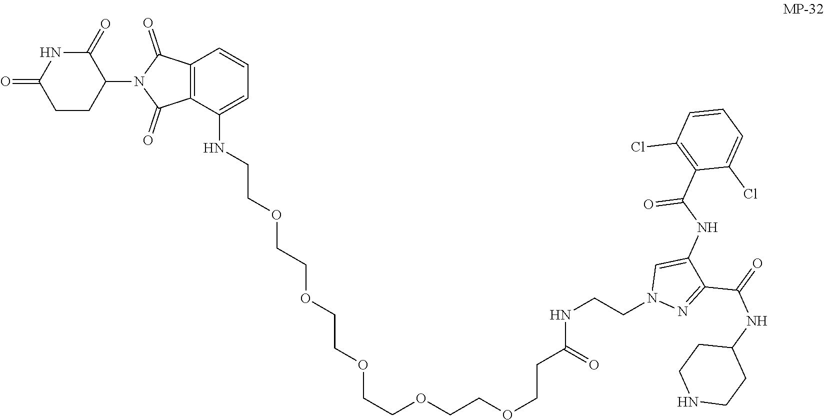 [SMILES: O=C(CCOCCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

In certain embodiments, the composition comprises MP-38:

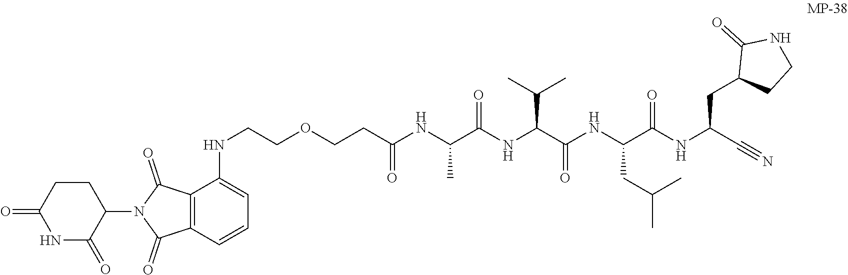 [SMILES: CC(C)C[C@H](NC(=O)[C@@H](NC(=O)[C@H](C)NC(=O)CCOCCNc1cccc2c1C(=O)N(C1CCC(=O)NC1=O)C2=O)C(C)C)C(=O)N[C@H](C#N)C[C@@H]1CCNC1=O]

In certain embodiments, the composition comprises MP-40:

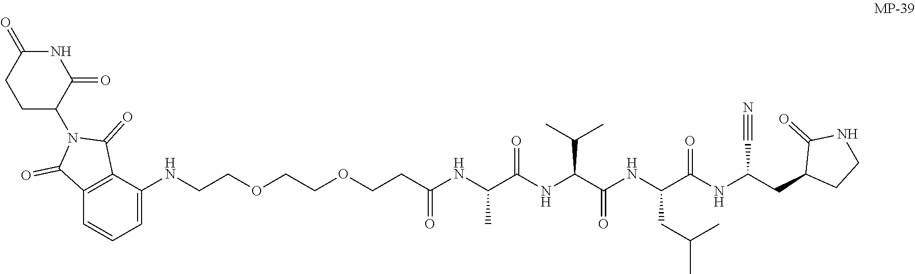 [SMILES: CC(C)C[C@H](NC(=O)[C@@H](NC(=O)[C@H](C)NC(=O)CCOCCOCCNc1cccc2c1C(=O)N(C1CCC(=O)NC1=O)C2=O)C(C)C)C(=O)N[C@H](C#N)C[C@@H]1CCNC1=O]

In certain embodiments, the composition comprises MP-41:

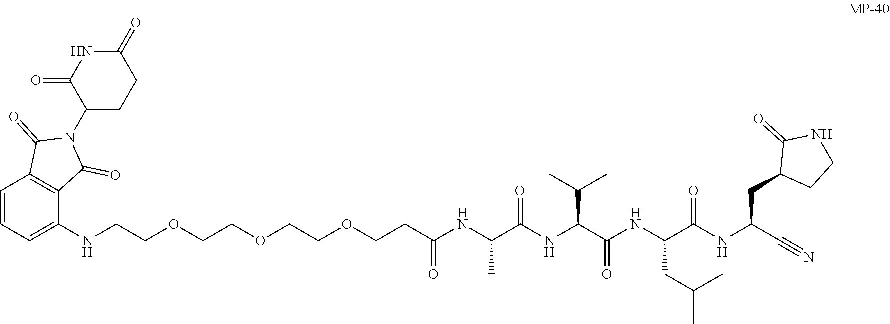 [SMILES: CC(C)C[C@H](NC(=O)[C@@H](NC(=O)[C@H](C)NC(=O)CCOCCOCCOCCNc1cccc2c1C(=O)N(C1CCC(=O)NC1=O)C2=O)C(C)C)C(=O)N[C@H](C#N)C[C@@H]1CCNC1=O]

In certain embodiments, the composition comprises MP-42:

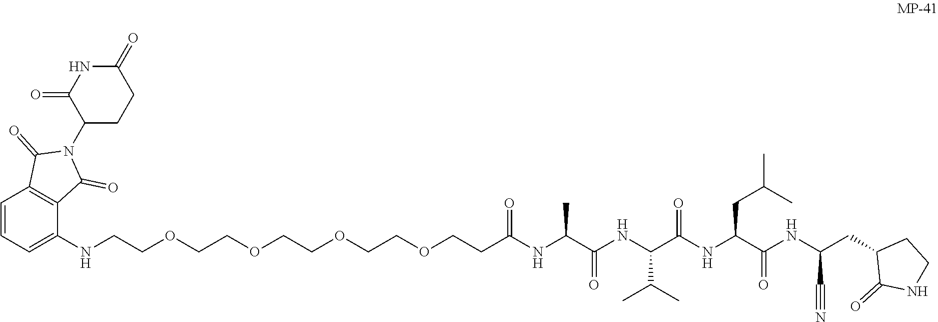 [SMILES: CC(C)C[C@H](NC(=O)[C@@H](NC(=O)[C@H](C)NC(=O)CCOCCOCCOCCOCCNc1cccc2c1C(=O)N(C1CCC(=O)NC1=O)C2=O)C(C)C)C(=O)N[C@H](C#N)C[C@@H]1CCNC1=O]

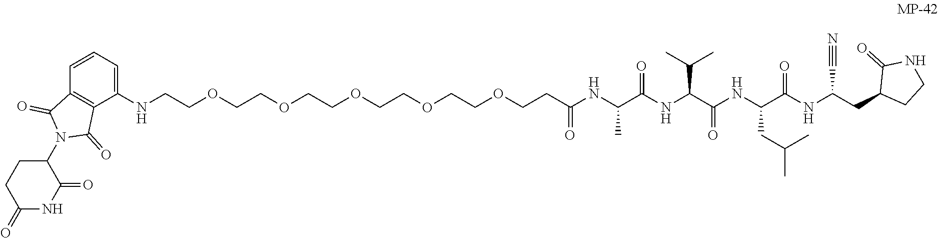 [SMILES: CC(C)C[C@H](NC(=O)[C@@H](NC(=O)[C@H](C)NC(=O)CCOCCOCCOCCOCCOCCNc1cccc2c1C(=O)N(C1CCC(=O)NC1=O)C2=O)C(C)C)C(=O)N[C@H](C#N)C[C@@H]1CCNC1=O]

In certain embodiments, the composition comprises MP-C-4N:

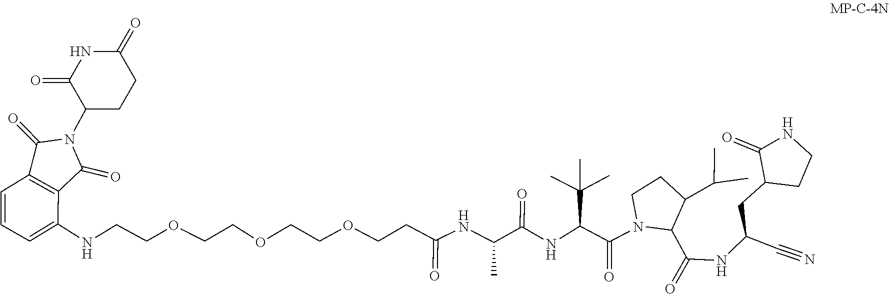 [SMILES: CC(C)C1CCN(C(=O)[C@@H](NC(=O)[C@H](C)NC(=O)CCOCCOCCOCCNc2cccc3c2C(=O)N(C2CCC(=O)NC2=O)C3=O)C(C)(C)C)C1C(=O)N[C@H](C#N)CC1CCNC1=O]

In certain embodiments, the composition further comprises a PLpro inhibitor attached to the linker.

In particular embodiments, the PLpro inhibitor comprises PL-1:

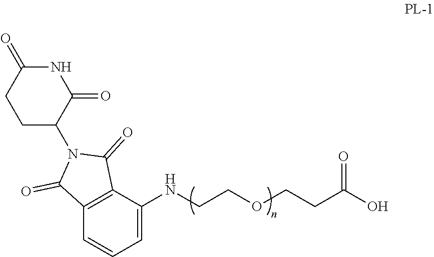

In particular embodiments the PLpro inhibitor comprises PL-2:

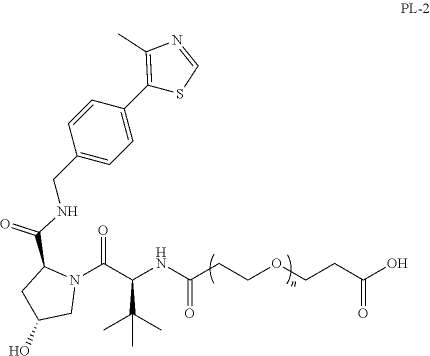

In particular embodiments, the PLpro inhibitor comprises PL-3:

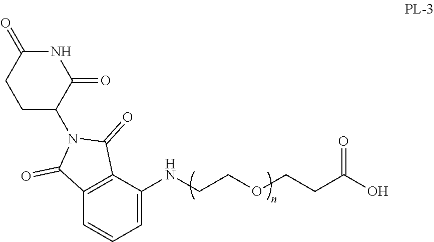

In particular embodiments the PLpro inhibitor comprises PL-4:

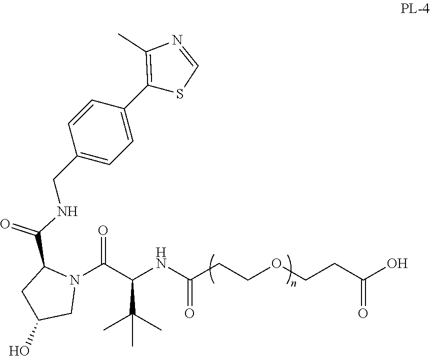

In particular embodiments the PLpro inhibitor comprises PL-5:

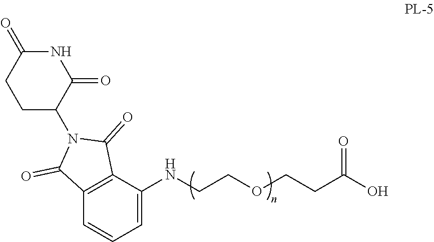

In particular embodiments, the PLpro inhibitor comprises PL-6:

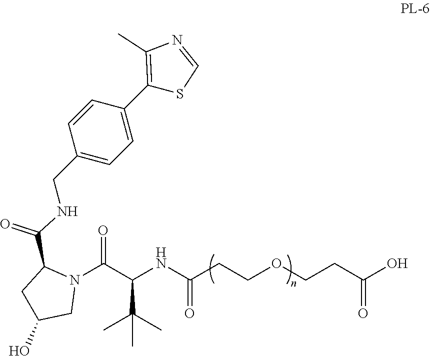

In particular embodiments, the composition comprises structure DT-1:

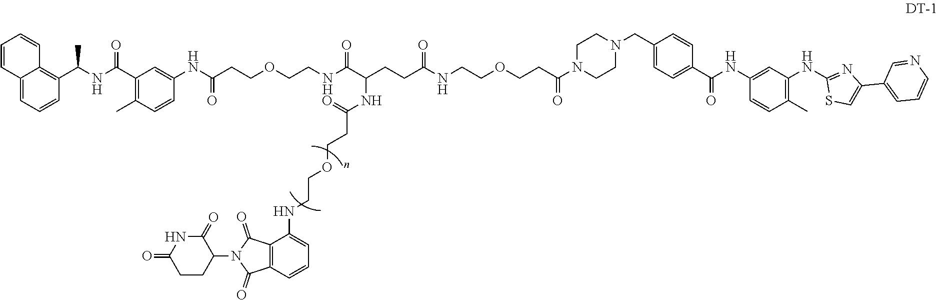

wherein n=1, 2, 3, or 4.

In particular embodiments the composition comprises structure DT-2:

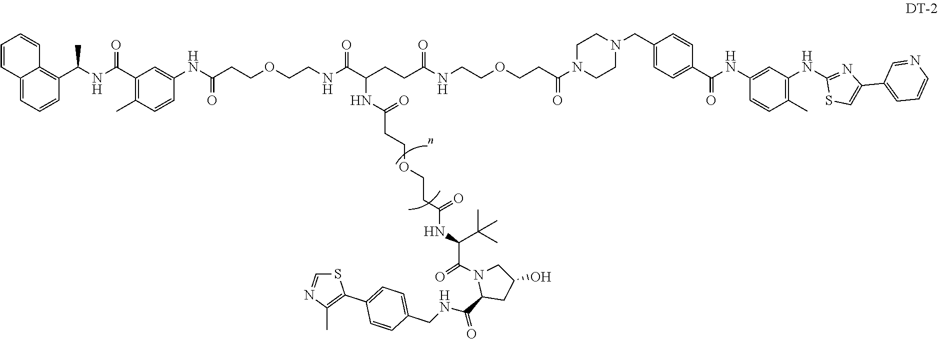

wherein n is 1, 2, 3, or 4.

In particular embodiments, the composition comprises structure DT-3:

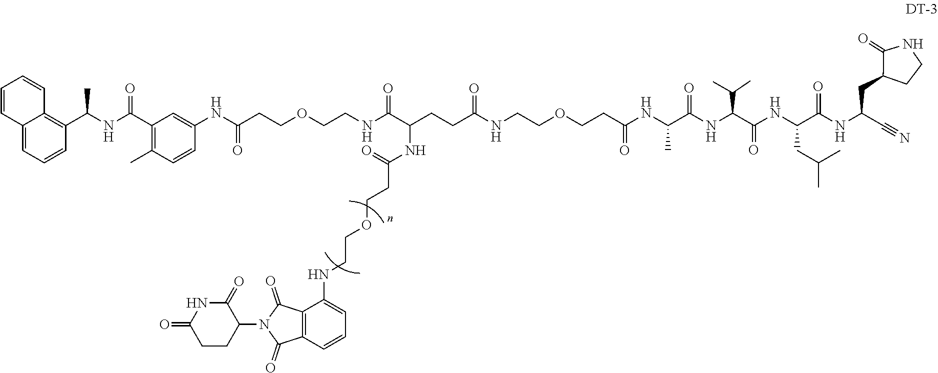

wherein n is 1, 2, 3, or 4.

In particular embodiments, the composition comprises structure DT-4:

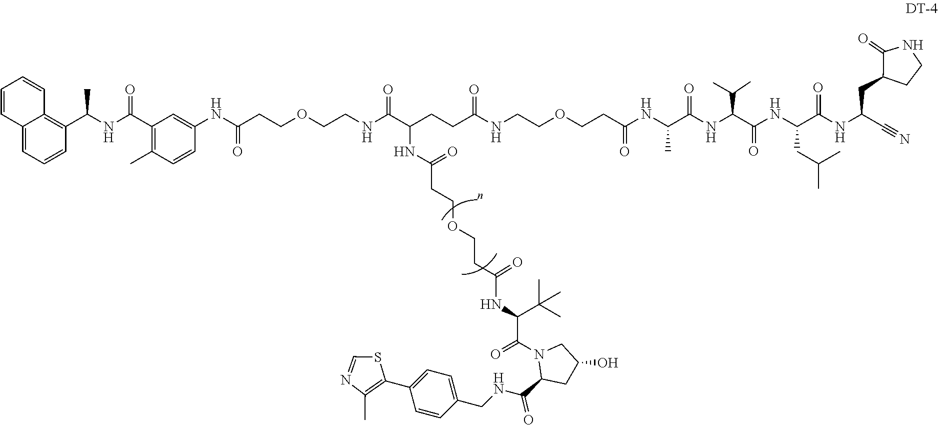

wherein n is 1, 2, 3, or 4.

In particular embodiments the composition comprises structure DT-5:

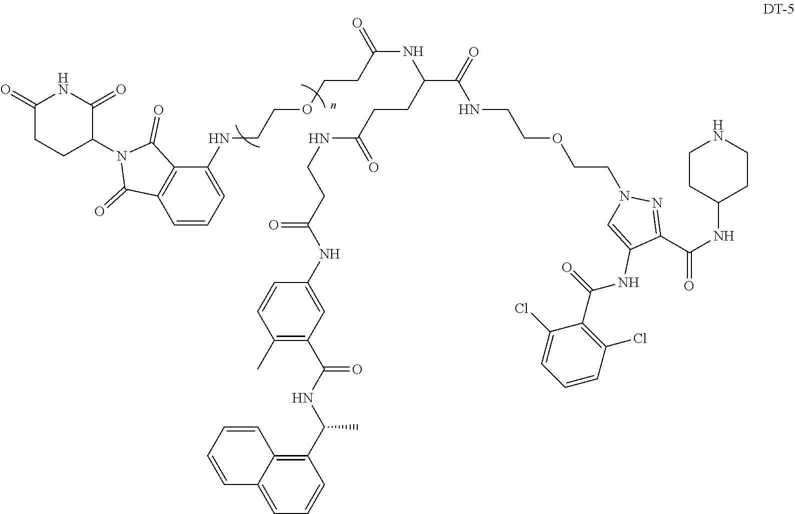

wherein n is 1, 2, 3, or 4.

In particular embodiments, the composition comprises structure DT-6:

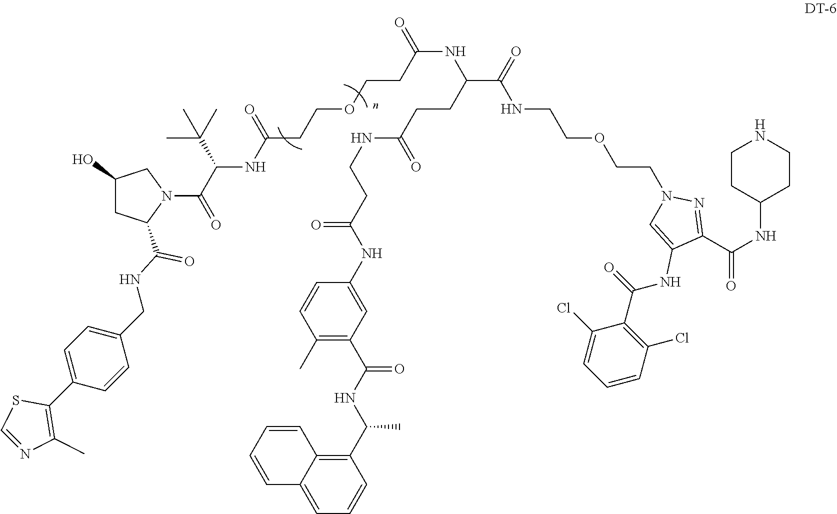

wherein n is 1,2,3, or 4.

Further provided is a method of treating a coronavirus infection, the method comprising administering to a subject having a coronavirus infection a PROTAC compound capable of binding to a Mpro protein of the coronavirus and causing degradation of the Mpro protein by E3 ligase to prevent viral replication and thereby treat the coronavirus infection. The PROTAC compound comprises a composition as disclosed herein. In certain embodiments, the coronavirus is SARS-CoV-2.

Further provided is a pharmaceutical composition comprising a PROTAC compound comprising a composition as disclosed herein, and a pharmaceutically acceptable carrier, adjuvant, or diluent.

Further provided is a method of degrading Mpro activity in a cell infected with a coronavirus, the method comprising contacting the cell with an effective amount of a composition comprising a PROTAC compound disclosed herein, and degrading Mpro activity in the cell. In certain embodiments, the coronavirus is SARS-CoV-2.

Further provided is a method of degrading PLpro activity in a cell infected with a coronavirus, the method comprising contacting the cell with an effective amount of a dual targeting PROTAC composition disclosed herein, and degrading PLpro activity in the cell. The dual targeting PROTAC composition comprises an E3 ligase ligand attached to a linker, a Mpro ligand attached to the linker, and a PLpro inhibitor attached to the linker. In certain embodiments, the coronavirus is SARS-CoV-2.

Further provided is a kit for making a PROTAC compound, the kit comprising a first container housing a Mpro ligand, and a second container housing a E3 ligase ligand. In certain embodiments, the kit further comprises a PLpro inhibitor. In certain embodiments, the kit further comprises a linker.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: Schematic representations of protein degradation mediated by PROTACs.

FIG. 2: Non-limiting example Mpro ligands.

FIG. 3: Non-limiting example Mpro ligands.

FIG. 4: Non-limiting example synthesis routes for Mpro ligands.

FIG. 5: General scheme for synthesizing a non-limiting example PROTAC of Class 1.

FIG. 6: General scheme for synthesizing a non-limiting example PROTAC of Class 2.

FIG. 7: General scheme for synthesizing a non-limiting example PROTAC of Class 3.

FIG. 8: Non-limiting example PROTACs of Class 1.

FIGS. 9A-9F: Schemes showing the synthesis of compounds MP-48 through MP-71.

FIGS. 10A-10D: Synthesis (FIG. 10A), ¹H NMR spectrum (FIG. 10B), ¹³C NMR spectrum (FIG. 10C), and HRMS spectrum (FIG. 10D) of PLP-TA-1 (GRL-0617), (R)-5-amino-2-methyl-N-(1-(naphthalen-1-yl)ethyl)benzamide).

FIG. 11: Scheme showing the synthesis of methyl 3-(((tert-butoxycarbonyl)amino)methyl)oxirane-2-carboxylate (10).

FIGS. 12A-12D: Synthesis (FIG. 12A), ¹H NMR spectrum (FIG. 12B), ¹³C NMR spectrum (FIG. 12C), and HRMS spectrum (FIG. 12D) of methyl (Z)-4-((tert-butoxycarbonyl)amino)but-2-enoate.

FIG. 13A-13D: Synthesis (FIG. 13A), ¹H NMR spectrum (FIG. 13B), ¹³C NMR spectrum (FIG. 13C), and HRMS spectrum (FIG. 13D) of methyl (Z)-4-((((S)-3-acetamido-2-((S)-2-amino-4-(4-hydroxyphenyl)butanamido)propanoyl)glycyl)oxy)but-2-enoate (PLP-TA-2).

FIG. 14A-14D: Synthesis (FIG. 14A), ¹H NMR spectrum (FIG. 14B), ¹³C NMR spectrum (FIG. 14C), and HRMS spectrum (FIG. 14D) of methyl (16S,19S,Z)-19-(acetamidomethyl)-1-((2-(2,6-dioxopiperidin-3-yl)-1,3-dioxoisoindolin-4-yl)amino)-16-(4-hydroxyphenethyl)-14,17,20,23-tetraoxo-3,6,9,12-tetraoxa-15,18,21,24-tetraazaoctacos-26-en-28-oate (PLP-5).

FIG. 15A-15D: Synthesis (FIG. 15A), ¹H NMR spectrum (FIG. 15B), ¹³C NMR spectrum (FIG. 15C), and HRMS spectrum (FIG. 15D) of methyl (3S,25S,28S,Z)-28-(acetamidomethyl)-3-((2S,4R)-4-hydroxy-2-((4-(4-methylthiazol-5-yl)benzyl)carbamoyl)pyrrolidine-1-carbonyl)-25-(4-hydroxyphenethyl)-2,2-dimethyl-5,23,26,29,32-pentaoxo-8,11,14,17,20-pentaoxa-4,24,27,30,33-pentaazaheptatriacont-35-en-37-oate (PLP-6).

FIG. 16: Activity of PROTACS-1 on PLpro expression.

FIG. 17: HRMS spectrum of Boc-AVL-OH.

FIG. 18A-18D: Synthesis (FIG. 18A), ¹H NMR spectrum (FIG. 18B), ¹³C NMR spectrum (FIG. 18C), and HRMS spectrum (FIG. 18D) of ethyl (S,E)-4-((tert-butoxycarbonyl)amino)-5-((S)-2-oxopyrrolidin-3-yl)pent-2-enoate (MP-M-1).

FIG. 19A-19D: Synthesis (FIG. 19A), ¹H NMR spectrum (FIG. 19B), ¹³C NMR spectrum (FIG. 19C), and HRMS spectrum (FIG. 19D) of benzyl (S,E)-4-((tert-butoxycarbonyl)amino)-5-((S)-2-oxopyrrolidin-3-yl)pent-2-enoate (MP-M-2).

FIG. 20A-20D: Synthesis (FIG. 20A), ¹H NMR spectrum (FIG. 20B), ¹³C NMR spectrum (FIG. 20C), and HRMS spectrum (FIG. 20D) of ethyl (S,E)-4-((S)-2-((S)-2-((S)-2-aminopropanamido)-3-methylbutanamido)-4-methylpentanamido)-5-((S)-2-oxopyrrolidin-3-yl)pent-2-enoate (MP-TA-1).

FIG. 21A-21D: Synthesis (FIG. 21A), ¹H NMR spectrum (FIG. 21B), ¹³C NMR spectrum (FIG. 21C), and HRMS spectrum (FIG. 21D) of benzyl (S,E)-4-((S)-2-((S)-2-((S)-2-aminopropanamido)-3-methylbutanamido)-4-methylpentanamido)-5-((S)-2-oxopyrrolidin-3-yl)pent-2-enoate (MP-TA-2).

FIG. 22A-22D: Synthesis (FIG. 22A), ¹H NMR spectrum (FIG. 22B), ¹³C NMR spectrum (FIG. 22C), and HRMS spectrum (FIG. 22D) of (2S)-2-((S)-2-((S)-2-aminopropanamido)-3-methylbutanamido)-N-(1-hydroxy-3-((S)-2-oxopyrrolidin-3-yl)propan-2-yl)-4-methylpentanamide (MP-TA-3).

FIG. 23A-23D: Synthesis (FIG. 23A), ¹H NMR spectrum (FIG. 23B), ¹³C NMR spectrum (FIG. 23C), and HRMS spectrum (FIG. 23D) of ethyl (S,E)-4-((S)-2-amino-4-methylpentanamido)-5-((S)-2-oxopyrrolidin-3-yl)pent-2-enoate (MP-TA-4).

FIG. 24A-24D: Synthesis (FIG. 24A), ¹H NMR spectrum (FIG. 24B), ¹³C NMR spectrum (FIG. 24C), and HRMS spectrum (FIG. 24D) of 7-(2-(2-((2-(2,6-dioxopiperidin-3-yl)-1,3-dioxo-2,3-dihydro-1H-inden-4-yl)amino)-2-oxoethoxy)ethoxy)-N-((S)-1-(((S)-1-(((S)-1-(((S)-1-hydroxy-3-((S)-2-oxopyrrolidin-3-yl)propan-2-yl)amino)-4-methyl-1-oxopentan-2-yl)amino)-3-methyl-1-oxobutan-2-yl)amino)-1-oxopropan-2-yl)heptanamide (MP-5).

FIG. 25A-25D: Synthesis (FIG. 25A), ¹H NMR spectrum (FIG. 25B), ¹³C NMR spectrum (FIG. 25C), and HRMS spectrum (FIG. 25D) of (2S,4R)-1-((2S,5S,8S,11S,27S)-27-(tert-butyl)-1-hydroxy-5-isobutyl-8-isopropyl-11-methyl-4,7,10,13,25-pentaoxo-2-(((S)-2-oxopyrrolidin-3-yl)methyl)-20,23-dioxa-3,6,9,12,26-pentaazaoctacosan-28-oyl)-4-hydroxy-N-(4-(4-methylthiazol-5-yl)benzyl)pyrrolidine-2-carboxamide (MP-6).

FIG. 26A-26D: Synthesis (FIG. 26A), ¹H NMR spectrum (FIG. 26B), ¹³C NMR spectrum (FIG. 26C), and HRMS spectrum (FIG. 26D) of ethyl (15S,18S,21S,24S,E)-1-((2-(2,6-dioxopiperidin-3-yl)-1,3-dioxo-2,3-dihydro-1H-inden-4-yl)amino)-21-isobutyl-18-isopropyl-15-methyl-1,13,16,19,22-pentaoxo-24-(((S)-2-oxopyrrolidin-3-yl)methyl)-3,6-dioxa-14,17,20,23-tetraazaheptacos-25-en-27-oate (MP-7).

FIG. 27A-27D: Synthesis (FIG. 27A), ¹H NMR spectrum (FIG. 27B), ¹³C NMR spectrum (FIG. 27C), and HRMS spectrum (FIG. 27D) of ethyl (3S,25S,28S,31S,34S,E)-3-((2S,4R)-4-hydroxy-2-((4-(4-methylthiazol-5-yl)benzyl)carbamoyl)pyrrolidine-1-carbonyl)-31-isobutyl-28-isopropyl-2,2,25-trimethyl-5,23,26,29,32-pentaoxo-34-(((S)-2-oxopyrrolidin-3-yl)methyl)-8,11,14,17,20-pentaoxa-4,24,27,30,33-pentaazaheptatriacont-35-en-37-oate (MP-8).

FIG. 28A-28D: Synthesis (FIG. 28A), ¹H NMR spectrum (FIG. 28B), ¹³C NMR spectrum (FIG. 28C), and HRMS spectrum (FIG. 28D) of ethyl (3S,6S,9S,12S,E)-9-isobutyl-6-isopropyl-3-methyl-1,4,7,10-tetraoxo-12-(((S)-2-oxopyrrolidin-3-yl)methyl)-1-(3-(pyridin-2-yl)phenyl)-2,5,8,11-tetraazapentadec-13-en-15-oate (MP-9).

FIG. 29A-29D: Synthesis (FIG. 29A), ¹H NMR spectrum (FIG. 29B), ¹³C NMR spectrum (FIG. 29C), and HRMS spectrum (FIG. 29D) of ethyl (3S,6S,9S,E)-9-isobutyl-6-isopropyl-3-methyl-1,4,7,10-tetraoxo-12-(((S)-2-oxopyrrolidin-3-yl)methyl)-1-(4-(pyridin-2-yl)phenyl)-2,5,8,11-tetraazapentadec-13-en-15-oate (MP-10).

FIG. 30A-30D: Synthesis (FIG. 30A), ¹H NMR spectrum (FIG. 30B), ¹³C NMR spectrum (FIG. 30C), and HRMS spectrum (FIG. 30D) of ethyl (16S,19S,E)-1-((2-(2,6-dioxopiperidin-3-yl)-1,3-dioxoisoindolin-4-yl)amino)-16-isobutyl-14,17-dioxo-19-(((S)-2-oxopyrolidin-3-yl)methyl)-3,6,9,12-tetraoxa-15,18-diazadocos-20-en-22-oate (MP-11).

FIG. 31A-31D: Synthesis (FIG. 31A), ¹H NMR spectrum (FIG. 31B), ¹³C NMR spectrum (FIG. 31C), and HRMS spectrum (FIG. 31D) of ethyl (3S,25S,28S,E)-3-((2S,4R)-4-hydroxy-2-((4-(4-methylthiazol-5-yl)benzyl)carbamoyl)pyrrolidine-1-carbonyl)-25-isobutyl-2,2-dimethyl-5,23,26-trioxo-28-(((S)-2-oxopyrrolidin-3-yl)methyl)-8,11,14,17,20-pentaoxa-4,24,27-triazahentriacont-29-en-31-oate (MP-12).

FIG. 32A-32D: Synthesis (FIG. 32A), ¹H NMR spectrum (FIG. 32B), ¹³C NMR spectrum (FIG. 32C), and HRMS spectrum (FIG. 32D) of benzyl (16S,19S,22S,25S,E)-1-((2-(2,6-dioxopiperidin-3-yl)-1,3-dioxoisoindolin-4-yl)amino)-22-isobutyl-19-isopropyl-16-methyl-14,17,20,23-tetraoxo-25-(((S)-2-oxopyrrolidin-3-yl)methyl)-3,6,9,12-tetraoxa-15,18,21,24-tetraazaoctacos-26-en-28-oate (MP-13).

FIG. 33A-33D: Synthesis (FIG. 33A), ¹H NMR spectrum (FIG. 33B), ¹³C NMR spectrum (FIG. 33C), and HRMS spectrum (FIG. 33D) of benzyl (3S,25S,28S,31S,34S,E)-3-((2S,4R)-4-hydroxy-2-((4-(4-methylthiazol-5-yl)benzyl)carbamoyl)pyrrolidine-1-carbonyl)-31-isobutyl-28-isopropyl-2,2,25-trimethyl-5,23,26,29,32-pentaoxo-34-(((S)-2-oxopyrrolidin-3-yl)methyl)-8,11,14,17,20-pentaoxa-4,24,27,30,33-pentaazaheptatriacont-35-en-37-oate (MP-14).

FIG. 34A-34D: Synthesis (FIG. 34A), ¹H NMR spectrum (FIG. 34B), ¹³C NMR spectrum (FIG. 34C), and HRMS spectrum (FIG. 34D) of ethyl (16S,19S,22S,25S,E)-1-((2-(2,6-dioxopiperidin-3-yl)-1,3-dioxoisoindolin-4-yl)amino)-22-isobutyl-19-isopropyl-16-methyl-14,17,20,23-tetraoxo-25-(((S)-2-oxopyirolidin-3-yl)methyl)-3,6,9,12-tetraoxa-15,18,21,24-tetraazaoctacos-26-en-28-oate (MP-15).

FIG. 35A-35D: Synthesis (FIG. 35A), ¹H NMR spectrum (FIG. 35B), ¹³C NMR spectrum (FIG. 35C), and HRMS spectrum (FIG. 35D) of benzyl (3S,6S,9S,12S,E)-9-isobutyl-6-isopropyl-3-methyl-1,4,7,10-tetraoxo-12-(((S)-2-oxopyrrolidin-3-yl)methyl)-1-(3-(pyridin-2-yl)phenyl)-2,5,8,11-tetraazapentadec-13-en-15-oate (MP-16).

FIG. 36: Scheme showing the synthesis of N-(4-methyl-3-((4-(pyridin-3-yl)thiazol-2-yl)amino)phenyl)-4-(piperazin-1-ylmethyl)benzamide (MP-TA-5).

FIG. 37: Scheme showing the synthesis of synthesis of 4-((4-(3-(2-((2-(2,6-dioxopiperidin-3-yl)-1,3-dioxoisoindolin-4-yl)amino)ethoxy)propanoyl)piperazin-1-yl)methyl)-N-(4-methyl-3-((4-(pyridin-3-yl)thiazol-2-yl)amino)phenyl)benzanide (MP-18).

FIG. 38: Scheme showing the synthesis of benzyl 4-(4-(2,6-dichlorobenzaimido)-1H-pyrazole-3-carboxamido)piperidine-1-carboxylate (TA-3).

FIG. 39: Scheme showing the synthesis of benzyl 4-(1-(2-aminoethyl)-4-(2,6-dichlorobenzamido)-1H-pyrazole-3-carboxamido)piperidine-1-carboxylate (TA-6).

FIG. 40: Scheme showing the synthesis of 4-(2,6-dichlorobenzamido)-1-(2-(3-(2-((2-(2,6-dioxopiperidin-3-yl)-1,3-dioxoisoindolin-4-yl)amino)ethoxy)propanamido)ethyl)-N-(piperidin-4-yl)-1H-pyrazole-3-carboxamide (MP-28).

FIG. 41: Scheme showing the synthesis of MP-TA-6.

FIG. 42: Scheme showing the synthesis of MP-38.

FIGS. 43A-43C: Synthesis (FIG. 43A), ¹H NMR spectrum (FIG. 43B), and HRMS spectrum (FIG. 43C) of (S)-5-amino-2-methyl-N-(1-(naphthalen-1-yl)ethyl)benzamide.

FIGS. 44A-44D: Synthesis (FIG. 44A), ¹H NMR spectrum (FIG. 44B), ¹³C NMR spectrum (FIG. 44C), and HRMS spectrum (FIG. 44D) of 5-(4-((2-(2,6-dioxopiperidin-3-yl)-1,3-dioxoisoindolin-4-yl)amino)butanamido)-2-methyl-N-((S)-1-(naphthalen-1-yl)ethyl)benzamide (PLP-1).

FIGS. 45A-45D: Synthesis (FIG. 45A), ¹H NMR spectrum (FIG. 45B), ¹³C NMR spectrum (FIG. 45C), and HRMS spectrum (FIG. 45B) of 5-(4-((2-(2,6-dioxopiperidin-3-yl)-1,3-dioxoisoindolin-4-yl)amino)butanamido)-2-methyl-N-((R)-1-(naphthalen-1-yl)ethyl)benzamide (PLP-2).

FIGS. 46A-46D: Synthesis (FIG. 46A), ¹H NMR spectrum (FIG. 46B), ¹³C NMR spectrum (FIG. 46C), and HRMS spectrum (FIG. 46D) of 14-((2-(2,6-dioxopiperidin-3-yl)-1,3-dioxoisoindolin-4-yl)amino)-N-(4-methyl-3-(((R)-1-(naphthalen-1-yl)ethyl)carbamoyl)phenyl)-3,6,9,12-tetraoxatetradecanamide (PLP-3).

FIGS. 47A-47D: Synthesis (FIG. 47A), ¹H NMR spectrum (FIG. 47B), ¹³C NMR spectrum (FIG. 47C), and HRMS spectrum (FIG. 47D) of N¹-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N¹⁹-(4-methyl-3-(((R)-1-(naphthalen-1-yl)ethyl)carbamoyl)phenyl)-4,7,10,13,16-pentaoxanonadecanediamide (PLP-4).

FIG. 48: HRMS spectrum of Boc-HoTyr-(Ac)Dap-G-OH.

FIG. 49A-49D: Synthesis (FIG. 49A), ¹H NMR spectrum (FIG. 49B), ¹³C NMR spectrum (FIG. 49C), and HRMS spectrum (FIG. 49D) of methyl (3S,6S,Z)-6-(acetamidomethyl)-3-(4-hydroxyphenethyl)-1,4,7,10-tetraoxo-1-(3-(pyridin-2-yl)phenyl)-2,5,8,11-tetraazapentadec-13-en-15-oate (PLP-3PY).

FIG. 50A-50D: Synthesis (FIG. 50A), ¹H NMR spectrum (FIG. 50B), ¹³C NMR spectrum (FIG. 50C), and HRMS spectrum (FIG. 50D) of methyl (3S,6S,Z)-6-(acetamidomethyl)-3-(4-hydroxyphenethyl)-1,4,7,10-tetraoxo-1-(4-(pyridin-2-yl)phenyl)-2,5,8,11-tetraazapentadec-13-en-15-oate.

FIGS. 51A-51B: Illustration of non-limiting example dual targeting PROTAC compound referred to as DTP (FIG. 51A), and a scheme showing the synthesis of dual targeting PROTAC compounds (FIG. 51B).

FIG. 52: Scheme showing the synthesis of MP-TA-7.

FIG. 53: Scheme showing the synthesis of MP-TA-9.

FIG. 54A-54D: Synthesis (FIG. 54A), ¹H NMR spectrum (FIG. 54B), ¹³C NMR spectrum (FIG. 54C), and HRMS spectrum (FIG. 54D) of benzyl (3S,6S,9S,12S,E)-9-isobutyl-6-isopropyl-3-methyl-1,4,7,10-tetraoxo-12-(((S)-2-oxopyrrolidin-3-yl)methyl)-1-(4-(pyridin-2-yl)phenyl)-2,5,8,11-tetraazapentadec-13-en-15-oate (MP-17).

FIG. 55: Mpro degradation by the PROTAC compound MP-C-4N.

FIGS. 56A-56B: Results of testing the dual targeting PROTAC compound DTP. FIG. 56A shows a concentration-dependent inhibition of the dual targeting PROTAC compound on PLpro. FIG. 56B shows the dose-dependent effects of the dual targeting PROTAC compound on Mpro-T7 level.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

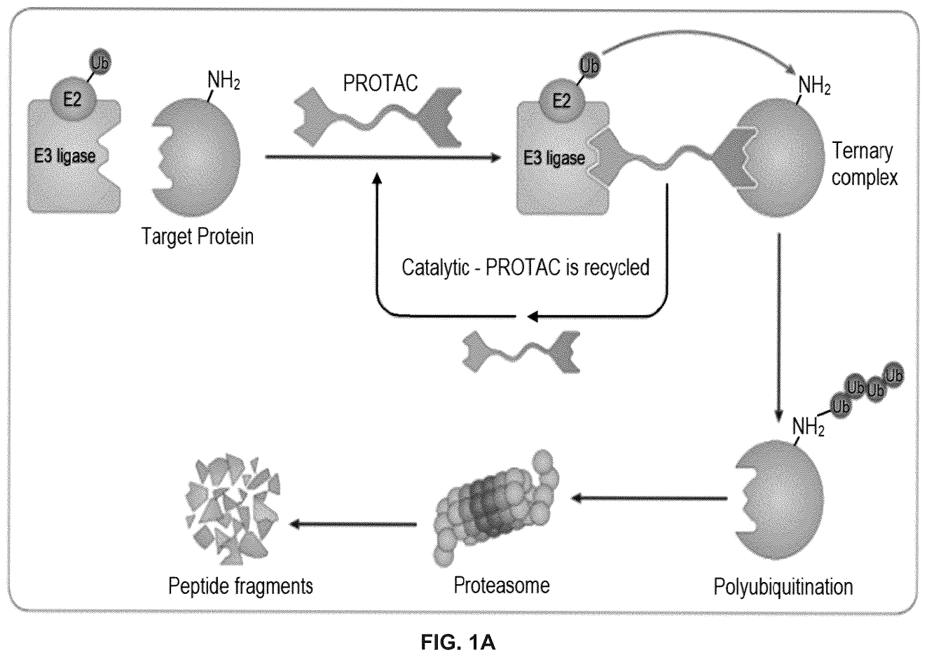

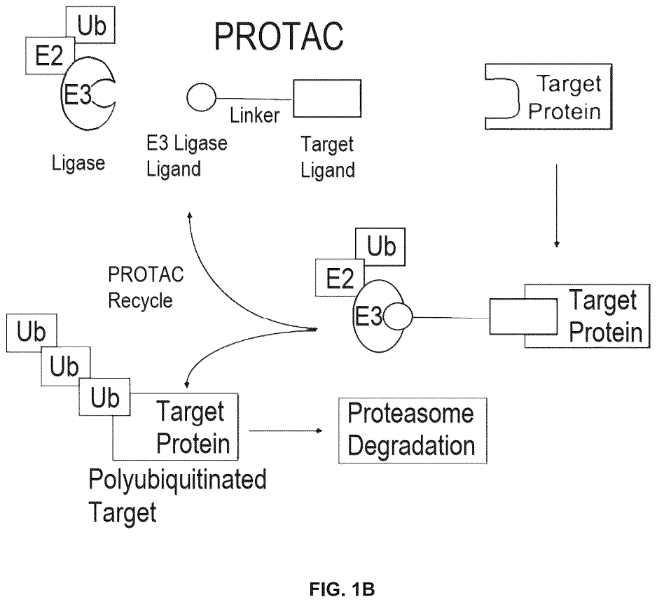

PROteolysis TArgeting Chimeras (PROTACs) (FIGS. 1A-1B) are useful in treating various diseases, yet have not been used for treating infectious diseases such as COVID-19. In accordance with the present disclosure, PROTACs that selectively degrade Mpro, a protease responsible for SARS-CoV-2 virus replication and inhibition of host immune responses, have been developed, and can be used as safe and stable therapeutics for treating COVID-19 and its various mutants. The PROTACs can be utilized as therapeutics for treating COVID-19 wild and mutated strains with the advantages of being non-toxic and low dosing, as well as it not being easy to develop drug resistance to the PROTACs.

One of the best-characterized drug targets among coronaviruses is the main protease (Mpro, also called 3-chymotrypsin-like Cys protease, or 3CLpro). Mpro is essential for the cleavage of the two critical replicase peptides in coronavirus proliferation. Mpro is a deubiquitinating protease that inhibits the NF-ηB pathway and thus immune responses to viral infection. Mpro functions as a cysteine protease, engaging in the proteolytic cleavage of the viral precursor polyprotein to a series of functional proteins required for coronavirus replication, and is a useful target for designing anti-SARS agents. After invading cells, the virus synthesizes two ultra-long replicase enzyme polypeptides (pp1a and pp1ab), which function in its replication. These two replicase polypeptides are cleaved into multiple parts (such as RNA-dependent RNA polymerase, helicase, etc.), which initiates replication of a large number of copies of its genetic material. The cleavage of two replicase peptides entails exceptional precision, and this important work is done by the main protease Mpro. Therefore, the inhibition of this enzyme prevents virus replication, making this enzyme an important target for the development of anti-coronavirus drugs. Furthermore, because no human proteases with a similar cleavage specificity are known, such inhibitors are unlikely to be toxic.

The crystal structure of Mpro has been resolved, making it possible to design small molecule compounds which specifically bind to the Mpro active site. However, traditional small molecule drugs function as inhibitors, and those highly-specific active site binding inhibitors can be slow-acting and prone to losing efficacy due to viral mutation, Traditional small molecule inhibitors exert their inhibition efficacy by binding to the target protein's active site or allosteric site, preventing the protein from binding to its specific substrate or reducing its catalytic activity. This entails a high affinity between the drug and the target protein and a long half-life to ensure that the inhibitor has a long enough time to interact with the target protein. In contrast, PROTACs can achieve degradation of the enzyme with moderate binding force to any site of the target protein. Also, direct degradation of the target protein has been shown to prevent muitations, avoiding the development of drug resistance. Furthermore, PROTACs act like catalysts that are released after initiating ubiquitination of a target protein and will continue to label the next target protein. Given this catalytic property, PROTACs can degrade target protein at low doses, which is less prone to cause side effects.

Accordingly, in accordance with the present disclosure, PROTACs technology can be used to build bifunctional ligands containing a Mpro targeting compound connected by a chemical linker to an E3 ubiquitin ligase recruiting ligand. These molecules facilitate bringing the target Mpro protein close to an E3 ligase of interest, causing consequent degradation of the Mpro. Direct degradation of the target protein prevents the virus from continuing mutation and evolution, thus reducing the development of drug resistance. In addition, these Mpro-targeting PROTACs are released after degrading Mpro and recruited to the next target. Therefore, the PROTACs can degrade the target protein at low doses.

PROTACs have been previously explored to fight various diseases, including cancer, immune disorders, neuronal disease, and others. PROTACs generally feature a bifunctional molecule that binds to the target protein at one end and recruits E3 ligase at the other end. By recruiting target protein and E3 ligase at the same time with close proximity, the targeted protein is labeled with ubiquitin through E3 ligase and subjected for proteolysis.

Provided herein are PROTACs which include an E3 ligase ligand attached to a linker, and a Mpro ligand attached to the linker. The PROTACs specifically target and degrade the SARS-CoV-2 main protease, Mpro. Based on the known crystal structure of Mpro, small molecules targeting Mpro were screened and designed, and were linked to an E3 ligase ligand through a linker to generate a series of Mpro targeting PROTACs that were tailored for ideal activity. As shown in the examples herein, several of the PROTACs sufficiently degrade Mpro in human cells and prevent viral replication. The advantages of the designed PROTACs include (1) directly binding to and degrading Mpro; (2) not needing to bind to the activity site of Mpro, allowing for molecules with high affinity and selectivity to be used; and (3) having a catalytic effect on the degradation process, allowing for relatively low doses to be effective and show relatively no toxicity.

The PROTACs include an E3 ligase ligand attached to a linker, and a Mpro ligand attached to the linker. In some embodiments, the PROTACs specifically target and degrade Mpro from SARS-CoV-2.

The E3 ligase ligand can be a known moiety useful as a ligand for E3 ligase. Non-limiting examples of E3 ligase ligands include N-alkylated pomalidomide, an acetylated pomalidomide, 4-hydroxythalidomide, alkyl-connected thalidomide derivatives, lenalidomide, 5-aminothalidomide derivatives, AHPC-PEGₙ-butyl COOH (where n=2-10), pomalidomide-PEGₙ-COOH (where n=2-10), pomalidomide-Cₙ-COOH (where n=3-10), other derivatives of pomalidomide, thalidomide, methylbestatin, LCL161 derivative, the von Hippel-Lindau (VHL) ligand, nutilin-3, alkyl-connected thalidomide derivatives, or derivatives of 5-aminothalidomide.

In some example embodiments, the E3 ligase ligand is pomalidomide, which has the following structure:

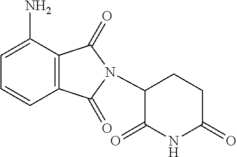 [SMILES: Nc1cccc2c1C(=O)N(C1CCC(=O)NC1=O)C2=O]

In other example embodiments, the E3 ligase ligand is the VHL ligand, which has the following structure:

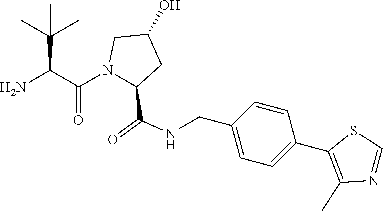 [SMILES: Cc1ncsc1-c1ccc(CNC(=O)[C@@H]2C[C@@H](O)CN2C(=O)[C@@H](N)C(C)(C)C)cc1]

However, many other E3 ligase ligands are possible and encompassed within the scope of the present disclosure.

The linker can be a moiety that serves to connect the E3 ligase ligand to the Mpro ligand. Non-limiting examples of suitable linkers include alkyl groups, alkoxy groups, ethers, PEG chains or segments, extended glycol chains, alkyl groups containing a PEG segment, alkyelene groups (such as straight chain alkyelene groups of from about 4 to 16 carbons), heterocyclic groups, primary amines, alkynes, triazoles, saturated heterocycles such as piperazine and piperidine, or combinations thereof. In some embodiments, the linker includes 3-4 PEG units. When the linker contains PEG, the linker may also include modifications of the individual glycol units, incorporating additional methylene moieties to access different chain lengths.

The linker can be attached to the E3 ligase ligand through a suitable process, such as by utilizing various acyl chloride-bearing linkers in THF under reflux with pomalidomide for a sufficient amount of time, or reacting pomalidomide with bromoacetyl chloride. As another example, the linker attachment may be through amide bond formation. The manner of connecting the E3 ligase ligand to the linker is not limited.

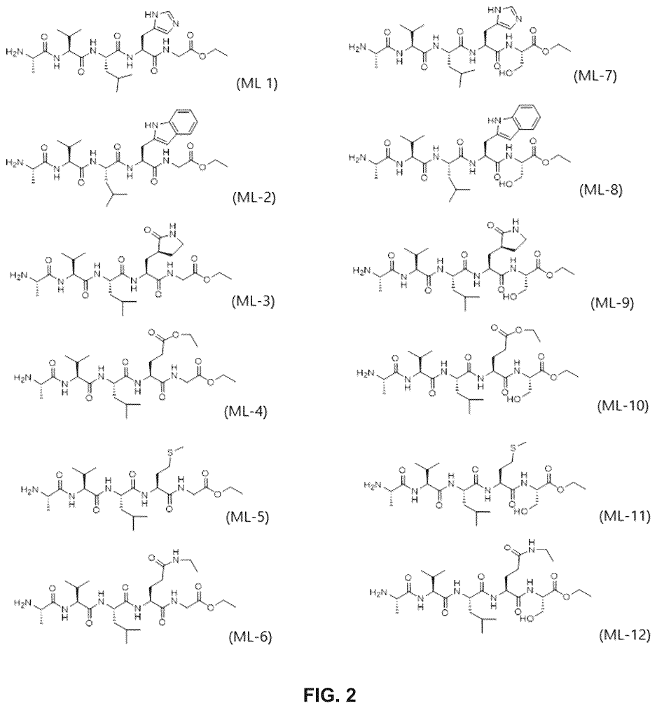

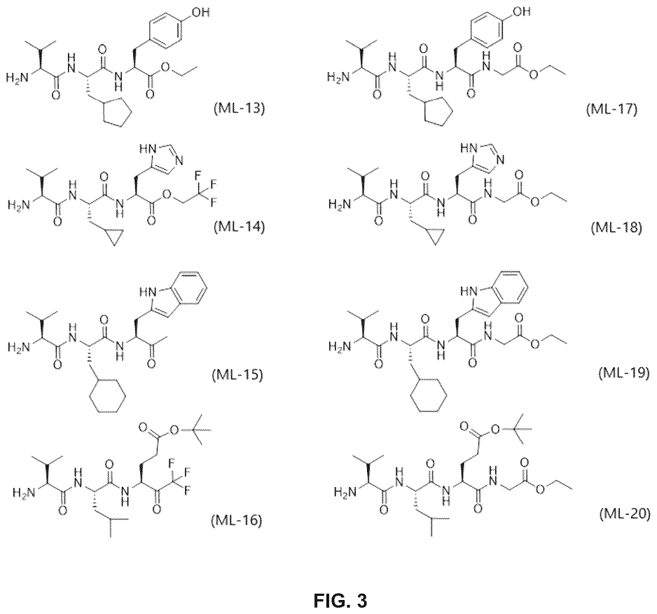

The Mpro ligand can be a moiety capable of binding to the Mpro of a coronavirus, such as the Mpro of SARS-CoV-2. Non-limiting examples of Mpro ligands include the structures shown in FIGS. 2-3, referred to as ML-1, ML-2, ML-3, ML-4, ML-4, ML-5, ML-6, ML-7, ML-8, ML-9, ML-10, ML-11, ML-12, ML-13, ML-14, ML-15, ML-16, ML-17, ML-18, ML-19, and ML-20. A further non-limiting example of an Mpro ligand is the compound referred to herein as PLP-TA-1, which has the following structure:

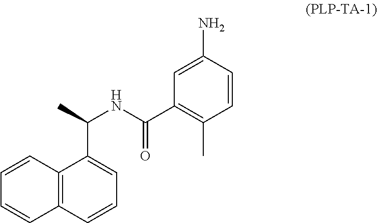 [SMILES: Cc1ccc(N)cc1C(=O)N[C@H](C)c1cccc2ccccc12]

However, many other Mpro ligands are possible and encompassed within the scope of the present disclosure.

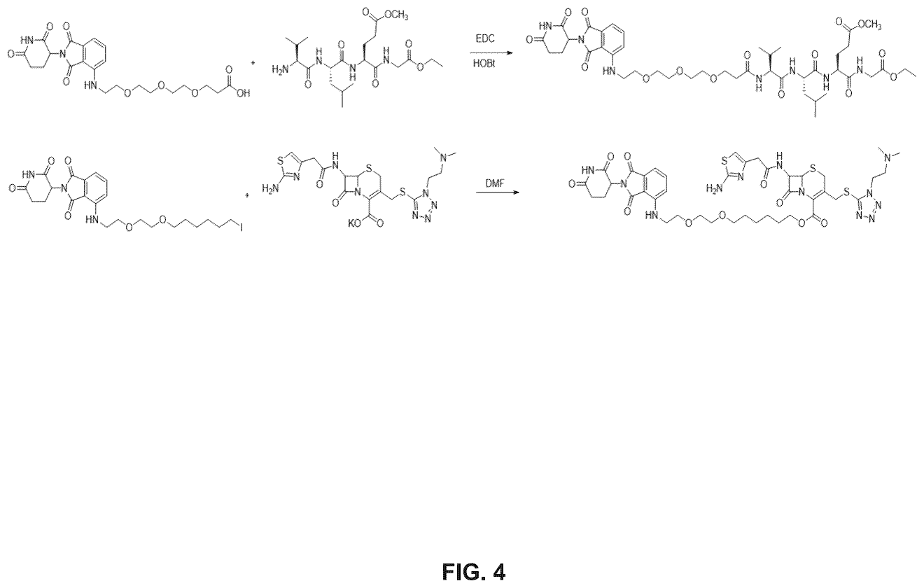

FIG. 4 depicts two non-limiting example synthetic routes for the preparation for PROTAC compounds, where a a compound containing the E3 ligand bound to the linker is reacted with the Mpro ligand to obtain the PROTAC compound. Many other synthetic routes are possible, as described in the examples herein, and encompassed within the scope of the present disclosure.

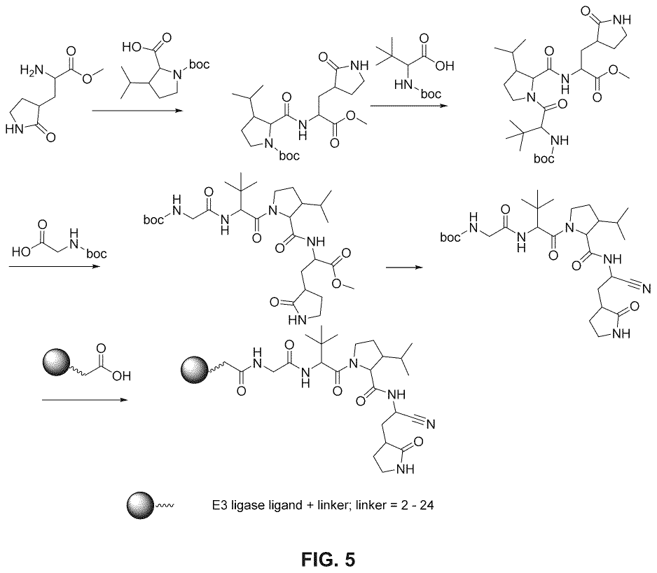

A first class of PROTACs, referred to herein as Class 1, may be synthesized according to the general scheme depicted in FIG. 5. The Class 1 PROTACs have the following general structural formula I:

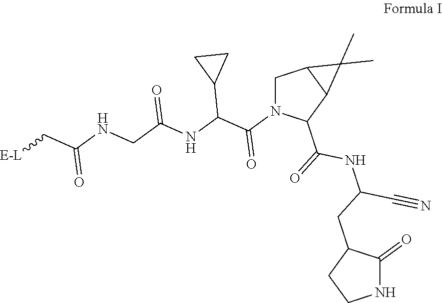

where E is the E3 ligase ligand, and L is the linker.

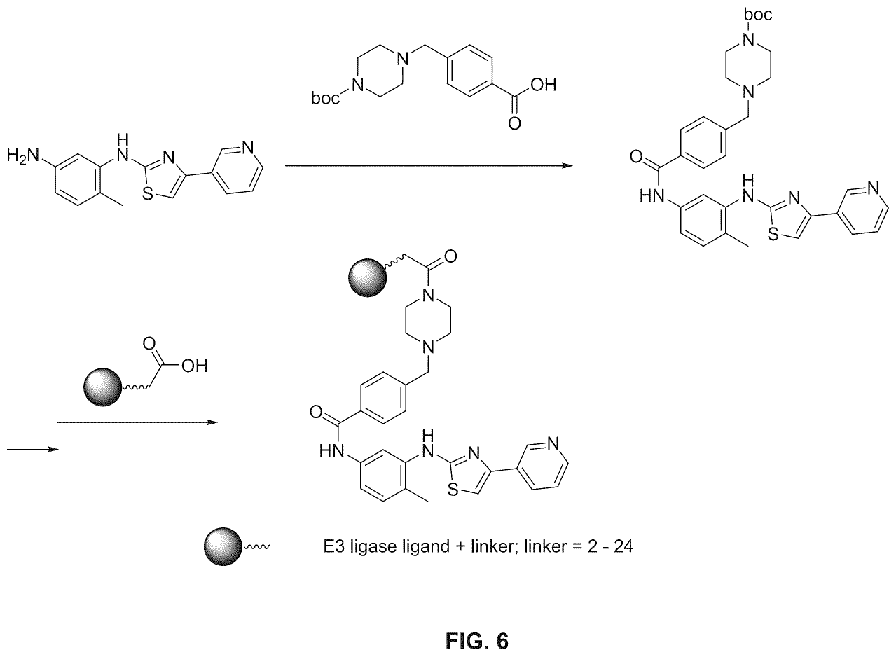

A second class of PROTACs, referred to herein as Class 2, may be synthesized according to the general scheme depicted in FIG. 6. The Class 2 PROTACs have the following general structural formula II:

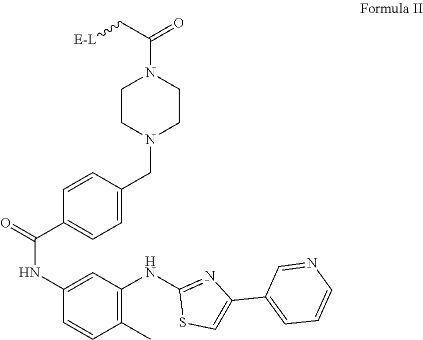

where E is the E3 ligase ligand, and L is the linker.

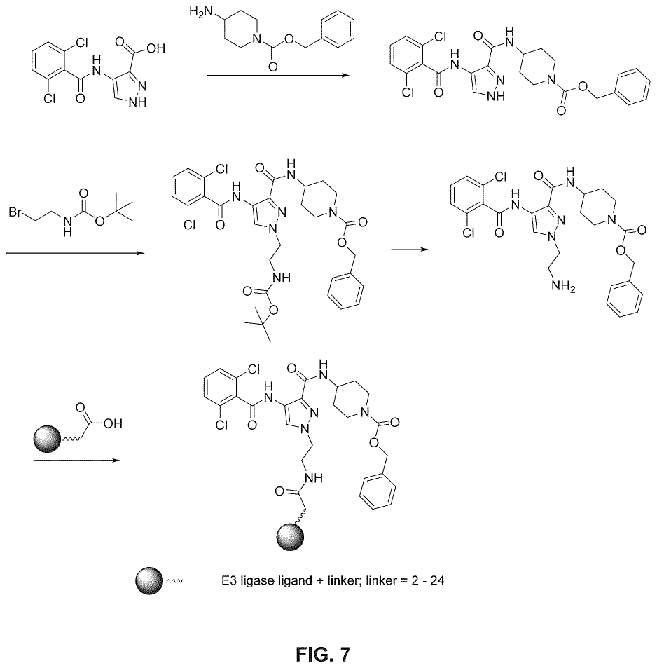

A third class of PROTACs, referred to herein as Class 3, may be synthesized according to the general scheme depicted in FIG. 7. The Class 3 PROTACs have the following general structural formula III:

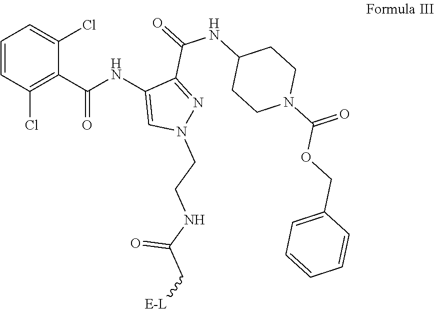

where E is the E3 ligase ligand, and L is the linker.

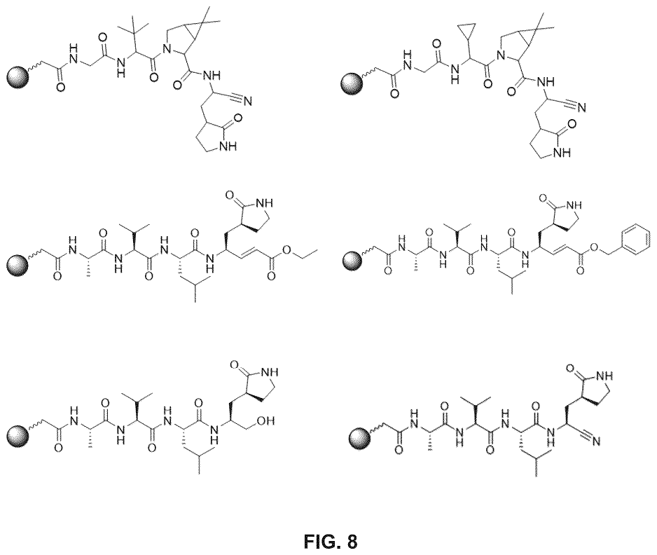

Some additional non-limiting classes of PROTACs are depicted in FIG. 8.

Some non-limiting examples of the PROTAC compounds include PROTACS-1, MP-18, MP-19, MP-20, MP-21, MP-22, MP-23, MP-24, MP-25, MP-25, MP-26, and MP-27, as shown below and described in the examples herein.

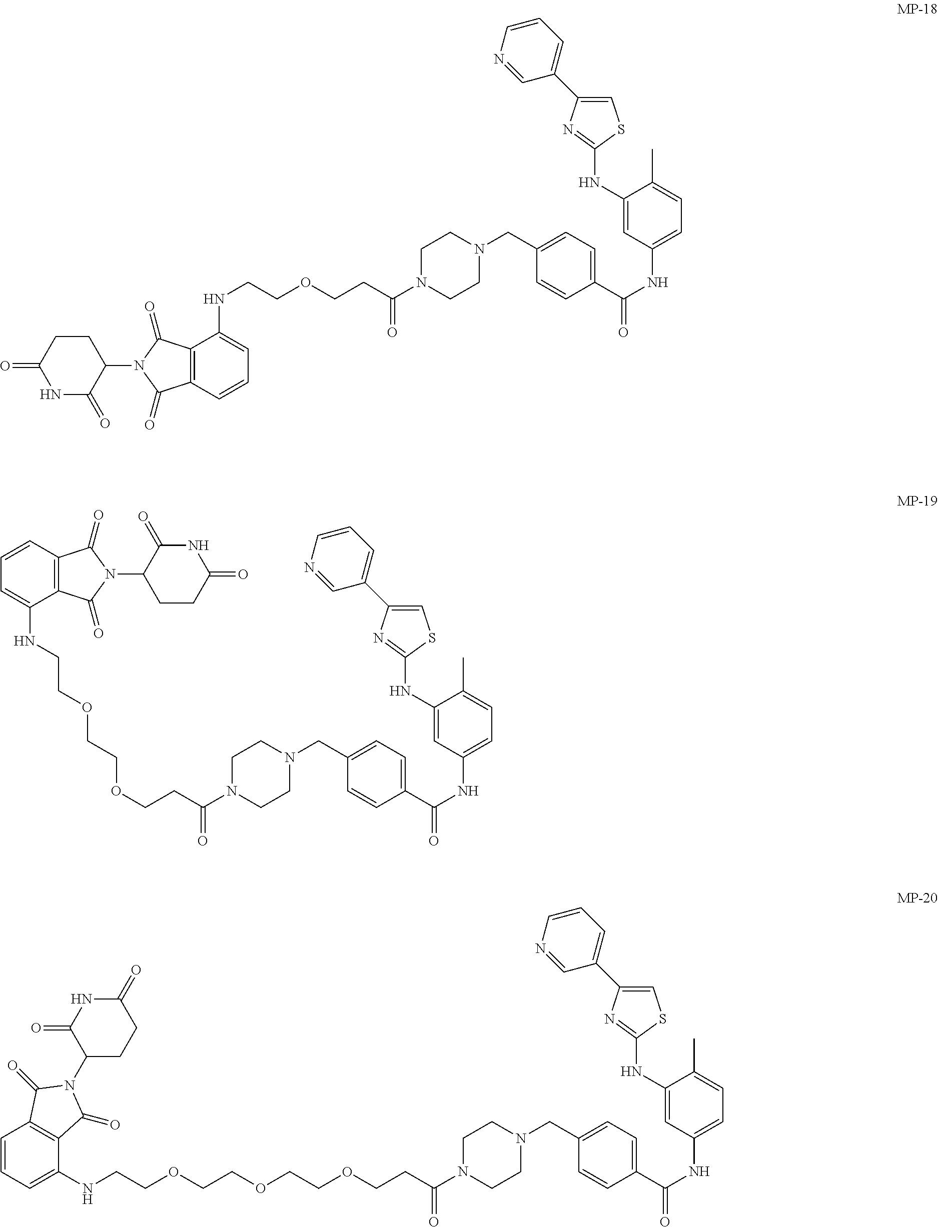 [SMILES: Cc1ccc(NC(=O)c2ccc(CN3CCN(C(=O)CCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCNc4cccc5c4C(=O)N(C4CCC(=O)NC4=O)C5=O)CC3)cc2)cc1Nc1nc(-c2cccnc2)cs1]

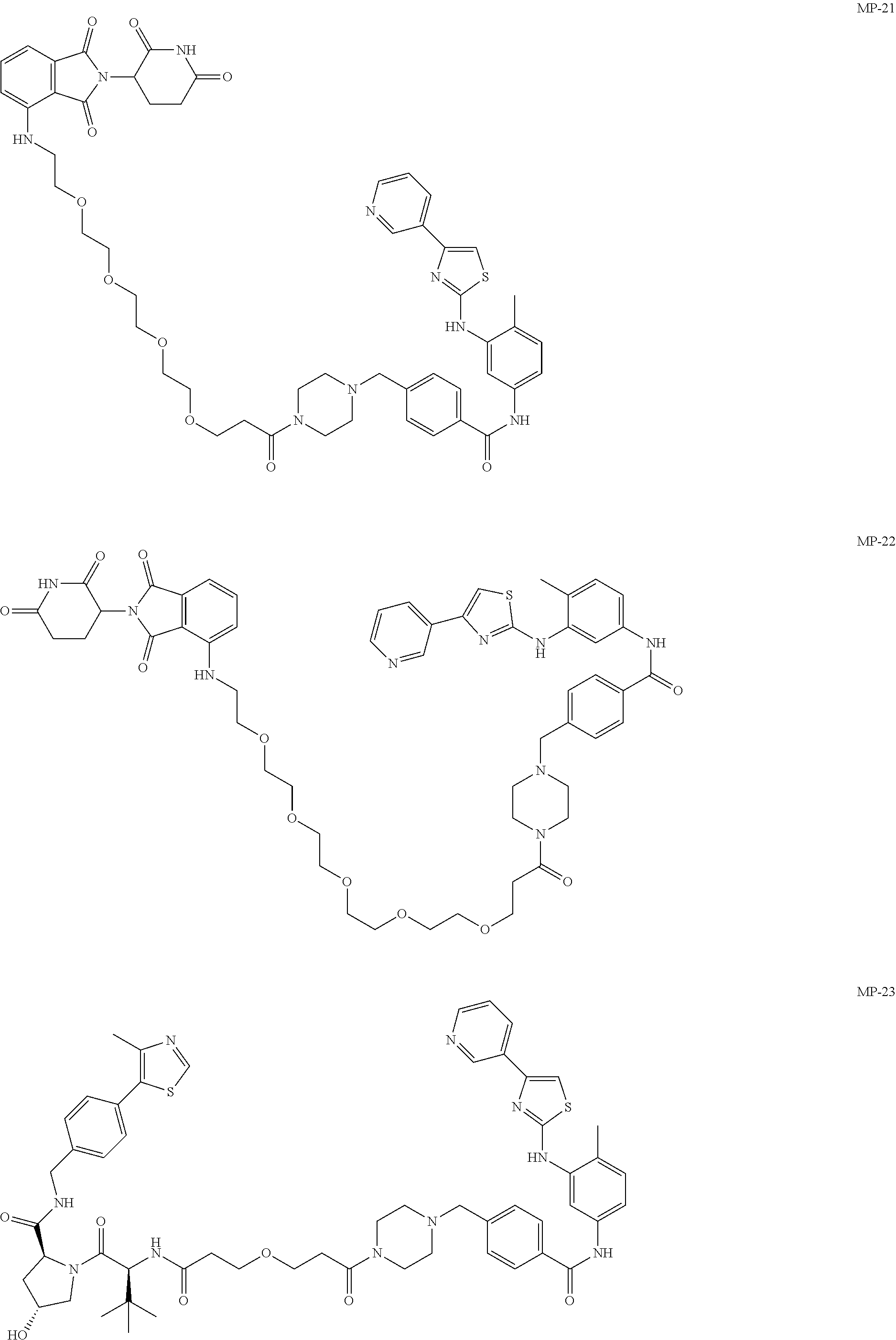 [SMILES: Cc1ccc(NC(=O)c2ccc(CN3CCN(C(=O)CCOCCOCCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCC(=O)N[C@H](C(=O)N4C[C@H](O)C[C@H]4C(=O)NCc4ccc(-c5scnc5C)cc4)C(C)(C)C)CC3)cc2)cc1Nc1nc(-c2cccnc2)cs1]

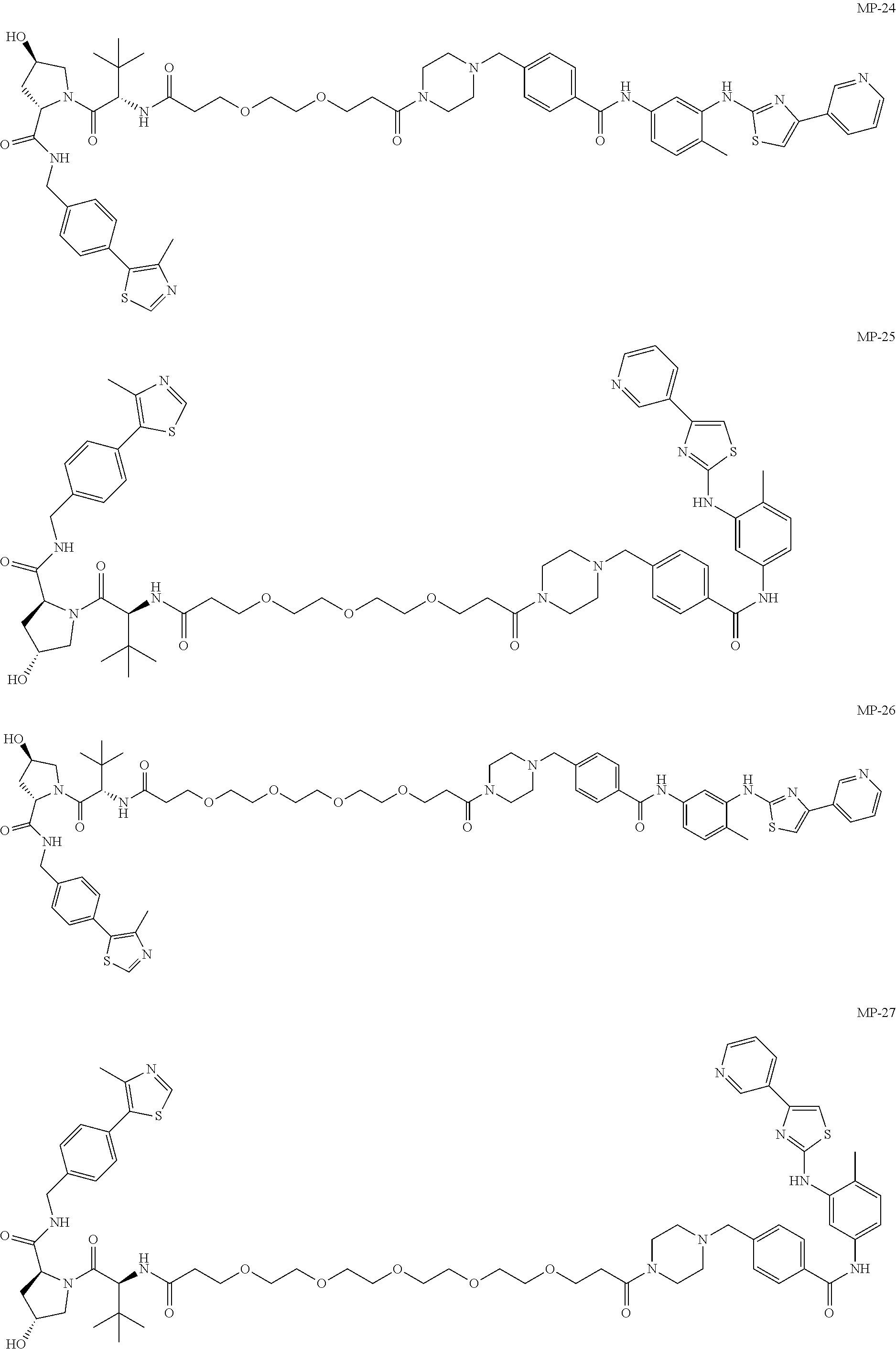

Each of MP-19, MP-20, MP-21, and MP-22 includes an E3 ligase ligand of pomalidomide and a linker comprising a PEG chain or segment. Each of MP-23, MP-24, MP-25, and MP-26 includes an E3 ligase ligand of the VHL ligand and a linker comprising a PEG chain or segment. However, many other combinations of E3 ligase ligand, Mpro ligand, and linker are possible and encompassed within the scope of the present disclosure.

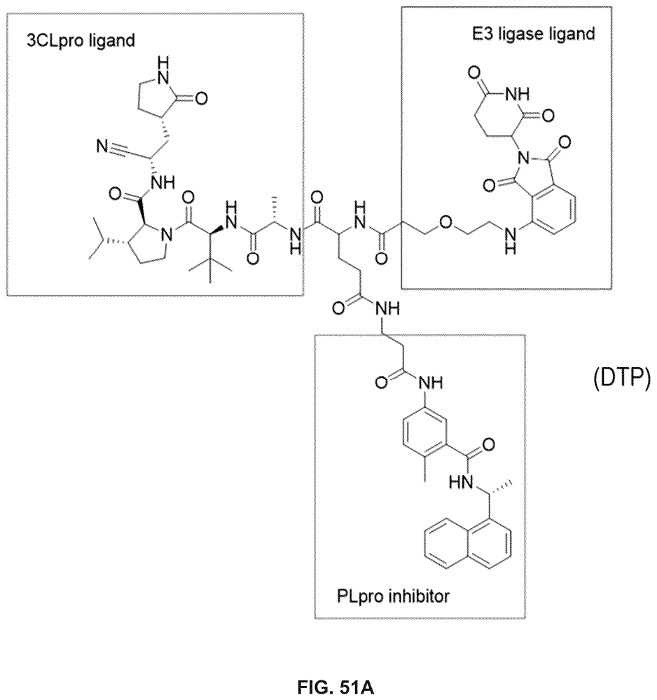

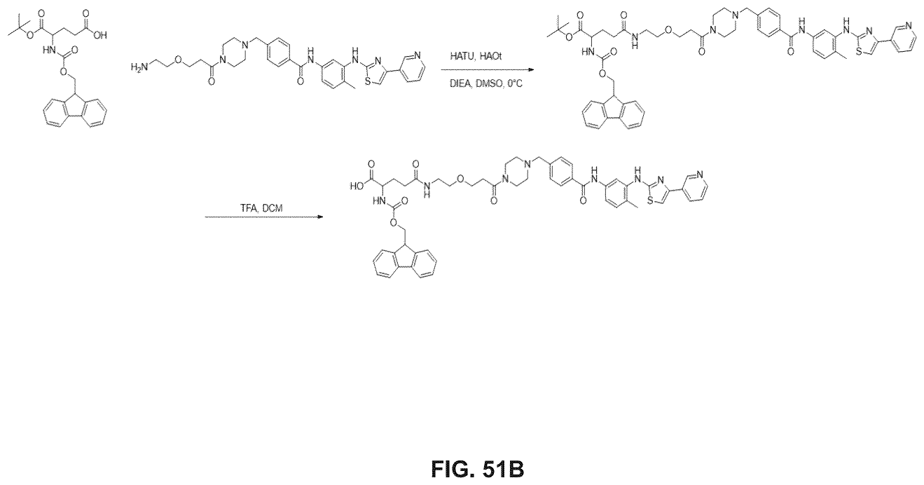

In other embodiments, provided herein are dual targeting PROTAC compounds which include an Mpro ligand attached to a linker, an E3 ligase ligand attached to the linker, and a papain-like protease (PLpro) inhibitor attached to the linker. (FIGS. 51A-51B.) The PLpro is a coronavirus enzyme required for processing viral polyproteins to generate a functional replicase complex and produce viral spread. PLpro is also implicated in cleaving proteinaceous post-translational modifications on host proteins as an evasion mechanism against host anti-viral immune responses. Although the crystal structure of PLpro is available to facilitate the design of small molecule inhibitors, the small molecules are sensitive to viral mutations and induce the development of drug resistance. PROTACs technology can be utilized to develop targeted therapeutics by building bifunctional ligands containing PLpro inhibitors connected by a chemical linker to a ligand that recruits E3 ubiquitin ligase. The PROTACs can promote the formation of a complex between PLpro and E3 ubiquitin ligase to induce degradation of the PLpro by the ubiquitin system. PROTACs technology provides methods to treat viral infections with advantages over treatments using traditional small molecules, acting at very low doses and being less sensitive to mutations compared to current therapeutic treatments. Combining SARS-CoV-2 key protein targeting drugs with the ubiquitin-proteasome system facilitates precise targeting of SARS-CoV-2 therapies, it can enhance the efficacy of the drugs, and it can minimize the propensity of the virus to develop resistance.

The same linkers, E3 ligase ligands, and Mpro ligands discussed above can be utilized in the dual targeting PROTAC compounds. However, it is also possible for the dual targeting PROTAC compounds to include two or more linkers, such as a first linker connecting the Mpro ligand to the E3 ligase ligand and a second linker connecting the PLpro ligand to the first linker. Non-limiting examples of PLpro inhibitors for use in the dual targeting PROTAC compounds include quinine, Osimertinib, digoxin, metergoline, epicriptine, ergometrine, bosutinib, citalopram, methdilazine, and the following structures referred to as PL-1, PL-2, PL-3, PL-4, PL-5, and PL-6:

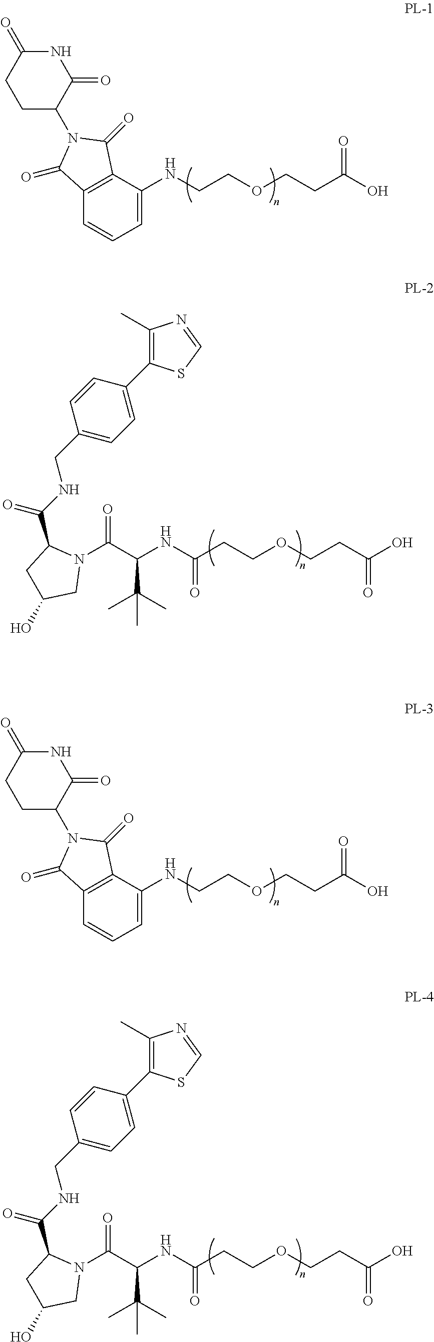

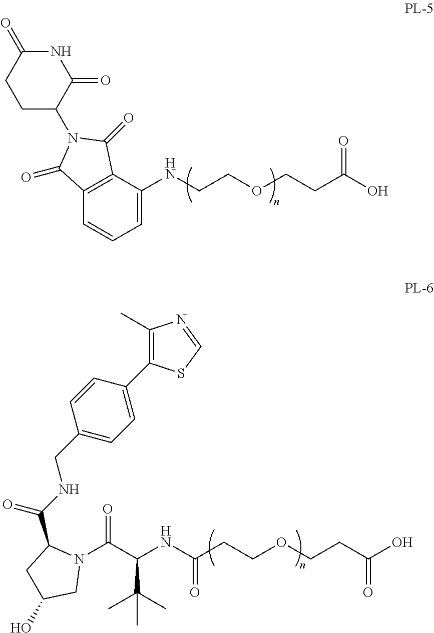

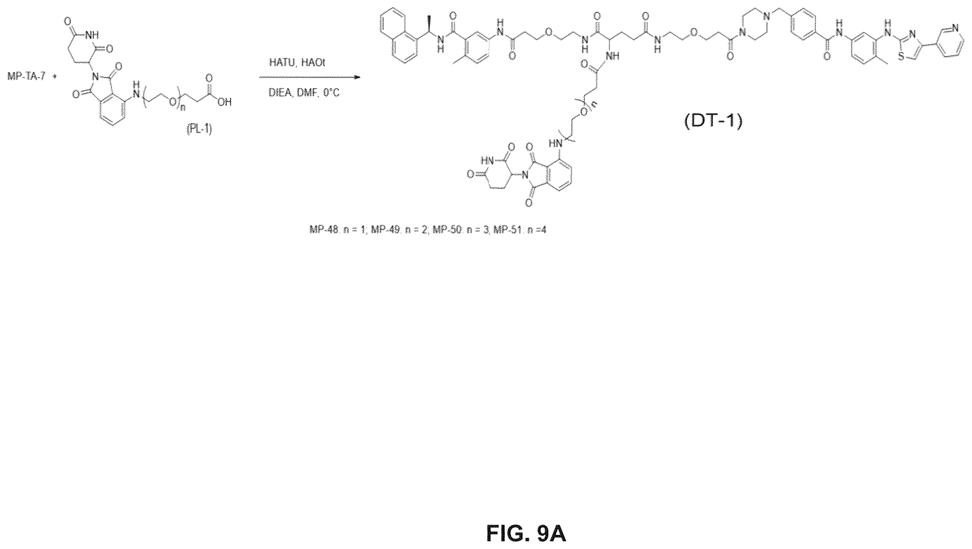

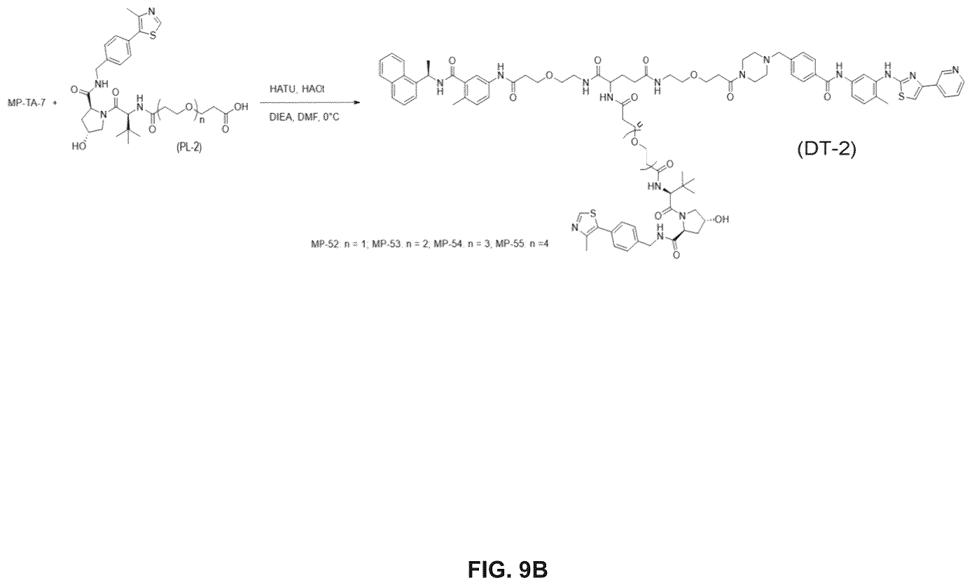

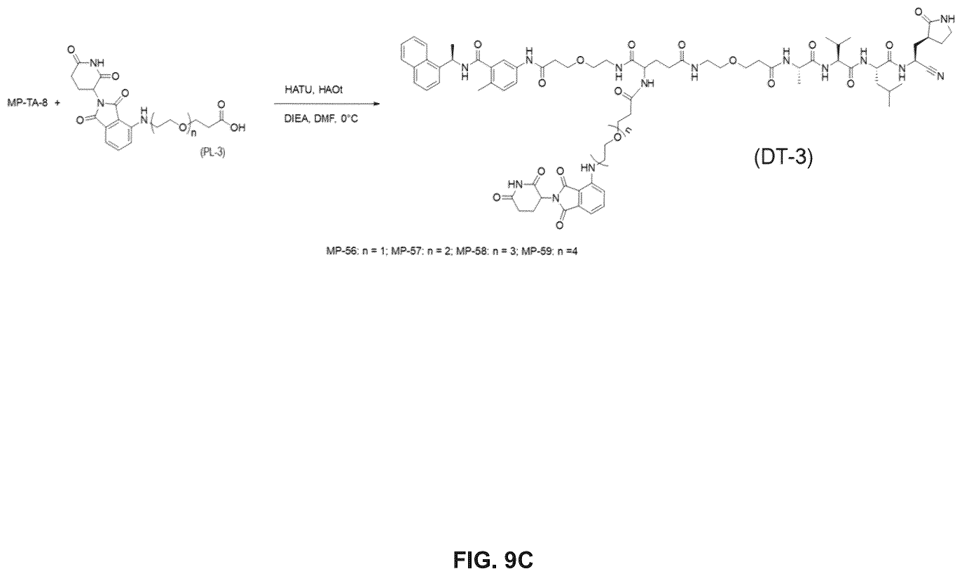

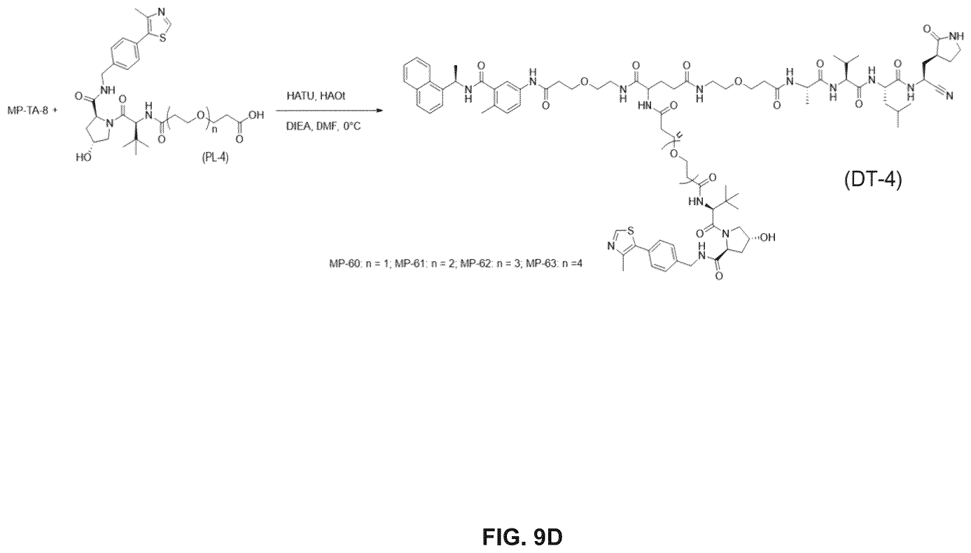

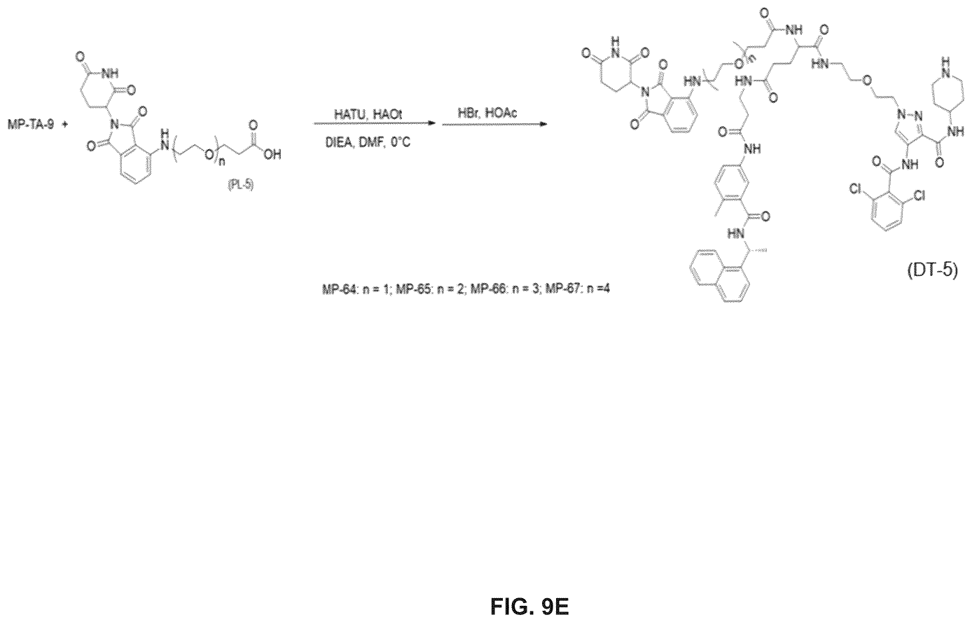

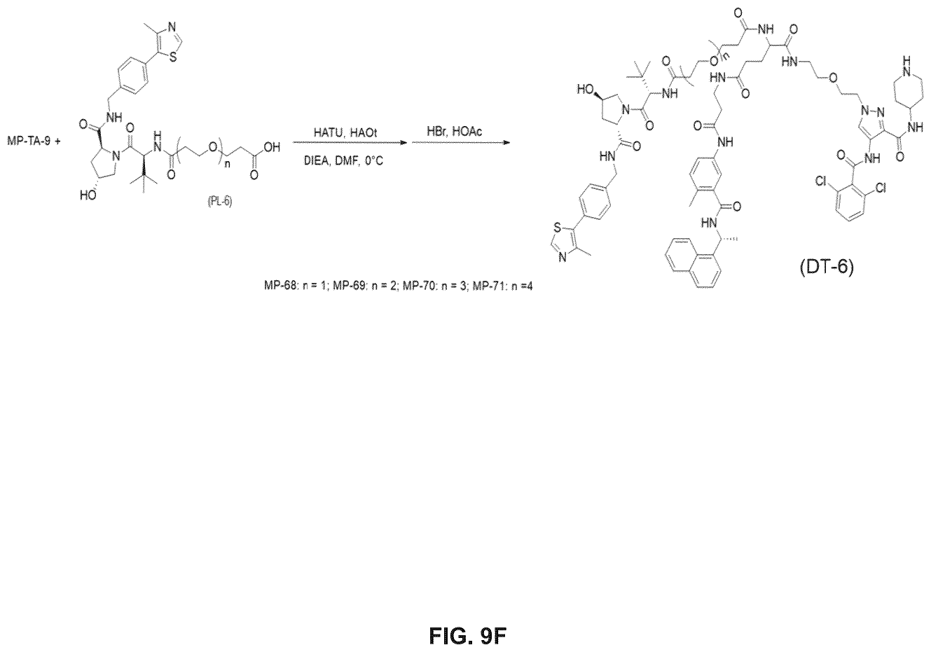

Non-limiting example dual targeting PROTAC compounds include those of the formulas DT-1, DT-2, DT-3, and DT-4:

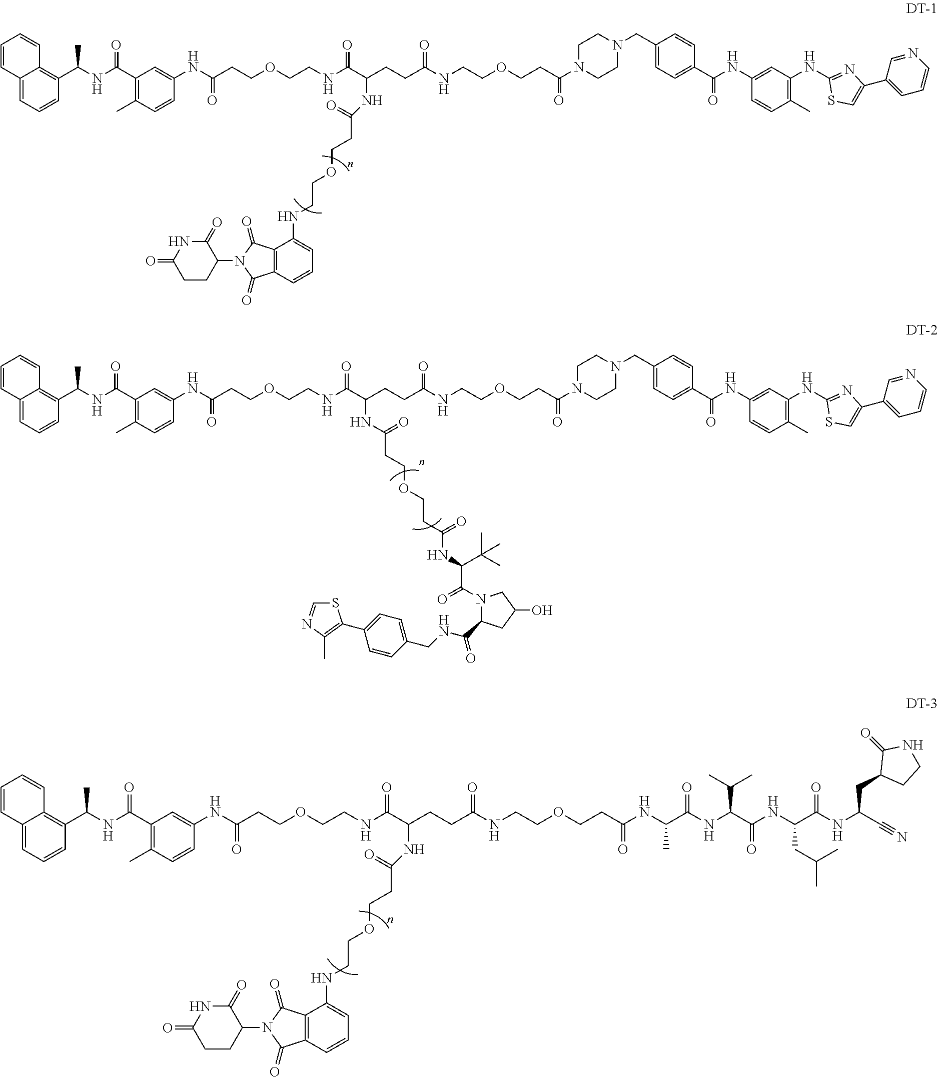

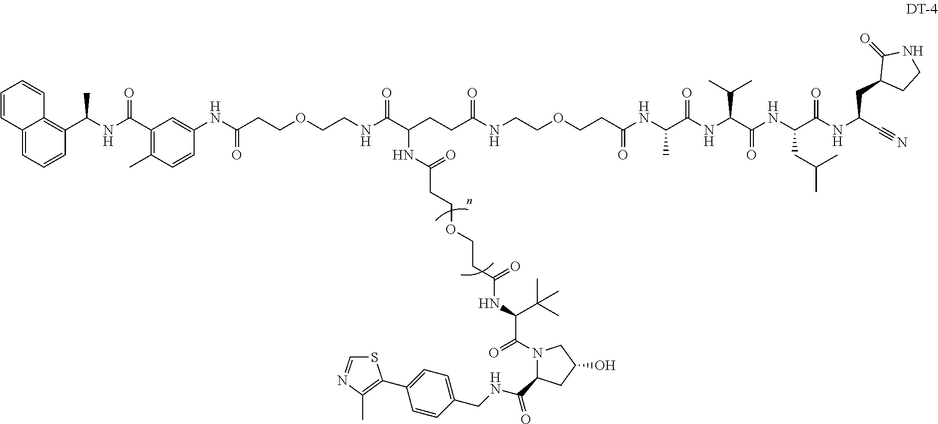

where n in each of DT-1, DT-2, DT-3, and DT-4 can be from 1-4. FIGS. 9A-9F show non-limiting example synthetic routes for preparing compounds having the general structures DT-1, DT-2, DT-3, and DT-4.

The PROTACs described herein are based on non-toxic compounds. Advantageously, the PROTACs are useful in treating COVID-19, and are non-toxic and low-dosing, making it difficult to develop drug resistance against them. Targeting virus-specific proteins may, at the same time, minimize the possibility of side effects.

Pharmaceutical compositions of the present disclosure may comprise an effective amount of a PROTAC compound (an “active ingredient”), optionally with additional agents, dissolved or dispersed in a pharmaceutically acceptable carrier, optionally with an additional cancer therapeutic drug. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other methods or a combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration can determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and ranges derivable therein. The amount of active compound(s) in each therapeutically useful composition may be prepared is such a way that a suitable dosage will be obtained in a given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and ranges derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form must be sterile and must be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration can determine the appropriate dose for the individual subject.

Sterile injectable solutions are prepared by incorporating the compositions in the determined amount in the appropriate solvent with other ingredients enumerated above, as appropriate, followed by filtered sterilization. Generally, dispersions are prepared by incorporating the various sterilized compositions into a sterile vehicle which contains the basic dispersion medium and other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via alternate routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating coronavirus infections. As described herein, the compounds and compositions herein can be used in combination therapies. That is, the compounds and compositions can be administered concurrently with, prior to, or subsequent to one or more other desired therapeutic or medical procedures or drugs, such as other coronavirus infection treatments. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

It is further envisioned that the compounds and methods described herein can be embodied in the form of a kit or kits. A non-limiting example of such a kit is a kit for making a PROTAC compound comprising an E3 ligase ligand, a linker, and a Mpro ligand in separate containers, where the containers may or may not be present in a combined configuration. Many other kits are possible, such as kits further comprising a PLpro inhibitor and/or a linker, or further comprising a pharmaceutically acceptable carrier, diluent, or excipient. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

A series of Mpro targeting PROTACs has been developed and tested for the ability to degrade Mpro activity.

### In Silico Design, Screening of Mpro Ligands, and the Synthesis of Mpro Targeting PROTACs

A series of Mpro ligands was created based on the crystal structure of Mpro through virtual screening and rational drug design. These Mpro ligands were screened from compound libraries that are either approved by the FDA or those currently in clinical use, so that the molecules have undergone safety assays and are ready to be used. The binding affinities and kinetics of the candidate Mpro ligands were determined using an in vitro assay and surface plasma resonance (SPR) technology. The ligands with high binding affinity and kinetics were connected with the E3 ligase ligands through a linker. PROTACs were synthesized by different combinations of Mpro ligands, crosslinker, and E3 ligase ligands orthogonally.

### Study of the Interaction and Degradation Activity of PROTACs Against Mpro

A heterologous expression system in mammalian cells was established for the evaluation degradation activity of synthesized PROTACs. The synthesized PROTACs were tested for their cytotoxicity to different cell lines, and binding affinity and kinetics against Mpro. The activity of PROTACs in degradation of Mpro was evaluated in Mpro expression mammalian cells.

### Initial Screening and General Methods

The compound N1, an irreversible inhibitor of MPro that can bind well to target proteins, was linked to the E3 ligand pomalidomide via PEG linkers, and it was found that a linker length between 3-4 PEG units causes degradation of the Mpro target protein. The compound N1 has the following structure:

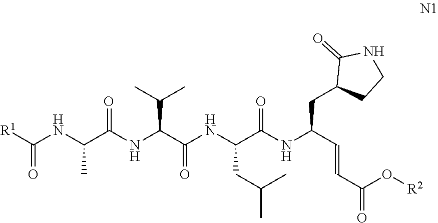

where R¹ is

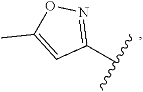 [SMILES: *c1cc(C)on1]

and R² is ethyl.

After selecting the chain length, the same method was used to screen for the effects of the E3 ligand, and the following four compounds 101-104 were identified:

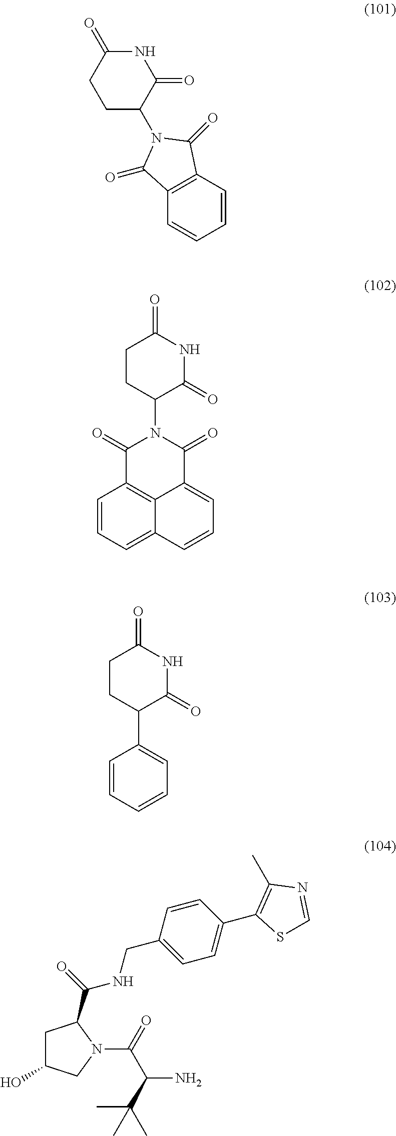 [SMILES: O=C1CCC(N2C(=O)c3ccccc3C2=O)C(=O)N1 ; O=C1CCC(N2C(=O)c3cccc4cccc(c34)C2=O)C(=O)N1 ; O=C1CCC(c2ccccc2)C(=O)N1 ; Cc1ncsc1-c1ccc(CNC(=O)[C@@H]2C[C@@H](O)CN2C(=O)[C@@H](N)C(C)(C)C)cc1]

Compounds 101-103 caused a similar degree of protein degradation, while compound 104 was slightly less active. Next, given that the double bonds in the compounds form irreversible covalent bonds with Cys in the target protein (which can affect the reuse of PROTACs), they were transposed to the cyano group. The presence of the cyano group makes the binding of the compound to the target site semi-reversible, which is conducive to the reuse of PROTACs in the cells. This compound was named MP-TA-N, and has the following structure:

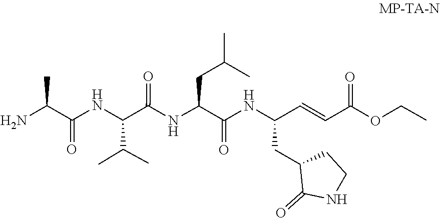 [SMILES: CCOC(=O)/C=C/[C@H](C[C@@H]1CCNC1=O)NC(=O)[C@H](CC(C)C)NC(=O)[C@@H](NC(=O)[C@H](C)N)C(C)C]

The structure of other amino acids in N1 was then modified to obtain compounds with better activity. Modifications included (i) replacing the Leu residue with a more hydrophobic group such as phenol or a cyclopropyl group; (ii) replacing the Val residue with a more hydrophobic group such as tertiary butyl or a cyclopropyl group; and (iii) replacing the methyl group of Ala residue to trifluoromethyl group. PEG₄ was used as a linker, and the E3 ligand was linked to it to obtain the compound MP-C-4N:

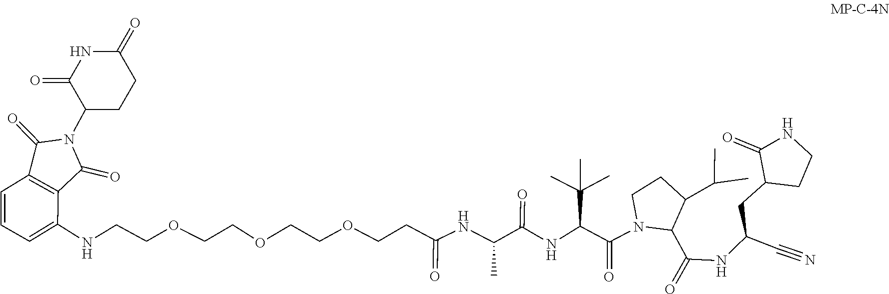 [SMILES: CC(C)C1CCN(C(=O)[C@@H](NC(=O)[C@H](C)NC(=O)CCOCCOCCOCCNc2cccc3c2C(=O)N(C2CCC(=O)NC2=O)C3=O)C(C)(C)C)C1C(=O)N[C@H](C#N)CC1CCNC1=O]

Through virtual screening, several candidates that can inhibit Mpro were found in FDA-approved drugs. Their structures were re-optimized following the Computer-Aided Drug Design (CADD) method to make them suitable for the subsequent synthesis of PROTACs. The E3 ligase ligand and PEG linker were purchased and conjugated with the Mpro ligand through condensation. For some compounds, it was found that a linker length between 3-4 PEG units causes degradation of the target protein, but for other compounds, a linker length between 1-2 PEG units causes degradation of the target protein.

To evaluate the degradation activity of MP-C-4N (initially, and additional PROTAC compounds later), a Mpro expression system was constructed in mammalian cells. A cell line stably expressing wild-type Mpro was constructed. According to the RNA sequence of wild-type Mpro, the corresponding DNA sequence was synthesized and codon-optimzied to be suitable for expression in HEK293 cells. After investigating the influence of different expression vectors and promoters on the protein expression level, pCW57.1-TetOn was selected as the expression vector, which contains a tetracycline-inducible promoter, and the target protein expression can be started or closed by controlling the content of tetracycline. With this cell line, the process of expressing MPro during virus infection in human cells was successfully simulated. This model was used to successfully test the degradation ability of candidate compounds on the target protein. Specifically, the cells were treated with doxycycline (1 μg/mL) for 12 h. After removing doxycycline, the cells were treated with PROTACs with indicated concentrations for 24 h and then harvested. The expression of Mpro was determined by Western Blot (WB) analysis using anti-T7 antibodies.

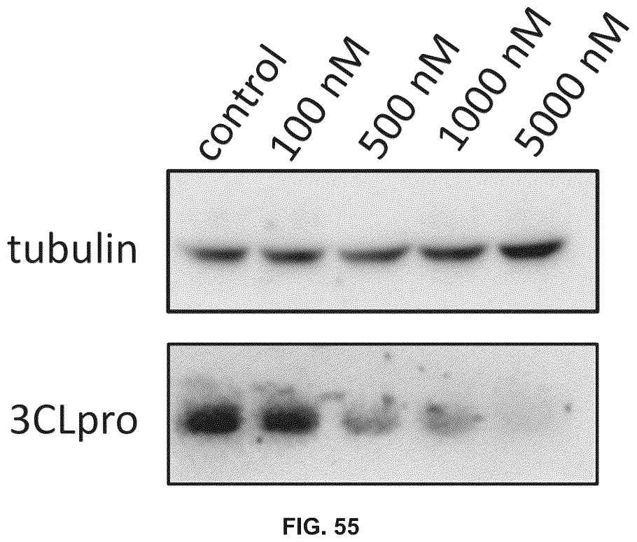

The cells were treated with MP-C-4N, and Mpro protein concentration was assessed using WB. The degradation of Mpro was detected when the concentration of MP-C-4N was at the 0.5-1.0 μM level, and the degradation activity was concentration-dependent. These results are shown in FIG. 55. This data indicates that MP-C-4N can mediate the degradation of Mpro at a nanomolar concentration when the protein is present in the cells, which indicates that MP-C-4N can be used for treating a coronavirus infection.

In addition, a rapid targeted protein degradation screening system was constructed using a GFP reporter with an RFP reporter as an internal reference. The GFP reporter and a targeted protein (Mpro and their mutants) join with a linker in between to increase the flexibility of the two proteins. The construct was stably transfected into HEK293 cells and used to evaluate the ability of PROTACs in degradation of fused targeted protein-GFP by quantitatively analysis of fluorescence intensity of GFP using a fluorescence plate reader.

To evaluate the anti-virus activity of the PROTACs, the low-virulence human coronavirus OC43 (HCoV-OC43) was used instead of SARS-CoV-2. Briefly, RD cells were infected with HCoV-OC43 and incubated at 33° C. for 1 h to allow virus adsorption. Then, the viral inoculum was removed. An overlay containing 0.2% Avicel supplemented with 2% FBS in DMEM containing serial concentrations of testing compounds was added and incubated in a 33° C. incubator for 4-5 days. The plaque formation was detected by staining the cell monolayer with crystal violet, and the plaque areas were quantified. EC₅₀ values were determined by plotting the percent CPE versus log₁₀ compound concentrations from best-fit dose-response curves with variable slope.

For all cell-based assay data, means between the groups with or without PROTAC treatment will be compared using the Student's (two-sample) t-test. A two-sided test with a significance level of <0.05 was considered statistically significant.

A library of additional PROTACs was synthesized and assessed for Mpro degradation ability, as further described in these examples.

### Synthesis of PLP-TA-1 (GRL-0617)

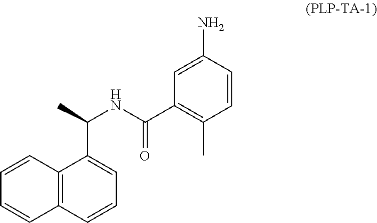 [SMILES: Cc1ccc(N)cc1C(=O)N[C@H](C)c1cccc2ccccc12]

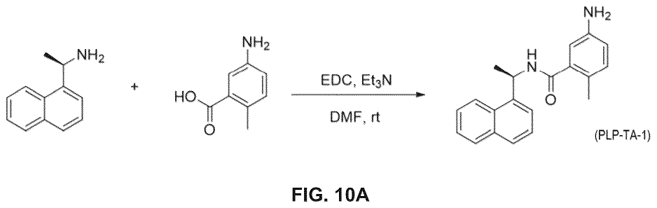

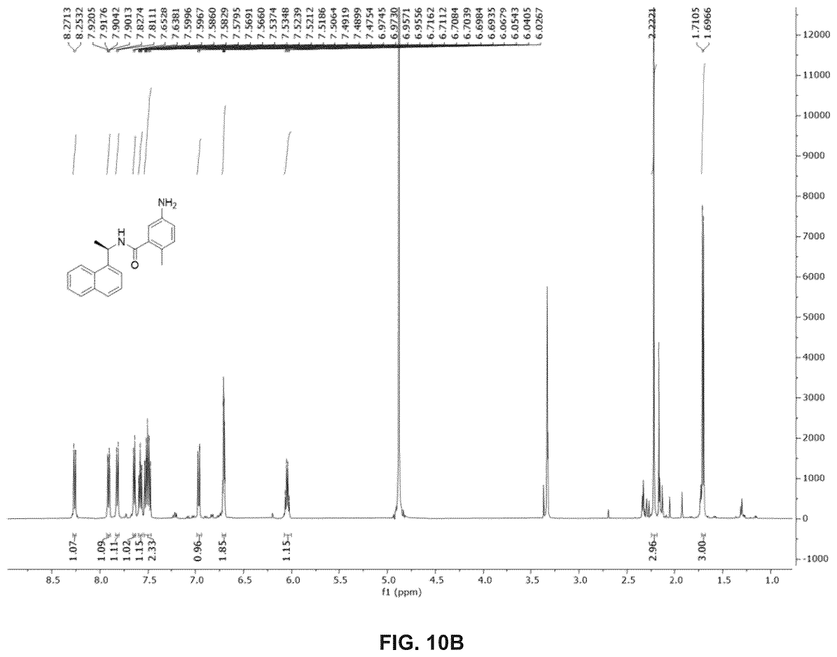

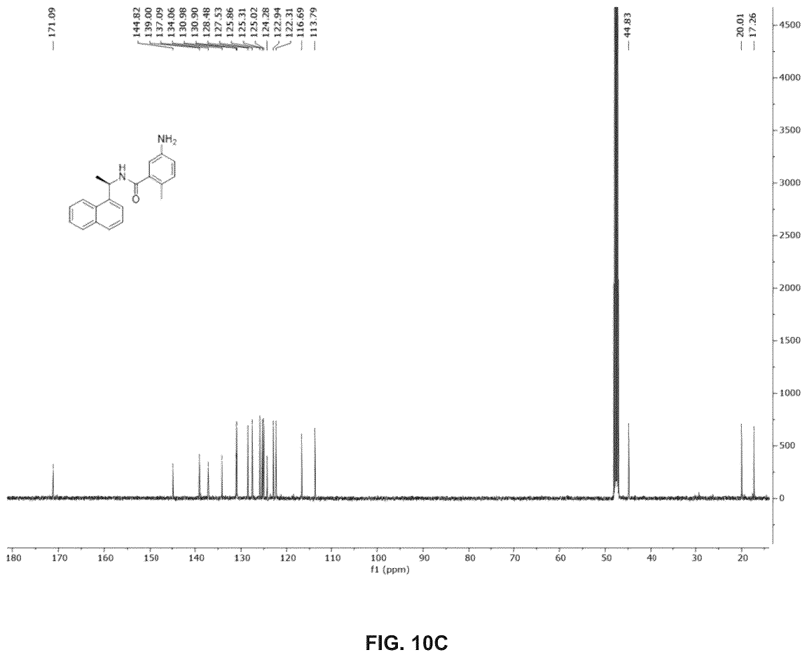

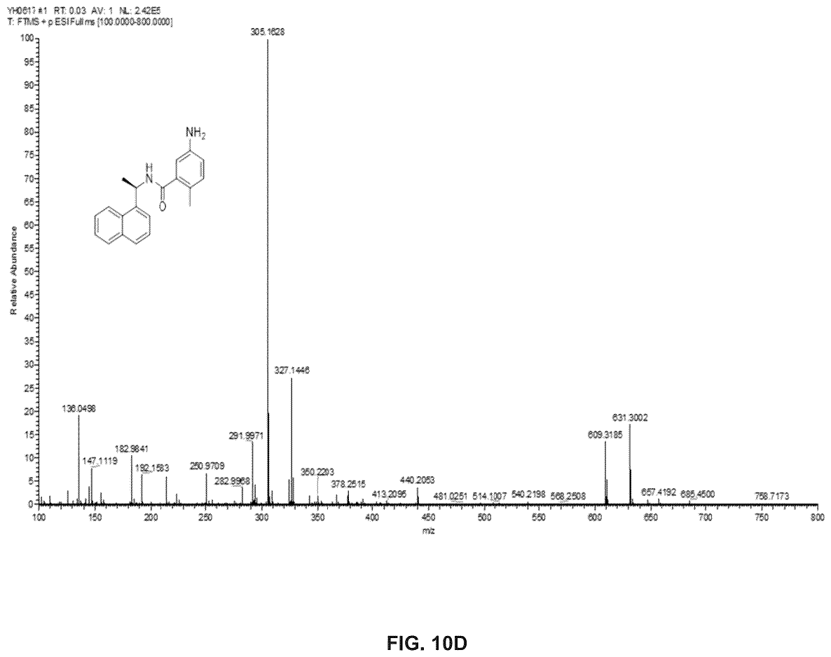

The synthesis of (R)-5-amino-2-methyl-N-(1-(naphthalen-1-yl)ethyl)benzamide (PLP-TA-1) is shown in FIG. 10A. To a solution of (R)-1-(naphthalen-1-yl)ethan-1-amine (0.17 g, 1.0 mmol) and 5-amino-2-methylbenzoic acid (0.15 g, 1.0 mmol) in DMF (10 ml), EDC (0.23 g, 1.2 mmol) and hydroxybenzotriazole (HOBt) (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.23 g, 78%). ¹H NMR (methanol-d₄, 500 MHz): 8.26 (d, J=8.5 Hz. 1H), 7.65 (d, J=7.4 Hz, 1H), 7.58 (ddd, J=8.4, 6.8, 1.5 Hz. 1H), 7.54-7.46 (m, 2H). 6.97 (dd, J=8.7, 0.8 Hz, 1H), 6.72-6.69 (m, 2H), 6.05 (q, 6.8 Hz, 1H), 2.22 (s, 3H), 1.70 (d, J=7.0 Hz, 3H), shown in FIG. 10B. ¹³C-NMR (methanol-d₄, 125 MHz): 171.1, 144.8, 139.0, 137.1, 134.1 131.0, 130.9, 128.5, 127.5, 125.9, 125.3 125.0, 124.3, 122.9, 122.3, 116.7, 113.8, 44.8, 20.0, 17.3, shown in FIG. 10C. HR-ESI-MS (m/z) calculated for 305.1650 (M+H)⁺, found 305.1628 (FIG. 10D).

### Synthesis of (R)-5-amino-2-hydroxy-N-(1-(naphthalen-1-yl)ethyl)benzanide (2)

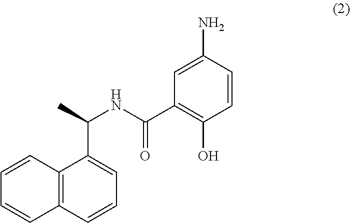 [SMILES: C[C@@H](NC(=O)c1cc(N)ccc1O)c1cccc2ccccc12]

To a solution (R)-1-(naphthalen-1-yl)ethan-1-amine (0.17 g, 1.0 mmol) and 5-amino-2-hydroxybenzoic acid (0.15 g, 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, dried with sodium sulfate, followed by concentration under reduced pressure. The crude product was then purified by silica gel (230-400 mesh) column chromatography yielding a light brown powder (0.23 g, 75%).

### Synthesis of (R)-5-amino-2-fluoro-N-(1-(naphthalen-1-yl)ethyl)benzamide (3)

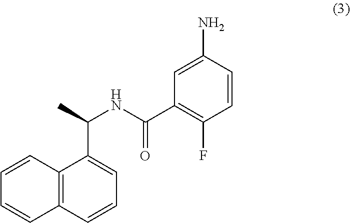 [SMILES: C[C@@H](NC(=O)c1cc(N)ccc1F)c1cccc2ccccc12]

To a solution of (R)-1-(naphthalen-1-yl)ethan-1-amine (2) (0.17 g, 1.0 mmol) and 5-amino-2-fluorobenzoic acid (0.16 g, 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.28 g, 86%).

### Synthesis of 5-amino-2-methyl-N-(quinoline-8-ylmethyl)benzamide (4)

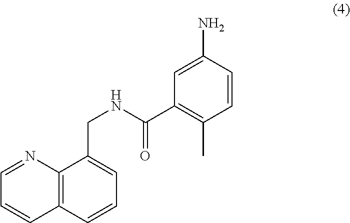 [SMILES: Cc1ccc(N)cc1C(=O)NCc1cccc2cccnc12]

To a solution quinolin-8-ylmethanamine (0.16 g, 1.0 mmol) and 5-amino-2-methylbenzoic acid (0.15 g. 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.23 g, 80%).

### Synthesis of 5-amino-2-hydroxy-N-(quinoline-8-ylmethyl)benzamide (5)

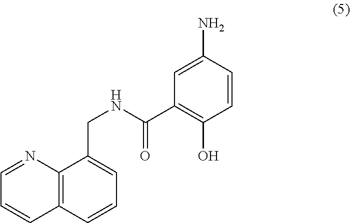 [SMILES: Nc1ccc(O)c(C(=O)NCc2cccc3cccnc23)c1]

To a solution quinolin-8-ylmethanainne (0.16 g, 1.0 mmol) and 5-amino-2-hydroxybenzoic acid (0.15 g. 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a light brown powder (0.20 g, 70%).

### Synthesis of 5-amino-2-fluoro-N-(quinoline-8-ylmethyl)benzamide (6)

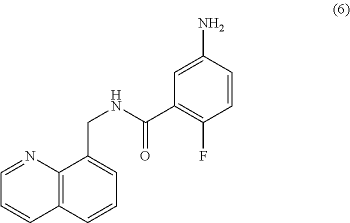 [SMILES: Nc1ccc(F)c(C(=O)NCc2cccc3cccnc23)c1]

To a solution of quinolin-8-ylmethanamine (0.16 g, 1.0 mmol) and 5-amino-2-fluorobenzoic acid (0.16 g, 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, dried with sodium sulfate, followed by concentration under reduced pressure. The crude product was then purified by silica gel (230-400 mesh) column chromatography yielding a white powder (0.28 g, 86%).

### Synthesis of 5-amino-2-methyl-N-(2-(napthalen-1-yl)ethyl)benzamide (7)

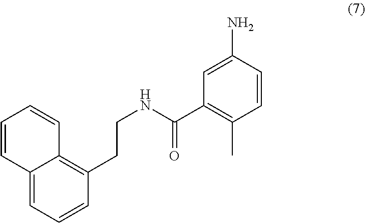 [SMILES: Cc1ccc(N)cc1C(=O)NCCc1cccc2ccccc12]

To a solution of quinolin-8-ylmethanamine (0.16 g, 1.0 mmol) and 5-amino-2-methylbenzoic acid (0.15 g, 1.0 mmol) in DMF(10 ml). EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.23 g, 80%).

### Synthesis of 5-amino-2-hydroxy-N-(2-(naphthalen-1-yl)ethyl)benzamide (8)

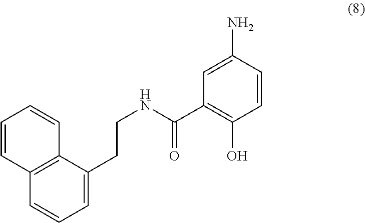 [SMILES: Nc1ccc(O)c(C(=O)NCCc2cccc3ccccc23)c1]

To a solution of quinolin-8-ylmethanamine (0.16 g. 1.0 mmol) and 5-amino-2-hydroxybenzoic acid (0.15 g, 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a light brown powder (0.20 g, 70%).

### Synthesis of 5-amino-2-fluoro-N-(2-(naphthalen-1-yl)ethyl)benzamide (9)

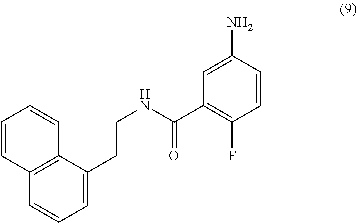 [SMILES: Nc1ccc(F)c(C(=O)NCCc2cccc3ccccc23)c1]

To a solution of quinolin-8-ylmethanamine (0.16 g, 1.0 mmol) and 5-amino-2-fluorobenzoic acid (0.16 g, 1.0 mmol) in DMF (10 ml), EDC (0.23 g, 1.2 mmol) and HOBt (0.16 g, 1.2 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.28 g, 86%).

### Synthesis of methyl 3-(((tert-butoxycarbonyl)amino)methyl)oxirane-2-carboxylate (10)

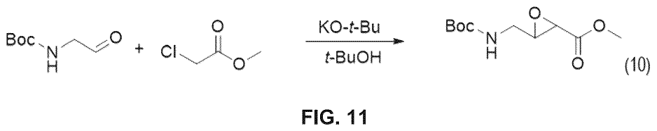

The synthesis of methyl 3-(((tert-butoxycarbonyl)amino)methyl)oxirane-2-carboxylate (10) is depicted in FIG. 11. A flask was charged with 0.16 g (0.1 mmol) of tert-butyl (2-oxoethyl) carbamate and 0.11 g. (0.1 mmole) of freshly distilled methyl 2-chloroacetate. A solution of 0.12 g (0.11 mmol) of potassium tert-butoxide in 5 ml of dry tert-butyl alcohol was added from the dropping funnel, the temperature of the reaction mixture being maintained at 10-15° C. After the addition was complete, the mixture was stirred for an additional 1-1.5 hours at about 10° C. Most of the tert-butyl alcohol was removed by distillation from the reaction flask at reduced pressure. The crude product was then purified by silica gel (230-400 mesh) column chromatography yielding a oil (0.21 g, 91%).

### Synthesis of methyl methyl 3-((2-((tert-butoxycarbonyl)amino)acetamido)methyl)oxirane-2-carboxylate (11)

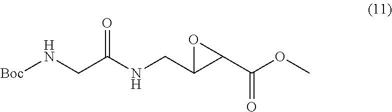 [SMILES: COC(=O)C1OC1CNC(=O)CNC(=O)OC(C)(C)C]

A flask with 2 ml TFA/CH₂Cl₂ (1:2) solution was charged with 0.21 g (0.09 mmol) of compound 10, then the mixture was stirred for 2 hours at ambient temperature followed by concentration under reduced pressure. Then, 0.16 g (0.09 mmol) Boc-glycine, 1 ml DMF, EDC (0.023 g, 0.12 mmol), and HOBt (0.016 g, 0.12 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.22 g, 85%).

### Synthesis of methyl 3-(7-(hydroxymethyl)-11,11-dimethyl-3, 6,9-trioxo-10-oxa-2,5,8-triazadodecyl)oxirane-2-carboxylate (12)

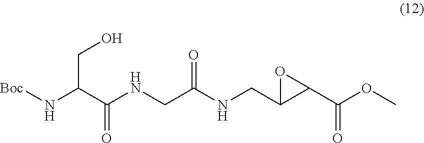 [SMILES: COC(=O)C1OC1CNC(=O)CNC(=O)C(CO)NC(=O)OC(C)(C)C]

A flask having 5 ml TFA/CH₂Cl₂ (1:2) solution was charged with 0.22 g (0.076 mmol) of compound 11, then was stirred for 2 hours at ambient temperature followed by concentration under reduced pressure. Then, 0.16 g (0.08 mmol) Boc-Ser, 1 ml DMF, EDC (0.023 g, 0.12 mmol), and HOBt (0.016 g, 0.12 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.25 g. 90%).

### Synthesis of methyl 3-(7-(hydroxymethyl)-10-(4-hydroxyphenethyl)-14,14-dimethyl-3,6,9,12-tetraoxo-13-oxa-2,5,8,11-tetraazapentadecyl)oxirane-2-carboxylate (13)

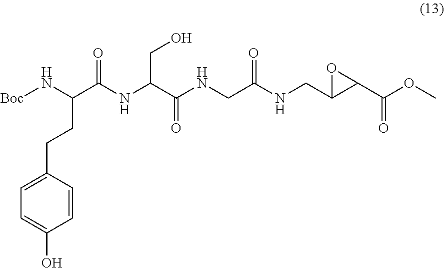 [SMILES: COC(=O)C1OC1CNC(=O)CNC(=O)C(CO)NC(=O)C(CCc1ccc(O)cc1)NC(=O)OC(C)(C)C]

A flask with 5 ml TFA/CH₂Cl₂ (1:2) solution was charged with 0.25 g (0.067 mmol) of compound 10, then stirred for 2 hours at ambient temperature followed by concentration under reduced pressure. Then, 0.20 g (0.067 mmol) 2-((tert-butoxycarbonyl)amino)-4-(4-hydroxyphenyl)butanoic acid, 1 ml DMF, EDC (0.023 g, 0.12 mmol), and HOBt (0.016 g, 0.12 nmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (0.50 g. 91%)

### Synthesis of Compounds 14-24

Compound 14 was synthesized similarly to compound 13:

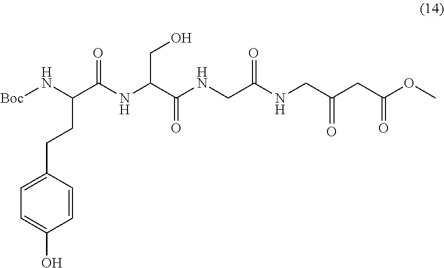 [SMILES: COC(=O)CC(=O)CNC(=O)CNC(=O)C(CO)NC(=O)C(CCc1ccc(O)cc1)NC(=O)OC(C)(C)C]

Compounds 15-24 were similarly synthesized:

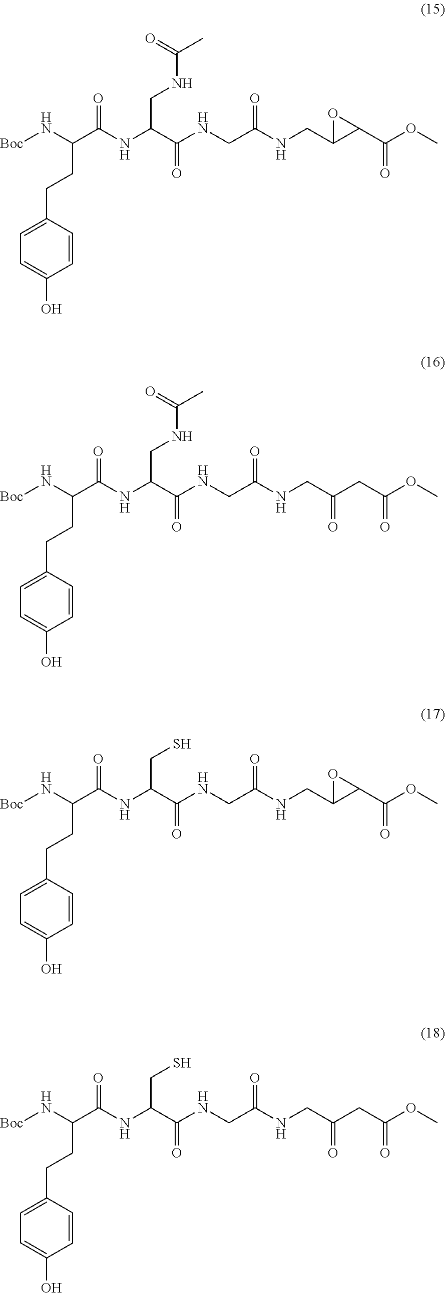 [SMILES: COC(=O)C1OC1CNC(=O)CNC(=O)C(CNC(C)=O)NC(=O)C(CCc1ccc(O)cc1)NC(=O)OC(C)(C)C ; COC(=O)CC(=O)CNC(=O)CNC(=O)C(CNC(C)=O)NC(=O)C(CCc1ccc(O)cc1)NC(=O)OC(C)(C)C ; COC(=O)C1OC1CNC(=O)CNC(=O)C(CS)NC(=O)C(CCc1ccc(O)cc1)NC(=O)OC(C)(C)C ; COC(=O)CC(=O)CNC(=O)CNC(=O)C(CS)NC(=O)C(CCc1ccc(O)cc1)NC(=O)OC(C)(C)C]

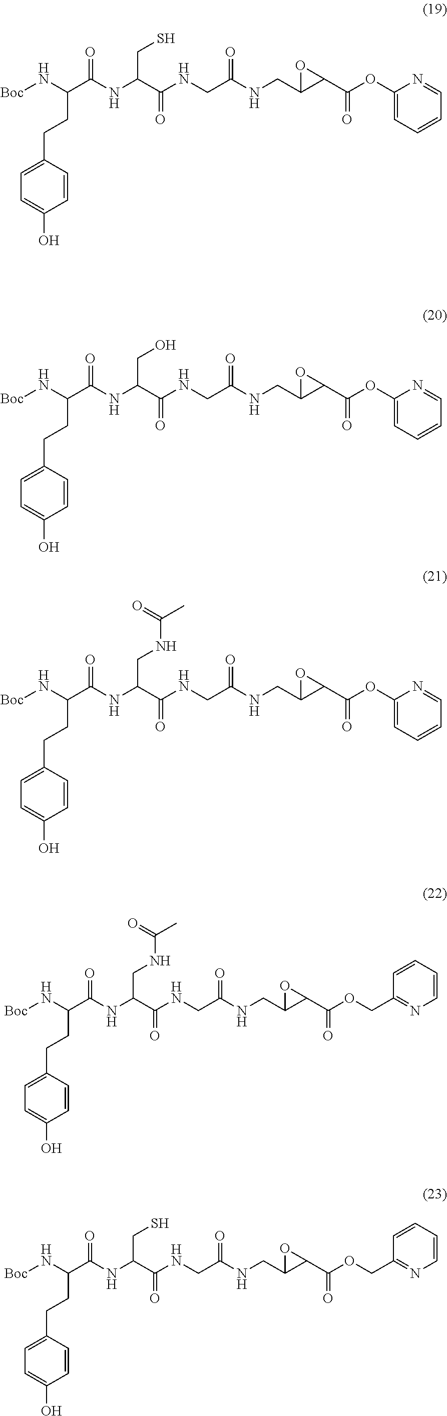 [SMILES: CC(C)(C)OC(=O)NC(CCc1ccc(O)cc1)C(=O)NC(CS)C(=O)NCC(=O)NCC1OC1C(=O)Oc1ccccn1 ; CC(C)(C)OC(=O)NC(CCc1ccc(O)cc1)C(=O)NC(CO)C(=O)NCC(=O)NCC1OC1C(=O)Oc1ccccn1 ; CC(=O)NCC(NC(=O)C(CCc1ccc(O)cc1)NC(=O)OC(C)(C)C)C(=O)NCC(=O)NCC1OC1C(=O)Oc1ccccn1 ; CC(=O)NCC(NC(=O)C(CCc1ccc(O)cc1)NC(=O)OC(C)(C)C)C(=O)NCC(=O)NCC1OC1C(=O)OCc1ccccn1 ; CC(C)(C)OC(=O)NC(CCc1ccc(O)cc1)C(=O)NC(CS)C(=O)NCC(=O)NCC1OC1C(=O)OCc1ccccn1]

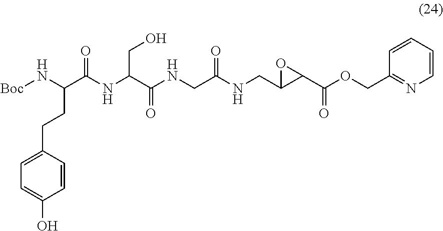 [SMILES: CC(C)(C)OC(=O)NC(CCc1ccc(O)cc1)C(=O)NC(CO)C(=O)NCC(=O)NCC1OC1C(=O)OCc1ccccn1]

### Synthesis of Compounds 25-33

To a solution compound PLP-TA-1 (6.0 mg, 0.02 mmol) and pomalidomide-C3-COOH (7.2 mg, 0.02 mmol) in DMF (10 ml), EDC (0.05 g, 0.03 mmol) and HOBt (0.04 g, 0.03 mmol) were added. The mixture was stirred for 24 hours at ambient temperature. The product was extracted with ethyl acetate, and dried with sodium sulfate, followed by concentration under reduced pressure. The crude product was then purified by silica gel (230-400 mesh) column chromatography yielding a white powder (9.6 mg, 80%).

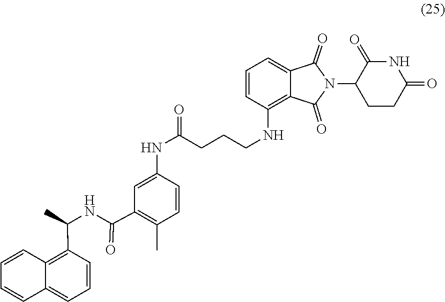 [SMILES: Cc1ccc(NC(=O)CCCNc2cccc3c2C(=O)N(C2CCC(=O)NC2=O)C3=O)cc1C(=O)N[C@H](C)c1cccc2ccccc12]

The same procedure was followed to synthesize compounds 26-33:

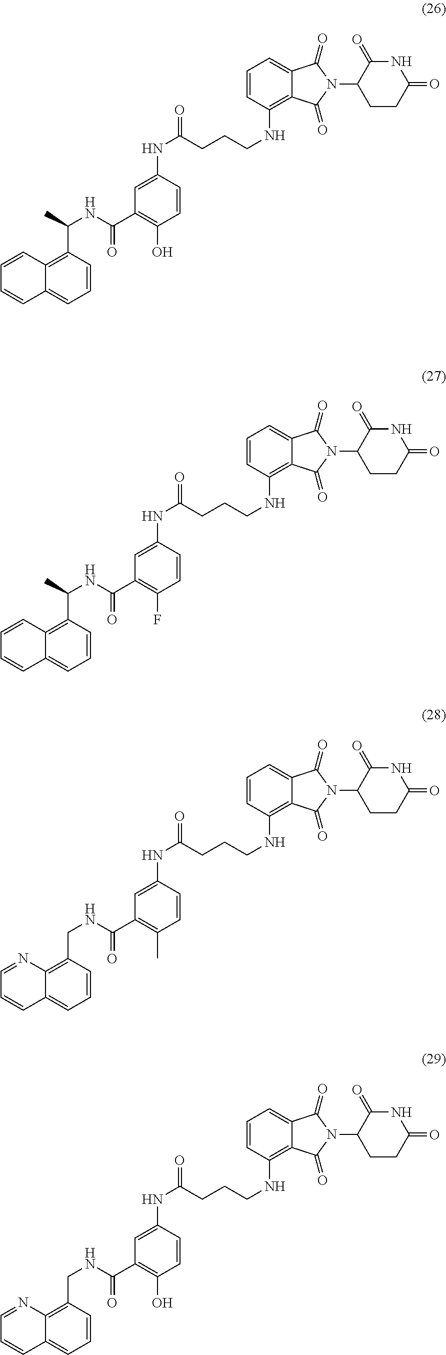 [SMILES: C[C@@H](NC(=O)c1cc(NC(=O)CCCNc2cccc3c2C(=O)N(C2CCC(=O)NC2=O)C3=O)ccc1O)c1cccc2ccccc12 ; C[C@@H](NC(=O)c1cc(NC(=O)CCCNc2cccc3c2C(=O)N(C2CCC(=O)NC2=O)C3=O)ccc1F)c1cccc2ccccc12 ; Cc1ccc(NC(=O)CCCNc2cccc3c2C(=O)N(C2CCC(=O)NC2=O)C3=O)cc1C(=O)NCc1cccc2cccnc12 ; O=C1CCC(N2C(=O)c3cccc(NCCCC(=O)Nc4ccc(O)c(C(=O)NCc5cccc6cccnc56)c4)c3C2=O)C(=O)N1]

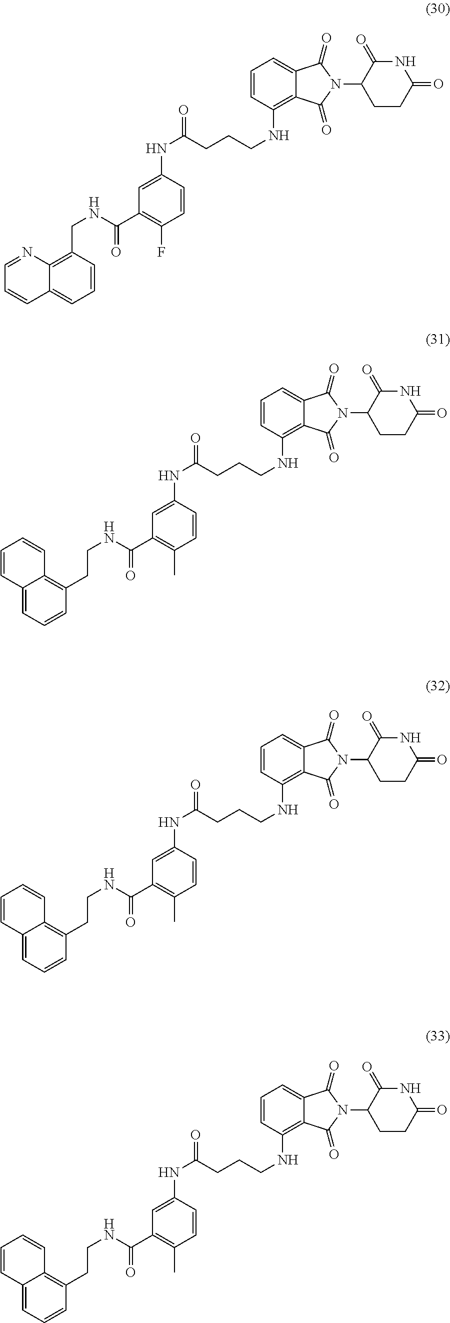 [SMILES: O=C1CCC(N2C(=O)c3cccc(NCCCC(=O)Nc4ccc(F)c(C(=O)NCc5cccc6cccnc56)c4)c3C2=O)C(=O)N1 ; Cc1ccc(NC(=O)CCCNc2cccc3c2C(=O)N(C2CCC(=O)NC2=O)C3=O)cc1C(=O)NCCc1cccc2ccccc12 ; Cc1ccc(NC(=O)CCCNc2cccc3c2C(=O)N(C2CCC(=O)NC2=O)C3=O)cc1C(=O)NCCc1cccc2ccccc12 ; Cc1ccc(NC(=O)CCCNc2cccc3c2C(=O)N(C2CCC(=O)NC2=O)C3=O)cc1C(=O)NCCc1cccc2ccccc12]

### Synthesis of Compounds 34-45

1 ml TFA/CH₂Cl₂ (1:2) solution was added to a flask charged with 11 mg (0.02 mmol) of compound 13, then the mixture was stirred for 2 hours at ambient temperature followed by concentration under reduced pressure. Then, pomalidomide-C3-COOH (7.2 mg, 0.02 mmol) in DMF (1 ml), EDC (0.05 g, 0.03 mmol), and HOBt (0.04 g, 0.03 mmol) were added. The mixture was stirred for 24 hours at ambient temperature. The product was extracted with ethyl acetate, and dried with sodium sulfate, followed by concentration under reduced pressure. The crude product was then purified by HPLC yielding a yellow powder (14 mg, 85%).

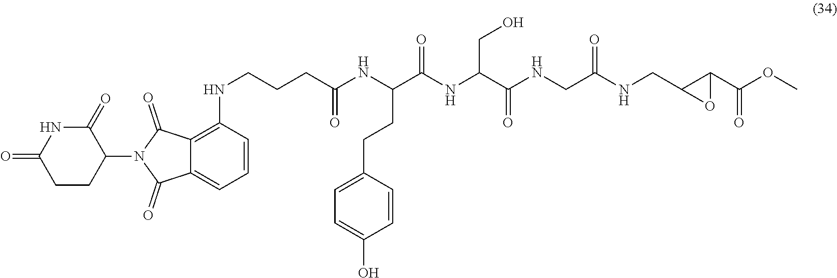 [SMILES: COC(=O)C1OC1CNC(=O)CNC(=O)C(CO)NC(=O)C(CCc1ccc(O)cc1)NC(=O)CCCNc1cccc2c1C(=O)N(C1CCC(=O)NC1=O)C2=O]

The same procedure was followed to synthesize compounds 35-45:

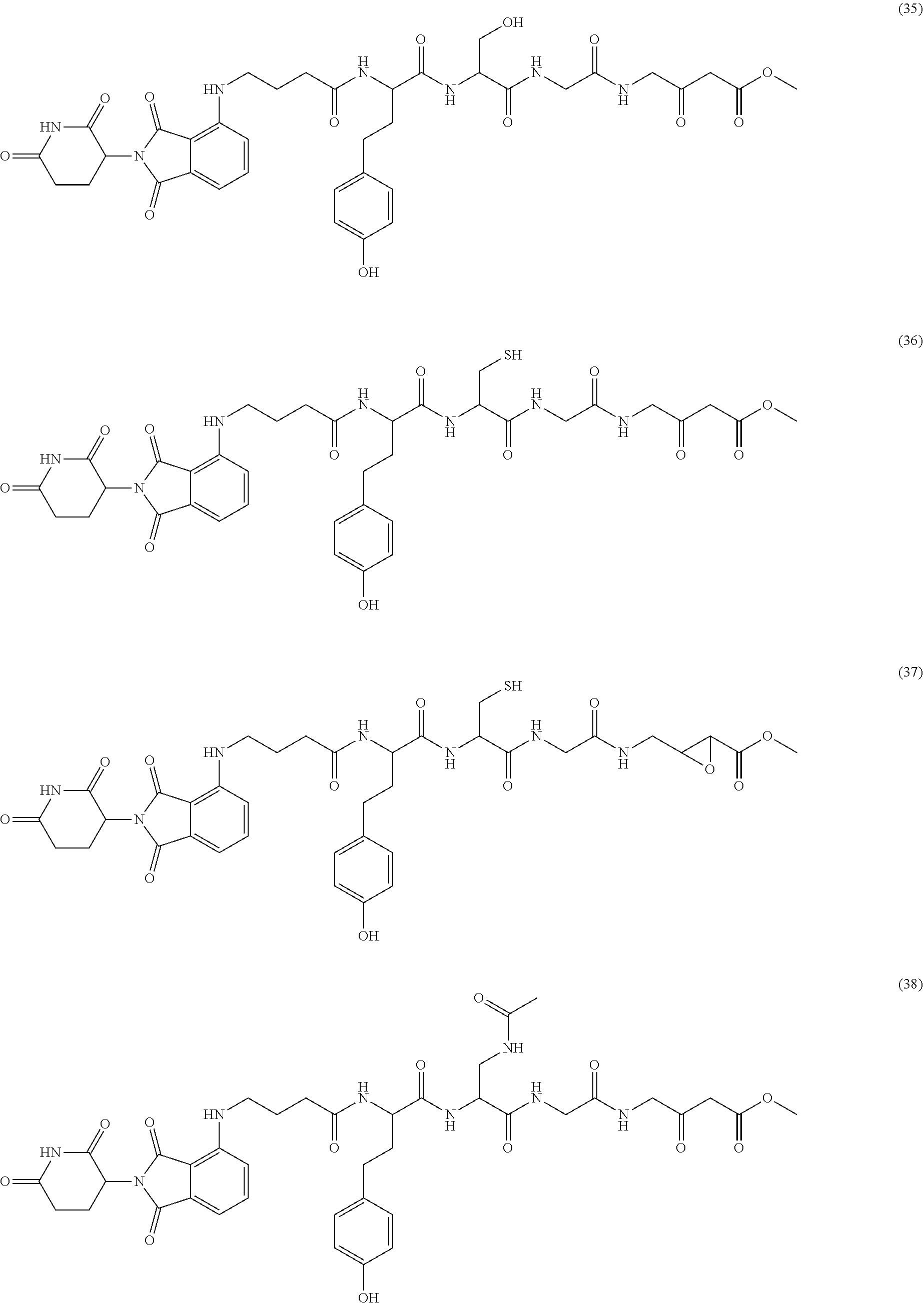 [SMILES: COC(=O)CC(=O)CNC(=O)CNC(=O)C(CO)NC(=O)C(CCc1ccc(O)cc1)NC(=O)CCCNc1cccc2c1C(=O)N(C1CCC(=O)NC1=O)C2=O ; COC(=O)CC(=O)CNC(=O)CNC(=O)C(CS)NC(=O)C(CCc1ccc(O)cc1)NC(=O)CCCNc1cccc2c1C(=O)N(C1CCC(=O)NC1=O)C2=O ; COC(=O)C1OC1CNC(=O)CNC(=O)C(CS)NC(=O)C(CCc1ccc(O)cc1)NC(=O)CCCNc1cccc2c1C(=O)N(C1CCC(=O)NC1=O)C2=O ; COC(=O)CC(=O)CNC(=O)CNC(=O)C(CNC(C)=O)NC(=O)C(CCc1ccc(O)cc1)NC(=O)CCCNc1cccc2c1C(=O)N(C1CCC(=O)NC1=O)C2=O]

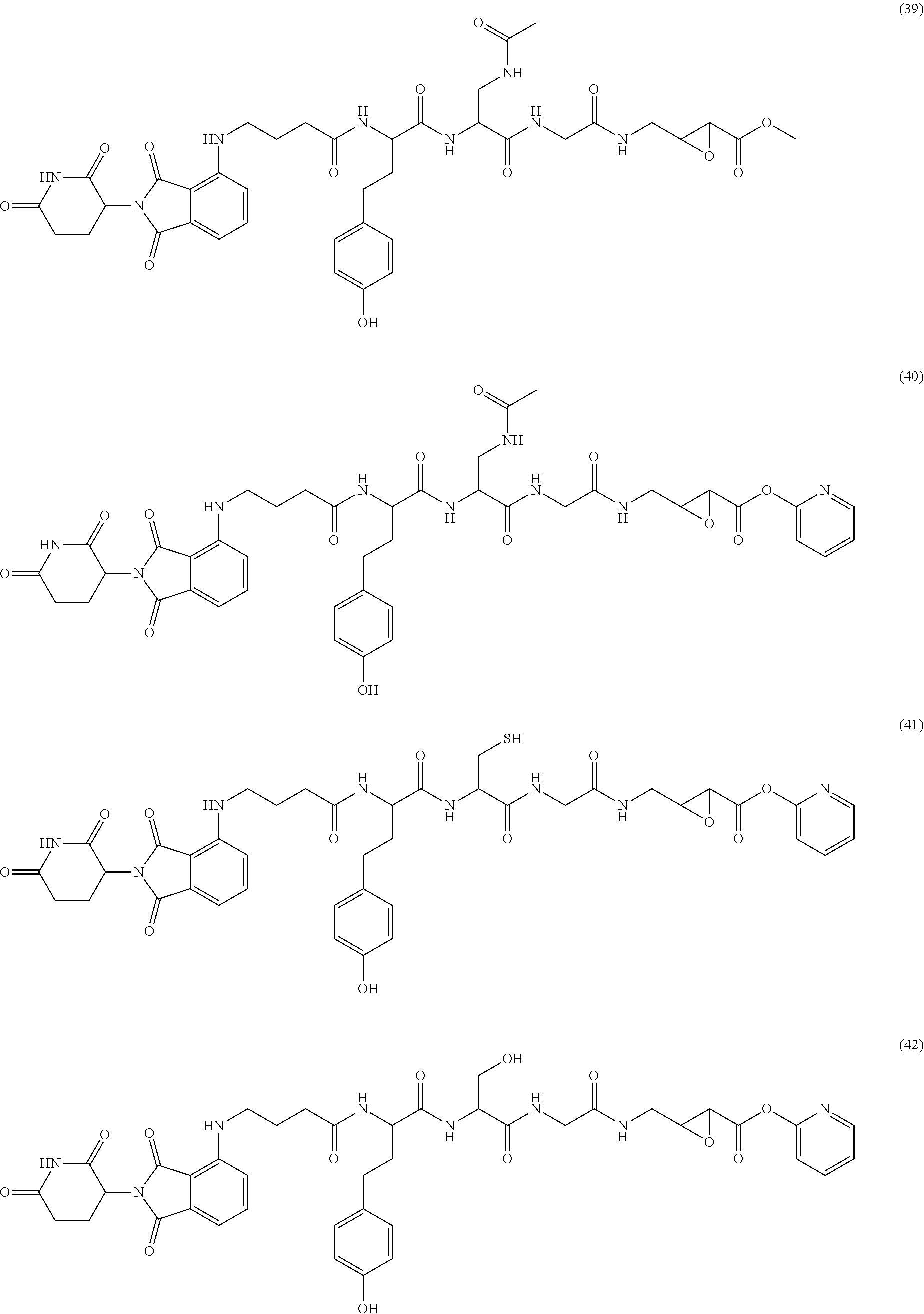 [SMILES: COC(=O)C1OC1CNC(=O)CNC(=O)C(CNC(C)=O)NC(=O)C(CCc1ccc(O)cc1)NC(=O)CCCNc1cccc2c1C(=O)N(C1CCC(=O)NC1=O)C2=O ; CC(=O)NCC(NC(=O)C(CCc1ccc(O)cc1)NC(=O)CCCNc1cccc2c1C(=O)N(C1CCC(=O)NC1=O)C2=O)C(=O)NCC(=O)NCC1OC1C(=O)Oc1ccccn1 ; O=C(CNC(=O)C(CS)NC(=O)C(CCc1ccc(O)cc1)NC(=O)CCCNc1cccc2c1C(=O)N(C1CCC(=O)NC1=O)C2=O)NCC1OC1C(=O)Oc1ccccn1 ; O=C(CNC(=O)C(CO)NC(=O)C(CCc1ccc(O)cc1)NC(=O)CCCNc1cccc2c1C(=O)N(C1CCC(=O)NC1=O)C2=O)NCC1OC1C(=O)Oc1ccccn1]

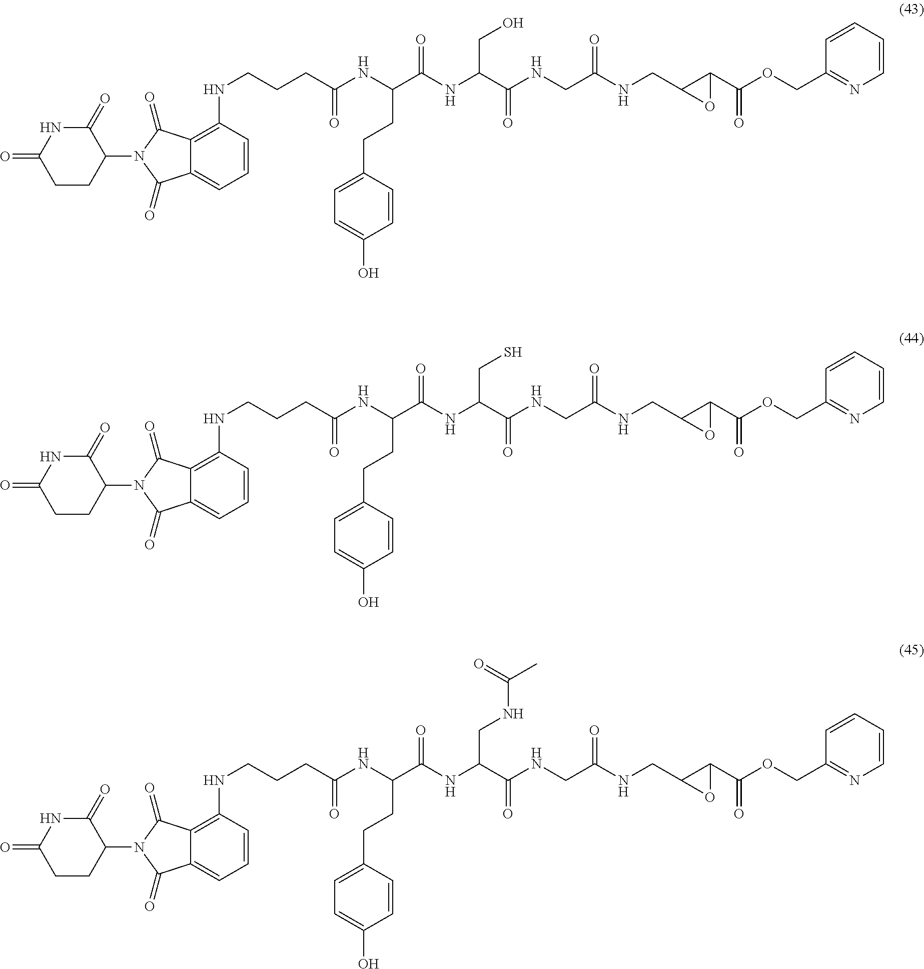 [SMILES: O=C(CNC(=O)C(CO)NC(=O)C(CCc1ccc(O)cc1)NC(=O)CCCNc1cccc2c1C(=O)N(C1CCC(=O)NC1=O)C2=O)NCC1OC1C(=O)OCc1ccccn1 ; O=C(CNC(=O)C(CS)NC(=O)C(CCc1ccc(O)cc1)NC(=O)CCCNc1cccc2c1C(=O)N(C1CCC(=O)NC1=O)C2=O)NCC1OC1C(=O)OCc1ccccn1 ; CC(=O)NCC(NC(=O)C(CCc1ccc(O)cc1)NC(=O)CCCNc1cccc2c1C(=O)N(C1CCC(=O)NC1=O)C2=O)C(=O)NCC(=O)NCC1OC1C(=O)OCc1ccccn1]

### Synthesis of methyl (Z)-4-((tert-butoxycarbonyl)amino)but-2-enoate

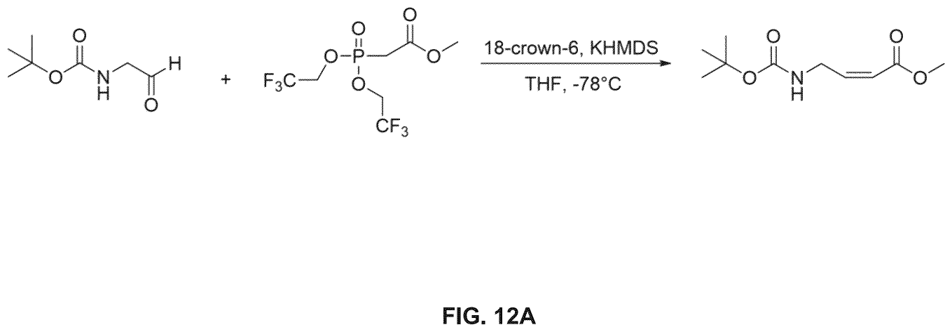

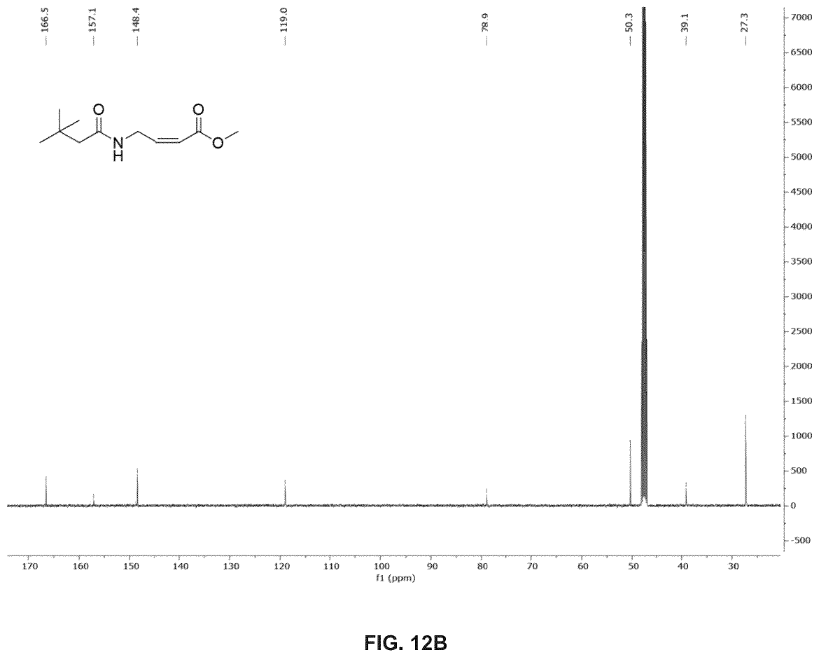

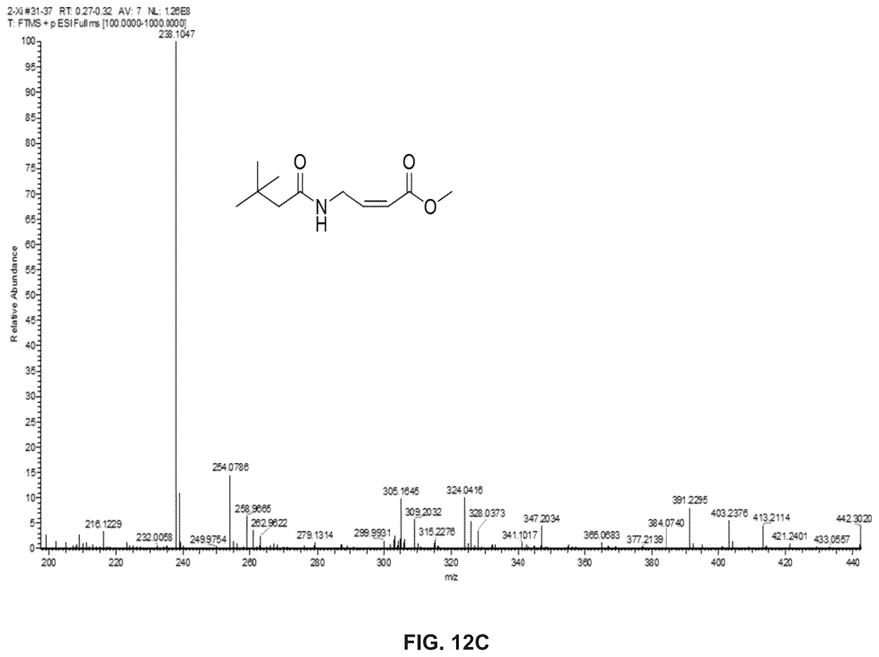

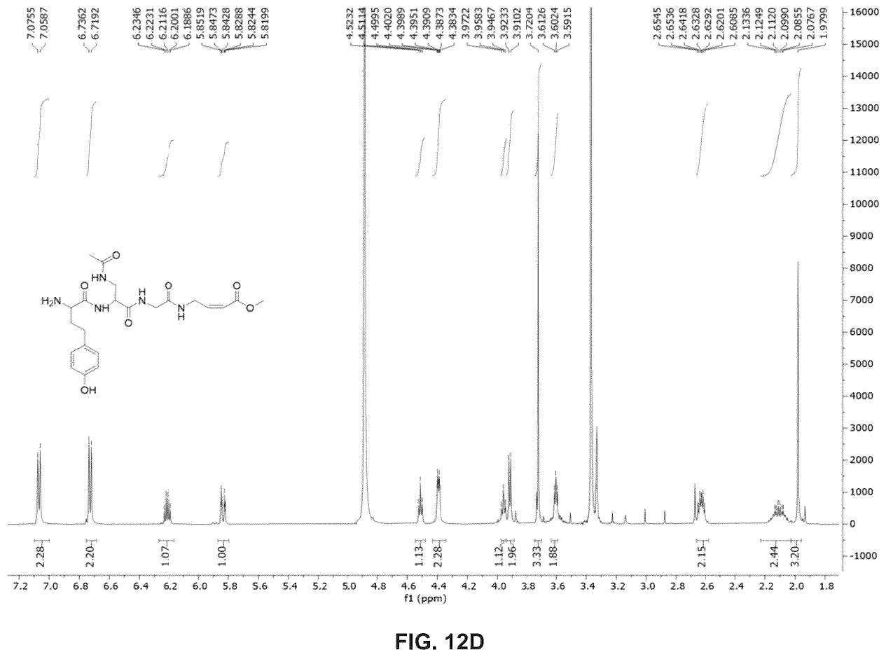

The synthesis of methyl (Z)-4-((tert-butoxycarbonyl)amino)but-2-enoate is shown in FIG. 12A. After 18-crown-6 ether (3.43 g, 13 mmol) was dissolved in 50 mL THF, potassium bis(trimethylsilyl)amide (KHMDS) (2.39 g, 12 mmol) was added. Then the mixture was cooled to −78° C. and methyl 2-[bis(2,2,2-trifluoroethoxy)phosphoryl]acetate (3.50 g, 11 mmol) was added. The solution was stirred at 0° C. for 5 min, then 2-(N-Boc)acetaldehyde (1.59 g, 10 mmol) was added and the mixture was stirred at −78° C. for 1 h. Then the cooling bath was removed, the solution was stirred at RT for 1 h, and the reaction was quenched by the addition of ethanol (30 mL). The solvent was removed under vacuum and the crude was purified by flash column chromatography (silica, 240 g, PE:EtOAc=5:1) to get product as a white solid (850 mg, 40%). ¹H NMR (methanol, 500 MHz): 6.13 (dt, J=11.5, 5.7 Hz, 1H), 5.73 (dt, J=11.5, 2.3 Hz, 1H), 4.10 (dd, J=5.8, 2.3 Hz, 1H), 3.61 (s, 3H), 1.34 (s, 9H), as shown in FIG. 12B. ¹³C-NMR (methanol, 125 MHz): 166.47, 157.10, 148.41, 119.03, 78.90, 50.34, 39.09, 27.32, as shown in FIG. 12C. HR-ESI-MS (m/z) calculated for 238.1050 (M+Na)⁺, found 238.1047 (FIG. 12D). Then, the Boc protecting group was removed using TFA/DCM (1/2, 120 min) to get methyl (Z)-4-aminobut-2-enoate.

### Synthesis of PLP-TA-2 (pep-2-ene)

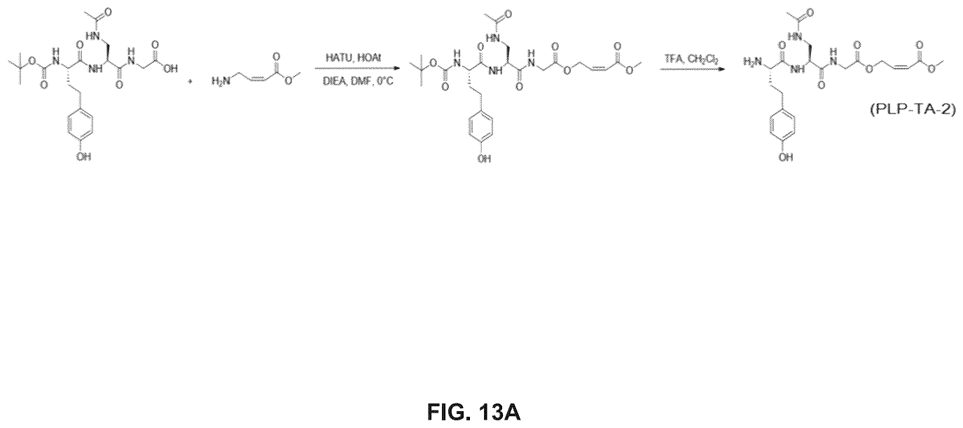

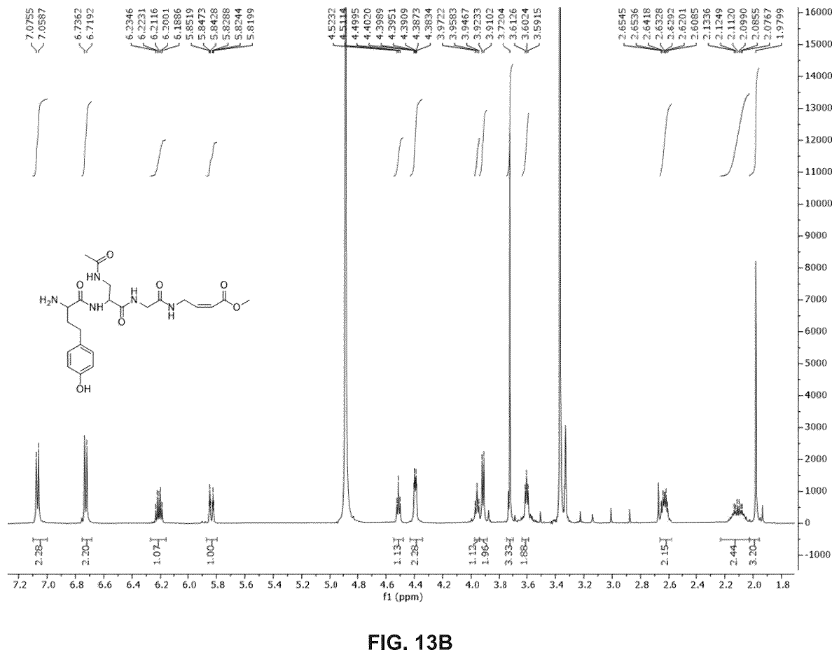

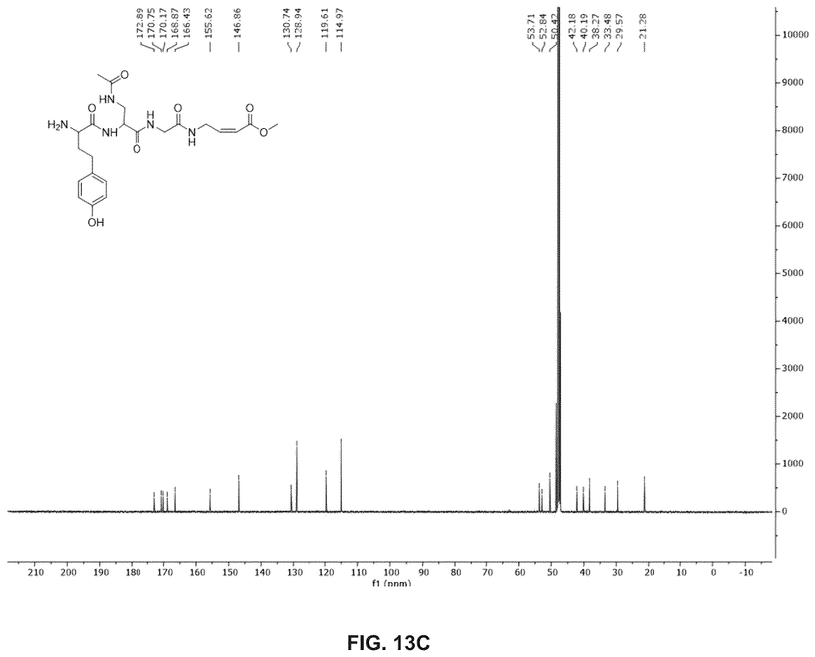

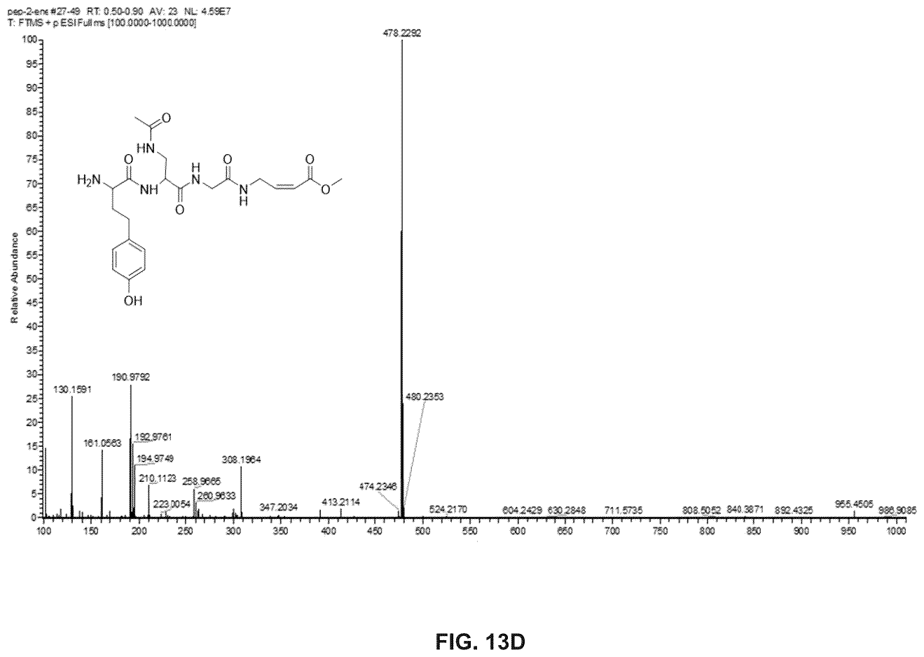

The synthesis of methyl (Z)-4-((((S)-3-acetamido-2-((S)-2-amino-4-(4-hydroxyphenyl)butanamido)propanoyl)glycyl)oxy)but-2-enoate (PLP-TA-2) is shown in FIG. 13A. To a solution of Boc-HoTyr-(Ac)Dap-G-OH (64 mg, 0.11 mmol) and methyl (Z)-4-aminobut-2-enoate (12.0 mg, 0.11 mmol) in 3 mL DMF, DIEA (87.5 μL, 0.5 mmol), HATU (76 mg, 0.2 mmol), and HOAt (3.0 mg, 0.02 mmol) were added under ice bath. The reaction was stirred at 0° C. for half an hour, then the mixture was purified with HPLC to get product as oil-like solid. Then, the Boc protecting group was removed using TFA/DCM (1/2, 120 min) to get product (48 mg, 91%). ¹H NMR (methanol-d₄, 500 MHz): 7.07 (d, J=8.4 Hz, 2H), 6.73 (d, J=8.5 Hz. 2H), 6.21 (dt, J=11.5, 5.7 Hz, 1H), 5.84 (dt, J=11.5, 2.3 Hz, 1H), 4.51 (t, J=5.9 Hz, 1H), 4.39 (dt, J=5.9, 1.7 Hz, 2H), 4.00-3.94 (m, 1H), 3.92 (d, J=6.6 Hz, 2H), 3.72 (s, 3H), 3.60 (t, J=5.3 Hz, 2H), 2.69-2.54 (m, 2H), 2.22-2.04 (m, 2H), 1.98 (s, 3H), as shown in FIG. 13B. ¹³C-NMR (methanol-d₄, 125 MHz): 172.9, 170.8, 170.2, 168.9, 166.4, 155.6, 146.9, 130.7, 128.9, 119.6, 115.0, 53.7, 52.8, 50.4, 42.2, 40.2, 38.3, 33.5, 29.6, 21.3, as shown in FIG. 13C. HR-ESI-MS (n/z) calculated for 478.2297 (M+H)⁺, found 478.2292 (FIG. 13D).

### Synthesis of PLP-5

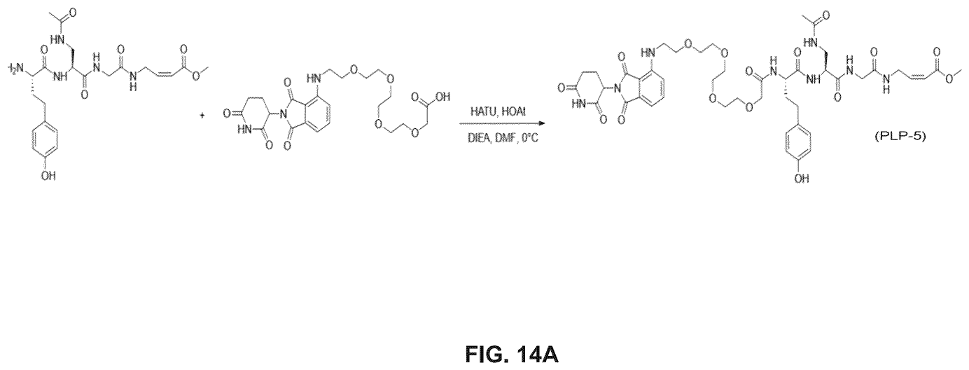

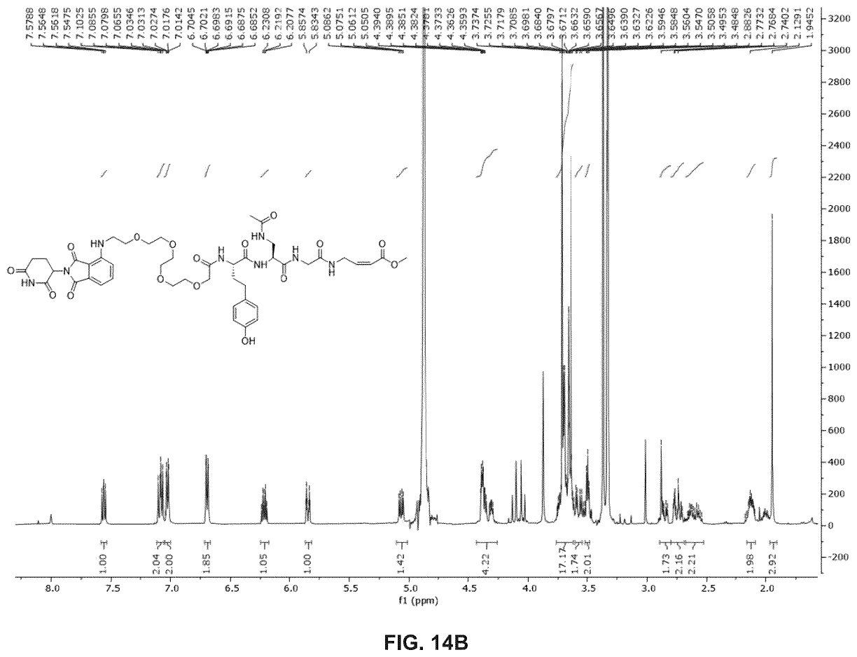

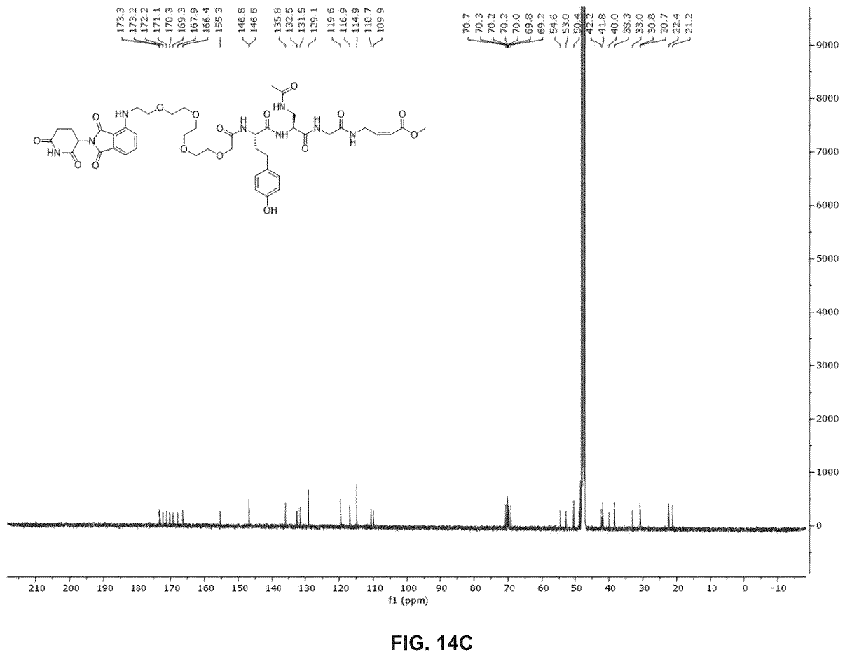

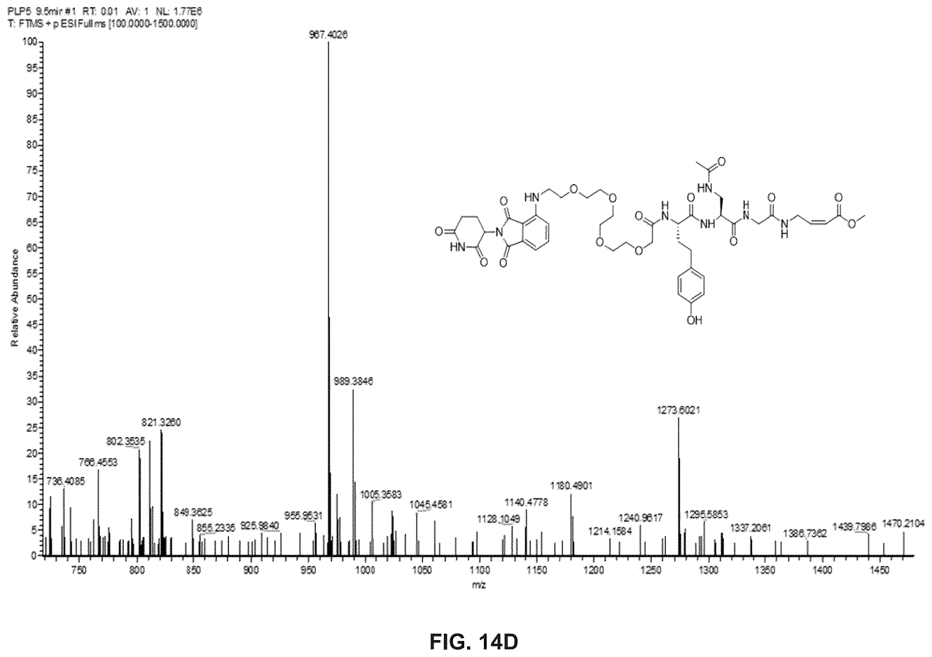

The synthesis of methyl (16S,19S,Z)-19-(acetamidomethyl)-1-((2-(2,6-dioxopiperidin-3-yl)-1,3-dioxoisoindolin-4-yl)amino)-16-(4-hydroxyphenethyl)-14,17,20,23-tetraoxo-3,6,9,12-tetraoxa-15,18,21,24-tetraazaoctacos-26-en-28-oate (PLP-5) is shown in FIG. 14A. To a solution of PLP-TA-2 (9.5 mg, 0.02 mmol) and pomalidomide-PEG₄-C—COOH (11.4 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half an hour, then the mixture was purified with HPLC (45% acetonitrile) to get product as yellow-green solid (13 mg, 68%). ¹H NMR (500 MHz, methanol-d₄) δ 7.56 (dd, J=8.6, 7.1 Hz, 1H), 7.09 (d, J=8.5 Hz. 1H), 7.07 (d, J=7.1 Hz, 1H), 7.02 (dd, J=8.5, 1.7 Hz. 2H), 6.69 (dd, J=8.5, 1.2 Hz, 2H), 6.22 (dt, J=11.5, 5.7 Hz, 1H), 5.85 (dt, J=1 1.5, 2.2 Hz, 1H), 5.07 (dd, J=12.4, 5.4 Hz, 1H), 4.41-4.33 (m, 3H), 4.31 (ddd, J=8.4, 5.5, 2.9 Hz, 1H), 3.77-3.61 (m, overlapped, 19H), 3.57 (dd, J=18.0, 5.8 Hz, 2H), 3.50 (t, J=5.3 Hz, 2H), 2.88-2.82 (m, 2H), 2.80-2.69 (m, 2H), 2.69-2.52 (m, 2H), 2.12 (d, J=5.3 Hz, 2H), 1.95 (s, 3H), as shown in FIG. 14B. ¹³C-NMR (methanol-d₄, 125 MHz): 173.3, 173.2, 172.2, 171.1, 170.3, 169.3, 167.9, 166.4, 155.3, 146.8, 146.8, 135.8, 132.5, 131.5, 129.1, 119.6, 116.9, 114.9, 110.7, 109.9, 70.7, 70.3-70.2 (overlapped), 70.0, 69.8, 69.2, 54.6, 53.0, 50.4, 42.2, 41.8, 40.0, 38.3, 33.1, 30.8, 30.7, 22.4, 21.2, as shown in FIG. 14C. HR-ESI-MS (m/z) calculated for 967.3987 (M+H)⁺, found 967.4026 (FIG. 14D).

### Synthesis of PLP-6

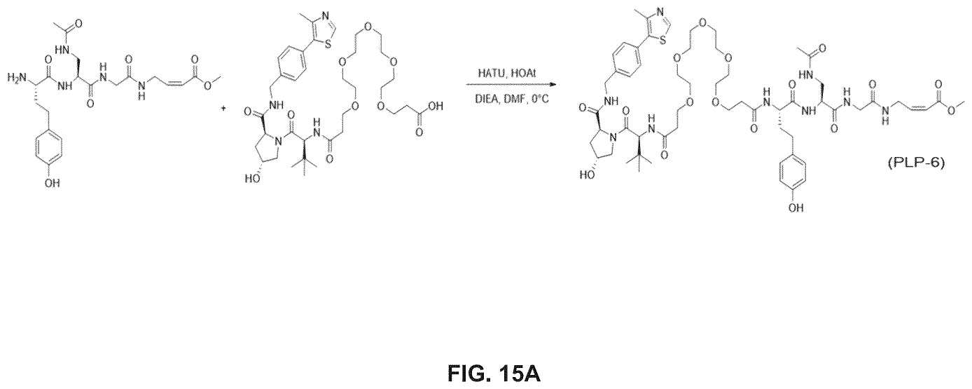

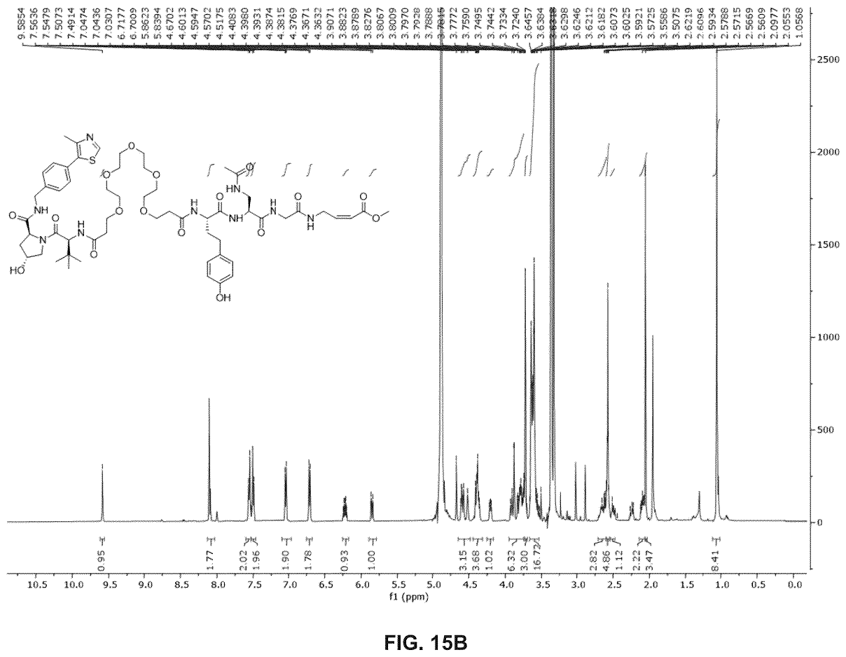

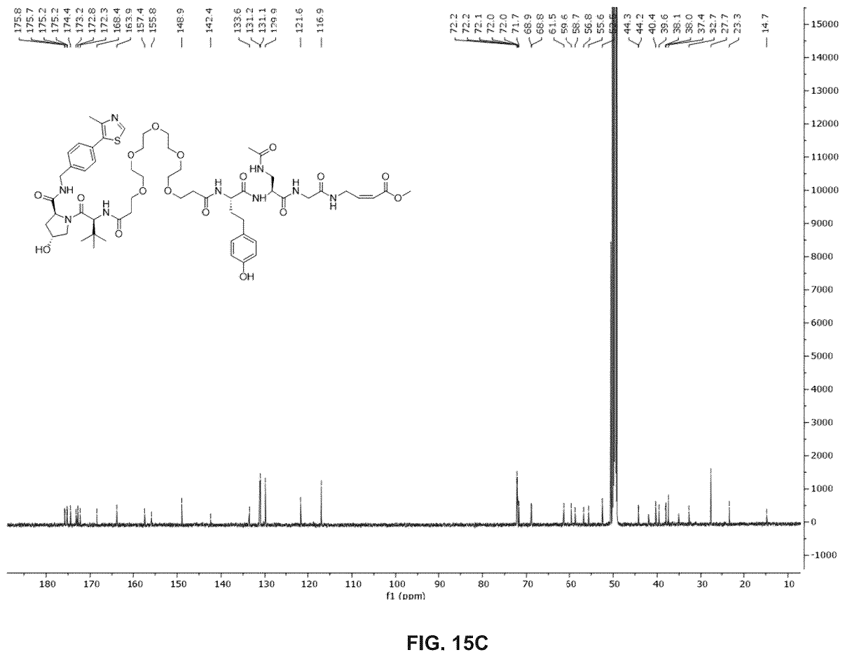

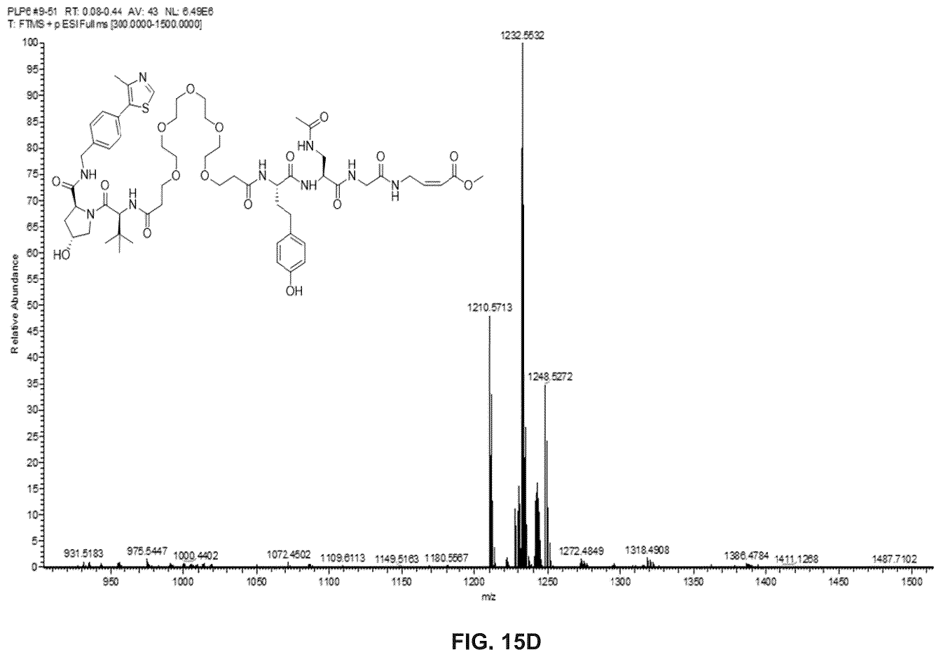

The synthesis of methyl (3S,25S,28S,Z)-28-(acetamidomethyl)-3-((2S,4R)-4-hydroxy-2-((4-(4-methylthiazol-5-yl)benzyl)carbamoyl)pyrrolidine-1-carbonyl)-25-(4-hydroxyphenethyl)-2,2-dimethyl-5,23,26,29,32-pentaoxo-8,11,14,17,20-pentaoxa-4,24,27,30,33-pentaazaheptatriacont-35-en-37-oate (PLP-6) is shown in FIG. 15A. To a solution of PLP-TA-2 (9.5 mg, 0.02 mmol) and (S,R,S)-AHPC-PEG₅-COOH (15.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half an hour, then the mixture was purified with HPLC (35% acetonitrile) to get product as a yellow solid (13.6 mg, 56%). ¹H NMR (methanol-d₄, 500 MHz): 9.59 (s, 1H), 7.56 (d, J=7.9 Hz, 2H), 7.50 (d, J=8.0 Hz, 2H), 7.04 (d, J=8.4 Hz, 2H), 6.71 (d, J=8.4 Hz, 2H), 6.22 (dt, J=11.4, 5.7 Hz, 11H), 5.85 (d, J=11.4 Hz, 11H), 4.63-4.50 (m, 3H), 4.42-4.34 (m, 4H), 4.21 (dd, J=9.3, 4.8 Hz, 1H), 3.98-3.74 (m, 7H), 3.72 (s, 3H), 3.67-3.50 (m, 18H), 2.75-2.59 (m, 3H), 2.57 (s, 3H), 2.49 (d, J=20.7 Hz, 1H), 2.28-2.20 (m, 3H), 2.06 (s, 3H), 1.06 (s, 9H), as shown in FIG. 15B. ¹³C-NMR (methanol-d₄, 125 MHz): 175.8, 175.7, 175.2, 175.2, 174.4, 173.2, 172.8, 172.3, 168.4, 163.9, 157.4, 155.8, 148.9, 142.4, 133.6, 131.2, 131.1, 129.9, 121.6, 116.9, 72.2-72.1 (overlapped), 72.0, 72.0, 71.7, 68.9, 68.8, 61.5, 59.6, 58.7, 56.8, 55.6, 52.5, 44.3, 44.3, 40.4, 39.6, 38.1, 38.0, 37.4, 32.7, 27.7, 23.3, 14.7, as shown in FIG. 15C. HR-ESI-MS (m/z) calculated for 1232.5350 (M+Na)⁺. found 1232.5532 (FIG. 15D).

Pomalidomide-C₃-COOH can be replaced with any compounds of the following: (S,R,S)-AHPC-PEGₙ-butyl COOH (n=2-10), pomalidomide-PEGₙ-COOH (n=2-10), or pomalidomide-Cₙ-COOH (n=3-10).

### Activity

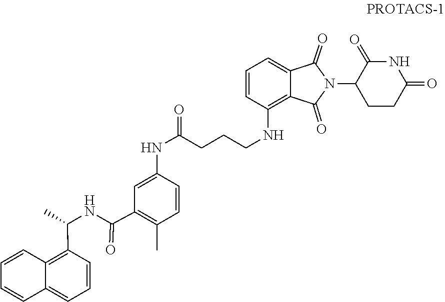 [SMILES: Cc1ccc(NC(=O)CCCNc2cccc3c2C(=O)N(C2CCC(=O)NC2=O)C3=O)cc1C(=O)N[C@@H](C)c1cccc2ccccc12]

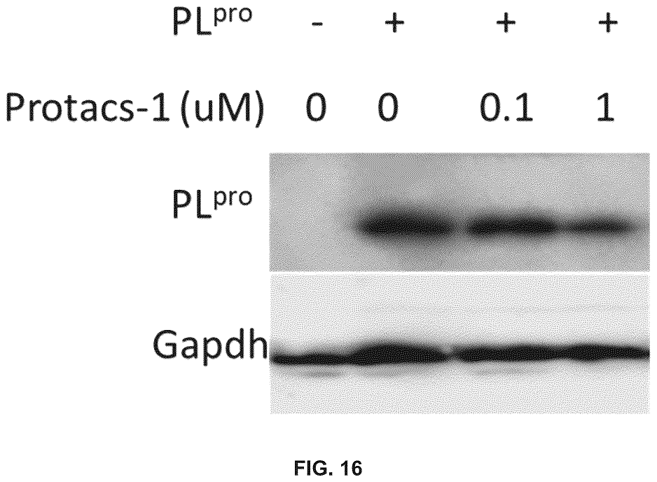

FIG. 16 shows the activity of PROTACS-1 on PLpro expression.

### Synthesis of Boc-AVL-OH

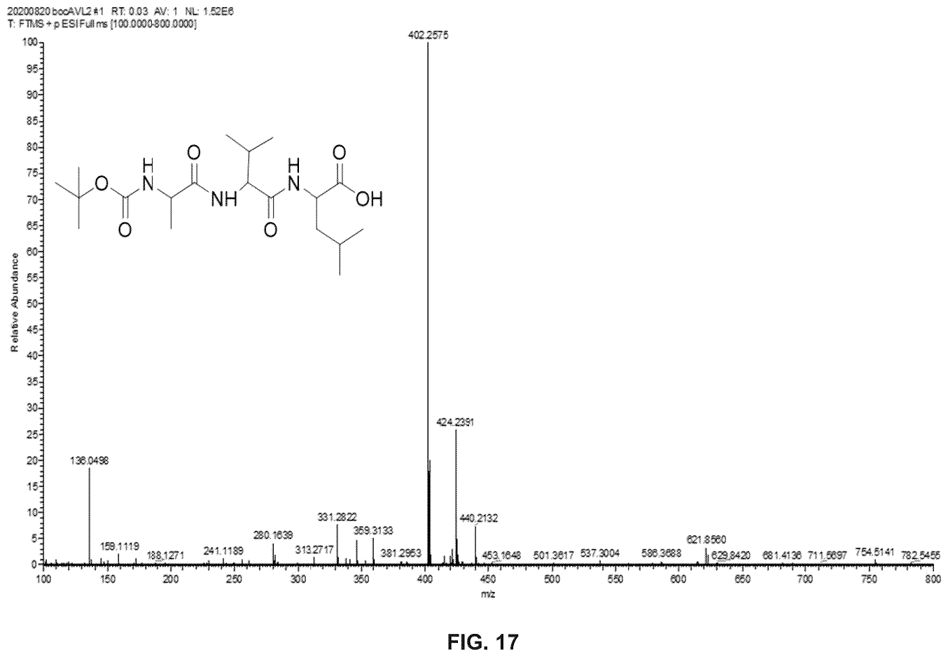

Boc-AVL-OH was synthesized according to the solid phase peptide synthesis method, as shown in FIG. 17A. Firstly, Fmoc-Val-OH (2.5 eq) was attached to H-Leu-Cl-Trt resin (0.687 mmol/g, 1.5 g) using HATU (2.5 eq), DIEA (5.0 eq), and HOAt (0.5 eq) in DMF as coupling reagents. Then, the Fmoc protecting group was removed using 20% piperidine in DMF (2 cycles: 5, and 15 min). Boc-Ala-OH (2.5 eq) was coupled to the H2N-(Ac)Dap-G-resin using HATU (2.5 eq), DIEA (5.0 eq), and HOAt (0.5 eq) in DMF. Then, the resin was washed five times with DMF, three times with DCM, and three times with McOH, and dried under vacuum. Next, the peptide was removed from the resin with a mixture of trifluoroethanol/DCM (v/v, 20/80), precipitated in Et₂O, purified on HPLC, and lyophilized. The purity of peptide was confirmed using HPLC. HR-ESI-MS (m/z) calculated for 402.2599 (M+H)⁺, found 402.2575 (FIG. 17B).

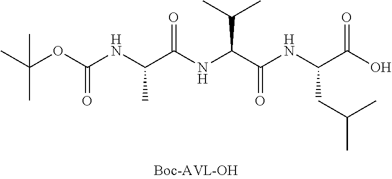 [SMILES: CC(C)C[C@H](NC(=O)[C@@H](NC(=O)[C@H](C)NC(=O)OC(C)(C)C)C(C)C)C(=O)O]

### Synthesis of MP-M-1

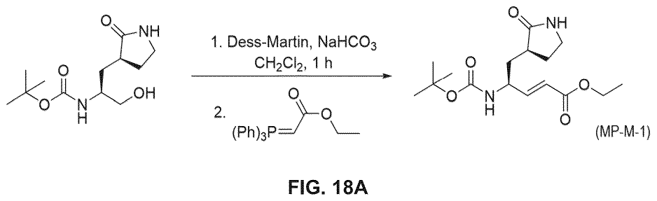

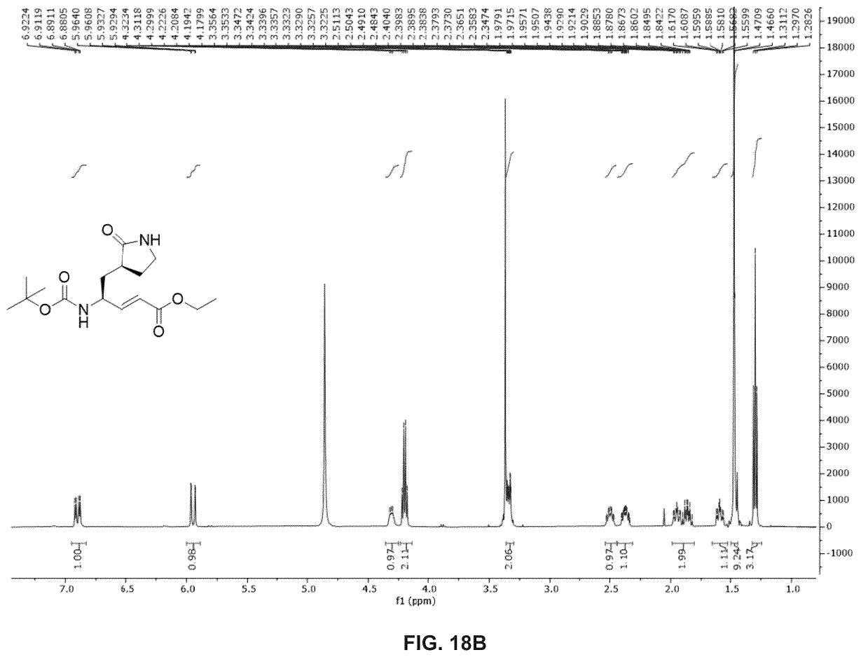

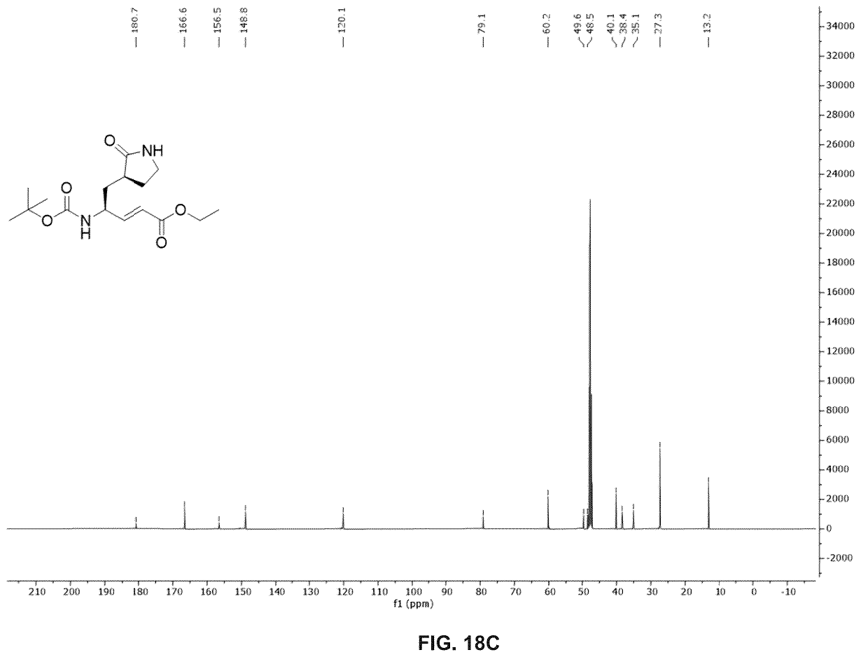

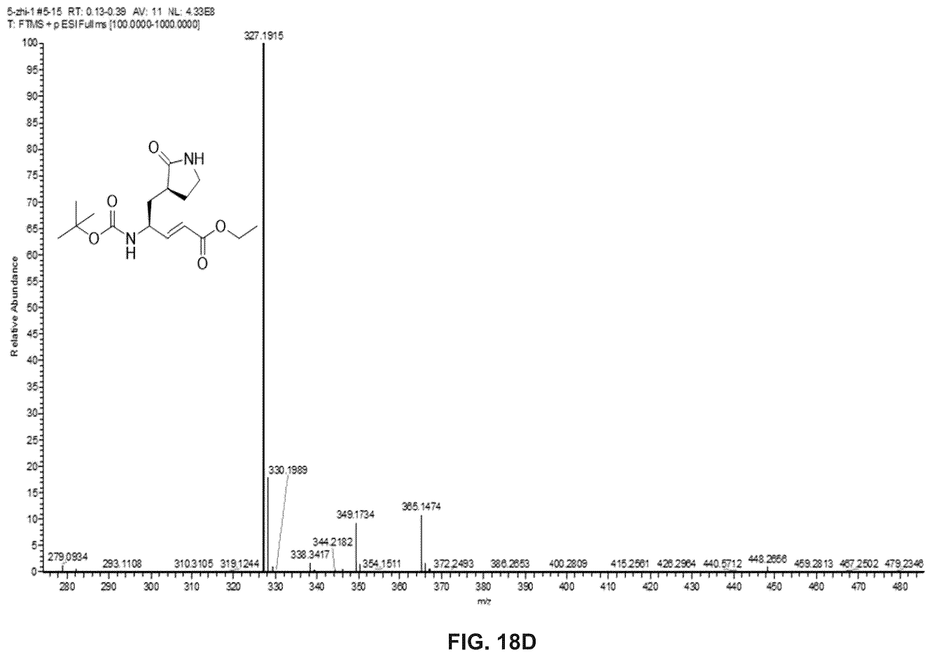

The synthesis of ethyl (S,E)-4-((tert-butoxycarbonyl)amino)-5-((S)-2-oxopyrrolidin-3-yl)pent-2-enoate (MP-M-1), is shown in FIG. 18A. (S)-3-[(S)-2-(Boc-amino)-3-hydroxypropyl]-2-pyrrolidinone (77.6 mg, 0.3 mmol) was dissolved in DCM (5 mL) then Dess-Martin periodinane (128.0 mg, 0.3 mmol) and NaHCO₃ (26.0 mg. 0.3 mmol) was added. The mixture was stirred at RT for 60 min. Ethyl 2-(triphenylphosphoranylidene)acetate (69.6 mg, 0.3 mmol) was added and the solution was stirred at RT for 24 h. The solvent was removed by rotary evaporation and the crude was purified by HPLC (45% acetonitrile) to yield the product as a white solid (104.0 mg, 75%). ¹H NMR (methanol-d₄, 500 MHz): 6.90 (dd, J=15.7, 5.3 Hz, 1H), 5.95 (dd, J=15.7, 1.6 Hz, 1H), 4.39-4.27 (m, 1H), 4.20 (q, J=7.1 Hz, 2H), 3.36-3.27 (m, 2H), 2.50 (qd, J=9.2, 3.5 Hz, 1H), 2.42-2.28 (m, 1H), 1.95 (ddd, J=14.7, 11.1, 3.8 Hz, 1H), 1.91-1.81 (m, 1H), 1.59 (ddd, J=14.3, 10.6, 4.1 Hz, 1H), 1.47 (s, 9H), 1.30 (t, J=7.2 Hz, 3H), as shown in FIG. 18B. ¹³C-NMR (methanol-d₄, 125 MHz): 180.7, 166.6, 156.5, 148.8, 120.1, 79.1, 60.2, 49.6, 48.5, 40.1, 38.4, 35.1, 27.4, 13.2), as shown in FIG. 18C. HR-ESI-MS (m/z) calculated for 327.1915 (M+H)*, found 327.1915 (FIG. 18D). Then, the Boc protecting group was removed using TFA/DCM (1/2, 120 min) to get ethyl (S,E)-4-amino-5-((S)-2-oxopyrrolidin-3-yl)pent-2-enoate.

### Synthesis of MP-M-2

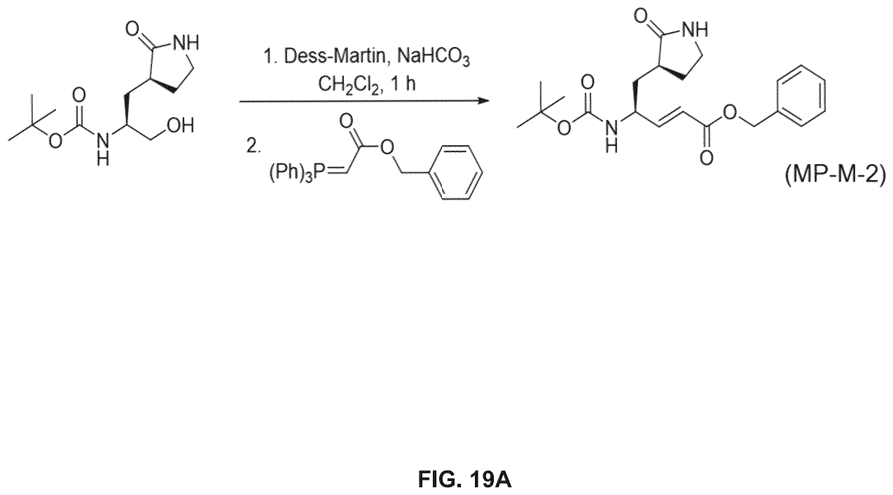

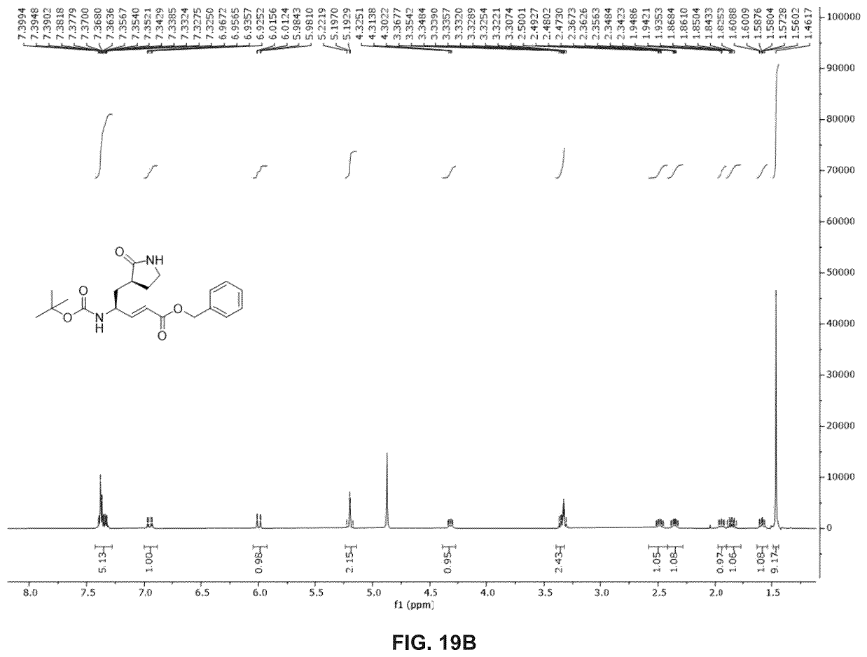

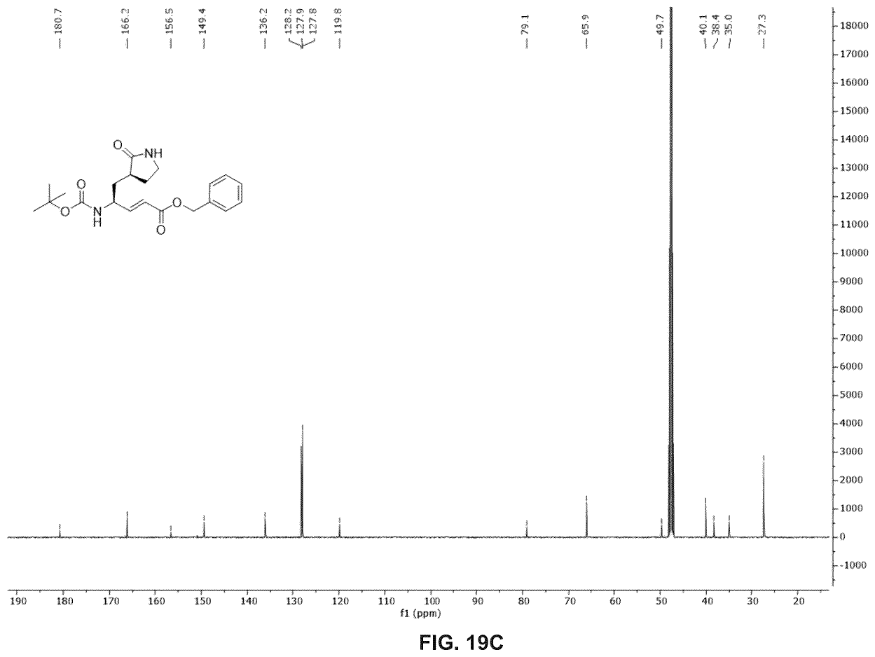

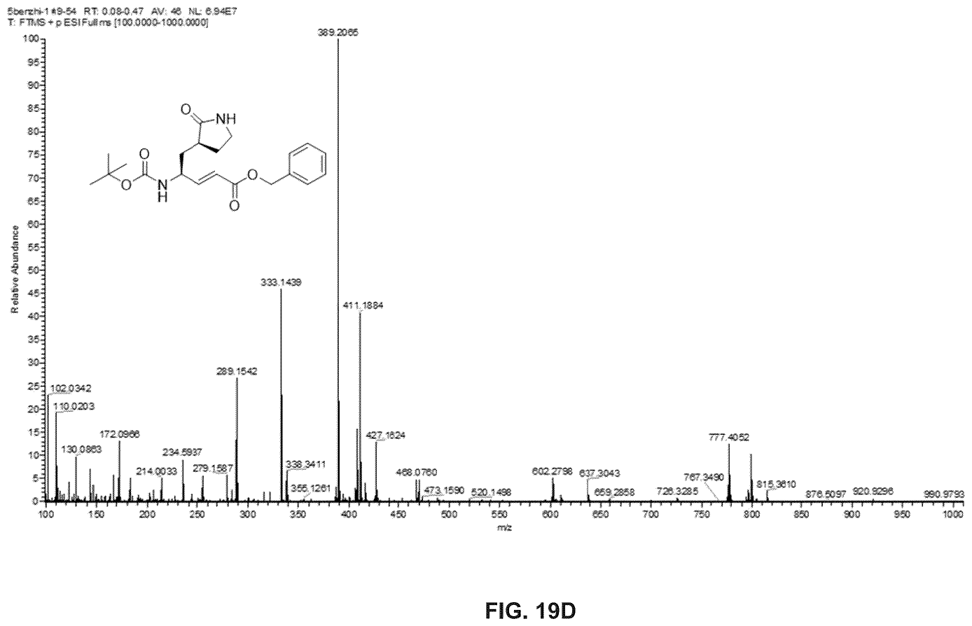

The synthesis of benzyl (S,E)-4-((tert-butoxycarbonyl)amino)-5-((S)-2-oxopyrrolidin-3-yl)pent-2-enoate (MP-M-2) is shown in FIG. 19A. (S)-3-[(S)-2-(Boc-amino)-3-hydroxypropyl]-2-pyrrolidinone (51.7 mg, 0.2 mmol) was dissolved in DCM (3 mL) then Dess-Martin periodinane (85.0 mg, 0.2 mmol) and NaHCO₃ (17.0 mg. 0.2 mmol) were added. The mixture was stirred at RT for 60 min. Benzyl 2-(triphenylphosphoranylidene)acetate (82.0 mg, 0.2 mmol) was added and the solution was stirred at RT for 24 h. The solvent was removed by rotary evaporation and the crude was purified by HPLC (55% acetonitrile) to yield the product as a white solid (56.0 mg, 72%). ¹H NMR (methanol-d₄, 500 MHz): 7.44-7.28 (m, 5H), 6.95 (dd, J=15.7, 5.3 Hz, 1H), 6.00 (dd, J=15.7, 1.6 Hz, 1H), 5.19 (d, J=2.0 Hz, 2H), 4.31 (dt, J=10.5, 4.6 Hz, 2H), 3.34-3.24 (m, 2H), 2.49 (qd, J=9.6, 9.0, 3.4 Hz, 1H), 2.42-2.31 (m, 1H), 1.94 (ddd, J=14.6, 11.2, 3.9 Hz, 1H), 1.85 (dq, J=12.6, 8.9 Hz, 1H), 1.59 (td, J=10.3, 5.1 Hz, 1H), 1.46 (s, 9H), as shown in FIG. 19B. ¹³C-NMR (methanol-d₄, 125 MHz): 180.7, 166.2, 156.6, 149.4, 136.2, 128.2, 127.9, 127.8, 119.8, 79.1, 65.9, 49.7, 40.1, 38.4, 35.0, 27.3, as shown in FIG. 19C. HR-ESI-MS (m/z) calculated for 389.2072 (M+H)⁺, found 389.2065 (FIG. 19D). Then, the Boc protecting group was removed using TFA/DCM (1/2, 120 min) to get benzyl (S,E)-4-amino-5-((S)-2-oxopyrrolidin-3-yl)pent-2-enoate.

### Synthesis of MP-TA-1

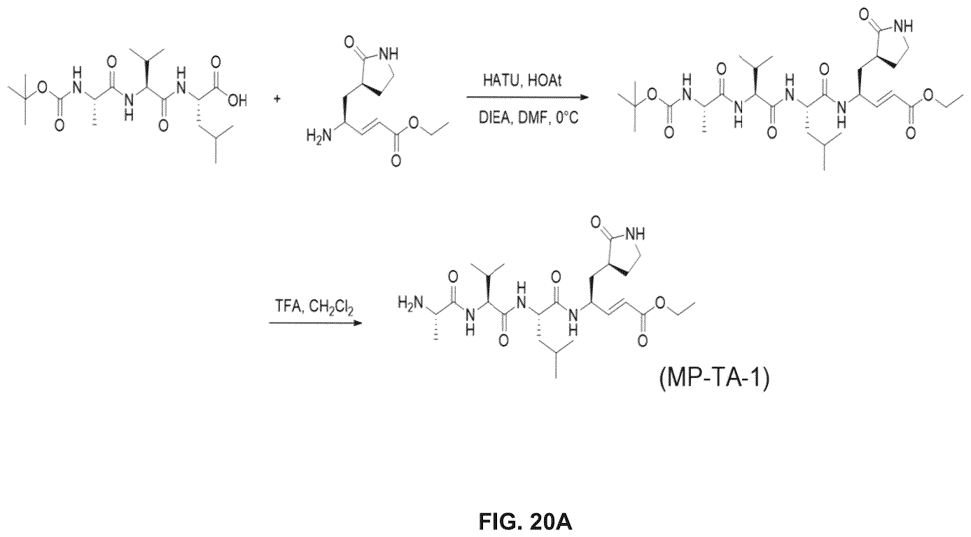

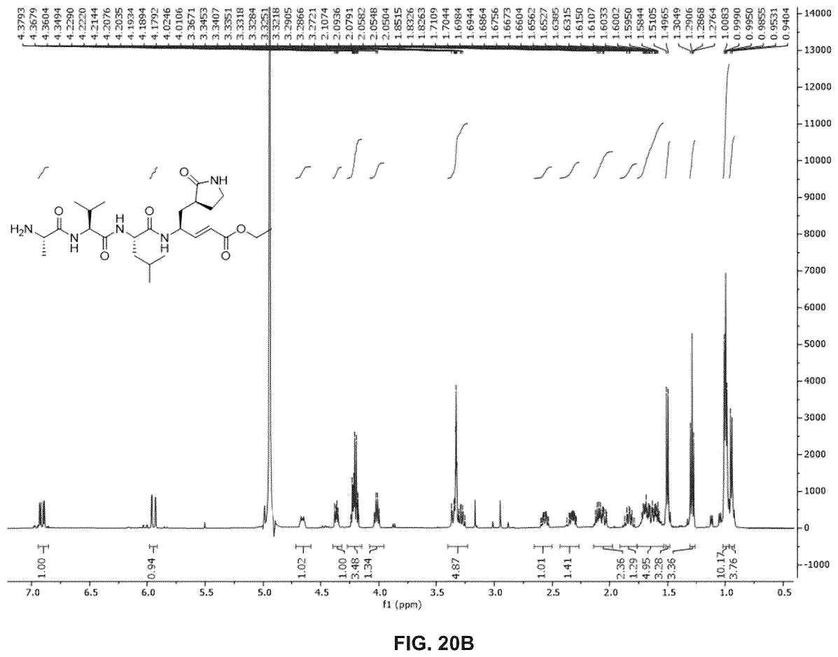

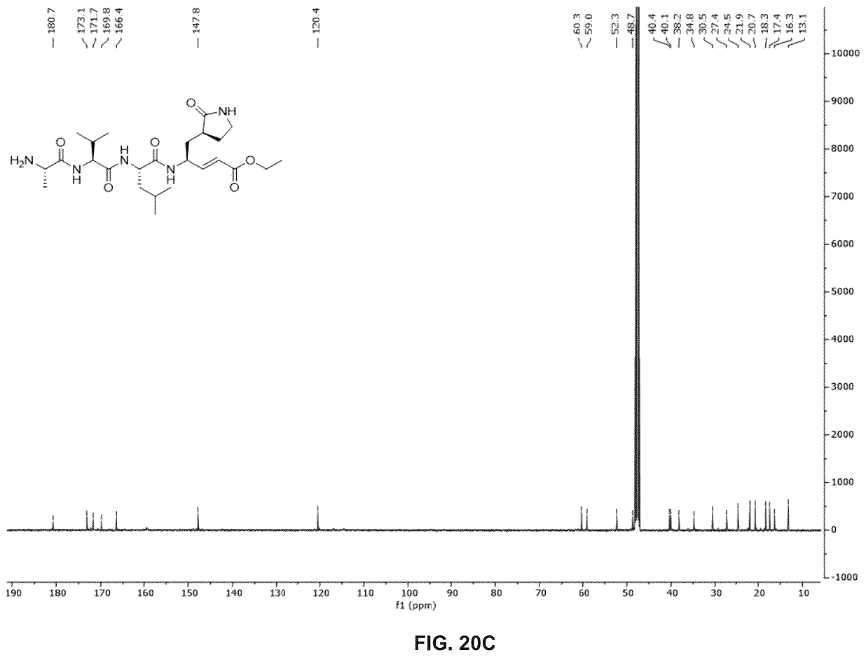

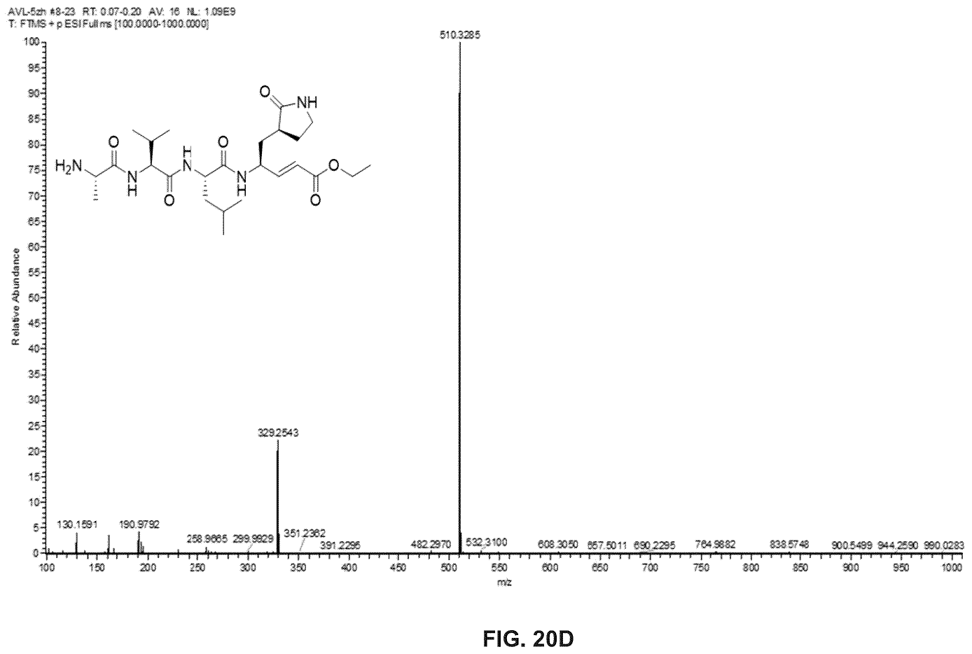

The synthesis of ethyl (S,E)-4-((S)-2-((S)-2-((S)-2-aminopropanamido)-3-methylbutanamido)-4-methylpentanamido)-5-((S)-2-oxopyrrolidin-3-yl)pent-2-enoate (MP-TA-1), is shown in FIG. 20A. To a solution of Boc-AVL-OH (61.0 mg, 0.15 mmol) and ethyl (S,E)-4-amino-5-((S)-2-oxopyrrolidin-3-yl)pent-2-enoate (33.9 mg, 0.15 mmol) in 3 mL DMF, DIEA (131 μL, 0.75 mmol), HATU (114 mg, 0.3 mmol), and HOAt (4.5 mg, 0.03 mmol) were added under ice bath. The reaction was stirred at 0° C. for half hour, then the mixture was purified with HPLC to get product as a white solid. Then, the Boc protecting group was removed using TFA/DCM (1/2, 120 min) to get product (63.5 mg, 83%). 1H NMR (methanol-d₄, 500 MHz): ¹H NMR (500 MHz, methanol-d₄): 6.91 (dd, J=15.7, 5.2 Hz, 1H), 5.94 (dd, J=15.7, 1.6 Hz, 1H), 4.68-4.63 (m, 1H), 4.36 (dd, J=9.3, 5.6 Hz, 1H), 4.25-4.13 (m, overlapped, 3H), 4.02 (q, J=6.9 Hz, 1H), 3.42-3.22 (m, overlapped, 2H), 2.57 (tdd, J=10.4, 8.4, 3.8 Hz, 1H), 2.41-2.24 (m, 1H), 2.17-1.99 (m, 2H), 1.92-1.77 (m, 1H), 1.75-1.55 (overlapped, 4H), 1.50 (d, J=7.0 Hz, 3H), 1.29 (t, J=7.1 Hz, 3H), 1.00 (overlapped, 9H), 0.95 (d, J=6.3 Hz, 3H), as shown in FIG. 20B. ¹³C-NMR (methanol-d₄, 125 MHz): 180.7, 173.1, 171.7, 169.8, 166.4, 147.8, 120.4, 60.3, 59.0, 52.3, 48.7, 40.4, 40.1, 38.2, 34.8, 30.5, 27.4, 24.6, 21.9, 20.7, 18.3, 17.4, 16.3, 13.1, as shown in FIG. 20C. HR-ESI-MS (m/z) calculated for 510.3287 (M+H)*, found 510.3285 (FIG. 20D).

### Synthesis of MP-TA-2

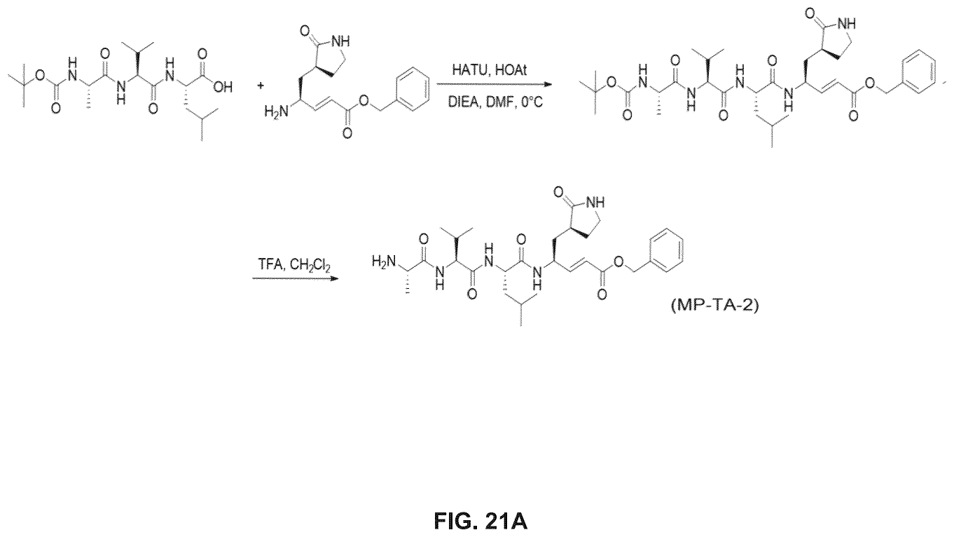

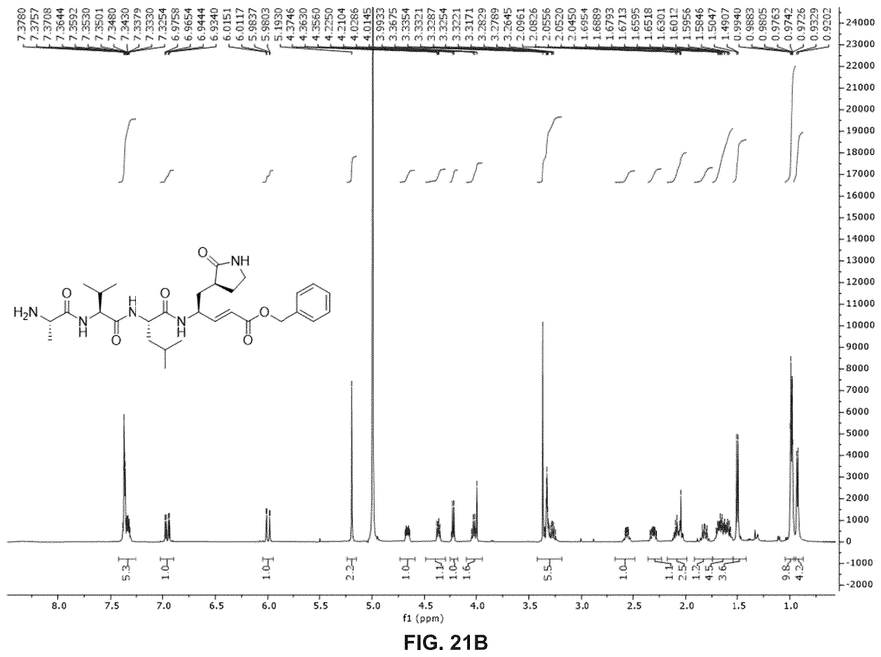

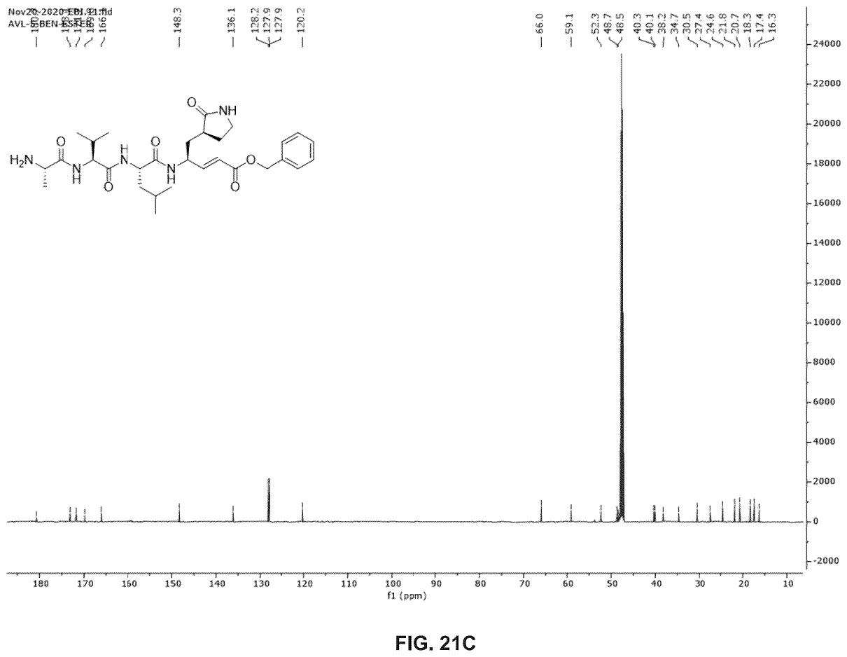

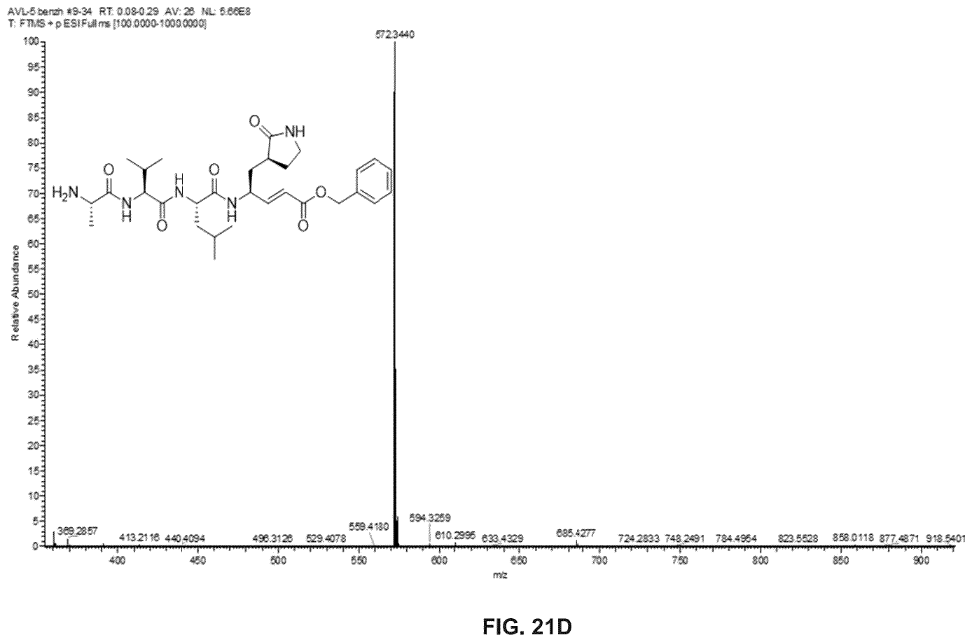

The synthesis of benzyl (S,E)-4-((S)-2-((S)-2-((S)-2-aminopropanamido)-3-methylbutanamido)-4-methylpentanamido)-5-((S)-2-oxopyrrolidin-3-yl)pent-2-enoate (MP-TA-2), as shown in FIG. 21A. To a solution of Boc-AVL-OH (61 mg, 0.15 mmol) and benzyl (S,E)-4-amino-5-((S)-2-oxopyrrolidin-3-yl)pent-2-enoate (41.5 mg, 0.144 mmol) in 3 mL DMF, DIEA (131 μL, 0.75 mmol), HATU (114 mg. 0.3 mmol), and HOAt (4.5 mg, 0.03 mmol) were added under ice bath. The reaction was stirred at 0° C. for half hour, then the mixture was purified with HPLC to get product as oil-like solid. Then, the Boc protecting group was removed using TFA/DCM (1/2, 120 min) to get product (70 mg, 85%). ¹H NMR (methanol-d₄, 500 MHz): ¹H NMR (500 MHz, methanol-d₄): 7.40-7.28 (m, 5H), 6.95 (dd, J=15.7, 5.2 Hz, 1H), 6.00 (dd, J=15.7, 1.7 Hz, 1H), 5.19 (s, 2H), 4.68-4.64 (m, 1H), 4.36 (dd, J=9.3, 5.7 Hz, 1H), 4.22 (d, J=7.3 Hz, 1H), 4.02 (q, J=7.0 Hz, 1H), 3.38-3.21 (m, 2H), 2.59-2.53 (m, 1H), 2.34-2.28 (m, 1H), 2.14-1.96 (m, 2H), 1.86-1.76 (m, 1H), 1.73-1.55 (m, 4H), 1.50 (d, J=7.0 Hz, 3H), 1.06-0.96 (m, 9H), 0.93 (d, J=6.3 Hz, 3H), as shown in FIG. 21B. ¹³C-NMR (methanol-d₄, 125 MHz): 180.7, 173.1, 171.7, 169.8, 166.1, 148.3, 136.1, 128.2, 127.9, 127.9, 120.2, 66.0, 59.1, 52.3, 48.7, 48.5, 40.3, 40.1, 38.2, 34.7, 30.5, 27.4, 24.6, 21.8, 20.7, 18.3, 17.4, 16.3, as shown in FIG. 21C. HR-ESI-MS (m/z) calculated for 572.3445 (M+H)⁺, found 572.3440 (FIG. 21D).

### Synthesis of MP-TA-3

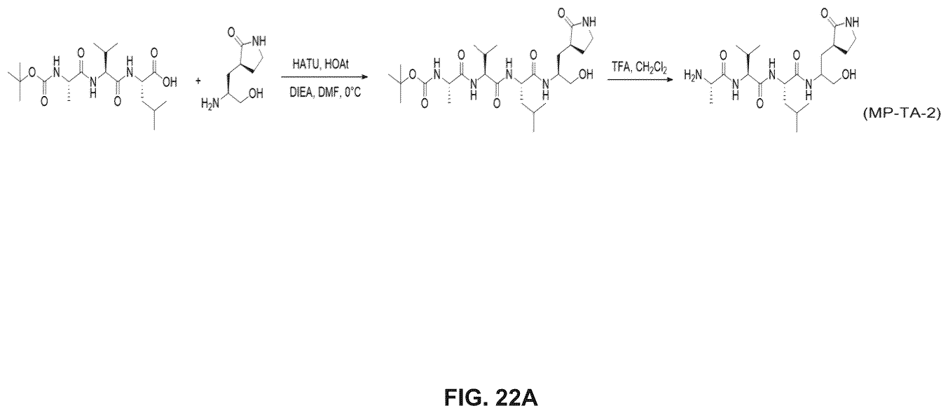

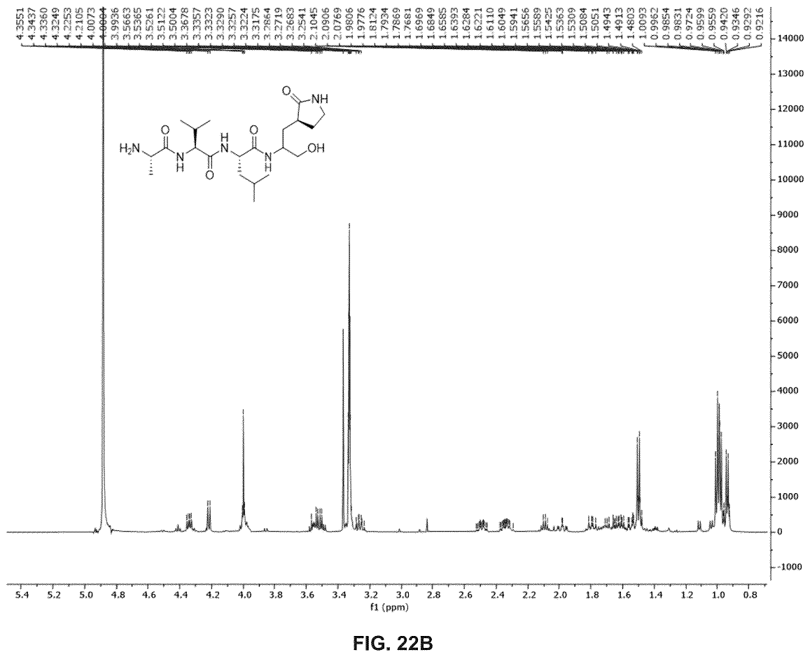

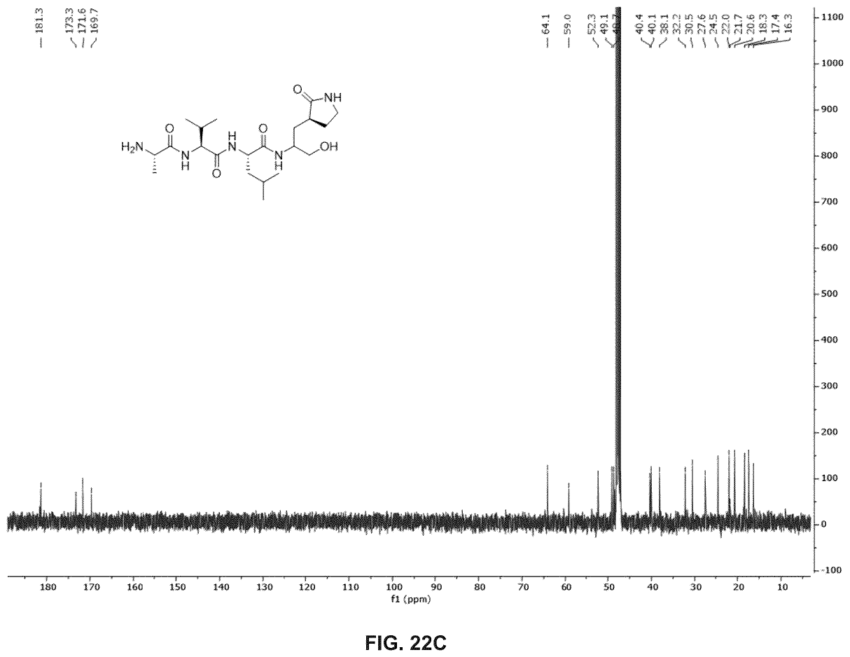

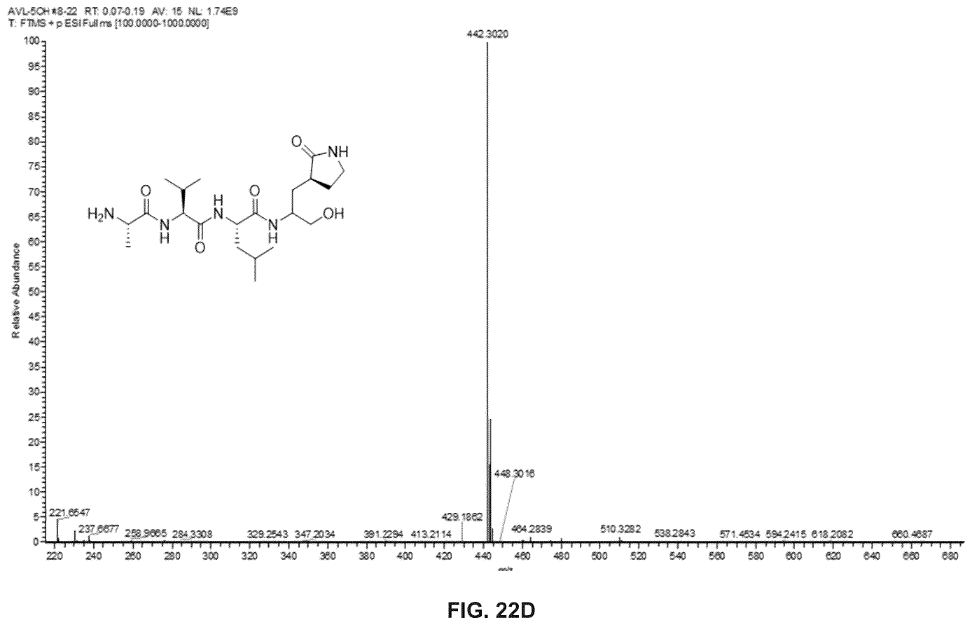

The synthesis of (2S)-2-((S)-2-((S)-2-aminopropanamido)-3-methylbutanamido)-N-(1-hydroxy-3-((S)-2-oxopyrrolidin-3-yl)propan-2-yl)-4-methylpentanamide (MP-TA-3) is shown in FIG. 22A. To a solution of Boc-AVL-OH (40 mg, 0.1 mmol) and (S)-3-((S)-2-amino-3-hydroxypropyl)pyrrolidin-2-one (16.0 mg, 0.1 mmol) in 3 mL DMF, DIEA (87.5 μL, 0.5 mmol), HATU (76 mg, 0.2 mmol), and HOAt (3.0 mg, 0.02 mmol) were added under ice bath. The reaction was stirred at 0° C. for half hour, then the mixture was purified with HPLC to get product as oil-like solid. Then, the Boc protecting group was removed using TFA/DCM (1/2, 120 min) to get product (32 mg, 73%). ¹H NMR (methanol-d₄, 500 MHz): 4.34 (dd, J=9.5, 5.6 Hz, 1H), 4.22 (d, J=7.4 Hz, 1H), 4.02-3.97 (m, 2H), 3.60-3.46 (m, 2H), 3.29-3.23 (m, 2H), 2.59-2.42 (m, 1H), 2.39-2.26 (m, 1H), 2.14-2.05 (m, 1H), 1.98 (d, J=1.5 Hz, 1H), 1.84-1.55 (m, 5H), 1.52-1.48 (m, 3H), 1.00-0.98 (m, 9H), 0.93 (d, J=6.5 Hz. 3H), as shown in FIG. 22B. ¹³C-NMR (methanol-d₄, 125 MHz): 181.3, 173.3, 171.6, 169.7, 64.1, 59.0, 52.3, 49.1, 48.7, 40.4, 40.1, 38.1, 32.2, 30.5, 27.6, 24.5, 22.0, 21.7, 20.6, 18.3, 17.4, 16.3, as shown in FIG. 22C. HR-ESI-MS (m/z) calculated for 442.3025 (M+H)⁺, found 442.3020 (FIG. 22C).

### Synthesis of MP-TA-4

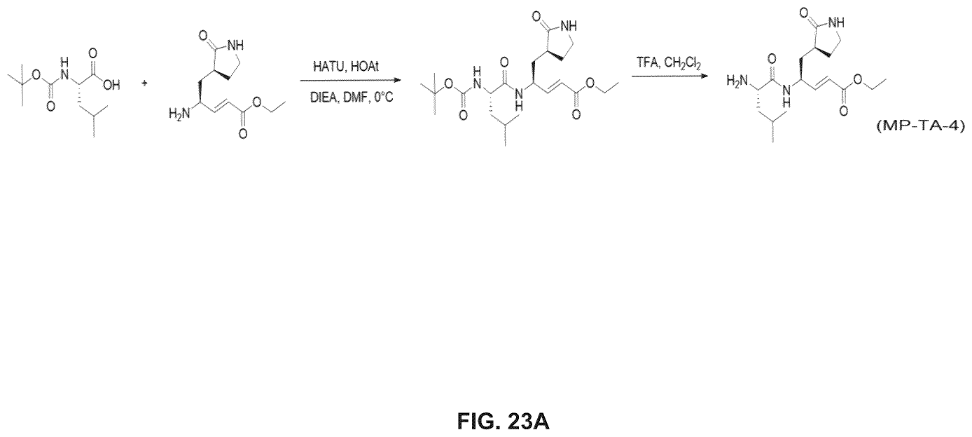

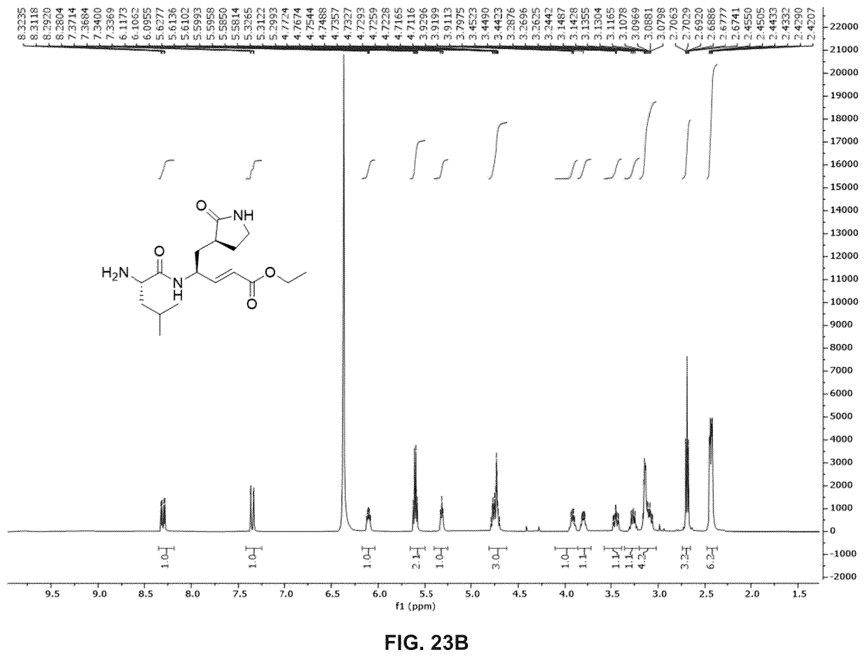

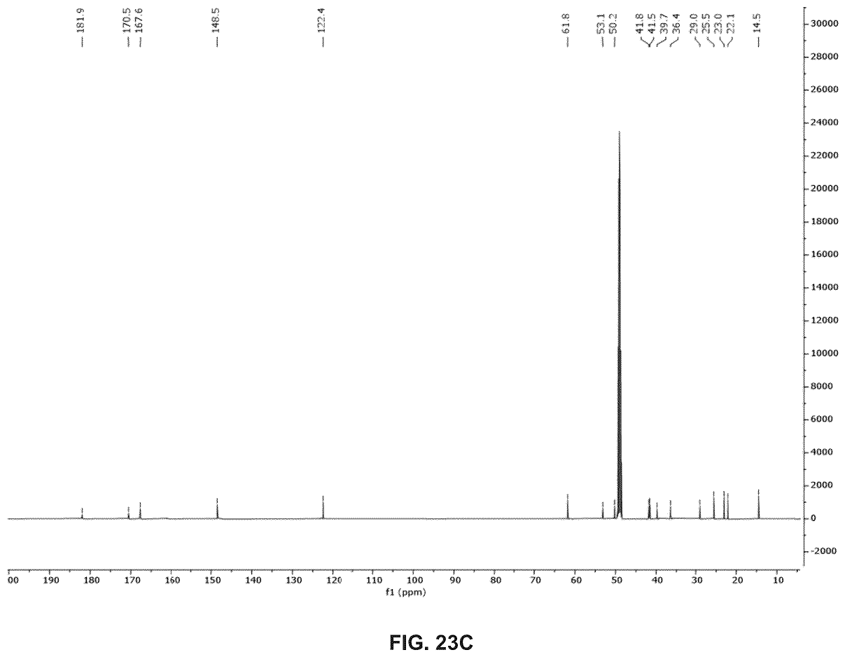

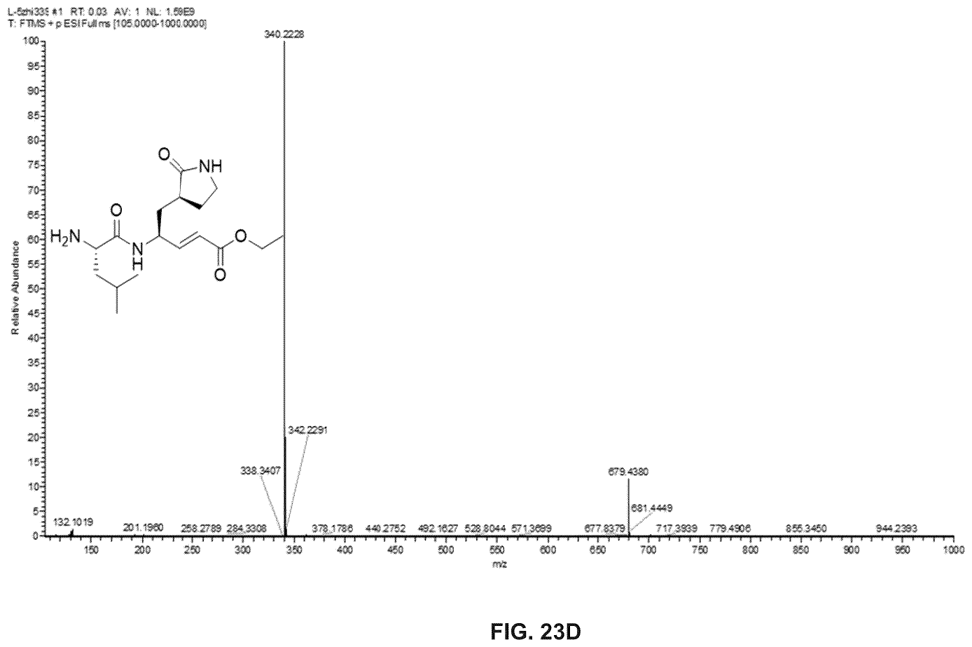

The synthesis of ethyl (S,E)-4-((S)-2-amino-4-methylpentananido)-5-((S)-2-oxopyrrolidin-3-yl)pent-2-enoate (MP-TA-4) is shown in FIG. 23A. To a solution of Boc-Leu-OH (23 mg, 0.1 mmol) and ethyl (S,E)-4-amino-5-((S)-2-oxopyrrolidin-3-yl)pent-2-enoate (22.5 mg, 0.1 mmol) in 3 mL DMF, DIEA (87.5 p L, 0.5 mmol), HATU (76 mg, 0.2 mmol), and HOAt (3.0 mg, 0.02 mmol) were added under ice bath. The reaction was stirred at 0° C. for half hour, then the mixture was purified with HPLC to get product as oil-like solid. Then, the Boc protecting group was removed using TFA/DCM (1/2, 120 min) to get product (27 mg, 80%). ¹H NMR (methanol-d₄, 500 MHz): ¹H NMR (500 MHz, methanol-d₄) δ 6.90 (dd, J=15.7, 5.3 Hz, 11H), 5.95 (dd, J=15.7, 1.6 Hz, 1H), 4.39-4.27 (m, 11H), 4.20 (q, J=7.1 Hz, 2H), 3.36-3.27 (m, 2H), 2.50 (qd, J=9.2, 3.5 Hz, 1H), 2.42-2.28 (m, 1H), 1.95 (ddd, J=14.7, 11.1, 3.8 Hz, 11H), 1.91-1.81 (m, 1H), 1.59 (ddd, J=14.3, 10.6, 4.1 Hz, 1H), 1.47 (s, 9H), 1.30 (t, J=7.2 Hz, 3H), as shown in FIG. 23B. ¹³C-NMR (methanol-d₄, 125 MHz): 180.7, 166.6, 156.5, 148.8, 120.1, 79.1, 60.2, 49.6, 48.5, 40.1, 38.4, 35.1, 27.4, 13.2, as shown in FIG. 23C. HR-ESI-MS (m/z) calculated for 340.2232 (M+H)+, found 340.2228 (FIG. 23D).

### Synthesis of MP-5

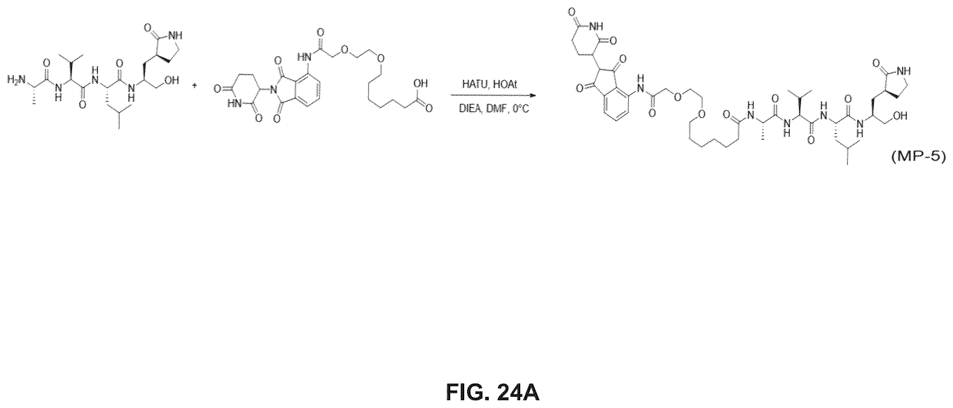

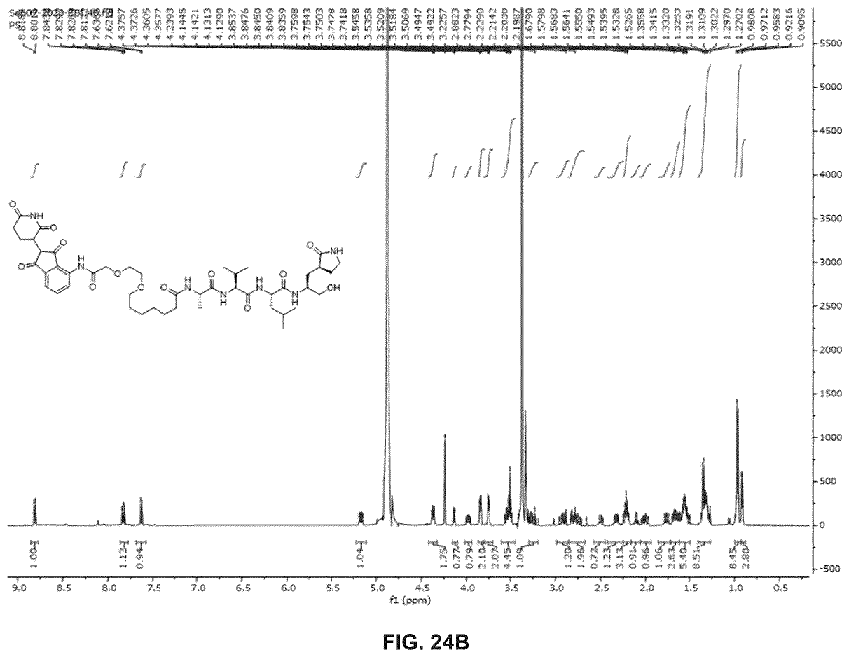

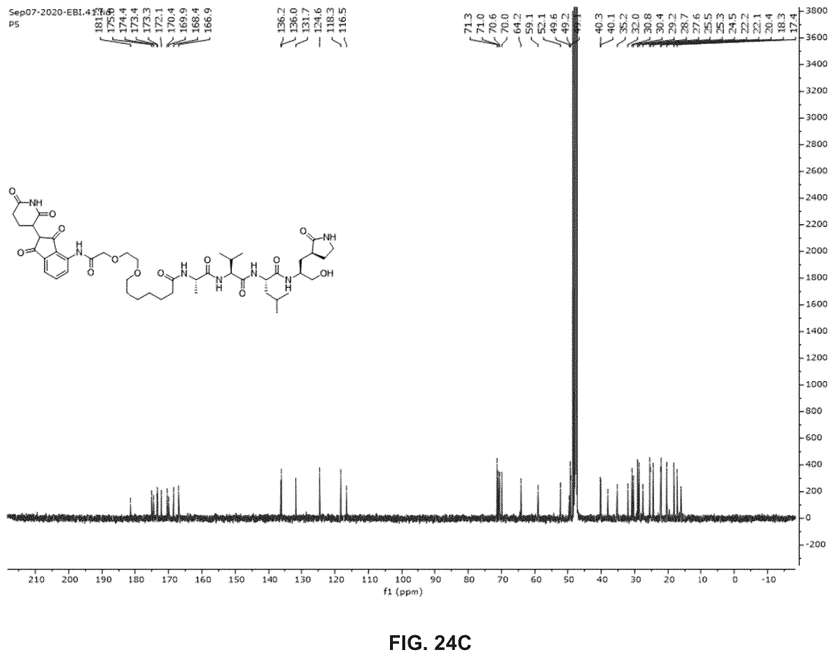

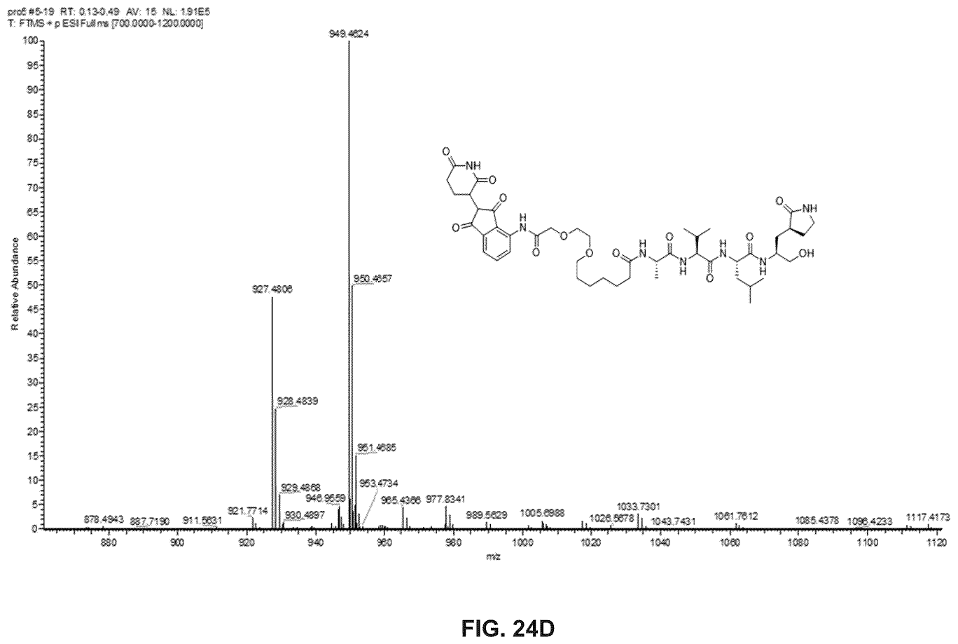

The synthesis of 7-(2-(2-((2-(2,6-dioxopiperidin-3-yl)-1,3-dioxo-2,3-dihydro-1H-inden-4-yl)amino)-2-oxoethoxy)ethoxy)-N-((S)-1-(((S)-1-(((S)-1-(((S)-1-hydroxy-3-((S)-2-oxopyrrolidin-3-yl)propan-2-yl)amino)-4-methyl-1-oxopentan-2-yl)amino)-3-methyl-1-oxobutan-2-yl)amino)-1-oxopropan-2-yl)heptanamide (MP-5) is shown in FIG. 24A. To a solution of MP-TA-3 (8.8 mg, 0.02 mmol) and pomalidomide-PEG2-butyl COOH (10.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (43% acetonitrile) to get product as light yellow solid (16 mg. 86%). ¹H NMR (methanol-d₄, 500 MHz): 1H NMR (500 MHz, methanol-d₄): 8.81 (d, J=8.4 Hz, 1H), 7.83 (dd, J=8.5, 7.3 Hz, 1H), 7.63 (d, J=7.3 Hz, 1H), 5.20-5.08 (m, 1H), 4.39-4.33 (m, 211), 4.24 (s, 111), 4.14 (dd, J=6.5, 1.2 Hz, 1H), 3.98 (dt, J=9.2, 3.2 Hz, 1H), 3.90-3.81 (m, 2H), 3.81-3.73 (m, 2H), 3.58-3.47 (m, 411), 3.29-3.20 (m, 211), 2.97-2.86 (m, 111), 2.83-2.72 (m, 2H), 2.53-2.47 (m, 1H), 2.35-2.28 (m, 1H), 2.24-2.14 (m, 3H), 2.10 (d, J=6.8 Hz, 1H), 2.03-1.97 (m, 1H), 1.81-1.72 (m, 111), 1.71-1.61 (m, 311), 1.59-1.50 (m, 5H), 1.37-1.28 (m, 9H), 0.96 (d, J=6.5 Hz, 9H), 0.92 (d, J=6.1 Hz. 311), as shown in FIG. 24B. ¹³C-NMR (methanol-d₄, 125 MHz): 181.3, 175.0, 174.4, 173.4, 173.3, 172.1, 170.4, 169.9, 168.4, 167.0, 136.2, 136.0, 131.7, 124.6, 118.3, 116.5, 71.4, 71.0, 70.6, 70.0, 64.2, 59.1, 52.1, 49.6, 49.2, 49.1, 40.3, 40.1, 38.0, 35.2, 32.0, 30.8, 30.4, 29.2, 28.7, 27.6, 25.5, 25.3, 24.5, 22.2, 22.1, 20.4, 18.3, 17.4, 16.2, as shown in FIG. 24C. HR-ESI-MS (m/z) calculated for 949.4687 (M+Na)⁺, found 949.4624 (FIG. 24D).

### Synthesis of MP-6

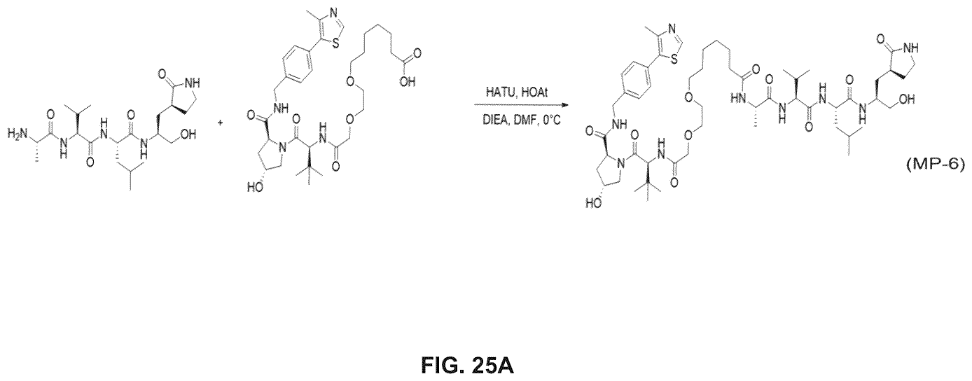

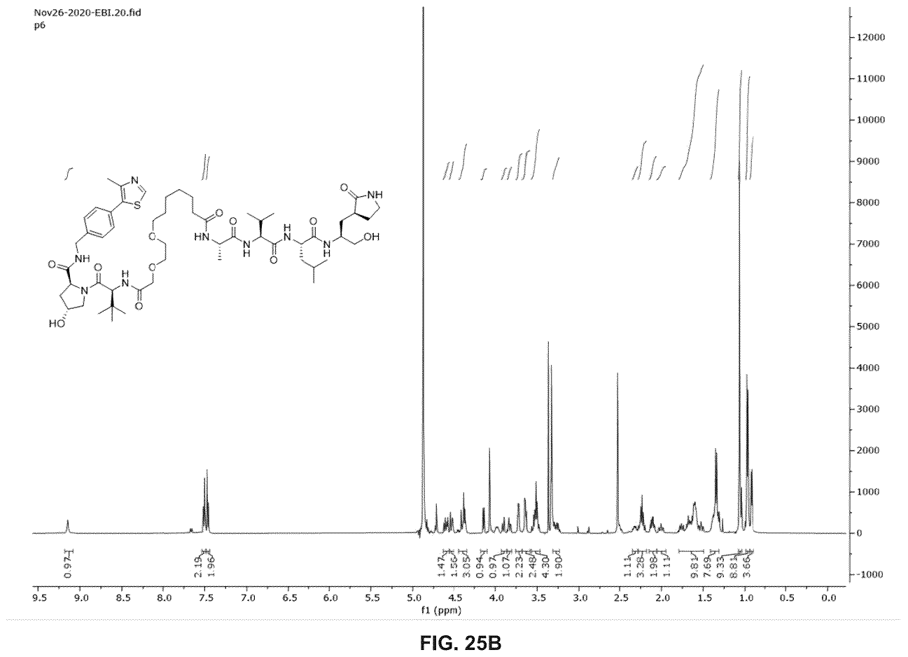

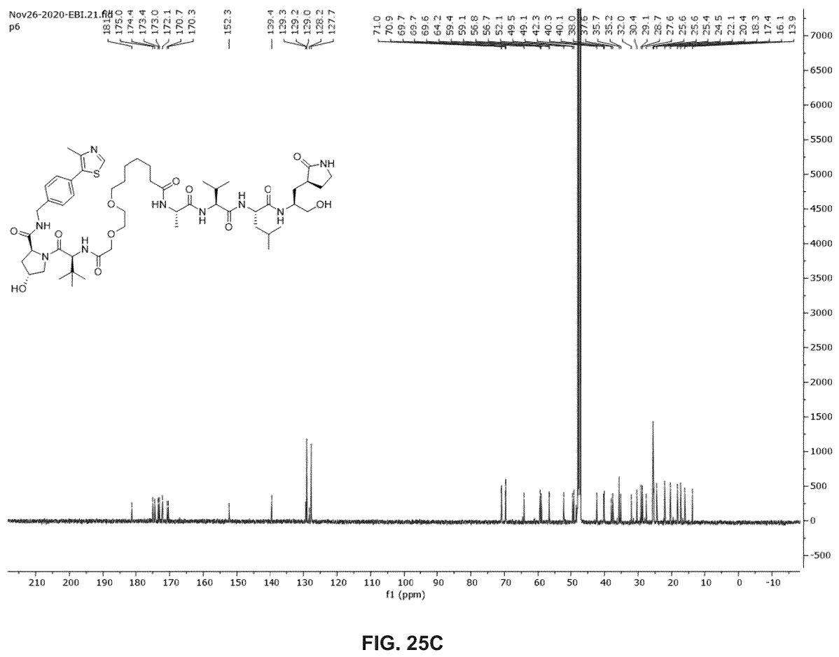

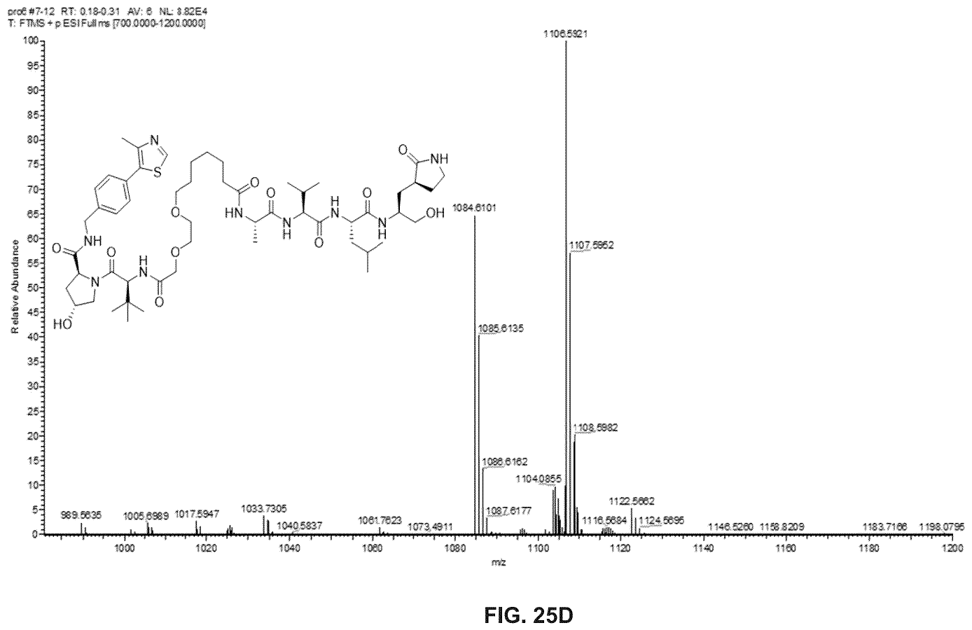

The synthesis of (2S,4R)-1-((2S,5S,8S,11S,27S)-27-(tert-butyl)-1-hydroxy-5-isobutyl-8-isopropyl-11-methyl-4,7,10,13,25-pentaoxo-2-(((S)-2-oxopyrrolidin-3-yl)methyl)-20,23-dioxa-3,6,9,12,26-pentaazaoctacosan-28-oyl)-4-hydroxy-N-(4-(4-methylthiazol-5-yl)benzyl)pyrrolidine-2-carboxamide (MP-6) is shown in FIG. 25A. To a solution of MP-TA-3 (8.8 mg, 0.02 mmol) and (S,R,S)-AHPC-PEG2-butyl COOH (13.2 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (38% acetonitrile) to get product as oil-like solid (13.1 mg, 61%). ¹H NMR (methanol-d₄, 500 MHz): 9.15 (s, 1H), 7.51 (d, J=8.2 Hz, 2H), 7.46 (d, J=8.4 Hz. 2H), 4.71 (s, 1H), 4.65-4.49 (m, 2H), 4.40-4.30 (m, 3H), 4.14 (d, J=6.5 Hz, 1H), 4.07 (s, 2H), 4.00-3.95 (m, 1H), 3.90 (d, J=11.1 Hz, 1H), 3.83 (dd, J=11.0, 3.8 Hz, 1H), 3.73-3.70 (m, 2H), 3.65-3.63 (m, 2H), 3.60-3.44 (m, 4H), 3.37 (s, 2H), 3.30-3.21 (m, 2H), 2.52 (s, 3H), 2.35-2.29 (m, 1H), 2.27-2.19 (m, 3H), 2.16-2.06 (m, 2H), 2.00 (ddd, J=13.9, 11.9, 3.5 Hz, 1H), 1.82-1.73 (m, 1H), 1.71-1.49 (m, 8H), 1.43-1.29 (m, 7H), 1.07 (s, 9H), 1.00-0.94 (m, 9H), 0.92 (d, J=6.2 Hz, 3H), as shown in FIG. 25B. ¹³C-NMR (methanol-d₄, 125 MHz): 181.2, 175.0, 174.4, 173.4, 173.0, 172.1, 170.7, 170.3, 152.3, 139.4, 129.3, 129.2, 129.0, 128.2, 127.7, 71.0, 70.9, 69.7, 69.7, 69.7, 64.2, 59.4, 59.1, 56.8, 56.7, 52.1, 49.5, 49.1, 42.3, 40.3, 40.1, 38.0, 37.6, 35.7, 35.2, 32.0, 30.4, 29.1, 28.7, 27.6, 25.6, 25.6, 25.4, 24.5, 22.1, 20.4, 18.3, 17.4, 16.1, 13.9, as shown in FIG. 25C. HR-ESI-MS (n/z) calculated for 1106.5904 (M+Na)⁺, found 1106.5921 (FIG. 25C).

### Synthesis of MP-7

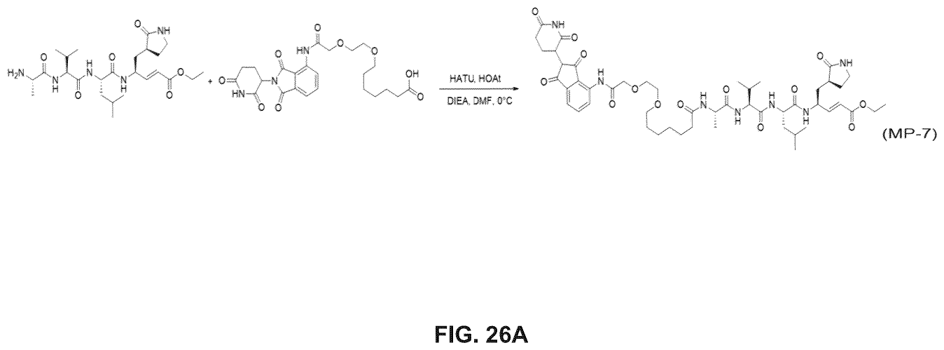

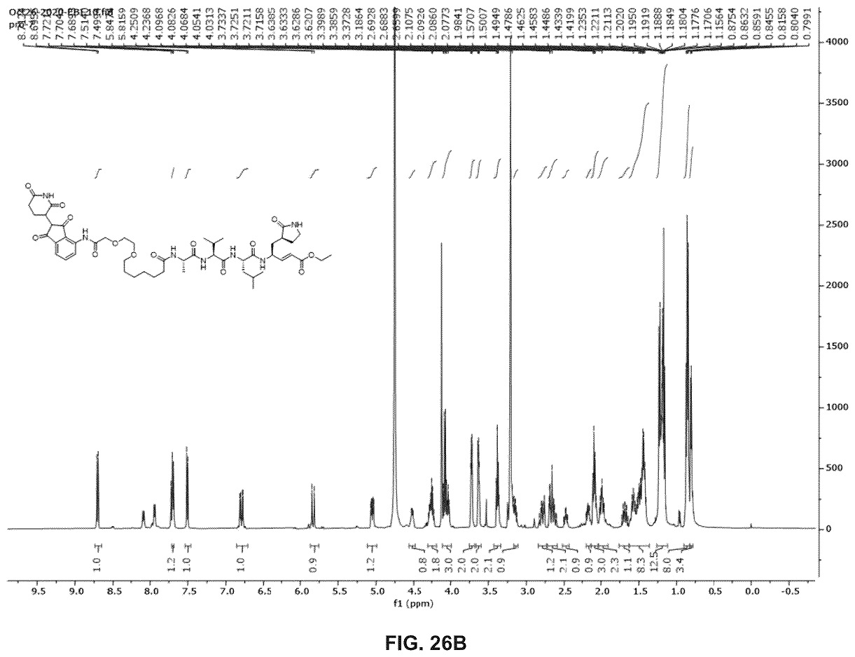

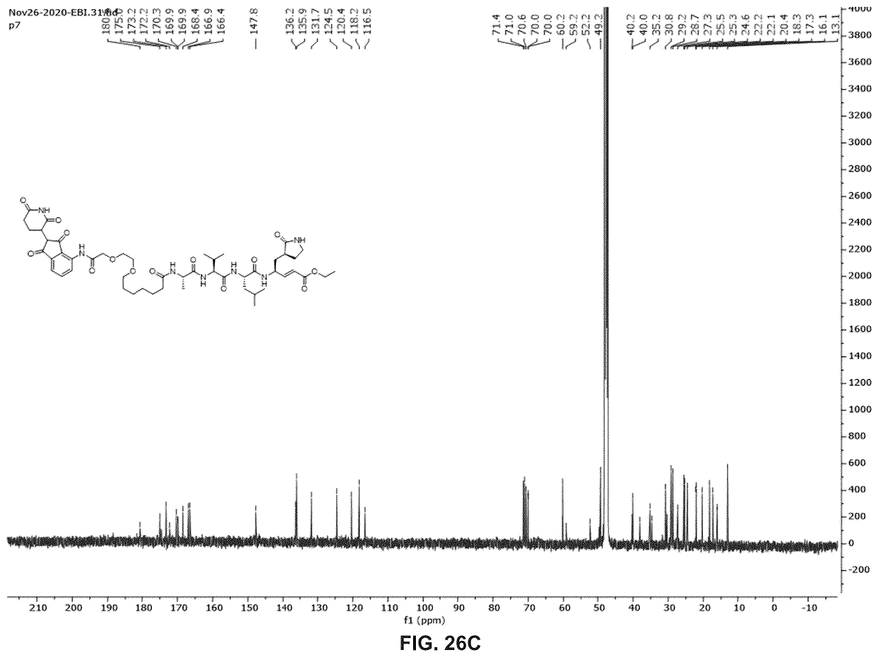

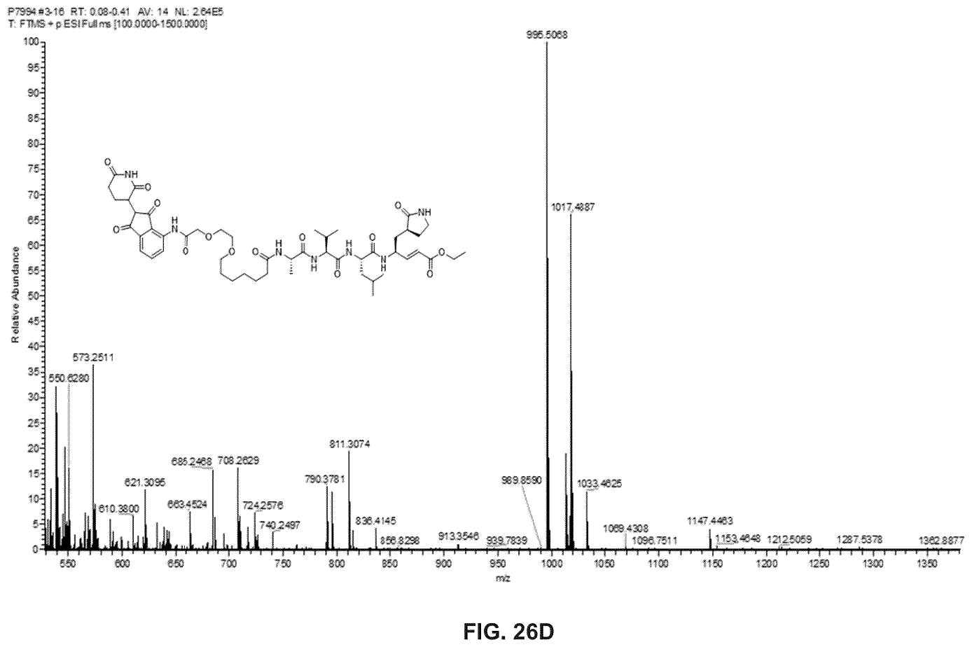

The synthesis of ethyl (15S,18S,21S,24S,E)-1-((2-(2,6-dioxopiperidin-3-yl)-1,3-dioxo-2,3-dihydro-1H-inden-4-yl)amino)-21-isobutyl-18-isopropyl-15-methyl-1,13,16,19,22-pentaoxo-24-(((S)-2-oxopyrrolidin-3-yl)methyl)-3,6-dioxa-14,17,20,23-tetraazaheptacos-25-en-27-oate (MP-7) is shown in FIG. 26A. To a solution of MP-TA-1 (10.2 mg, 0.02 mmol) and pomalidomide-PEG₂-butyl COOH (10.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (45% acetonitrile) to get product as yellow solid (15.5 mg, 78%). 1H NMR (methanol-d₄, 500 MHz): 8.70 (d, J=8.4 Hz, 1H), 7.70 (d, J=7.6 Hz 1H), 7.51 (d, J=7.3 Hz, 1H), 6.79 (dd, J=15.7, 5.1 Hz, 1H), 5.83 (d, J=15.7 Hz, 1H), 5.05 (ddd, J=12.4, 5.5, 2.5 Hz, 1H), 4.57-4.47 (m, 1H), 4.30-4.21 (m, 2H), 4.12 (s, 2H), 4.10-3.94 (m, 3H), 3.73-3.72 (m, 2H), 3.64-3.62 (m, 2H), 3.40-3.33 (m, 2H), 3.19-3.10 (m, 2H), 2.85-2.74 (m, 1H), 2.74-2.55 (m, 2H), 2.52-2.40 (m, 1H), 2.20-2.14 (m, 1H), 2.11-2.06 (m, 3H), 2.01-1.96 (m, 2H), 1.78-1.62 (m, 1H), 1.61-1.36 (m, 7H), 1.28-1.11 (m, 12H), 0.92-0.81 (m, 9H), 0.81 (d, J=5.9 Hz, 3H), as shown in FIG. 26B. ¹³C-NMR (methanol-d₄, 125 MHz): 180.6, 175.0, 173.2, 172.2, 170.3, 169.9, 169.9, 168.5, 166.9, 166.4, 147.8, 136.2, 135.9, 131.7, 124.5, 120.4, 118.2, 116.5, 71.4, 71.0, 70.6, 70.0, 70.0, 60.2, 59.2, 52.2, 49.2, 40.3, 40.1, 38.1, 35.2, 34.7, 30.8, 30.4, 29.2, 28.7, 27.3, 25.6, 25.3, 24.6, 22.2, 22.1, 20.4, 18.3, 17.4, 16.1, 13.1, as shown in FIG. 26C. HR-ESI-MS (m/z) calculated for 995.5110 (M+H)⁺, found 995.5068 (FIG. 26D).

### Synthesis of MP-8

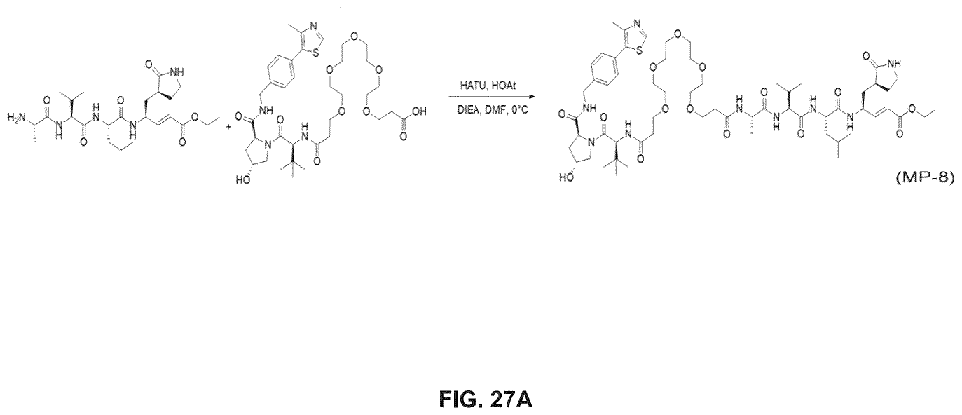

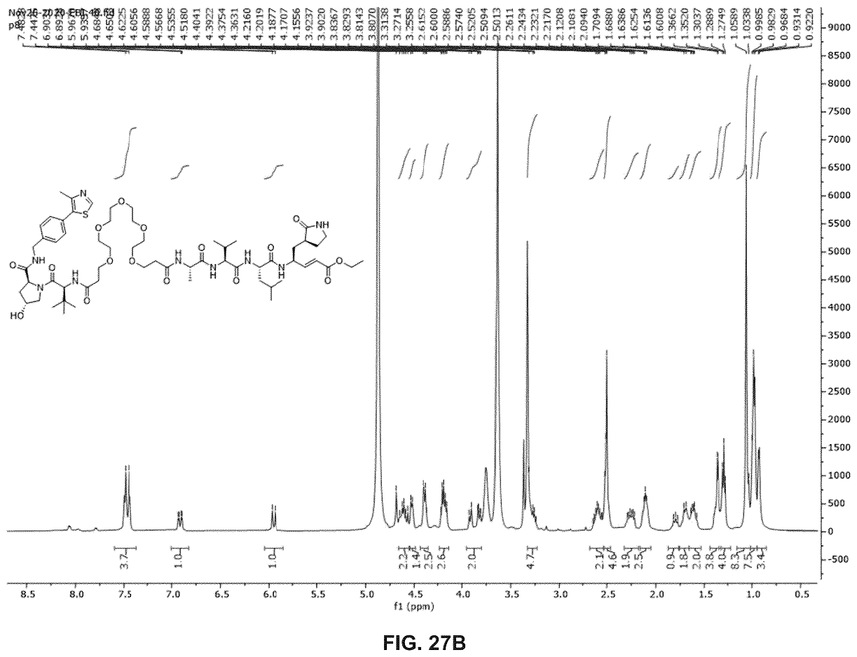

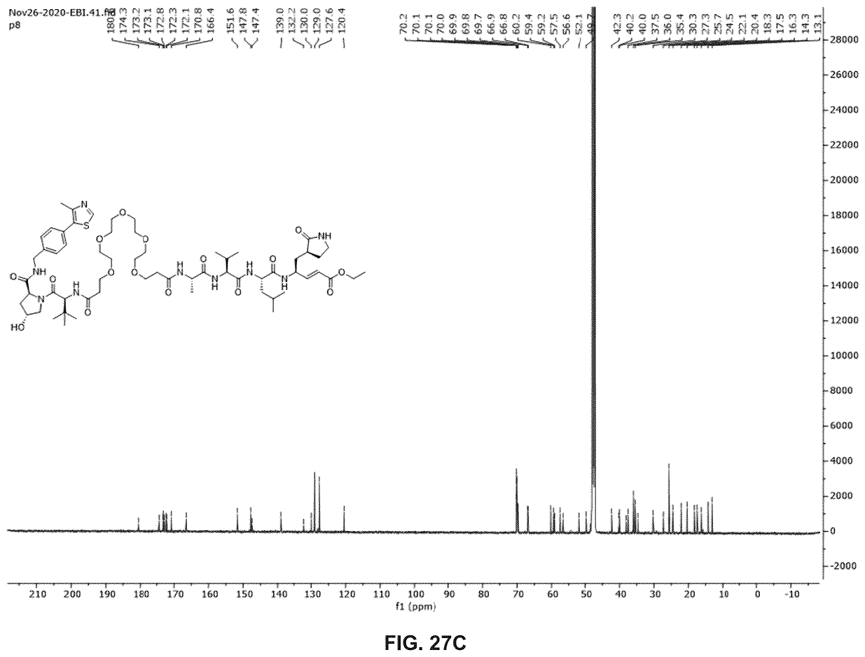

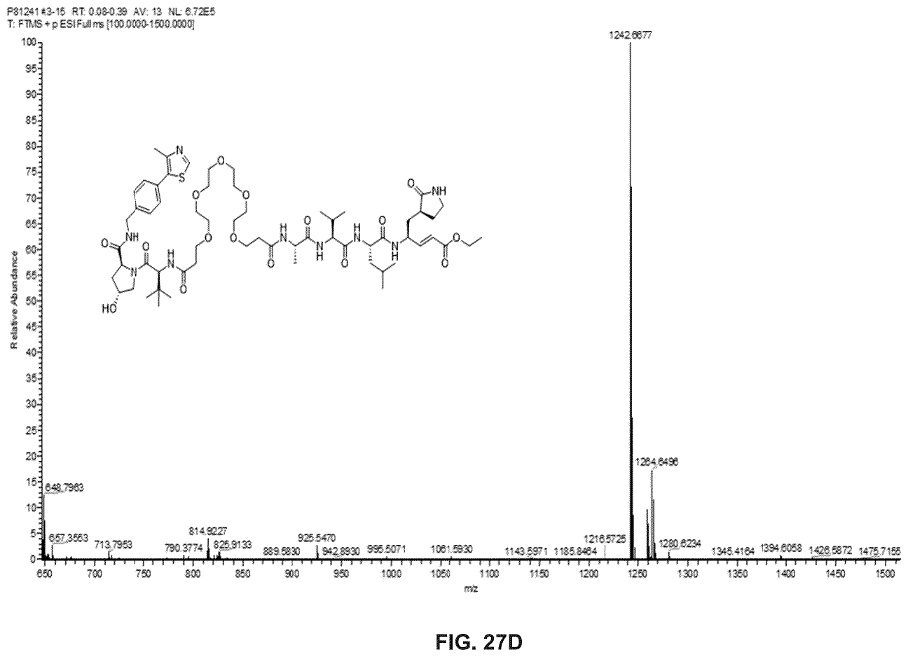

The synthesis of ethyl (3S,25S,28S,31S,34S,E)-3-((2S,4R)-4-hydroxy-2-((4-(4-methylthiazol-5-yl)benzyl)carbamoyl)pyrrolidine-1-carbonyl)-31-isobutyl-28-isopropyl-2,2,25-trimethyl-5,23,26,29,32-pentaoxo-34-(((S)-2-oxopyrrolidin-3-yl)methyl)-8,11,14,17,20-pentaoxa-4,24,27,30,33-pentaazaheptatriacont-35-en-37-oate (MP-8) is shown in in FIG. 27A. To a solution of MP-TA-1 (10.2 mg, 0.02 mmol) and (S,R,S)-AHPC-PEG5-COOH (15.0 mg. 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (38% acetonitrile) to get product as oil-like solid (14.3 mg, 67%). ¹H NMR (methanol-d₄, 500 MHz): 8.95 (s, 1H), 7.49 (d, J=7.8 Hz, 2H), 7.44 (d, J=7.8 Hz, 2H), 6.91 (dd, J=15.8, 5.1 Hz, 1H), 5.95 (d, J=15.7 Hz, 1H), 4.68-4.57 (m, 3H), 4.54-4.52 (m, 2H), 4.42-4.34 (m, 3H), 4.21-4.14 (m, 3H), 3.95-3.80 (m, 2H), 3.75 (m, 4H), 3.63 (m, 16H), 3.36-3.24 (m, 2H), 2.64-2.56 (m, 2H), 2.51 (m, 5H), 2.30-2.21 (m, 2H), 2.13-2.08 (m, 3H), 1.83-1.75 (m, 1H), 1.75-1.55 (m, 4H), 1.40-1.35 (m, 4H), 1.30-1.27 (m, 4H), 1.06 (s, 9H), 1.00-0.97 (m, 9H), 0.93 (d, J=4.7 Hz, 3H), as shown in in FIG. 27B. ¹³C-NMR (methanol-d₄, 125 MHz): 180.6, 174.3, 173.2, 173.1, 172.8, 172.3, 172.2, 170.8, 166.4, 151.6, 147.8, 147.4, 139.0, 132.2, 130.0, 129.0, 127.6, 120.4, 70.2, 70.2, 70.1, 70.0, 69.9, 69.8, 69.7, 66.9, 66.8, 60.2, 59.4, 59.2, 57.5, 56.6, 52.1, 49.7, 42.3, 40.2, 40.0, 38.1, 37.5, 36.0, 35.4, 34.7, 30.3, 27.3, 25.7, 25.6, 24.6, 22.1, 20.4, 18.3, 17.5, 16.3, 14.3, 13.1, as shown in in FIG. 27C. HR-ESI-MS (m/z) calculated for 1242.6621 (M+H)+, found 1242.6677 (FIG. 27D).

### Synthesis of MP-9

The synthesis of ethyl (3S,6S,9S,12S,E)-9-isobutyl-6-isopropyl-3-methyl-1,4,7,10-tetraoxo-12-(((S)-2-oxopyrrolidin-3-yl)methyl)-1-(3-(pyridin-2-yl)phenyl)-2,5,8,11-tetraazapentadec-13-en-15-oate (MP-9) is shown in FIG. 27A. To a solution of MP-TA-1 (10.2 mg, 0.02 mmol) and 3-(pyridin-2-yl)benzoic acid (4.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (32% acetonitrile) to get product as white solid (9.1 mg, 66%). ¹H NMR (methanol-d₄, 500 MHz): 8.67 (d, J=4.8 Hz, 1H), 8.51 (t, J=1.8 Hz, 1H), 8.17 (dt, J=7.9, 1.4 Hz, 1H), 8.00-7.92 (m, 3H), 7.63 (t, J=7.7 Hz, 1H), 7.43 (td, J=5.2, 3.0 Hz, 1H), 6.92 (dd, J=15.7, 5.1 Hz, 1H), 5.96 (dd, J=15.7, 1.7 Hz, 1H), 4.66-4.61 (m, 2H), 4.40 (dd, J=10.1, 5.0 Hz, 1H), 4.25-4.12 (m, 3H), 3.31-3.24 (m, 2H), 2.66-2.60 (m, 1H), 2.33-2.23 (m, 1H), 2.23-2.05 (m, 2H), 1.86-1.55 (m, 5H), 1.53 (d, J=7.2 Hz, 3H), 1.29 (m, J=7.2 Hz, 3H), 1.03-0.93 (m, 9H), 0.93 (d, J=4.7 Hz. 3H), as shown in FIG. 27B. ¹³C-NMR (methanol-d₄, 125 MHz): 180.6, 174.6, 173.2, 172.2, 168.8, 166.5, 156.5, 149.1, 147.8, 139.4, 137.6, 134.3, 130.0, 128.8, 127.8, 126.0, 122.8, 121.2, 120.4, 60.2, 59.3, 52.2, 50.5, 48.5, 48.1, 47.9, 47.8, 47.6, 47.4, 47.3, 47.1, 40.2, 40.0, 38.1, 34.6, 30.3, 27.3, 24.6, 22.0, 20.3, 18.2, 17.4, 16.1, 13.1, as shown in FIG. 27C. HR-ESI-MS (m/z) calculated for 691.3816 (M+H)+, found 691.3807 (FIG. 27D).

### Synthesis of MP-10

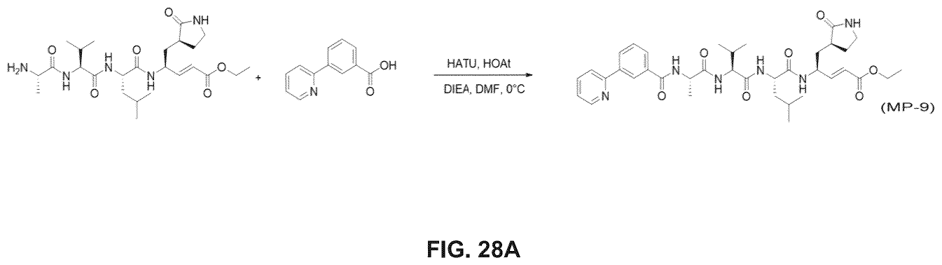

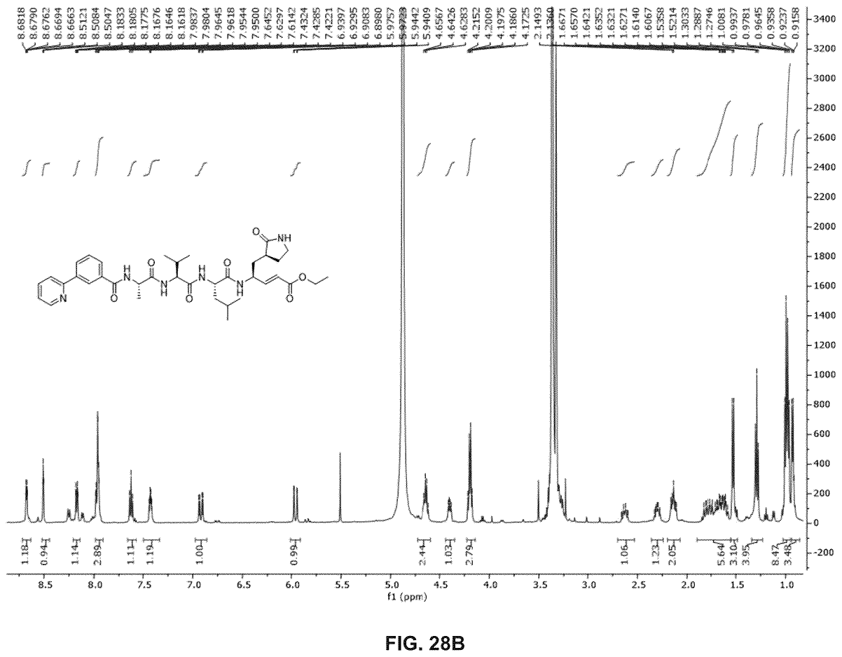

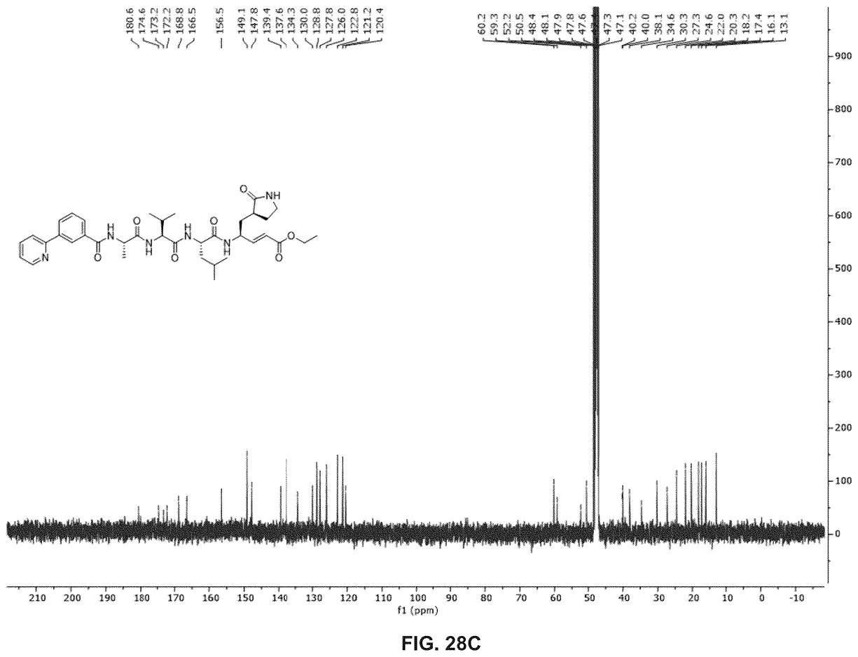

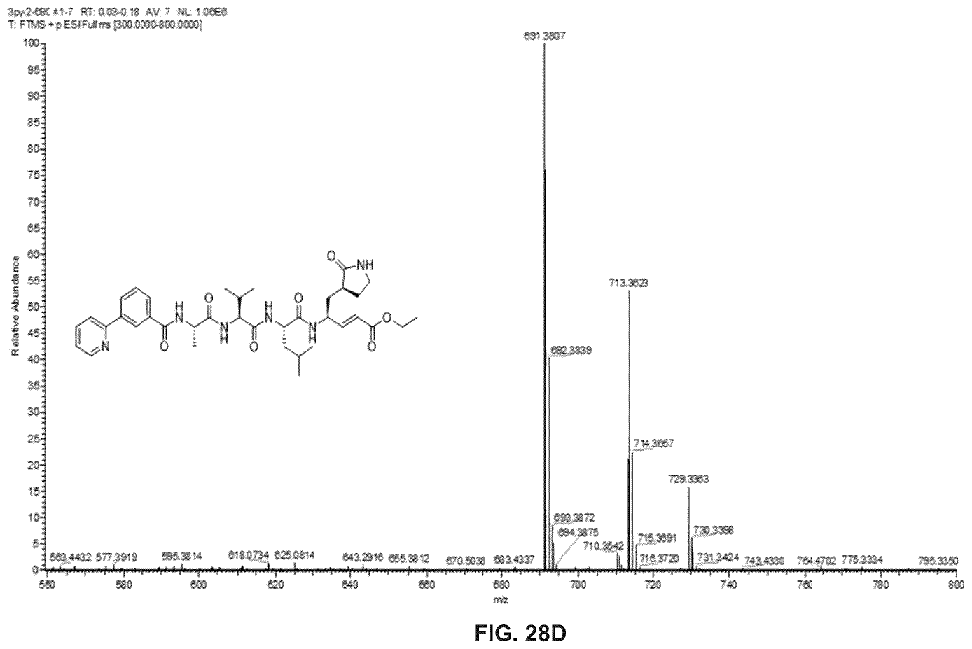

The synthesis of ethyl (3S,6S,9S,E)-9-isobutyl-6-isopropyl-3-methyl-1,4,7,10-tetraoxo-12-(((S)-2-oxopyrrolidin-3-yl)methyl)-1-(4-(pyridin-2-yl)phenyl)-2,5,8,11-tetraazapentadec-13-en-15-oate (MP-10) is shown in FIG. 28A. To a solution of MP-TA-1 (10.2 mg, 0.02 mmol) and 4-(pyridin-2-yl)benzoic acid (4.0 mg, 0.02 mmol) in 1 mL DMF, DIEA (17.5 p L, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (28% acetonitrile) to get product as light yellow solid (11.1 mg, 80%). ¹H NMR (methanol-d₄, 500 MHz): 8.65 (d, J=1.7 Hz. 1H), 8.17 (dt, J=7.8, 1.4 Hz, 1H), 8.06 (d, J=8.1 Hz, 1H), 7.99-7.93 (m, 4H), 7.63-7.60 (m, 1H), 6.80 (dd, J=15.7, 5.1 Hz, 1H), 5.84 (dd. J=15.7, 1.7 Hz, 1H), 4.55-4.50 (m, 2H), 4.26 (dd, J=10.0, 7.8 Hz, 1H), 4.10-4.06 (m, 3H), 3.18-3.13 (m, 2H), 2.54-2.47 (m, 1H), 2.22-2.16 (m, 1H), 2.04-1.97 (m, 2H), 1.75-1.46 (m, 5H), 1.40 (d, J=7.2 Hz, 3H), 1.17 (t, J=7.2 Hz, 3H), 0.92-0.81 (m, 9H), 0.81 (d, J=4.7 Hz, 3H), as shown in FIG. 28B. ¹³C-NMR (methanol-d₄, 125 MHz): 180.7, 174.4, 173.2, 172.1, 168.1, 166.4, 154.3, 147.8, 146.1, 141.8, 138.6, 135.4, 128.3, 128.1, 127.4, 124.3, 123.6, 120.4, 60.3, 59.1, 52.2, 50.4, 40.2, 40.1, 38.1, 34.7, 30.4, 27.3, 24.6, 22.0, 20.4, 18.3, 17.3, 16.1, 13.1, as shown in FIG. 28C. HR-ESI-MS (m/z) calculated for 691.3816 (M+H)⁺, found 691.3813 (FIG. 28C).

### Synthesis of MP-II

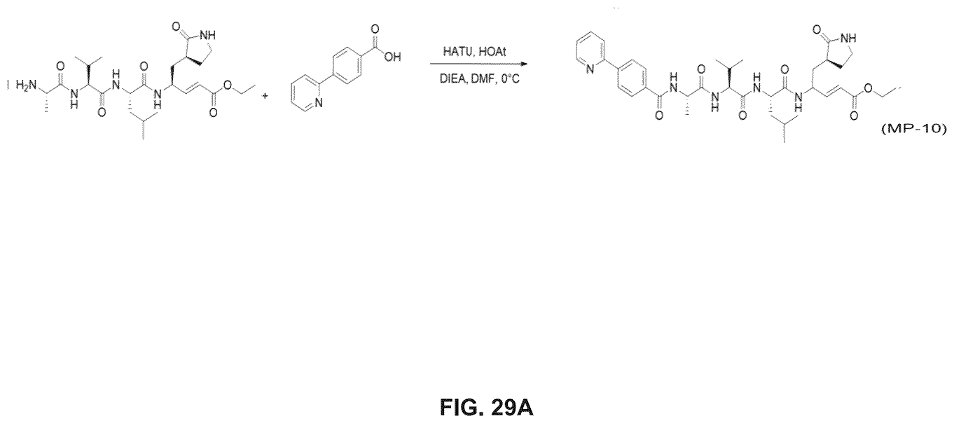

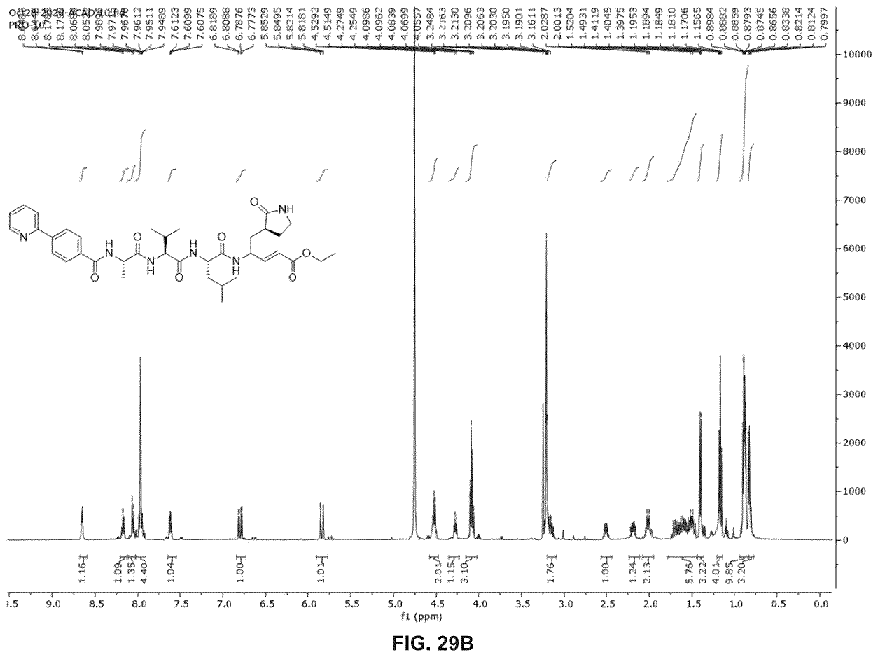

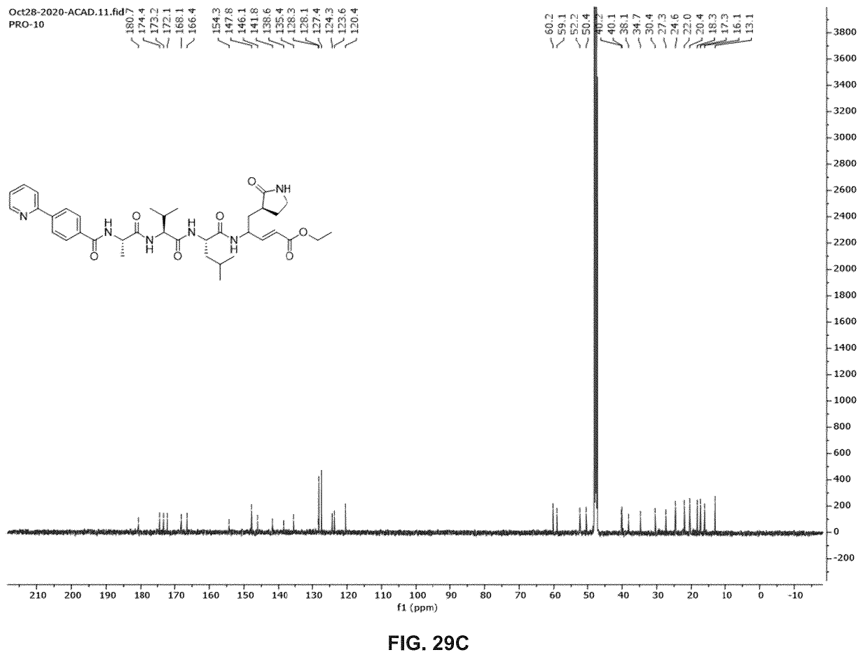

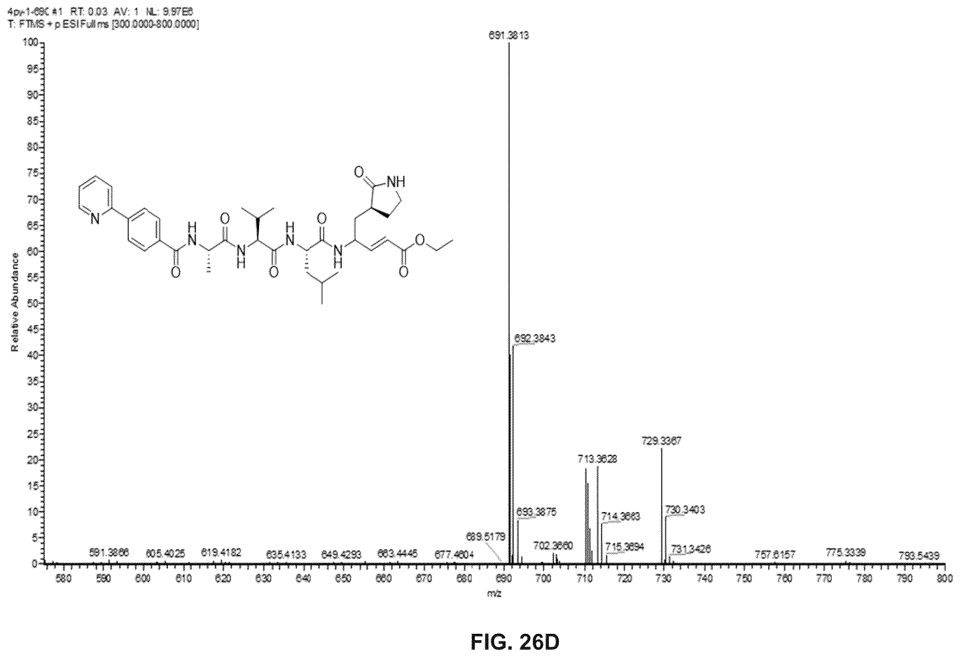

The synthesis of ethyl (16S,19S,E)-1-((2-(2,6-dioxopiperidin-3-yl)-1,3-dioxoisoindolin-4-yl)amino)-16-isobutyl-14,17-dioxo-19-(((S)-2-oxopyrrolidin-3-yl)methyl)-3,6,9,12-tetraoxa-15,18-diazadocos-20-en-22-oate (MP-11) is shown in FIG. 29A. To a solution of MP-TA-4 (6.8 mg, 0.02 mmol) and pomalidomide-PEG₄-C—COOH (11.4 mg, 0.02 mmol) in 1 mL DMF, DIEA (17.5 μL, 0.1 mmol), HATU (15.2 mg, 0.04 mmol), and HOAt (0.6 mg. 0.004 mmol) were added under ice bath. The reaction was stirred at 0° C. for half hour, then the mixture was purified with HPLC (42% acetonitrile) to get product as yellow-green solid (11.7 mg, 71%). ¹H NMR (methanol-d₄, 500 MHz): 7.45 (dd, J=8.5, 7.1 Hz, 1H), 7.00 (d, J=8.5 Hz, 1H), 6.96 (d, J=8.6 Hz, 1H), 6.78 (dd, J=15.7, 5.2 Hz, 1H), 5.80 (dd, J=15.7, 1.6 Hz, 1H), 4.95 (dd, J=12.5, 5.5 Hz, 1H), 4.56-4.51 (m, 1H), 4.35 (dd, J=9.4, 5.2 Hz, 1H), 4.07 (q, J=7.1 Hz, 2H), 3.95-3.88 (m, 2H), 3.62 (t, J=5.2 Hz, 2H), 3.57-3.53 (m, 10H), 3.25 (s, 2H), 3.19-3.14 (m, 2H), 2.77-2.69 (m, 1H), 2.67-2.57 (m, 2H), 2.45-2.38 (m, 1H), 2.22-2.16 (m, 1H), 2.04-1.99 (m, 1H), 1.94-1.88 (m, 1H), 1.69 (dq, J=12.6, 9.1 Hz, 1H), 1.60-1.48 (m, 3H), 1.17 (t, J=7.1 Hz, 3H), 0.88 (dd, J=6.1, 1.9 Hz, 3H), 0.84 (d, J=6.0 Hz, 3H), as shown in FIG. 29B. ¹³C-NMR (methanol-d₄, 125 MHz): 180.7, 173.3, 173.0, 171.4, 170.2, 169.3, 167.9, 166.4, 147.8, 146.8, 135.8, 132.5, 120.4, 116.9, 110.7, 109.9, 70.6, 70.3, 70.2, 70.2, 70.0, 69.7, 69.2, 60.3, 51.7, 48.870, 48.5, 41.9, 40.7, 40.1, 38.2, 34.7, 30.8, 27.4, 24.6, 22.4, 22.0, 20.7, 13.1, as shown in FIG. 29C. HR-ESI-MS (m/z) calculated for 829.3980 (M+H)⁺, found 829.3974 (FIG. 29D).

### Synthesis of MP-12

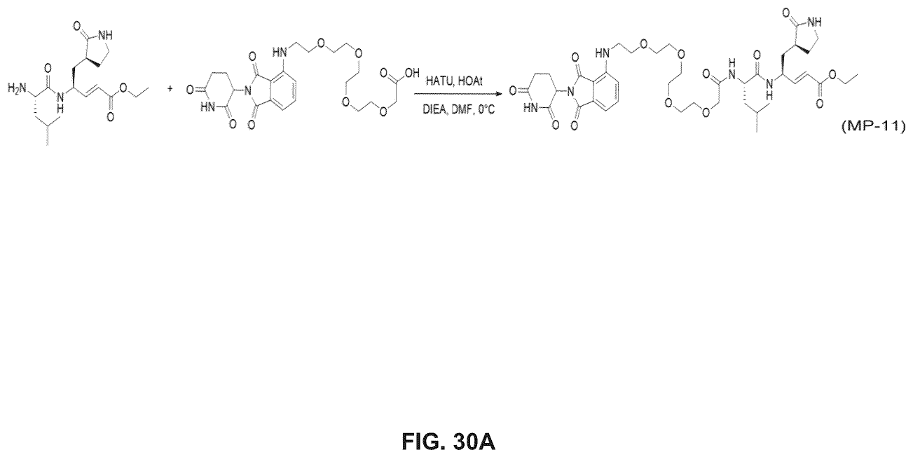

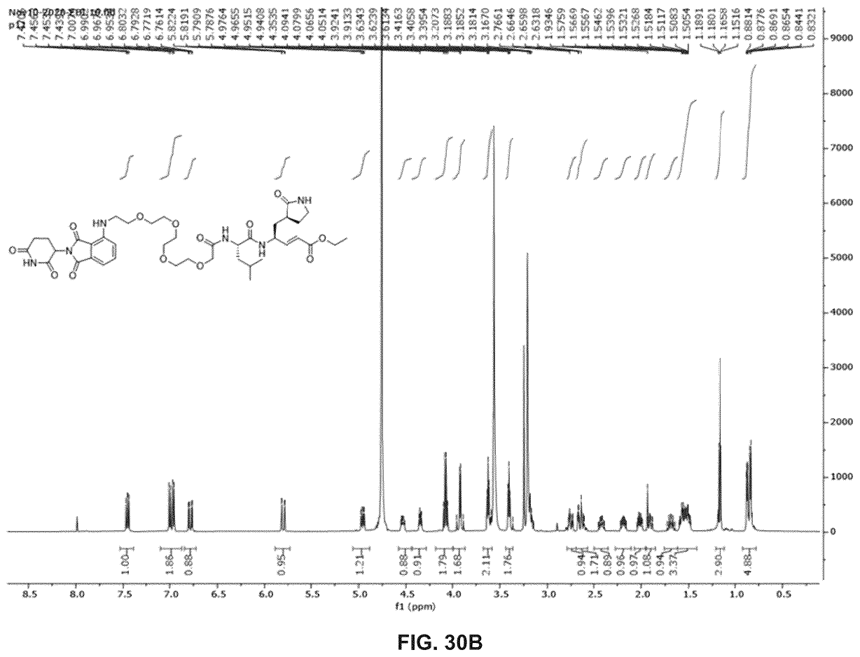

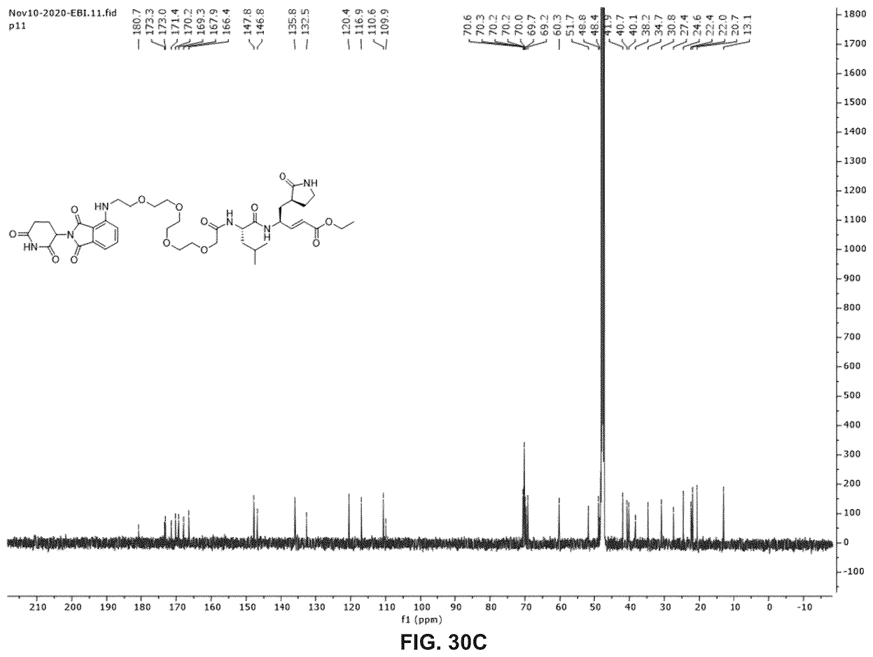

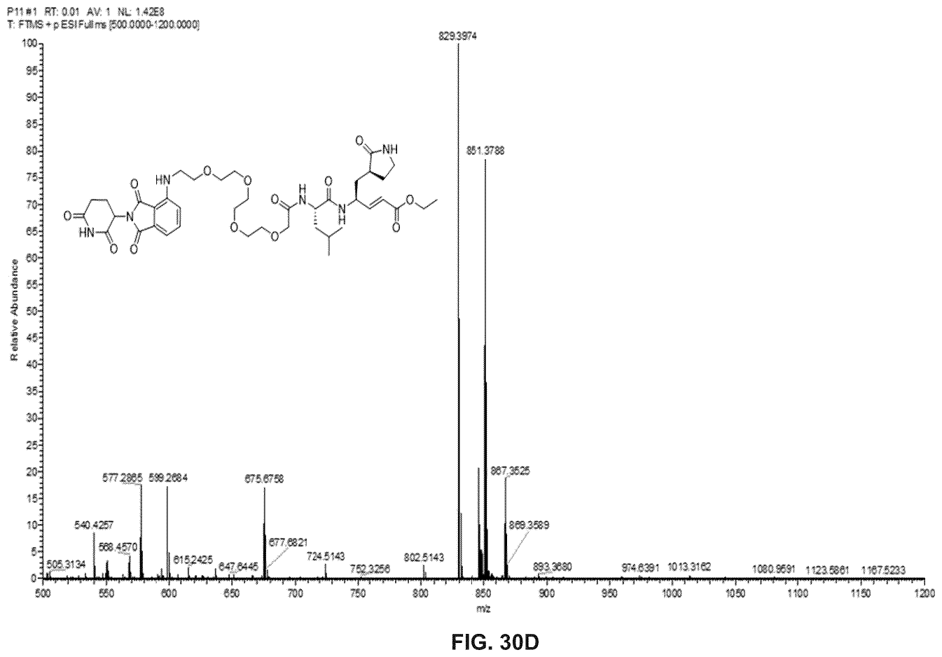

The synthesis of ethyl (3S,25S,28S,E)-3-((2S,4R)-4-hydroxy-2-((4-(4-methylthiazol-5-yl)benzyl)carbamoyl)pyrrolidine-1-carbonyl)-25-isobutyl-2,2-dimethyl-5,23,26-trioxo-28-(((S)-2-oxopyrrolidin-3-yl)methyl)-8,11,14,17,20-pentaoxa-4,24,27-triazahentriacont-29-en-31-oate (MP-12) is shown in FIG. 30A. To a solution of MP-TA-4 (6.8 mg, 0.02 mmol) and (S,R,S)-AHPC-PEG₅-COOH (15.0 mg, 0.02 mmol) in 1 mL DMF, DIEA (17.5 L, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (36% acetonitrile) to get product as light yellow solid (14.1 mg, 66%). ¹H NMR (methanol-d₄, 500 MHz): 9.15 (s, 1H), 7.41 (d, J=8.3 Hz, 2H), 7.35 (d, J=8.3 Hz, 2H), 6.79 (dd, J=15.7, 5.2 Hz, 1H), 5.81 (dd, J=15.7, 1.7 Hz, 1H), 4.57-4.51 (m, 2H), 4.49-4.44 (m, 2H), 4.40-4.39 (m, 1H), 4.29-4.21 (m, 2H), 4.08 (q, J=7.1 Hz, 2H), 3.80 (d, J=11.0 Hz, 1H), 3.70 (dd, J=11.0, 3.8 Hz, 1H), 3.66-3.59 (m, 4H), 3.51 (d, J=7.0 Hz, 16H), 3.19-3.16 (m, 2H), 2.52-2.32 (m, 7H), 2.22-2.16 (m, 1H), 2.14-2.10 (m, 1H), 2.01-1.86 (m, 2H), 1.69 (dq, J=12.5, 9.4 Hz, 1H), 1.63-1.57 (m, 1H), 1.56-1.45 (m, 3H), 1.17 (t, J=7.1 Hz, 3H), 0.94 (s, 9H), 0.88 (d, J=6.6 Hz, 3H), 0.84 (d, J=6.5 Hz, 3H), as shown in FIG. 30B. ¹³C-NMR (methanol-d₄, 125 MHz): 180.7, 173.5, 173.1, 172.8, 172.4, 170.7, 166.4, 152.7, 147.9, 139.7, 129.0, 127.8, 120.4, 70.2, 70.1, 70.1, 70.1, 70.0, 69.9, 69.7, 66.9, 66.9, 60.3, 59.4, 57.5, 56.6, 52.3, 42.3, 40.6, 40.1, 38.3, 37.5, 36.1, 36.0, 35.4, 34.7, 27.4, 25.6, 24.6, 22.0, 20.6, 13.5, 13.1, as shown in FIG. 30C. HR-ESI-MS (m/z) calculated for 1072.5609 (M+H)⁺, found 1072.5624 (FIG. 30D).

### Synthesis of MP-13

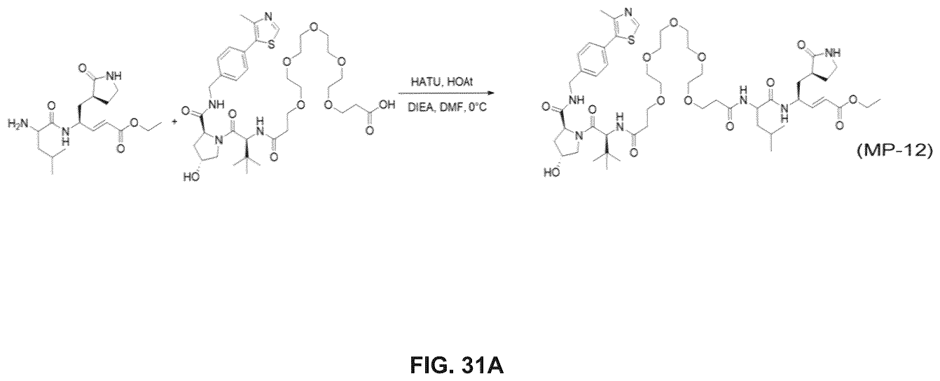

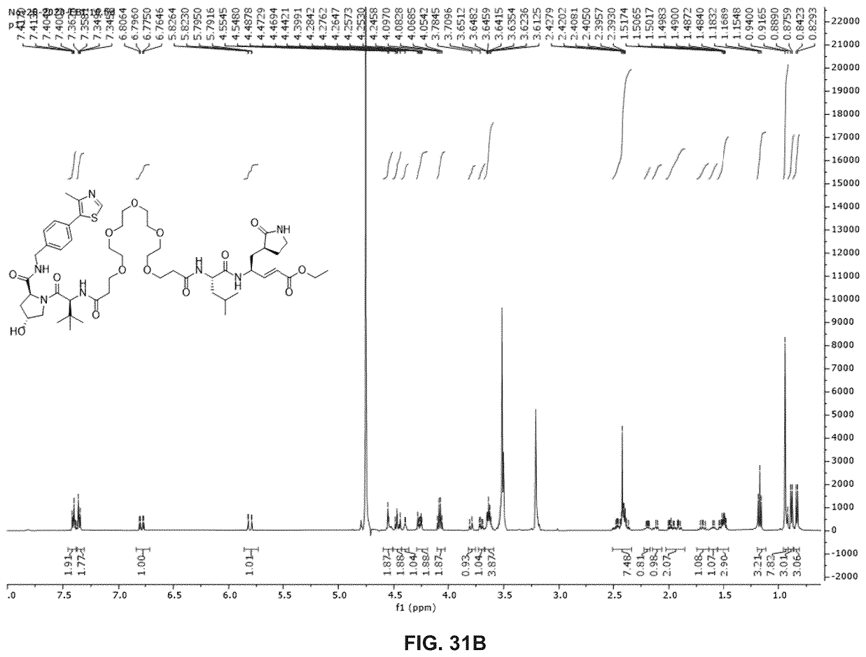

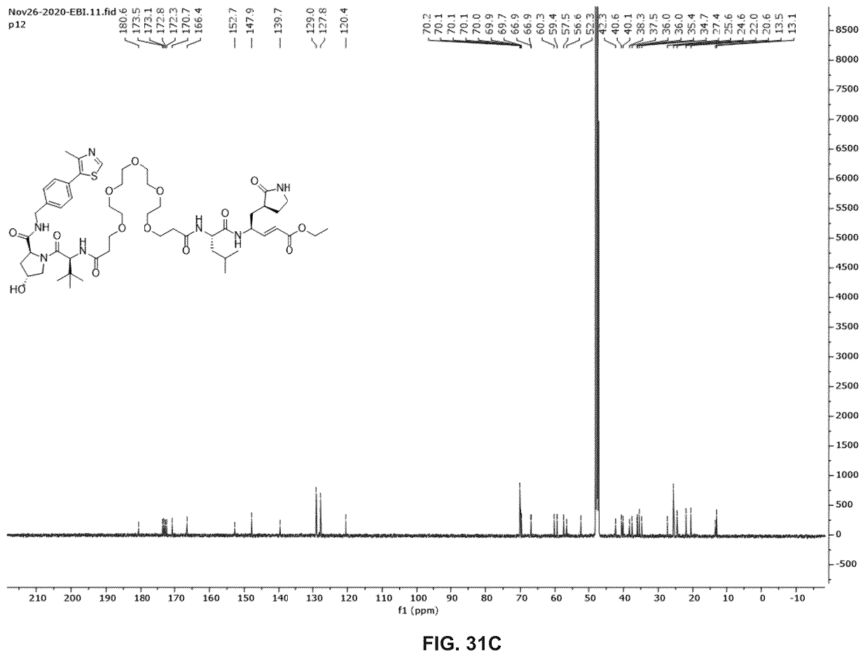

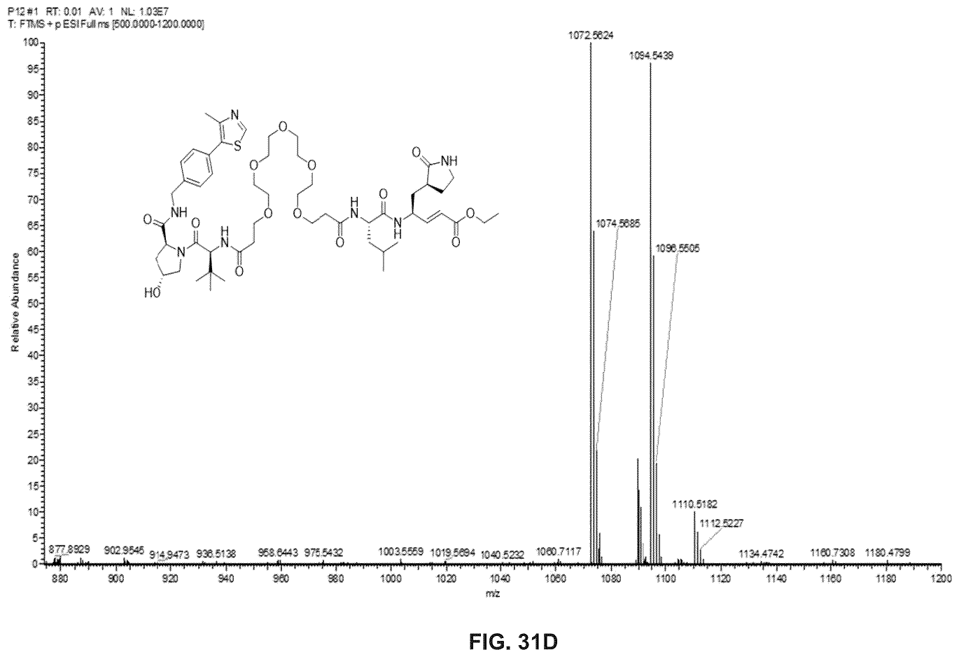

The synthesis of benzyl (16S,19S,22S,25S,E)-1-((2-(2,6-dioxopiperidin-3-yl)-1,3-dioxoisoindolin-4-yl)amino)-22-isobutyl-19-isopropyl-16-methyl-14,17,20,23-tetraoxo-25-(((S)-2-oxopyrrolidin-3-yl)methyl)-3,6,9,12-tetraoxa-15,18,21,24-tetraazaoctacos-26-en-28-oate (MP-13) is shown in FIG. 31A. To a solution of MP-TA-2 (11.4 mg, 0.02 mmol) and pomalidomide-PEG₄-C—COOH (11.4 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (45% acetonitrile) to get product as yellow-green solid (16.2 mg, 76%).

¹H NMR (methanol-d₄, 500 MHz): 7.56 (dd, J=8.6, 7.1 Hz, 1H), 7.43-7.24 (m, 5H), 7.10 (d, J=8.6 Hz, 1H), 7.07 (d, J=7.1 Hz, 1H), 6.96 (dd, J=15.7, 5.1 Hz, 1H), 6.00 (dd, J=15.7, 1.6 Hz, 1H), 5.19 (s, 2H), 5.08 (dd, J=12.4, 5.4 Hz, 1H), 4.71-4.60 (m, 1H), 4.45 (qd, J=6.8, 2.2 Hz, 1H), 4.39-4.35 (m, 1H), 4.18-4.15 (m, 1H), 4.01 (s, 2H), 3.73 (t, J=5.2 Hz, 2H), 3.67-3.66 (m, 12H), 3.51 (t, J=5.2 Hz, 2H), 3.31-3.22 (m, 2H), 2.93-2.82 (m, 1H), 2.79-2.66 (m, 2H), 2.60-2.53 (m, 1H), 2.33-2.24 (m, 1H), 2.16-2.01 (m, 3H), 1.83-1.75 (m, 1H), 1.68-1.57 (m, 4H), 1.39 (d, J=7.1 Hz, 3H), 1.00-0.88 (m, overlapped, 12H), as shown in FIG. 31B. ¹³C-NMR (methanol-d₄, 125 MHz): 180.6, 173.6, 173.3, 173.2, 172.0, 171.3, 170.3, 169.3, 167.9, 166.1, 148.3, 146.8, 136.2, 135.8, 132.5, 128.2, 127.8, 120.2, 116.9, 110.7, 109.9, 70.6, 70.3, 70.2, 69.9, 69.7, 69.2, 65.9, 58.8, 52.2, 48.8, 41.9, 40.3, 40.1, 38.2, 34.7, 30.8, 30.5, 30.5, 27.4, 24.6, 22.4, 21.9, 20.7, 18.3, 17.4, 17.4, 16.7, as shown in FIG. 31C. HR-ESI-MS (m/z) calculated for 1083.4988 (M+Na)⁺, found 1083.5018 (FIG. 31D).

### Synthesis of MP-14

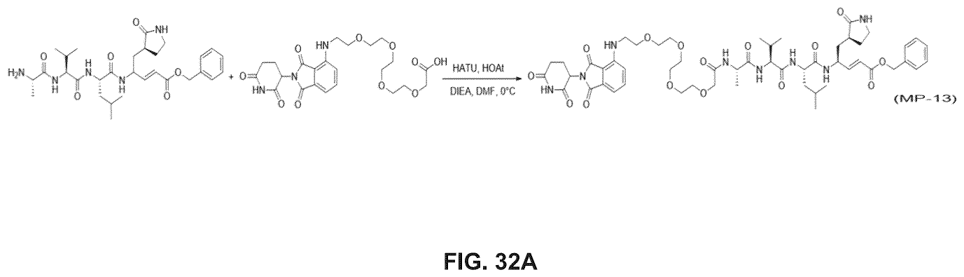

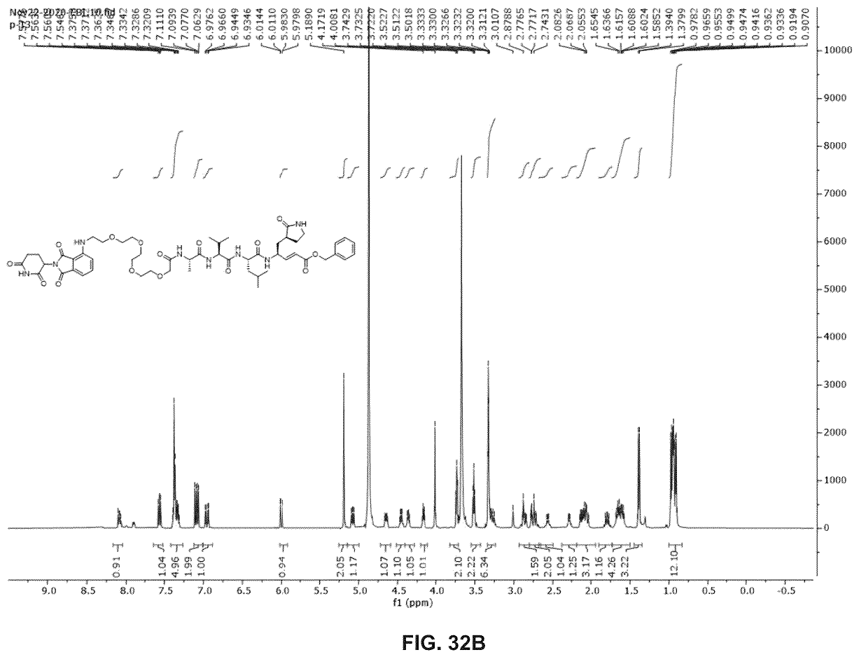

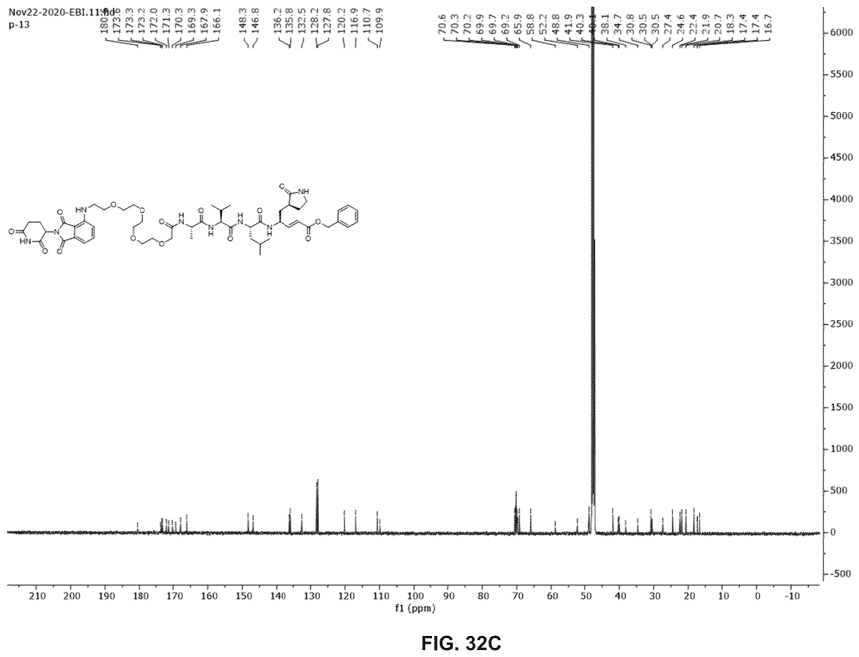

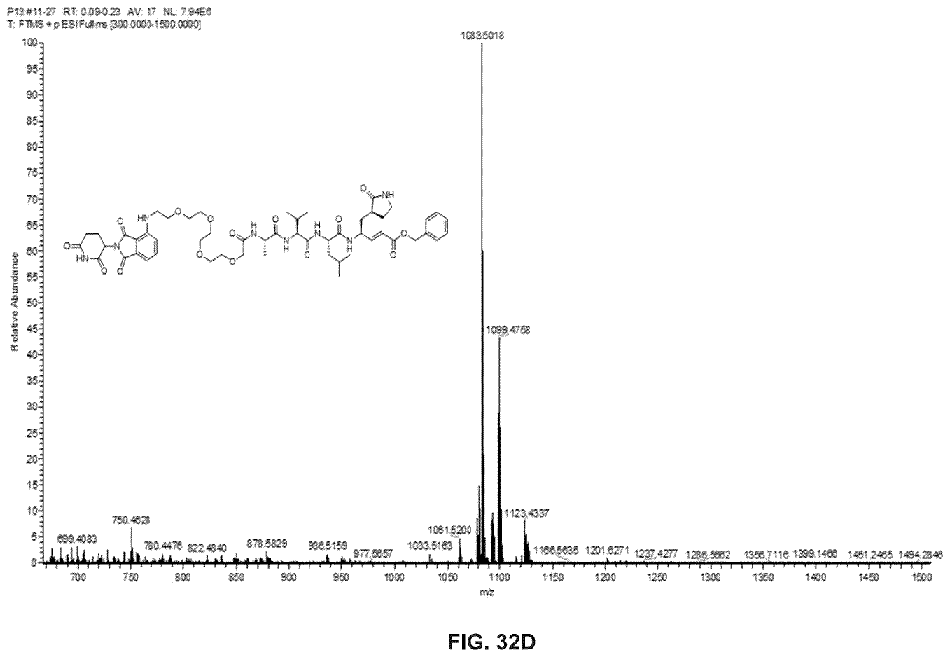

The synthesis of benzyl (3S,25S,28S,31S,34S,E)-3-((2S,4R)-4-hydroxy-2-((4-(4-methylthiazol-5-yl)benzyl)carbamoyl)pyrrolidine-1-carbonyl)-31-isobutyl-28-isopropyl-2,2,25-trimethyl-5,23,26,29,32-pentaoxo-34-(((S)-2-oxopyrrolidin-3-yl)methyl)-8,11,14,17,20-pentaoxa-4,24,27,30,33-pentaazaheptatriacont-35-en-37-oate (MP-14) is shown in FIG. 32A. To a solution of MP-TA-2 (11.4 mg, 0.02 mmol) and (S,R,S)-AHPC-PEG₅-COOH (15.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (40% acetonitrile) to get product as light yellow solid (15.7 mg, 60%). ¹H NMR (methanol-d₄, 500 MHz): 9.42 (s, 1H), 7.53 (d, J=8.3 Hz, 2H), 7.48 (d, J=8.3 Hz. 2H), 7.40-7.27 (m, 5H), 6.96 (dd, J=15.7, 5.1 Hz, 1H), 6.00 (dd, J=15.7, 1.7 Hz, 1H), 5.19 (s, 2H), 4.73-4.46 (m, 3H), 4.45-4.33 (m, 2H), 4.15 (d, J=6.6 Hz, 1H), 3.92-3.79 (m, 2H), 3.76-3.71 (m, 4H), 3.65-3.61 (m, 16H), 3.29-3.23 (m, 2H), 2.64-2.44 (m, 8H), 2.33-2.18 (m, 2H), 2.17-2.00 (m, 3H), 1.83-1.75 (m, 1H), 1.73-1.53 (m, 4H), 1.35 (d, J=7.2 Hz, 3H), 1.05 (s, 9H), 0.98-0.95 (m, 9H), 0.91 (d, J=6.0 Hz, 3H), as shown in FIG. 32B. ¹³C-NMR (methanol-d₄, 125 MHz): 180.5, 174.3, 173.2, 173.2, 172.8, 172.3, 172.2, 170.8, 166.1, 161.9, 153.3, 148.3, 140.0, 136.2, 129.0, 128.2, 127.8, 127.8, 120.2, 70.2, 70.2, 70.1, 70.0, 69.9, 69.7, 66.9, 66.8, 65.9, 59.4, 59.2, 57.5, 56.7, 52.1, 49.8, 42.3, 40.2, 40.0, 38.1, 37.6, 36.0, 35.4, 34.6, 30.3, 27.3, 25.7, 24.6, 22.1, 20.4, 18.3, 17.5, 16.3, 13.1, as shown in FIG. 32C. HR-ESI-MS (n/z) calculated for 1326.6597 (M+Na)⁺, found 1326.6682 (FIG. 32D).

### Synthesis of MP-15

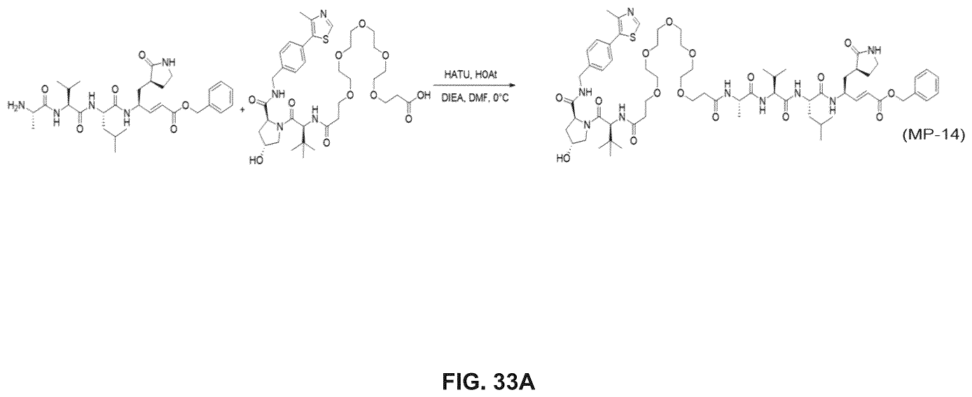

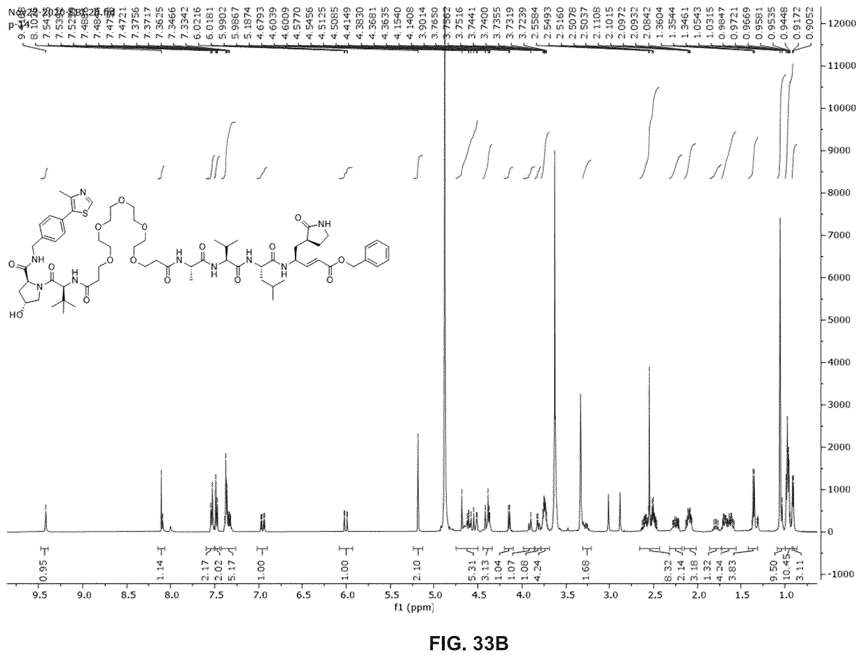

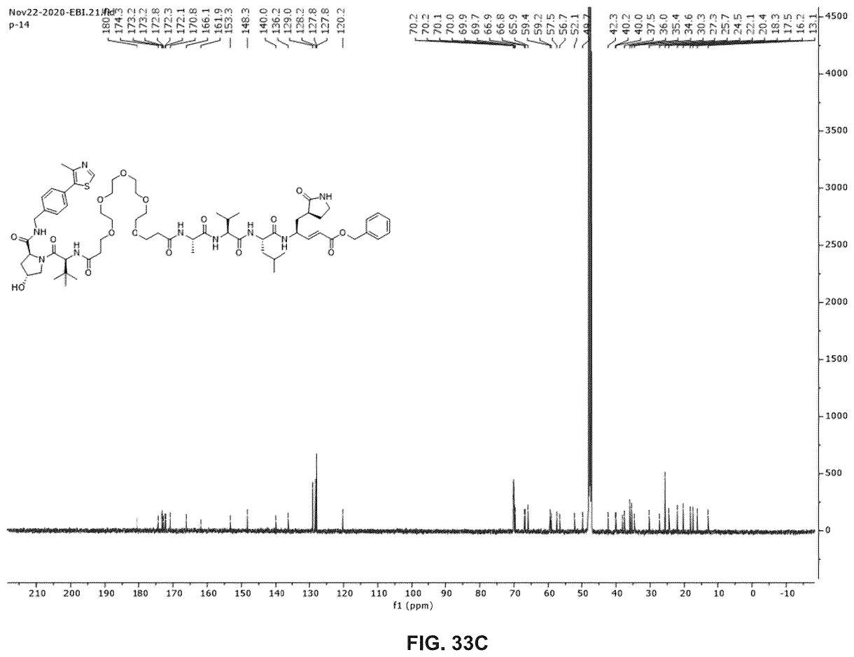

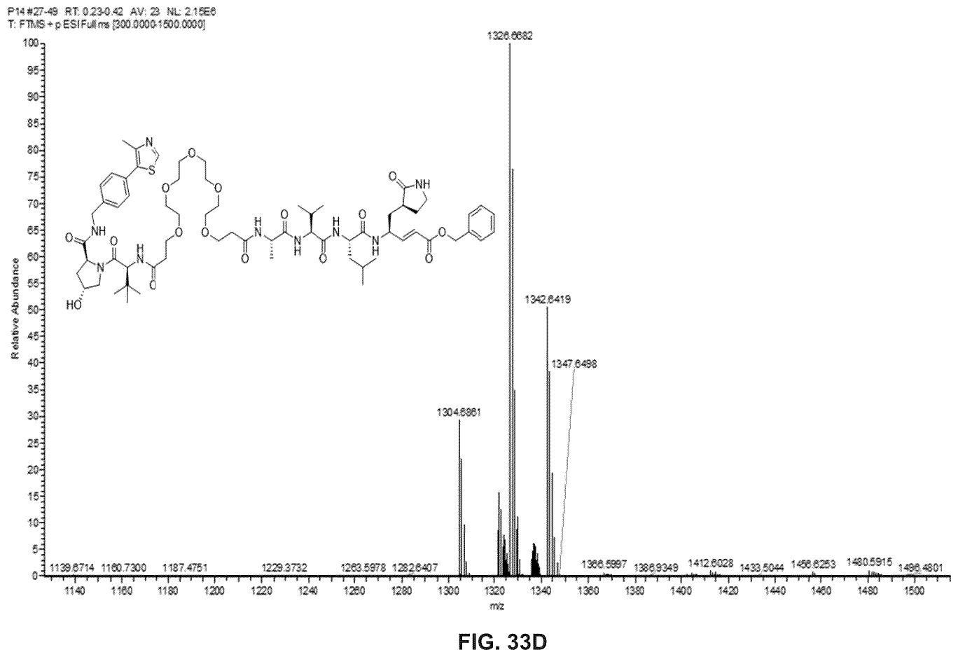

The synthesis of ethyl (16S,19S,22S,25S,E)-1-((2-(2,6-dioxopiperidin-3-yl)-1,3-dioxoisoindolin-4-yl)amino)-22-isobutyl-19-isopropyl-16-methyl-14,17,20,23-tetraoxo-25-(((S)-2-oxopyrrolidin-3-yl)methyl)-3,6,9,12-tetraoxa-15,18,21,24-tetraazaoctacos-26-en-28-oate (MP-15) is shown in FIG. 33A. To a solution of MP-TA-1 (10.2 mg, 0.02 mmol) and pomalidomide-PEG₄-C—COOH (11.4 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (45% acetonitrile) to get product as yellow-green solid (15.6 mg, 78%).

¹H NMR (methanol-d₄, 500 MHz): 8.19 (d, J=8.6 Hz, 1H), 7.96 (d, J=7.3 Hz, 1H), 7.79 (dd, J=7.8, 3.1 Hz, 1H), 7.46 (ddd, J=8.7, 7.0, 1.6 Hz, 1H), 7.00 (d, J=8.6 Hz, 1H), 6.96 (d, J=7.0 Hz, 1H), 6.79 (dd, J=15.7, 5.1 Hz, 1H), 5.82 (dd, J=15.7, 1.8 Hz, 1H), 5.02-4.88 (m, 1H), 4.58-4.48 (m, 1H), 4.36-4.32 (m, 1H), 4.29-4.21 (m, 1H), 4.10-4.04 (m, 3H), 3.90 (s, 2H), 3.62 (t, J=5.2 Hz, 2H), 3.57-3.54 (m, 10H), 3.41 (t, J=5.3 Hz, 2H), 3.19-3.08 (m, 2H), 2.81-2.72 (m, 1H), 2.70-2.54 (m, 2H), 2.53-2.39 (m, 1H), 2.22-2.11 (m, 1H), 2.05-1.88 (m, 3H), 1.76-1.62 (m, 1H), 1.61-1.40 (m, 4H), 1.28 (d, J=7.1 Hz, 3H), 1.17 (t, J=7.1 Hz, 3H), 0.88-0.81 (m, overlapped, 12H), as shown in FIG. 33B. ¹³C-NMR (methanol-d₄, 125 MHz): 180.6, 173.7, 173.6, 173.3, 173.3, 173.2, 172.1, 171.3, 170.3, 170.3, 169.3, 167.9, 166.4, 147.8, 147.8, 146.8, 135.8, 132.5, 120.4, 116.9, 110.6, 109.9, 70.6, 70.3, 70.2, 70.0, 69.7, 69.3, 60.3, 58.8, 52.3, 48.8, 40.4, 40.4, 40.1, 38.1, 34.8, 30.8, 30.6, 27.4, 24.6, 22.4, 21.9, 20.6, 18.4, 17.4, 17.3, 16.7, 13.1, as shown in FIG. 33C. HR-ESI-MS (n/z) calculated for 1021.4831 (M+Na)⁺, found 1021.4843 (FIG. 33D).

### Synthesis of MP-16

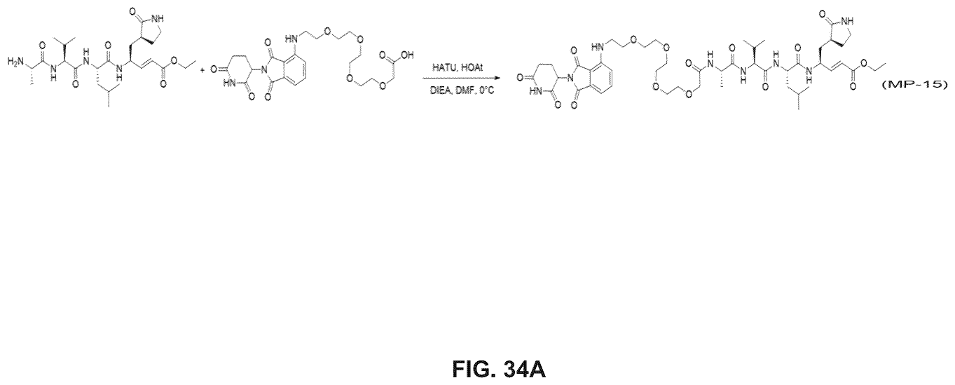

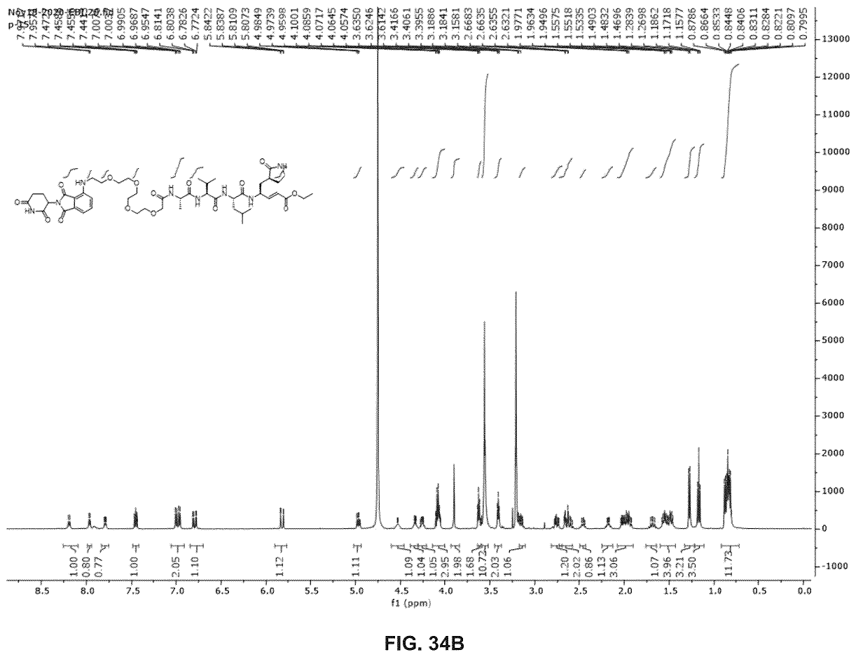

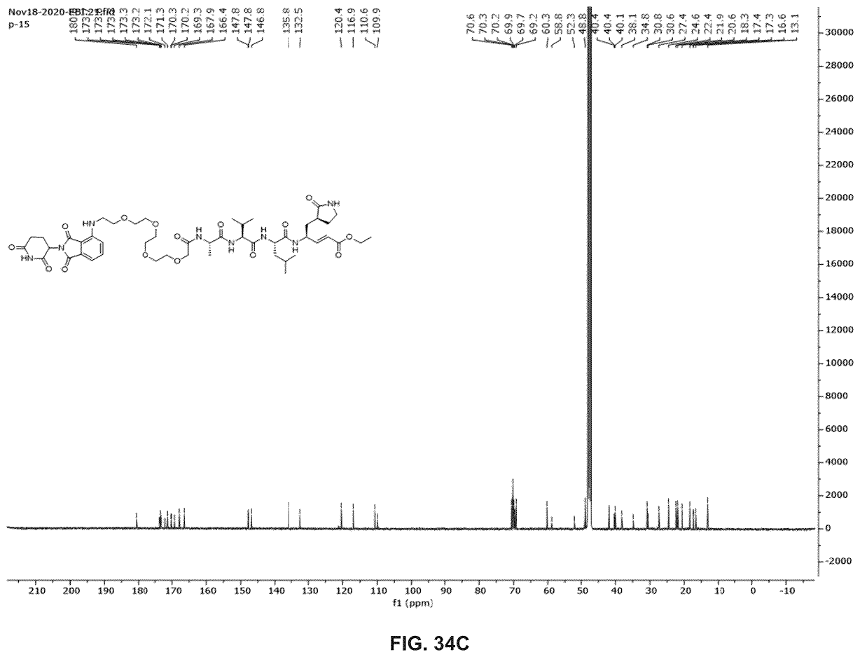

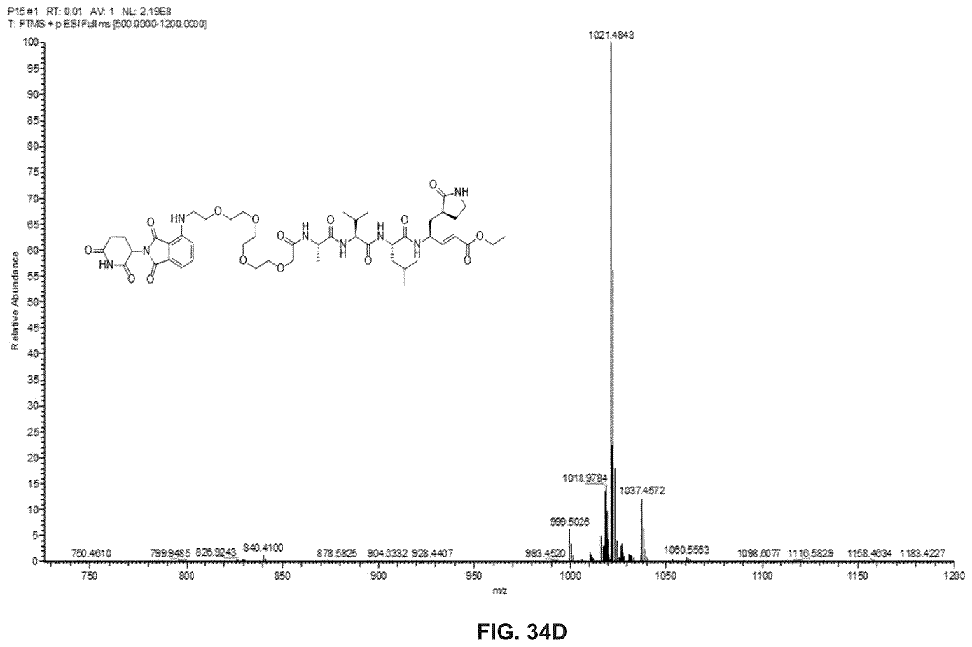

The synthesis of benzyl (3S,6S,9S,12S,E)-9-isobutyl-6-isopropyl-3-methyl-1,4,7,10-tetraoxo-12-(((S)-2-oxopyrrolidin-3-yl)methyl)-1-(3-(pyridin-2-yl)phenyl)-2,5,8,11-tetraazapentadec-13-en-15-oate (MP-16) is shown in FIG. 34A. To a solution of MP-TA-2 (11.4 mg, 0.02 mmol) and 3-(pyridin-2-yl)benzoic acid (4.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (35% acetonitrile) to get product as white solid (10.6 mg, 71%). ¹H NMR (methanol-d₄, 500 MHz): 8.68 (d, J=4.9 Hz, 1H), 8.50 (t, J=1.9 Hz, 1H), 8.16 (dt, J=7.9, 1.4 Hz, 1H), 8.11 (d, J=7.4 Hz, 1H), 8.04-7.86 (m, 3H), 7.63 (t, J=7.8 Hz, 1H), 7.40-7.28 (m, 5H), 6.96 (dd, J=15.7, 5.1 Hz, 1H), 6.01 (dd, J=15.7, 1.6 Hz, 1H), 5.19 (s, 2H), 4.72-4.57 (m, 2H), 4.46-4.30 (m, 1H), 4.22-4.13 (m, 1H), 3.30-3.22 (m, 2H), 2.62 (dd, J=8.7, 3.2 Hz, 1H), 2.32-2.21 (m, 1H), 2.13 (dq, J=11.7, 4.4 Hz, 2H), 1.90-1.55 (m, 5H), 1.52 (d, J=7.1 Hz, 3H), 1.01-0.94 (m, 9H), 0.91 (d, J=6.0 Hz, 3H), as shown in FIG. 34B. ¹³C-NMR (methanol-d₄, 125 MHz): 180.6, 174.7, 174.6, 173.3, 172.3, 168.7, 166.1, 156.3, 148.8, 148.3, 148.3, 139.0, 138.1, 136.2, 134.3, 130.1, 128.8, 128.2, 128.0, 127.8, 126.0, 123.0, 121.5, 120.2, 65.9, 59.4, 52.3, 50.5, 50.5, 40.2, 40.0, 38.1, 34.6, 30.3, 27.3, 24.6, 22.0, 20.4, 18.2, 17.4, 16.1, as shown in FIG. 34C. HR-ESI-MS (m/z) calculated for 753.3973 (M+H)+, found 753.3960 (FIG. 34D).

### Synthesis of MP-17

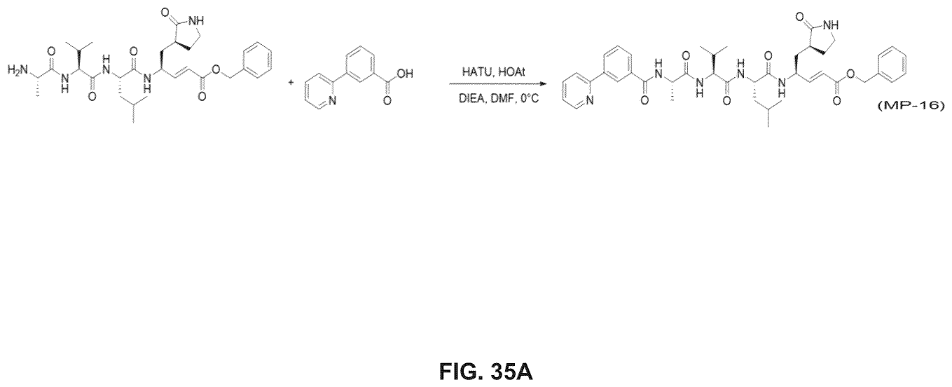

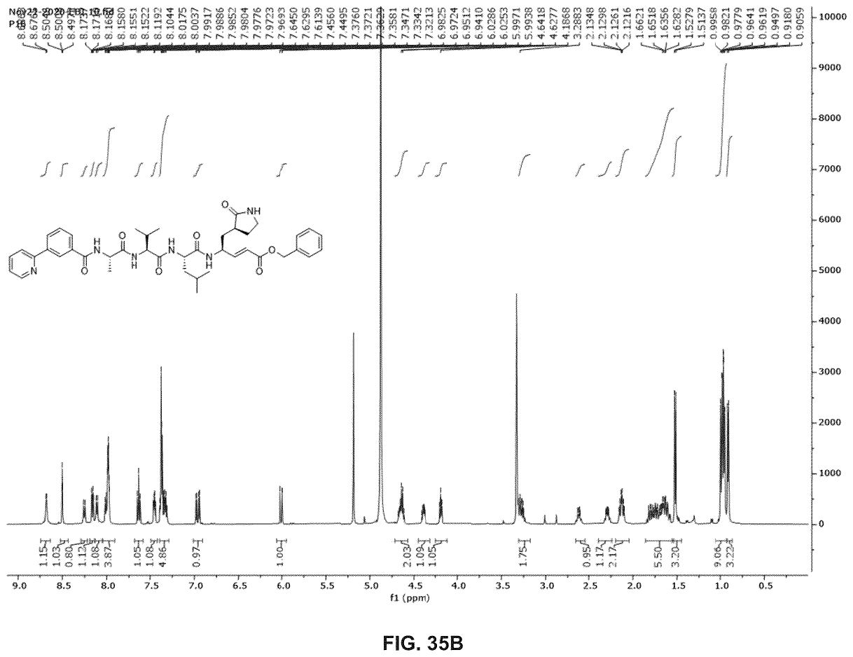

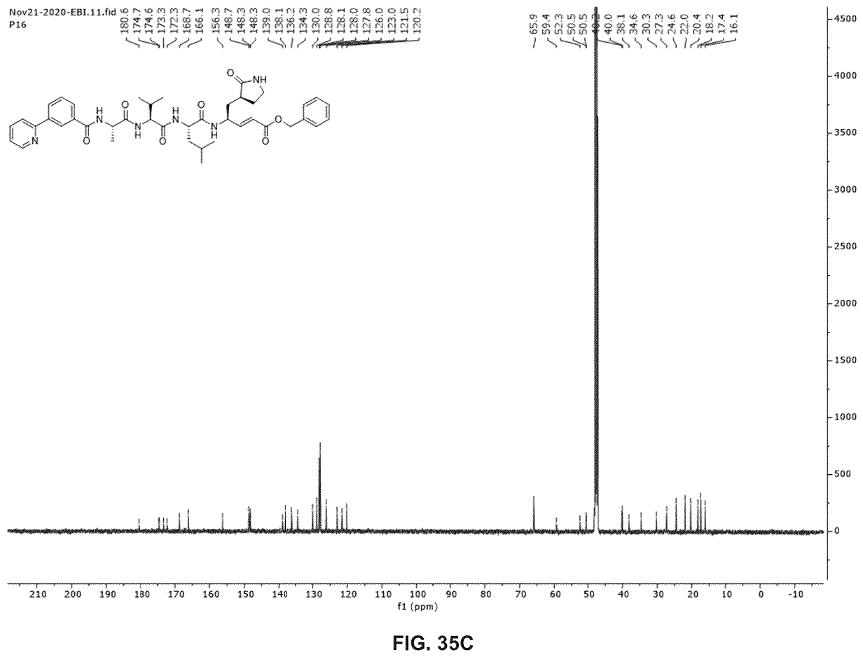

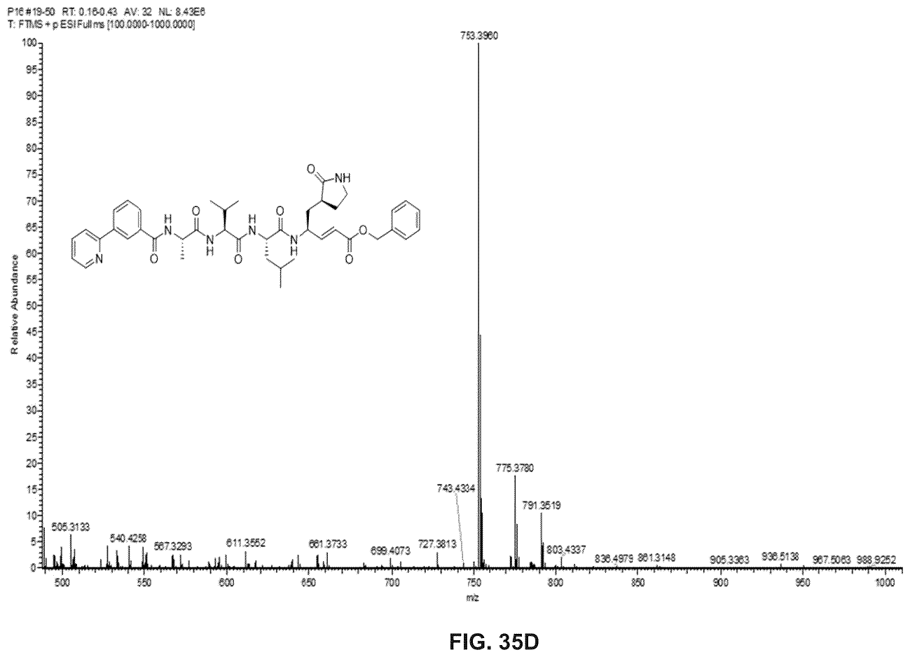

The synthesis of benzyl (3S,6S,9S,12S,E)-9-isobutyl-6-isopropyl-3-methyl-1,4,7,10-tetraoxo-12-(((S)-2-oxopyrrolidin-3-yl)methyl)-1-(4-(pyridin-2-yl)phenyl)-2,5,8,11-tetraazapentadec-13-en-15-oate (MP-17) is shown in FIG. 35A. To a solution of MP-TA-2 (11.4 mg, 0.02 mmol) and 4-(pyridin-2-yl)benzoic acid (4.0 mg, 0.02 mmol) in 1 mL DMF, DIEA (17.5 p L, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (35% acetonitrile) to get product as white solid (11.0 mg, 73%). ¹H NMR (methanol-d₄, 500 MHz): 8.67 (d, J=4.8 Hz, 1H), 8.33-7.82 (m, 6H), 7.46 (ddd, J=6.8, 4.9, 1.8 Hz, 1H), 7.40-7.29 (m, 5H), 6.97 (dd, J=15.7, 5.1 Hz, 1H), 6.01 (dd, J=15.7, 1.7 Hz, 1H), 5.19 (s, 2H), 4.71-4.57 (m, 2H), 4.39 (dd, J=10.0, 5.0 Hz, 1H), 4.18 (d, J=6.4 Hz, 1H), 3.31-3.24 (m, 2H), 2.67-2.56 (m, 1H), 2.34-2.23 (m, 1H), 2.16-2.06 (m, 2H), 1.86-1.55 (m, 5H), 1.52 (d, J=7.2 Hz, 3H), 1.00-0.95 (m, 9H), 0.92 (d, J=6.0 Hz, 3H), as shown in FIG. 35B. ¹³C-NMR (methanol-d₄, 125 MHz): 180.7, 174.6, 173.3, 172.2, 168.6, 166.3, 156.1, 148.9, 148.3, 142.0, 138.1, 136.1, 134.1, 128.2, 127.9, 127.9, 127.8, 126.9, 123.2, 121.7, 120.2, 66.0, 59.3, 52.2, 50.5, 40.2, 40.1, 38.2, 34.5, 30.3, 27.3, 24.6, 22.0, 20.4, 18.3, 17.4, 16.1, as shown in FIG. 35C. HR-ESI-MS (m/z) calculated for 753.3973 (M+H)⁺, found 753.3963 (FIG. 35D).

### Synthesis of MP-TA-5

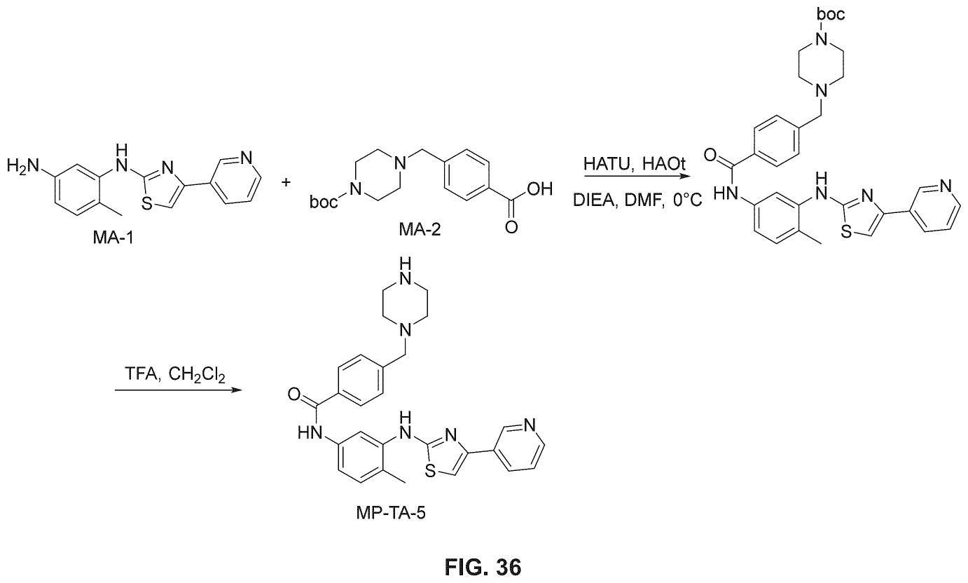

The synthesis of N-(4-methyl-3-((4-(pyridin-3-yl)thiazol-2-yl)amino)phenyl)-4-(piperazin-1-ylmethyl)benzamide (MP-TA-5) is shown in FIG. 36. To a solution of MA-1 (565 mg, 2 mmol) and MA-2 (440 mg, 2 mmol) in 30 mL DMF, DIEA (1750 μL, 4 mmol), HATU (1500 mg, 4 mmol), and HOAt (60 mg, 0.4 mmol) were added under ice bath. The reaction was stirred at 0° C. for half hour, then the mixture was purified with HPLC to get product as oil-like solid. Then, the Boc protecting group was removed using TFA/DCM (1/2, 120 min) to get product (726 mg, 75%).

### Synthesis of MP-18

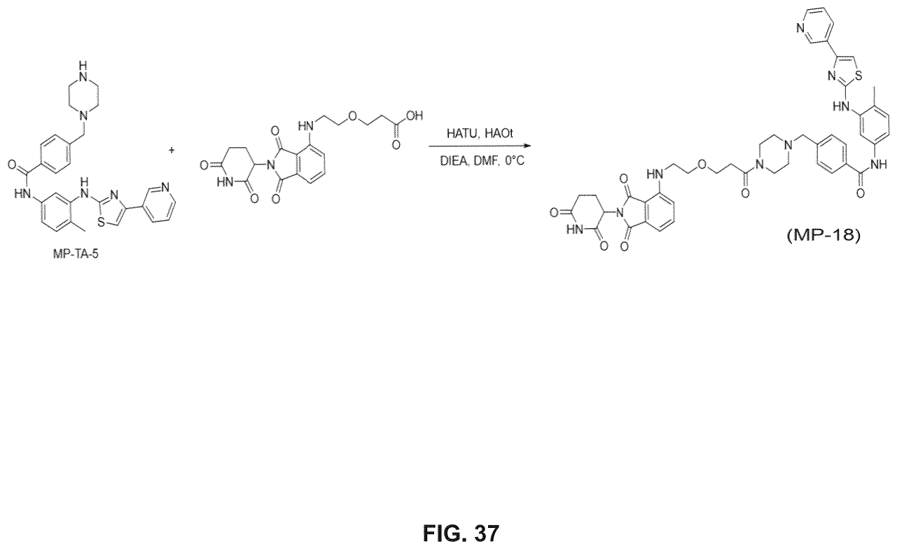

The synthesis of 4-((4-(3-(2-((2-(2,6-dioxopiperidin-3-yl)-1,3-dioxoisoindolin-4-yl)amino)ethoxy)propanoyl)piperazin-1-yl)methyl)-N-(4-methyl-3-((4-(pyridin-3-yl)thiazol-2-yl)amino)phenyl)benzamide (MP-18) is shown in FIG. 37.

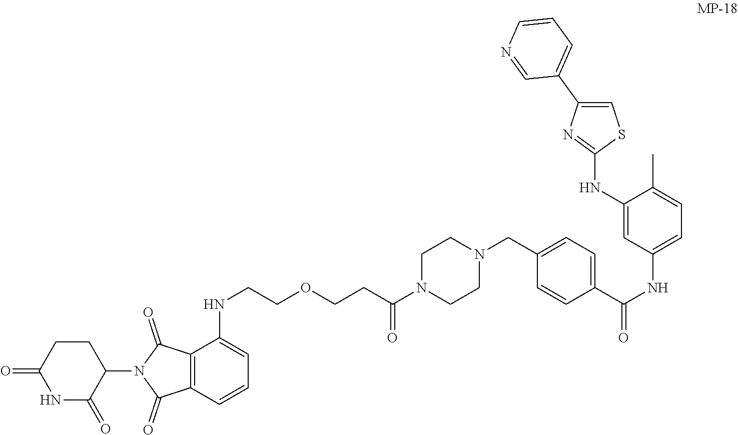 [SMILES: Cc1ccc(NC(=O)c2ccc(CN3CCN(C(=O)CCOCCNc4cccc5c4C(=O)N(C4CCC(=O)NC4=O)C5=O)CC3)cc2)cc1Nc1nc(-c2cccnc2)cs1]

To a solution of MP-TA-5 (9.6 mg, 0.02 mmol) and pomalidomide-PEG-acid (7.8 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half an hour, then the mixture was purified with HPLC (40% acetonitrile) to get product as yellow-green solid (11.9 mg, 70%).

### Synthesis of MP-19, MP-20, MP-21, MP-22, MP-23, MP-24, MP-25, MP-26, MP-27

The synthesis of MP-19, MP-20, MP-21, MP-22, MP-23, MP-24, MP-25, MP-26, and MP-27 was conducted following the same synthetic method as MP-18 detailed above. These compounds are depicted below:

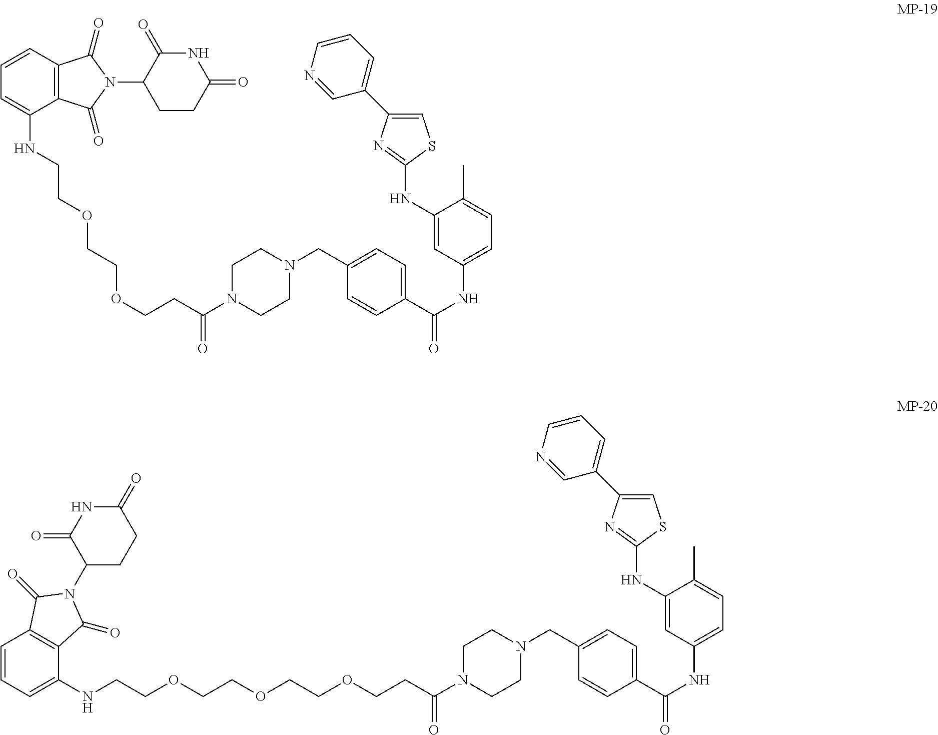 [SMILES: Cc1ccc(NC(=O)c2ccc(CN3CCN(C(=O)CCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCNc4cccc5c4C(=O)N(C4CCC(=O)NC4=O)C5=O)CC3)cc2)cc1Nc1nc(-c2cccnc2)cs1]

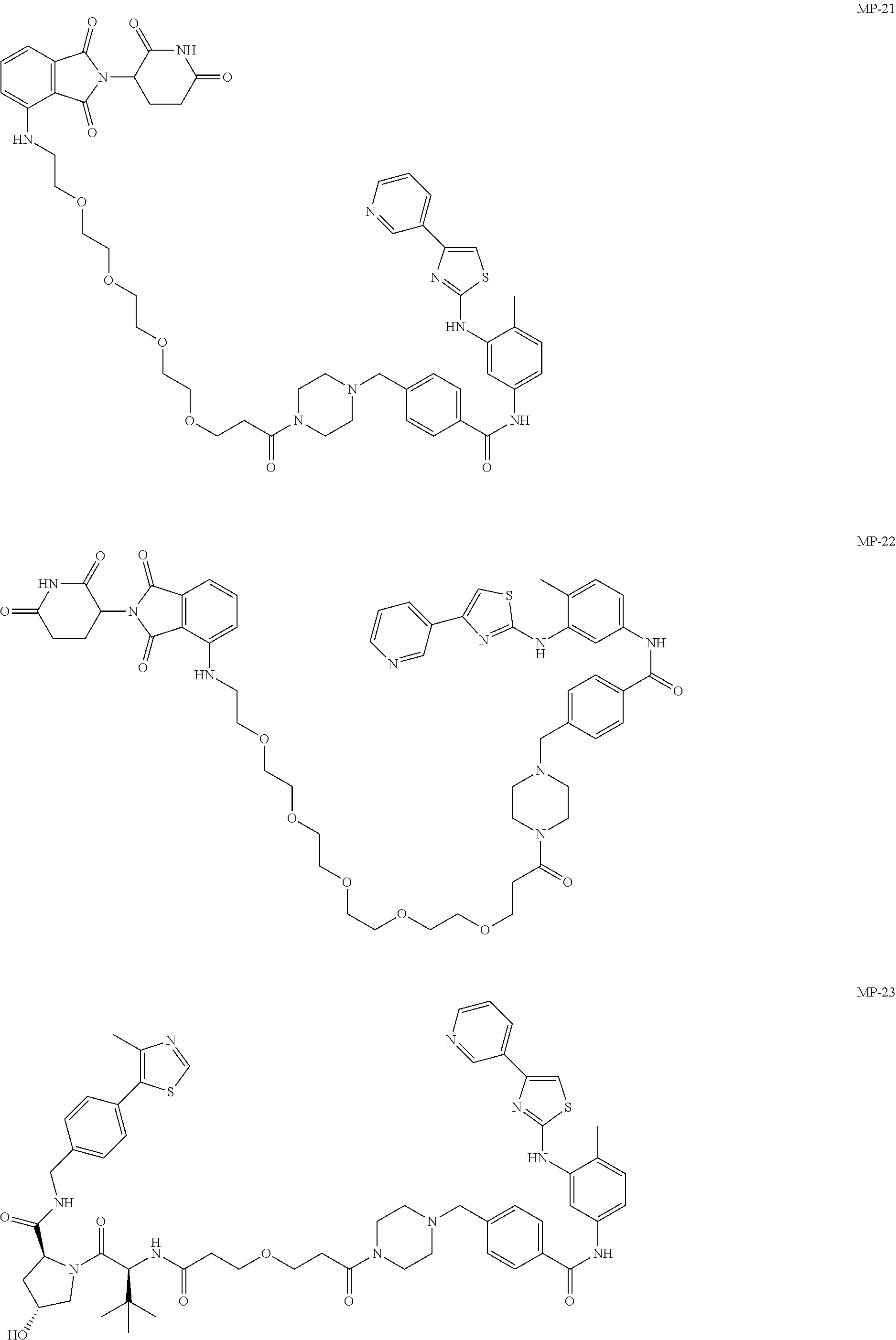 [SMILES: Cc1ccc(NC(=O)c2ccc(CN3CCN(C(=O)CCOCCOCCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCC(=O)N[C@H](C(=O)N4C[C@H](O)C[C@H]4C(=O)NCc4ccc(-c5scnc5C)cc4)C(C)(C)C)CC3)cc2)cc1Nc1nc(-c2cccnc2)cs1]

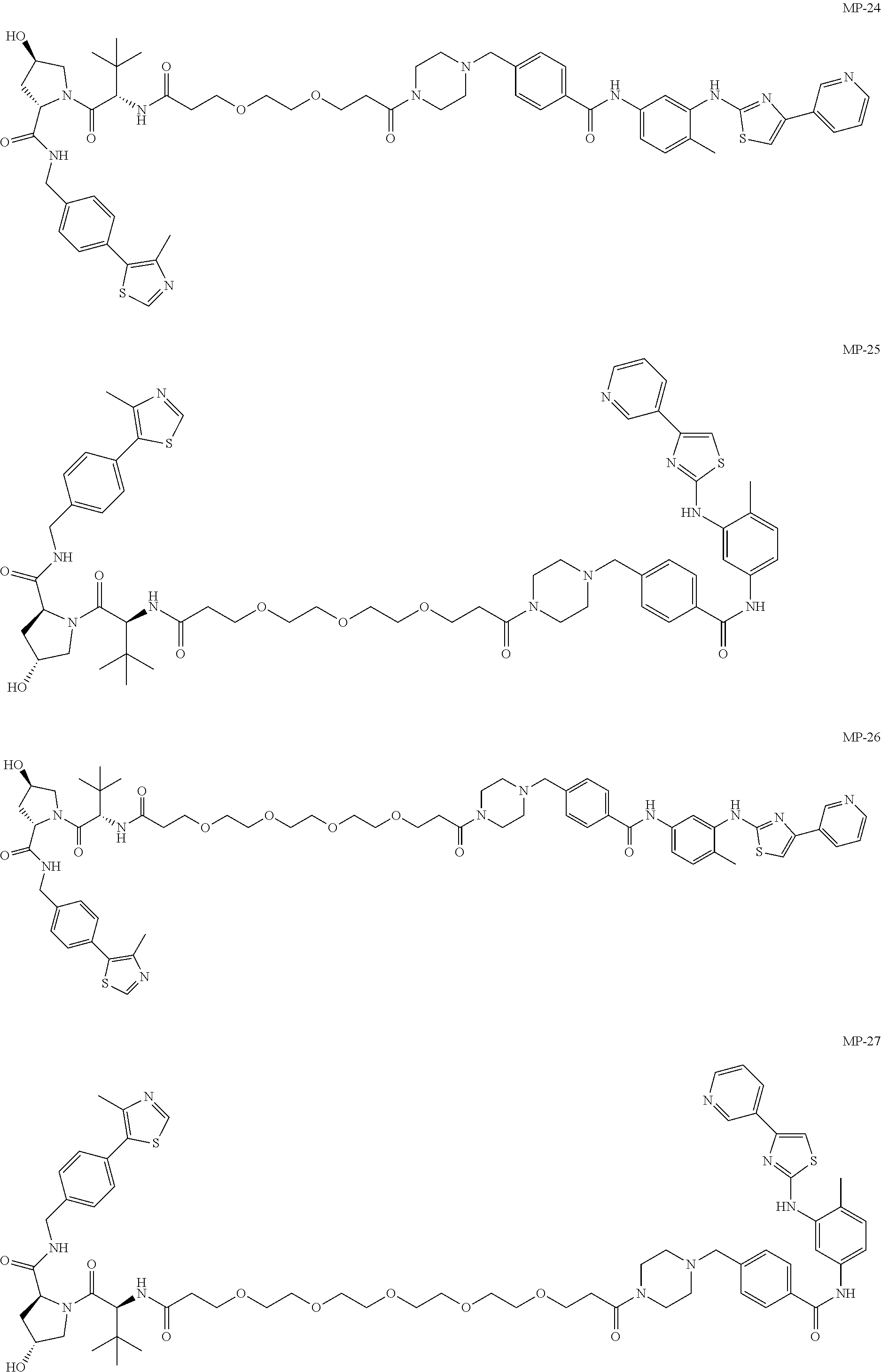

### Synthesis of TA-3

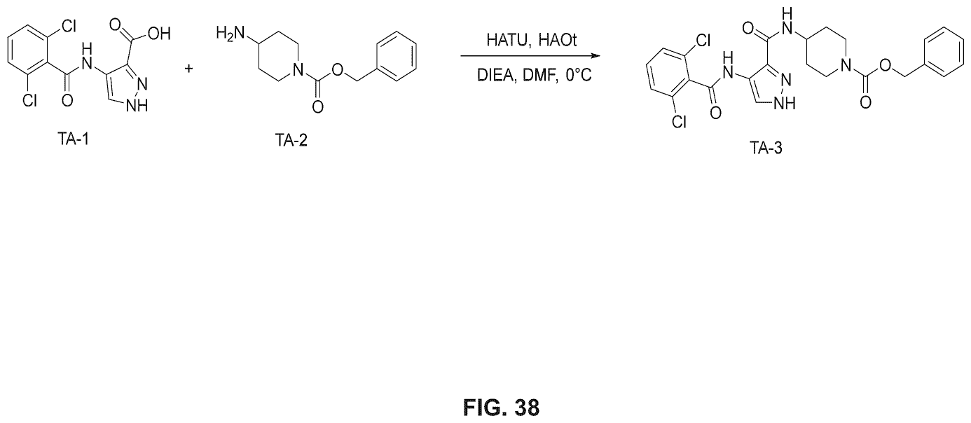

The synthesis of benzyl 4-(4-(2,6-dichlorobenzamido)-1H-pyrazole-3-carboxamido)piperidine-1-carboxylate (TA-3) is depicted in FIG. 38. To a solution of TA-1 (900 mg, 3 mmol) and TA-2 (695 mg. 3 mmol) in 30 mL DMF, DIEA (2625 μL, 6 mmol), HATU (2250 mg, 6 mmol), and HOAt (90 mg, 0.6 mmol) were added under ice bath. The reaction was stirred at 0° C. for half an hour, then the mixture was purified with HPLC to get product as white solid. (1223 mg, 79%).

### Synthesis of TA-6

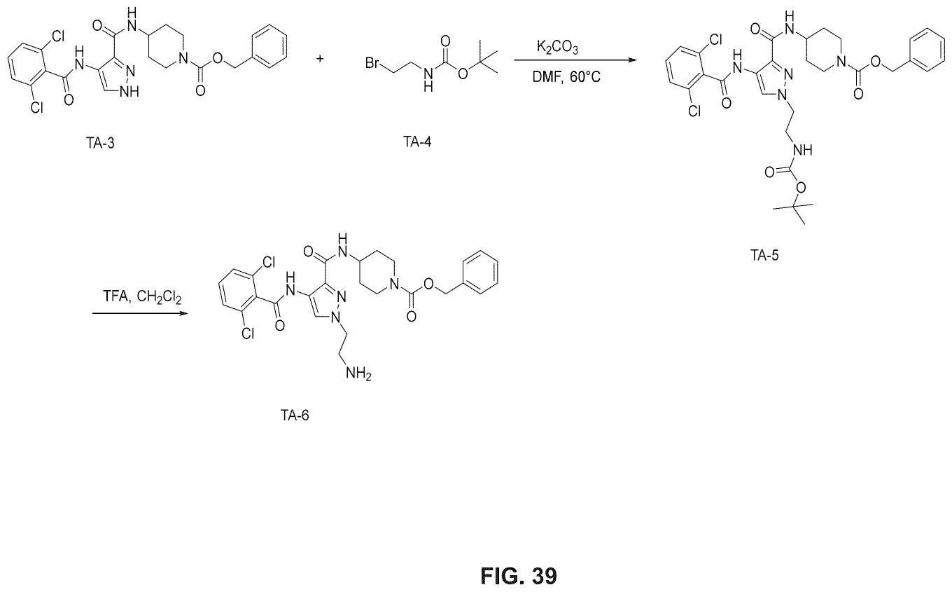

The synthesis of benzyl 4-(1-(2-aminoethyl)-4-(2,6-dichlorobenzamido)-1H-pyrazole-3-carboxamido)piperidine-1-carboxylate (TA-6) is depicted in FIG. 39. K₂CO₃ (1.38 g, 10 mmol) and TA-4 (1035 mg, 2 mmol) were added to a solution of TA-3 (448 mg, 2 mmol) in DMF (20 ml), and the reaction mixture was heated to 60° C. and stirred for 15 h. After diluting the reaction mixture with water, the reaction mixture was extracted with EtOAc, then the organic layer was washed with water and brine, and the mixture was purified with silica gel to get product as a white solid. Then, the Boc protecting group was removed using TFA/DCM (1/2, 120 min) to get product (693 mg, 62%).

### Synthesis of MP-28

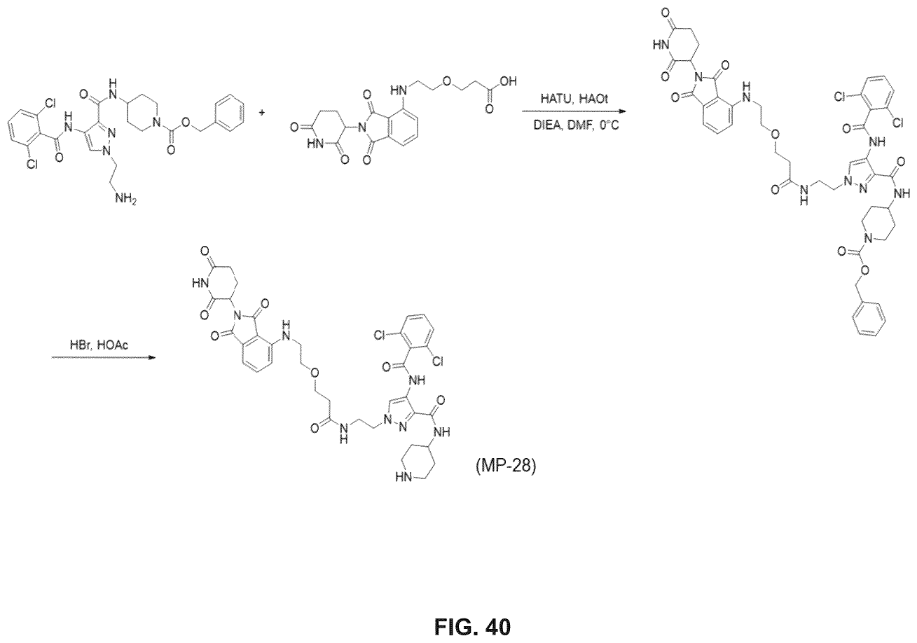

The synthesis of 4-(2,6-dichlorobenzamido)-1-(2-(3-(2-((2-(2,6-dioxopiperidin-3-yl)-1,3-dioxoisoindolin-4-yl)amino)ethoxy)propanamido)ethyl)-N-(piperidin-4-yl)-1H-pyrazole-3-carboxamide (MP-28) is depicted in FIG. 40. To a solution of MP-TA-6 (11.2 mg, 0.02 mmol) and pomalidomide-PEG-acid (7.8 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half an hour, then the mixture was purified with HPLC to get yellow-green solid. Then, the Cbz protecting group was removed using HBr/HOAc (30 min) to get product (12.5 mg, 79%).

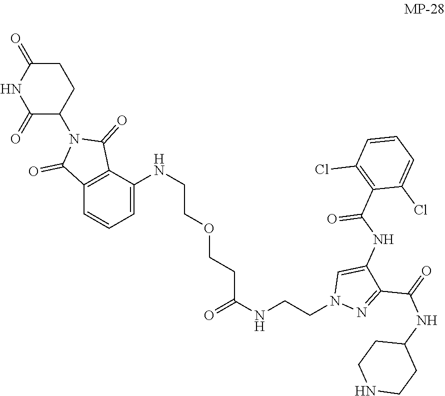 [SMILES: O=C(CCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

### Synthesis of MP-29, MP-30, MP-31, MP-32, MP-33, MP-34, MP-35, MP-36, and MP-37

The synthesis of MP-29, MP-30, MP-31, MP-32, MP-33, MP-34, MP-35, MP-36, and MP-37 was conducted following the same synthetic method as described above for MP-28. The structures of these compounds are as follows:

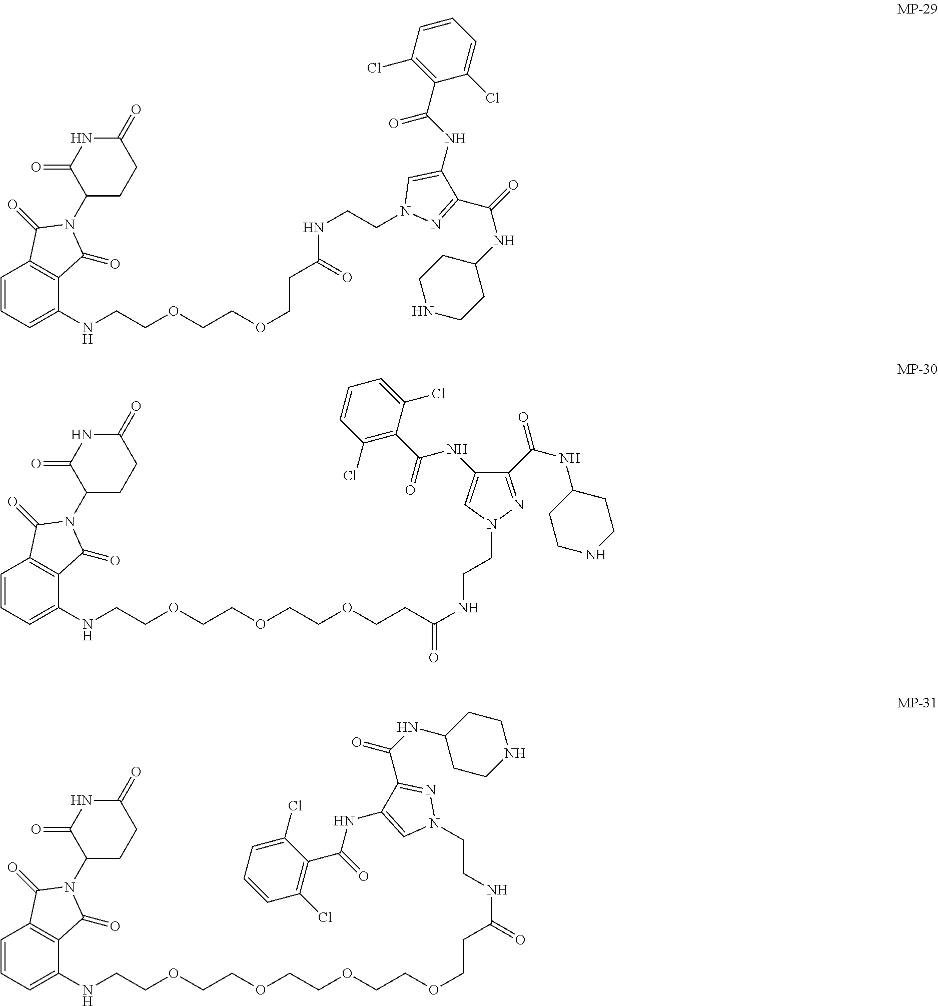 [SMILES: O=C(CCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; O=C(CCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; O=C(CCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

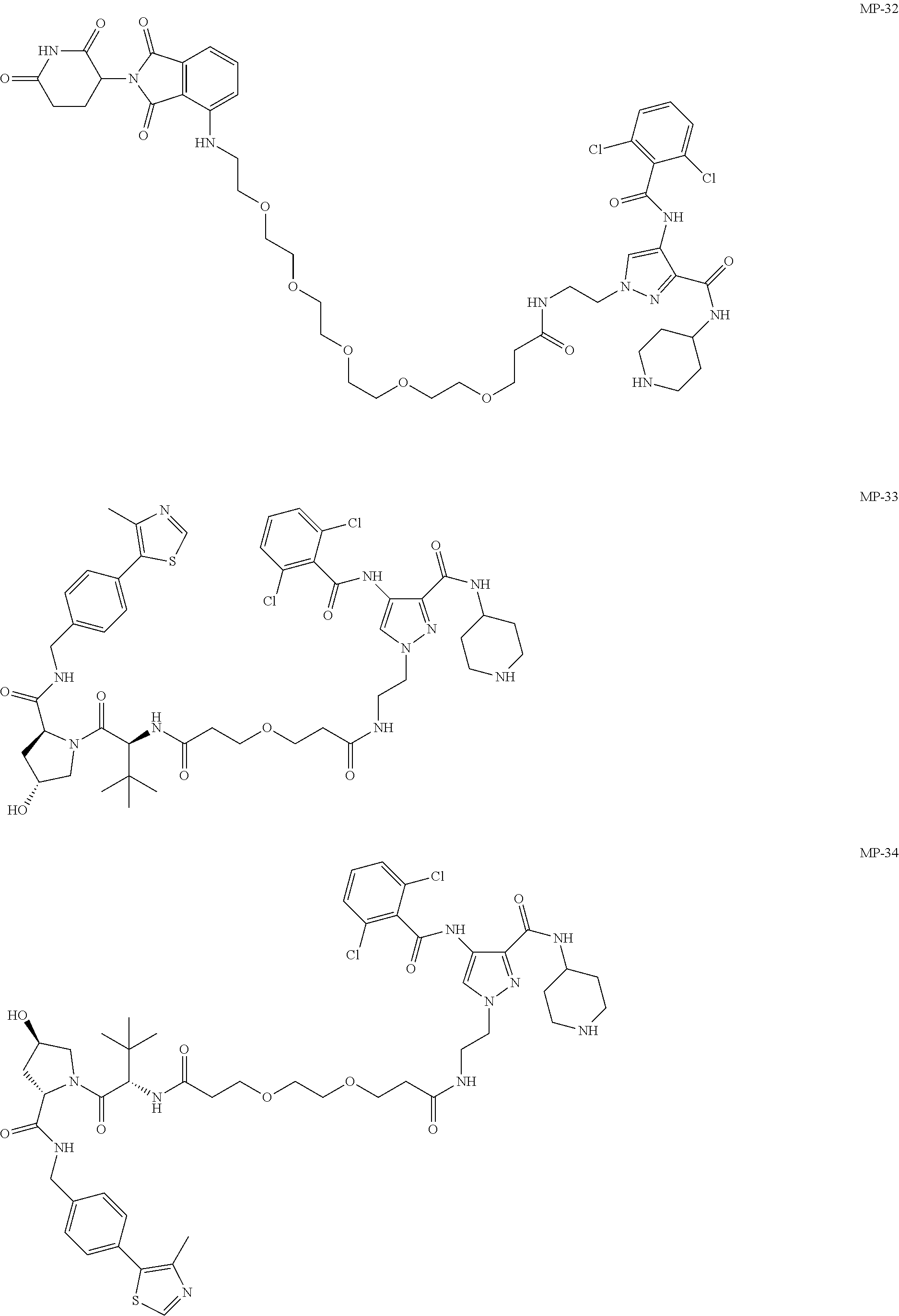 [SMILES: O=C(CCOCCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; Cc1ncsc1-c1ccc(CNC(=O)[C@@H]2C[C@@H](O)CN2C(=O)[C@@H](NC(=O)CCOCCC(=O)NCCn2cc(NC(=O)c3c(Cl)cccc3Cl)c(C(=O)NC3CCNCC3)n2)C(C)(C)C)cc1 ; Cc1ncsc1-c1ccc(CNC(=O)[C@@H]2C[C@@H](O)CN2C(=O)[C@@H](NC(=O)CCOCCOCCC(=O)NCCn2cc(NC(=O)c3c(Cl)cccc3Cl)c(C(=O)NC3CCNCC3)n2)C(C)(C)C)cc1]

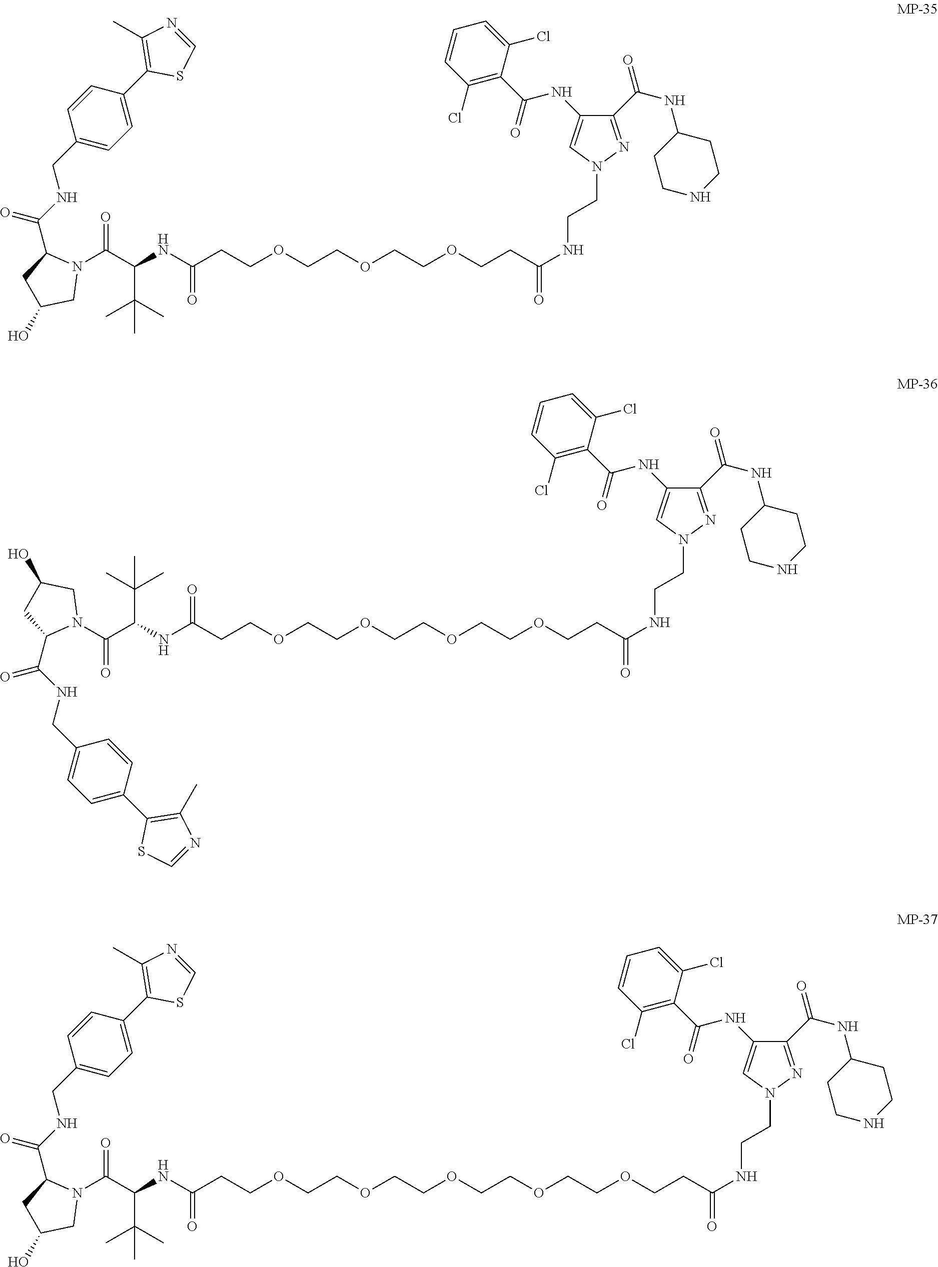 [SMILES: Cc1ncsc1-c1ccc(CNC(=O)[C@@H]2C[C@@H](O)CN2C(=O)[C@@H](NC(=O)CCOCCOCCOCCC(=O)NCCn2cc(NC(=O)c3c(Cl)cccc3Cl)c(C(=O)NC3CCNCC3)n2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)NCCn2cc(NC(=O)c3c(Cl)cccc3Cl)c(C(=O)NC3CCNCC3)n2)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)NCCn2cc(NC(=O)c3c(Cl)cccc3Cl)c(C(=O)NC3CCNCC3)n2)C(C)(C)C)cc1]

### Synthesis of MP-TA-6

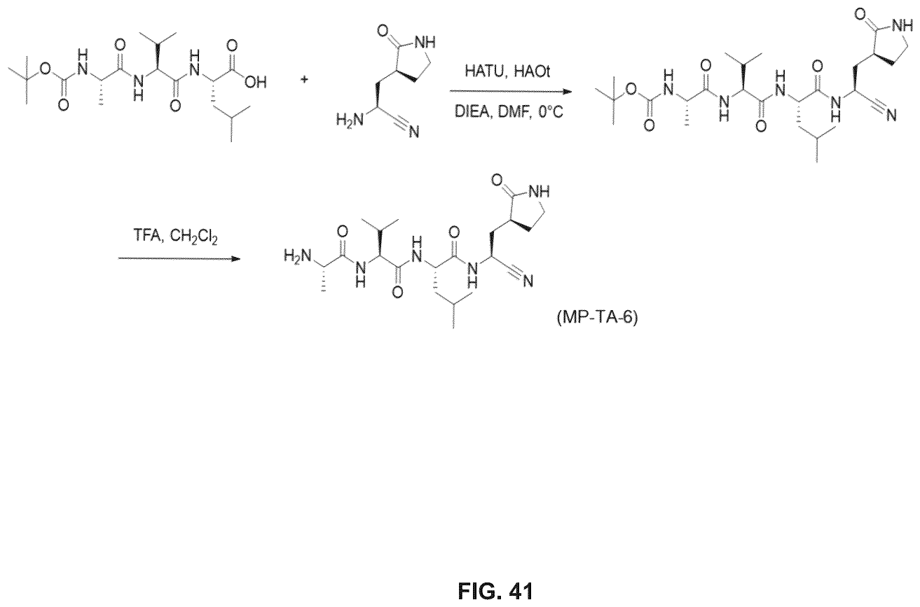

The synthesis of MP-TA-6 is depicted in FIG. 41. The same synthetic methods described above with respect to MP-TA-3 were utilized to synthesize MP-TA-6.

### Synthesis of MP-38

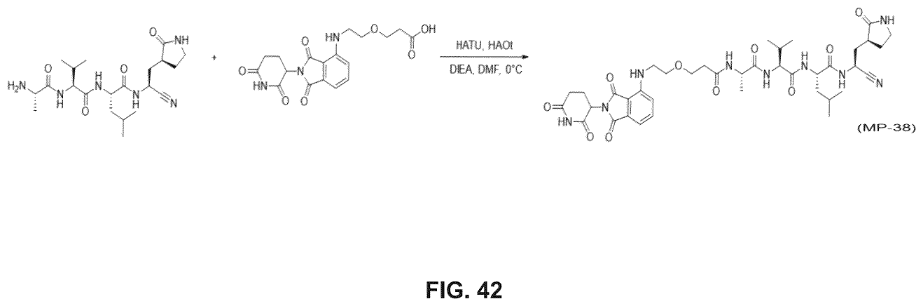

The synthesis of MP-38 is depicted in FIG. 42. The same synthetic methods described above with respect to MP-5 were utilized to synthesize MP-38.

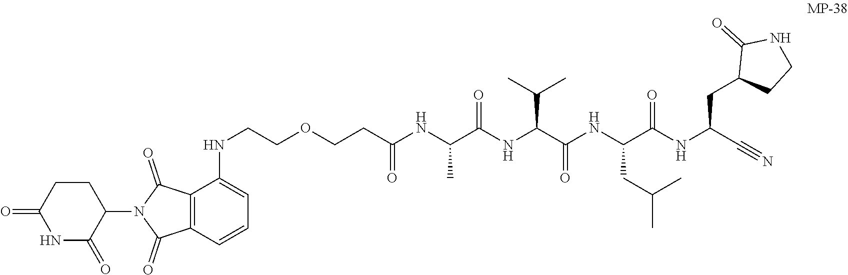 [SMILES: CC(C)C[C@H](NC(=O)[C@@H](NC(=O)[C@H](C)NC(=O)CCOCCNc1cccc2c1C(=O)N(C1CCC(=O)NC1=O)C2=O)C(C)C)C(=O)N[C@H](C#N)C[C@@H]1CCNC1=O]

### Synthesis of MP-39 through MP-47

The following structures were synthesized according to the same synthetic methods described above with respect to MP-38:

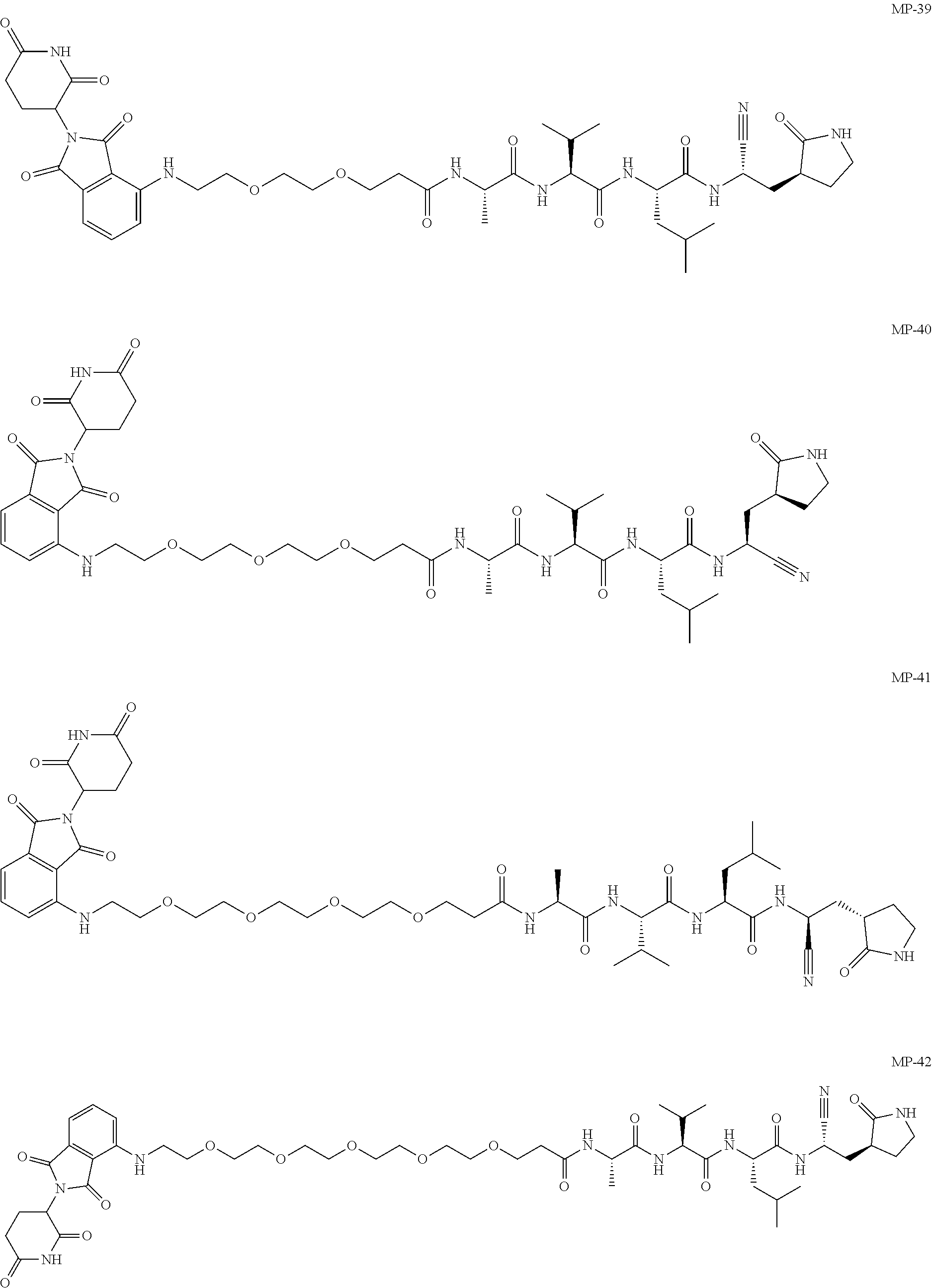

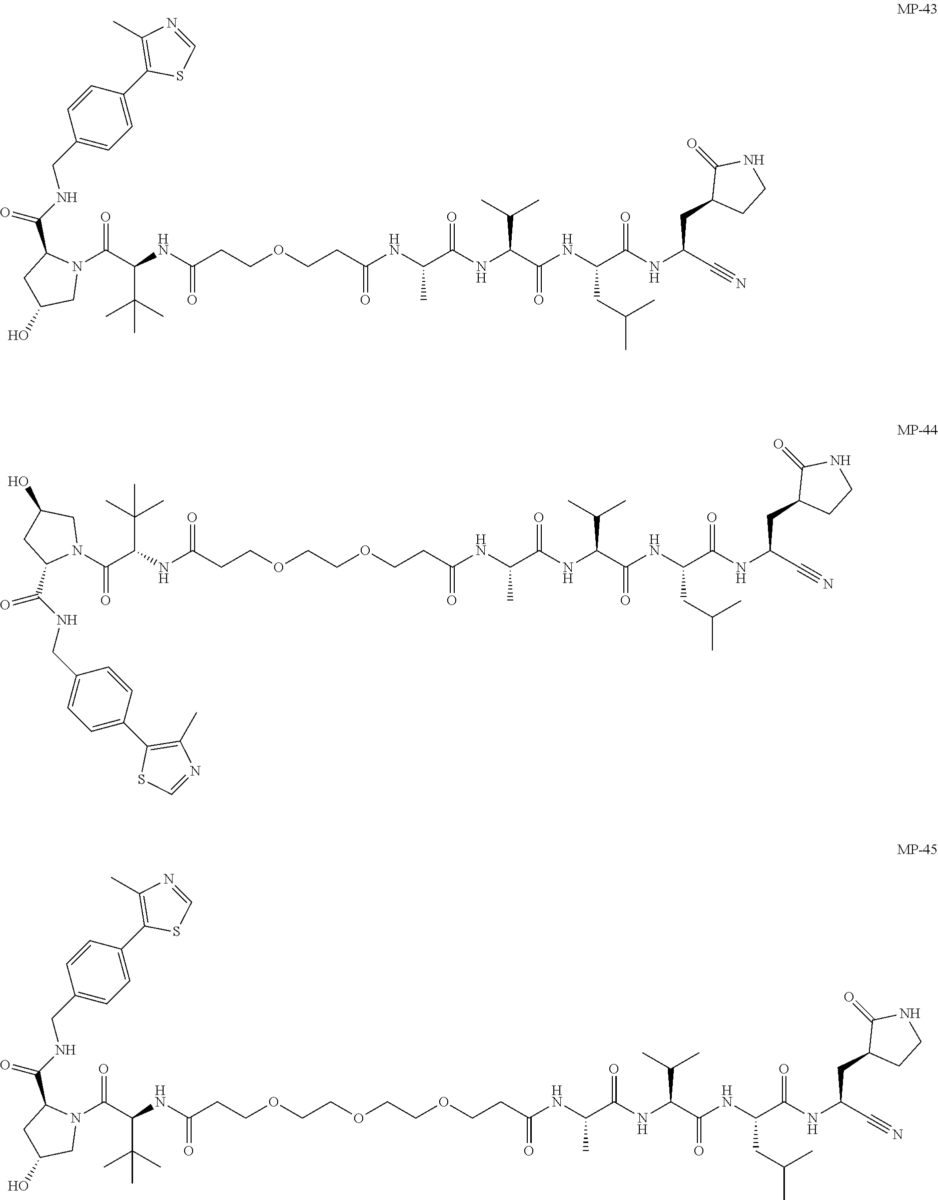

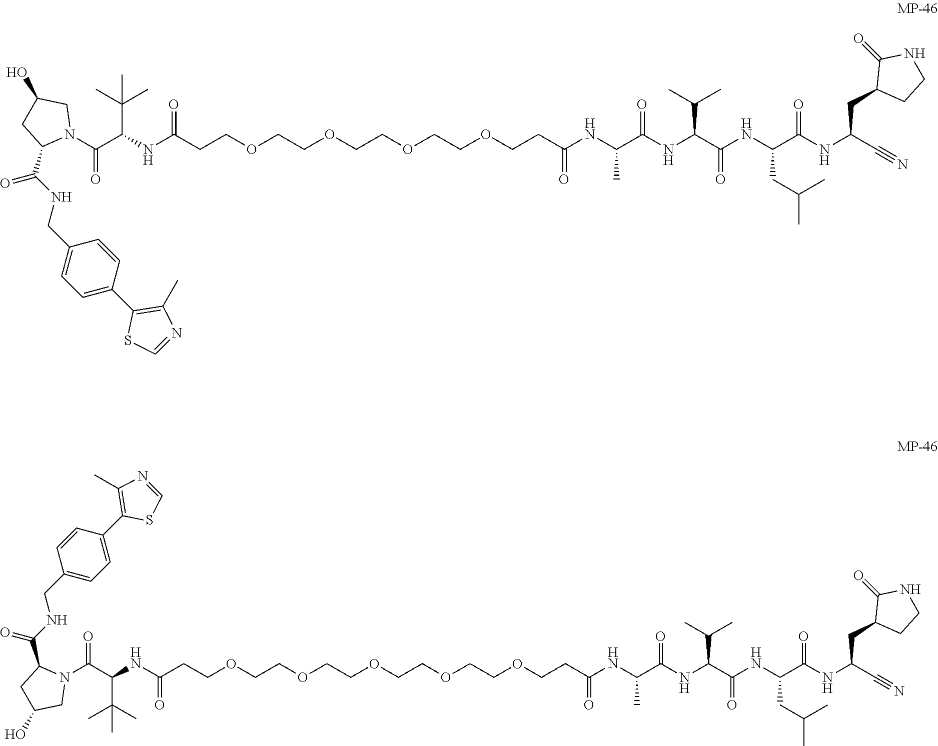 [SMILES: Cc1ncsc1-c1ccc(CNC(=O)[C@@H]2C[C@@H](O)CN2C(=O)[C@@H](NC(=O)CCOCCOCCOCCOCCC(=O)N[C@@H](C)C(=O)N[C@H](C(=O)N[C@@H](CC(C)C)C(=O)N[C@H](C#N)C[C@@H]2CCNC2=O)C(C)C)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)N[C@@H](C)C(=O)N[C@H](C(=O)N[C@@H](CC(C)C)C(=O)N[C@H](C#N)C[C@@H]2CCNC2=O)C(C)C)C(C)(C)C)cc1]

### Synthesis of (S)-5-amino-2-methyl-N-(1-(naphthalen-1-yl)ethyl)benzamide

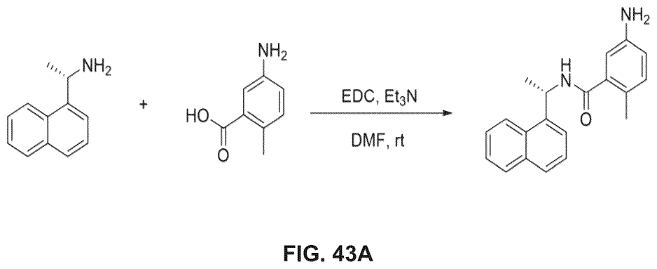

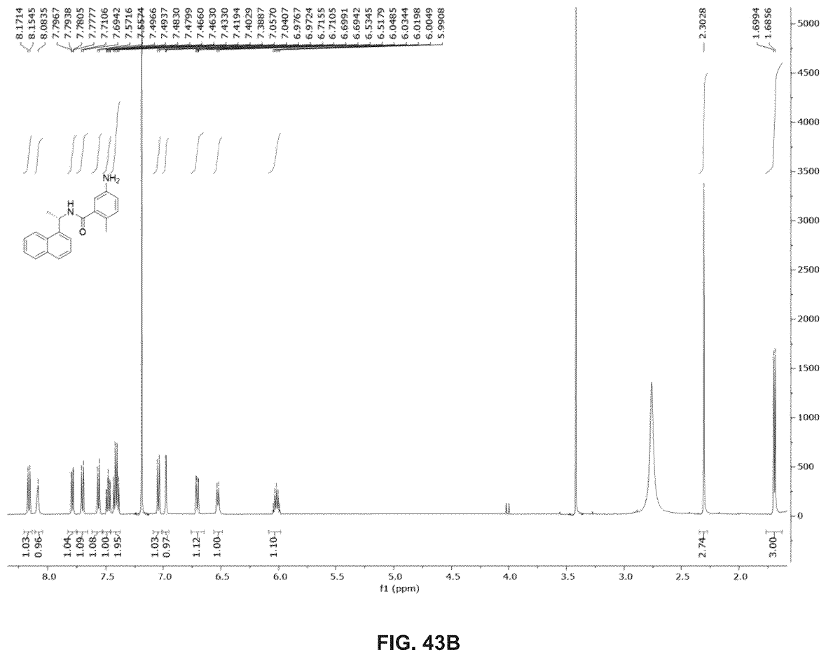

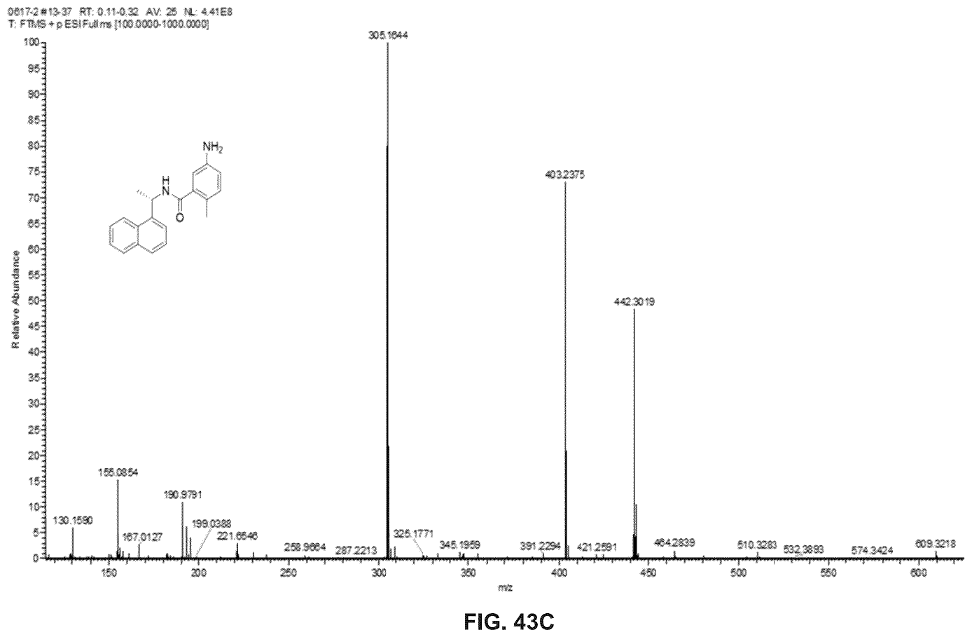

The synthesis of (S)-5-amino-2-methyl-N-(1-(naphthalen-1-yl)ethyl)benzamide is shown in FIG. 43A. The synthetic procedure of (S)-5-amino-2-methyl-N-(1-(naphthalen-1-yl)ethyl)benzamide was the same as PLP-TA-1. ¹H NMR (chloroform-d, 500 MHz): 8.16 (d, J=8.5 Hz, 1H), 8.08 (s, NH), 7.79 (dd, J=8.1, 1.4 Hz, 1H), 7.70 (d, J=8.2 Hz, 1H), 7.56 (d, J=7.1 Hz, 1H), 7.48 (dt, J=8.4, 6.8 Hz, 1H), 7.44-7.38 (m, 2H), 7.05 (d, J=8.2 Hz, 1H), 6.97 (d, J=2.4 Hz, NH₂), 6.70 (dd, J=8.2, 2.5 Hz, 1H), 6.53 (d, J=8.3 Hz, 1H), 6.11-5.95 (m, 1H), 2.30 (s, 3H), 1.69 (d, J=6.9 Hz, 3H), shown in FIG. 43B. HR-ESI-MS (ml) calculated for 305.1650 (M+H)⁺, found 305.1644 (FIG. 43C).

### Synthesis of PLP-1

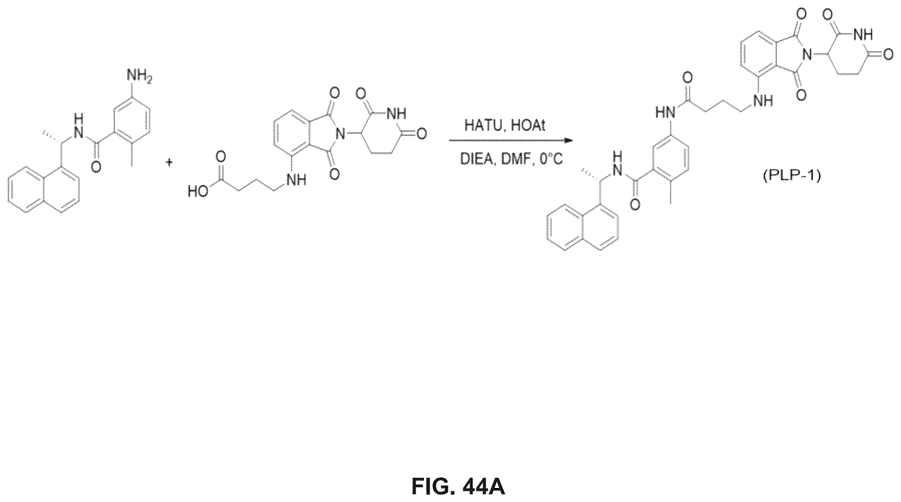

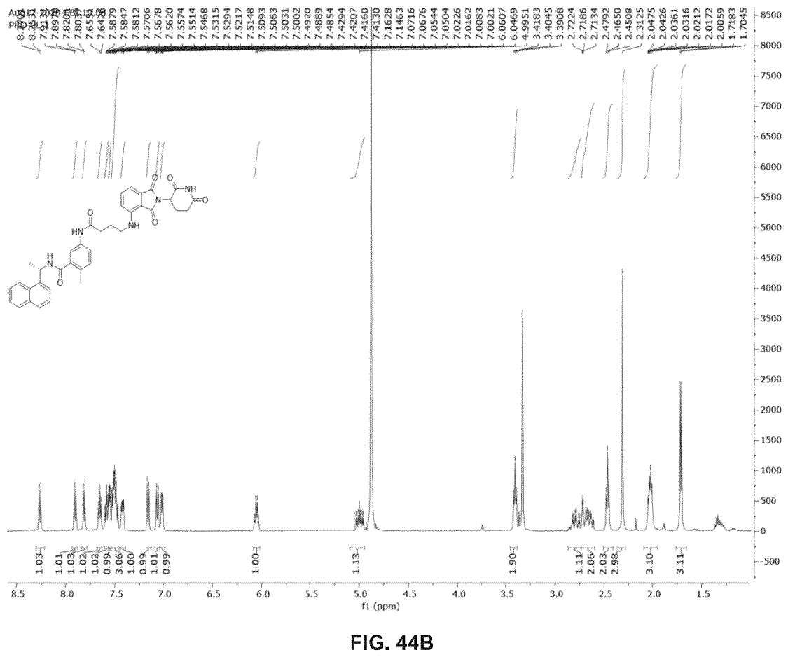

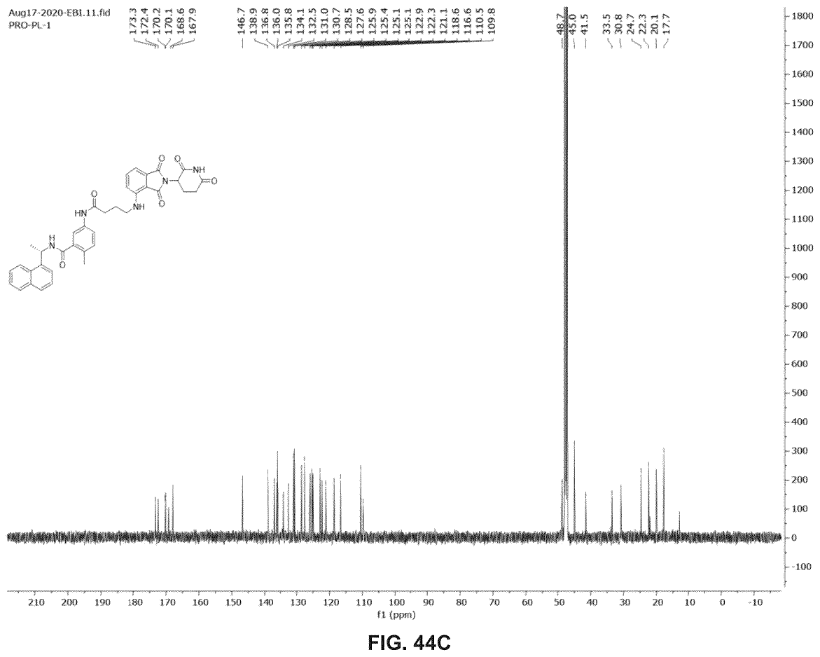

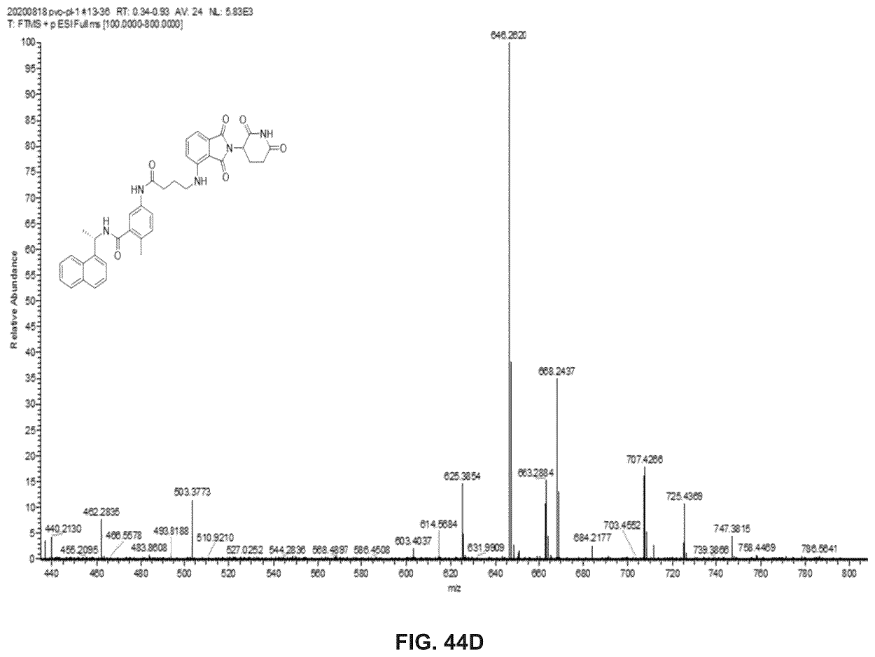

The synthesis of 5-(4-((2-(2,6-dioxopiperidin-3-yl)-1,3-dioxoisoindolin-4-yl)amino) butanamido)-2-methyl-N-((S)-1-(naphthalen-1-yl)ethyl)benzamide (PLP-1) is shown in FIG. 44A. To a solution of (S)-5-amino-2-methyl-N-(1-(naphthalen-1-yl)ethyl)benzamide (6.1 mg, 0.02 mmol) and pomalidomide-C3-COOH (7.2 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (43% acetonitrile) to get product as yellow-green solid (9.3 mg, 72%). ¹H NMR (methanol-d₄, 500 MHz): 8.26 (d, J=8.5 Hz, 1H), 7.91 (d, J=8.1 Hz. 1H), 7.81 (d, J=8.2 Hz, 1H), 7.66 (t, J=6.4 Hz, 1H), 7.58 (t, J=8.4 Hz, 1H), 7.55 (dd, J=5.3, 2.3 Hz, 1H), 7.53-7.47 (m, 3H), 7.44-7.41 (m, 1H), 7.15 (d, J=8.3 Hz, 1H), 7.06 (d, J=8.6 Hz, 1H), 7.01 (dd, J=7.1, 3.1 Hz, 1H), 6.05 (q, J=6.9 Hz, 1H), 5.00 (ddd, J=18.5, 12.7, 5.4 Hz, 1H), 3.40 (t, J=6.9 Hz, 2H), 2.90-2.73 (m, 1H), 2.74-2.57 (m, 1H), 2.46 (t, J=7.1 Hz, 2H), 2.31 (s, 3H), 2.07-1.99 (m, 3H), 1.71 (d, J=6.9 Hz. 3H), shown in FIG. 44B. ¹³C-NMR (methanol-d₄, 125 MHz): 173.3, 172.4, 170.3, 170.2, 168.6, 167.9, 146.7, 138.9, 136.8, 136.0, 135.8, 134.1, 132.5, 131.0, 130.7, 128.5, 127.6, 125.9, 125.4, 125.1, 125.1, 122.9, 122.3, 121.1, 118.6, 116.6, 110.5, 109.8, 48.7, 45.0, 41.5, 33.5, 30.8, 24.7, 22.3, 20.1, 17.7, shown in FIG. 44C. HR-ESI-MS (m/z) calculated for 646.2663 (M+H)⁺, found 646.2620 (FIG. 44D).

### Synthesis of PLP-2

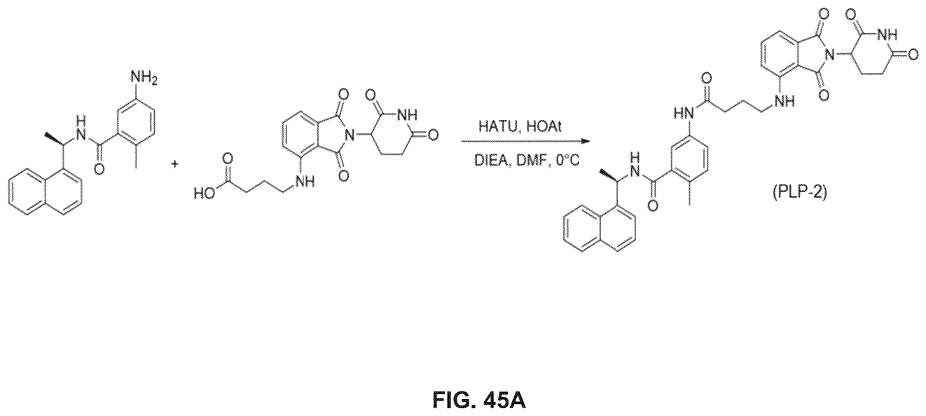

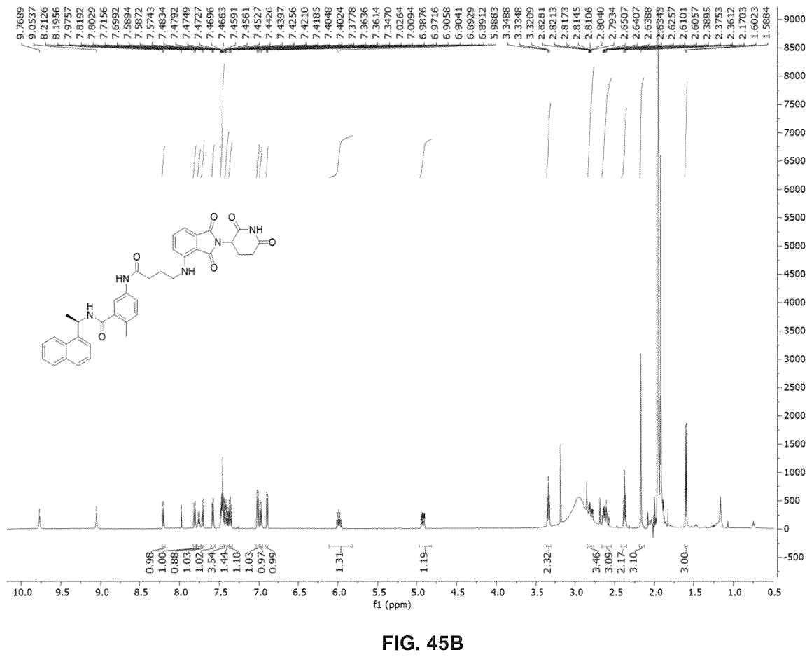

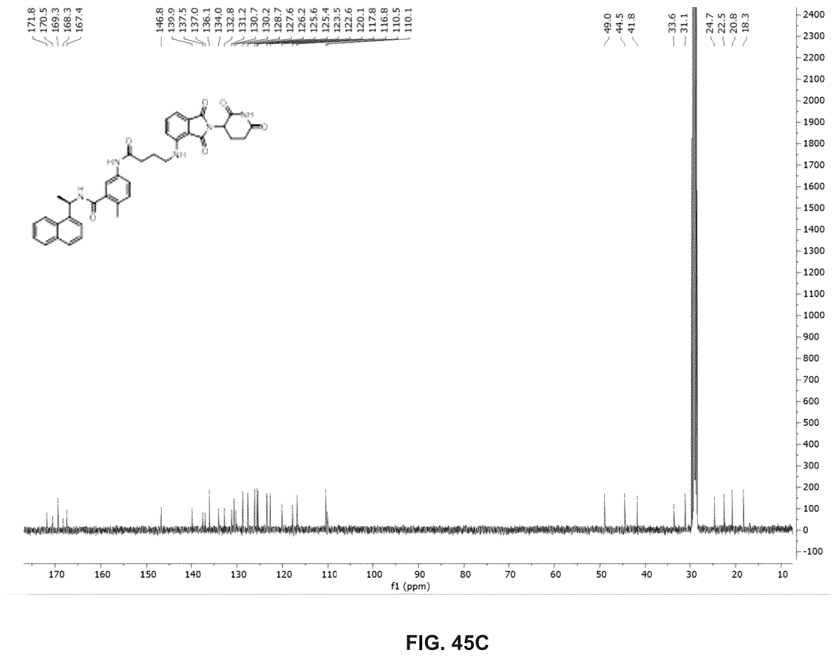

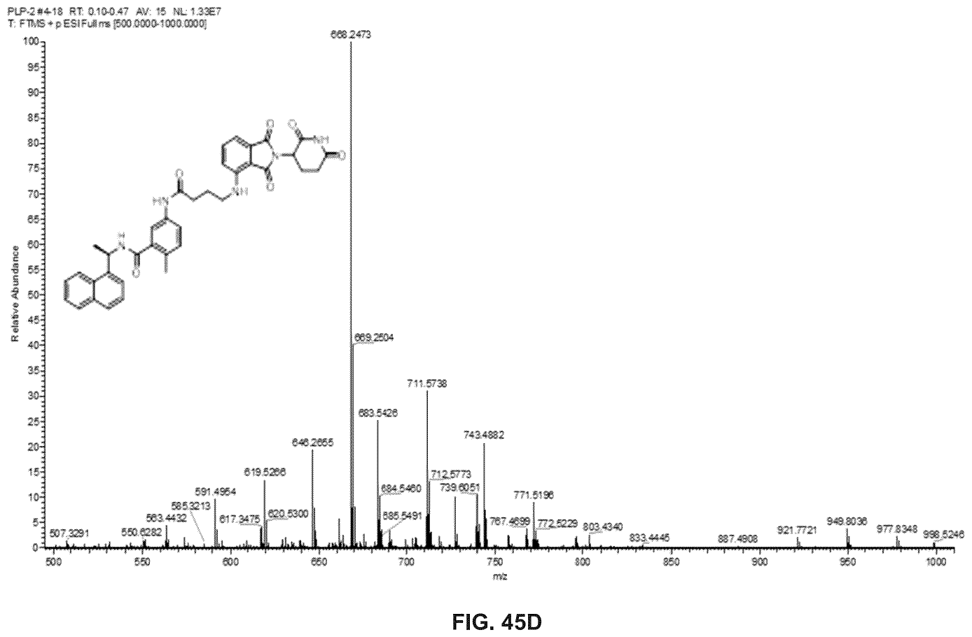

The synthesis of 5-(4-((2-(2,6-dioxopiperidin-3-yl)-1,3-dioxoisoindolin-4-yl)amino)butanamido)-2-methyl-N-((R)-1-(naphthalen-1-yl)ethyl)benzamide (PLP-2) is shown in FIG. 45A. To a solution of PLP-TA-1 (6.1 mg, 0.02 mmol) and pomalidomide-C₃-COOH (7.2 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half an hour, then the mixture was purified with HPLC (43% acetonitrile) to get product as yellow-green solid (9.3 mg, 72%). ¹H NMR (acetone-d₆, 500 MHz): 9.77 (s, 1H), 9.05 (s, 1H), 8.20 (d, J=8.5 Hz, 1H), 7.98 (s, 1H), 7.81 (d, J=8.5 Hz, 1H), 7.76 (d, J=8.3 Hz, 1H), 7.71 (d, J=8.3 Hz, 1H), 7.58 (d, J=6.8 Hz, 1H), 7.48-7.42 (overlapped, 4H), 7.40 (t, J=8.0 Hz, 1H), 7.36 (t, J=8.0 Hz, 1H), 7.02 (d, J=8.5 Hz, 1H), 6.98 (d, J=8.0 Hz, 1H), 6.90 (d, J=8.0 Hz, 1H), 5.99 (dt, J=14.8, 6.9 Hz, 1H), 4.92 (ddd, J=12.5, 5.5, 2.9 Hz, 1H), 3.33 (t, J=7.0 Hz, 2H), 2.83-2.77 (m, 3H), 2.66-2.57 (m, 3H), 2.38 (t, J=7.1 Hz, 2H), 2.17 (s, 3H), 1.60 (d, J=6.9 Hz. 3H), as shown in FIG. 45B. ¹³C-NMR (acetone-d₆, 125 MHz): 171.8, 170.5, 169.4, 168.3, 167.4, 146.8, 139.9, 137.5, 137.0, 136.1, 134.0, 132.8, 131.2, 130.7, 130.2, 128.7, 127.6, 126.2, 125.6, 125.4, 123.5, 122.7, 120.1, 117.8, 116.8, 110.5, 110.1, 49.0, 44.5, 41.8, 33.6, 31.1, 24.7, 22.5, 20.8, 18.3, as shown in FIG. 45C. HR-ESI-MS (m/z) calculated for 668.2482 (M+Na)*, found 668.2473 (FIG. 45D).

### Synthesis of PLP-3

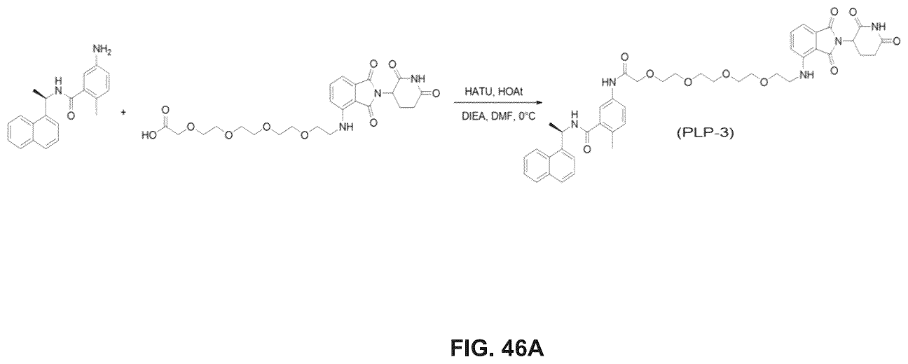

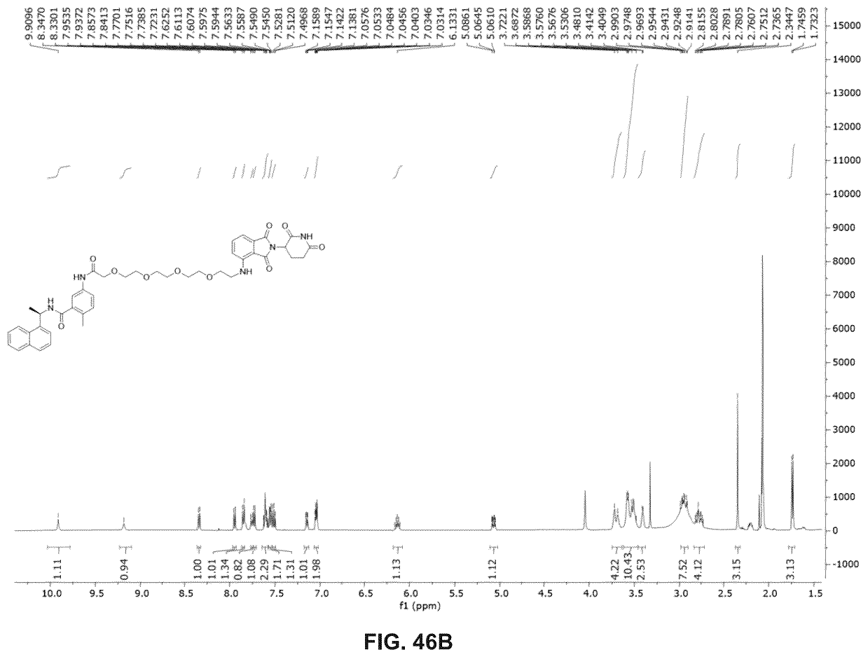

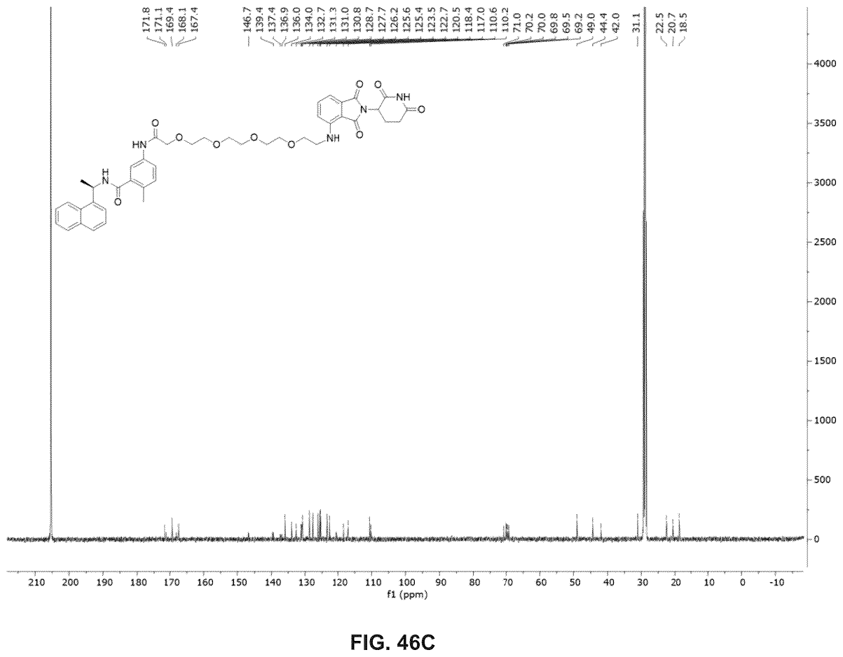

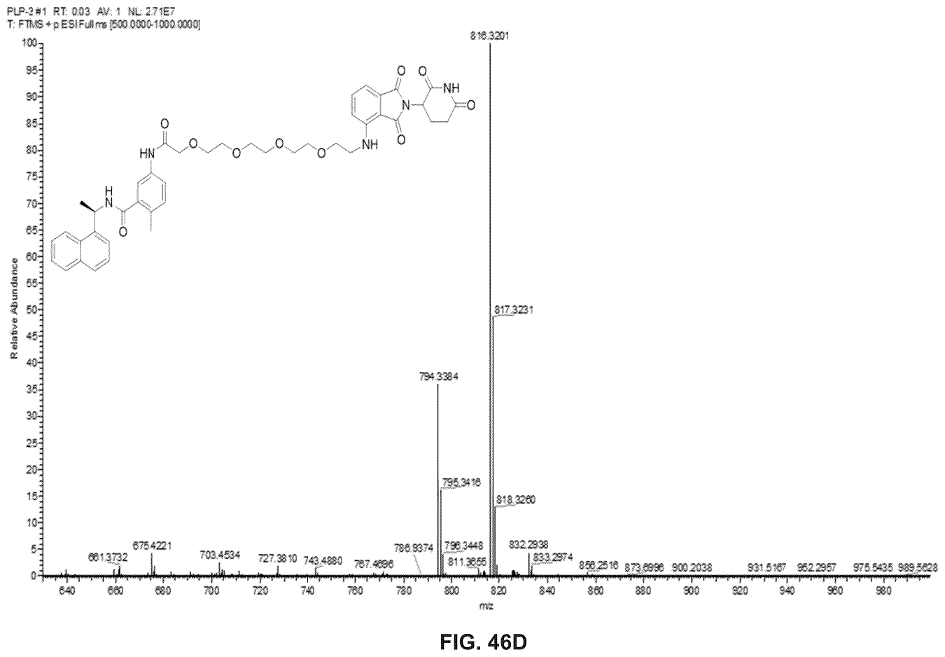

The synthesis of 14-((2-(2,6-dioxopiperidin-3-yl)-1,3-dioxoisoindolin-4-yl)amino)-N-(4-methyl-3-(((R)-1-(naphthalen-1-yl)ethyl)carbamoyl)phenyl)-3,6,9,12-tetraoxatetradecanamide (PLP-3) is shown in FIG. 46A. To a solution of GLR-06GLR-0617 (6.1 mg. 0.02 mmol) and pomalidomide-PEG₄-C-COOH (11.4 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (45% acetonitrile) to get product as yellow-green solid (10.4 mg, 66%). ¹H NMR (acetone-d₆, 500 MHz): 9.91 (s, 1H), 9.18 (s, 1H), 8.34 (d, J=8.5 Hz, 1H), 7.95 (d, J=8.2 Hz, 1H), 7.85 (d, J=8.0 Hz, 1H), 7.76 (d, J=9.3 Hz, 1H), 7.73 (d, J=7.7 Hz, 1H), 7.83 (s, 1H), 7.64-7.59 (m, overlapped, 2H), 7.56-7.54 (m, overlapped, 2H), 7.51 (t, J=7.8 Hz, 1H). 7.15 (dd, J=8.3, 2.1 Hz, 1H), 7.06-7.00 (m, 2H), 6.13 (p, J=7.1 Hz, 1H), 5.07 (ddd, J=12.8, 5.4, 1.8 Hz, 1H), 3.72-3.40 (m, 10H), 2.99-2.91 (m, 2H), 2.82-2.73 (m, 2H), 2.34 (s, 3H), 1.74 (d, J=6.8 Hz, 3H), as shown in FIG. 46B. ¹³C-NMR (acetone-d₆, 125 MHz): 171.8, 171.1, 169.4, 168.1, 167.4, 146.7, 139.4, 137.4, 136.9, 136.0, 134.0, 132.7, 131.3, 131.0, 130.8, 128.7, 127.7, 126.2, 125.7, 125.4, 123.5, 122.8, 120.5, 118.4, 117.0, 110.6, 110.2, 71.0, 70.3, 70.0, 69.9, 69.5, 69.2, 49.020, 44.4, 42.0, 31.1, 22.5, 20.7, 18.5, as shown in FIG. 46C. HR-ESI-MS (m/z) calculated for 816.3218 (M+Na)⁺, found 816.3201 (FIG. 46D).

### Synthesis of PLP-4

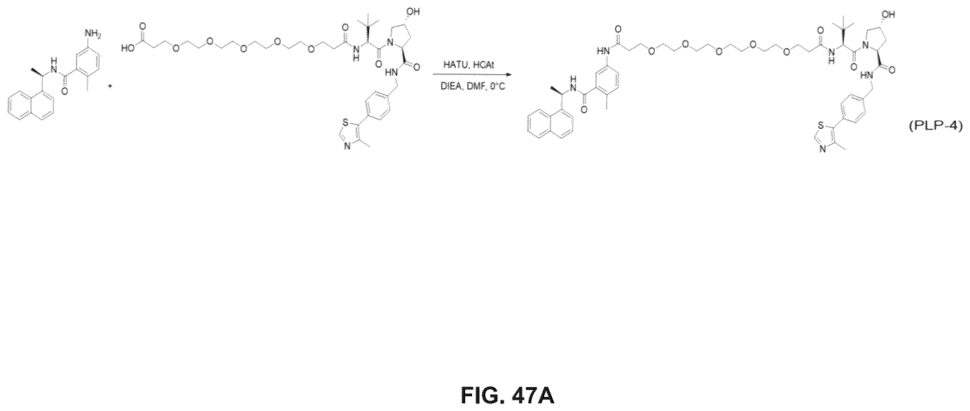

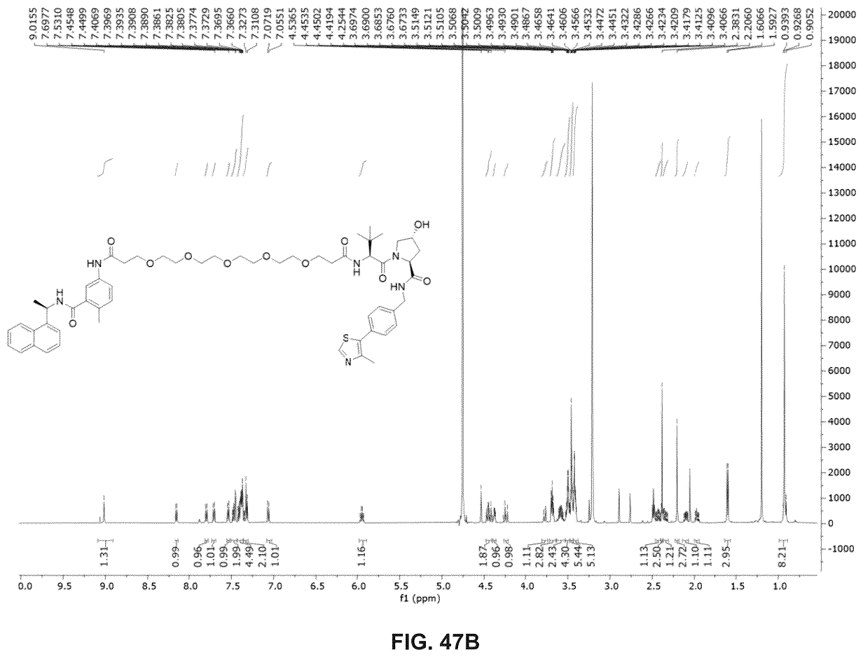

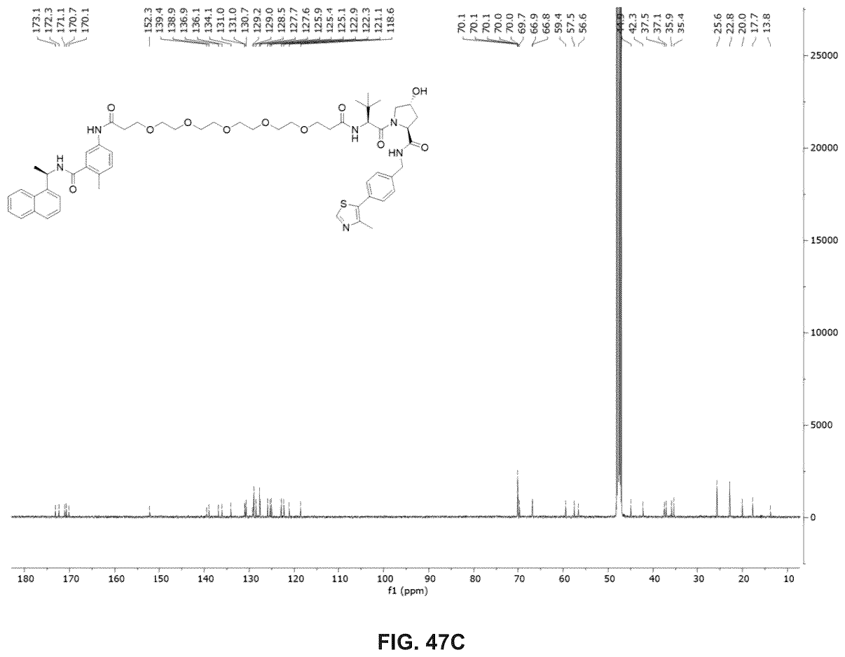

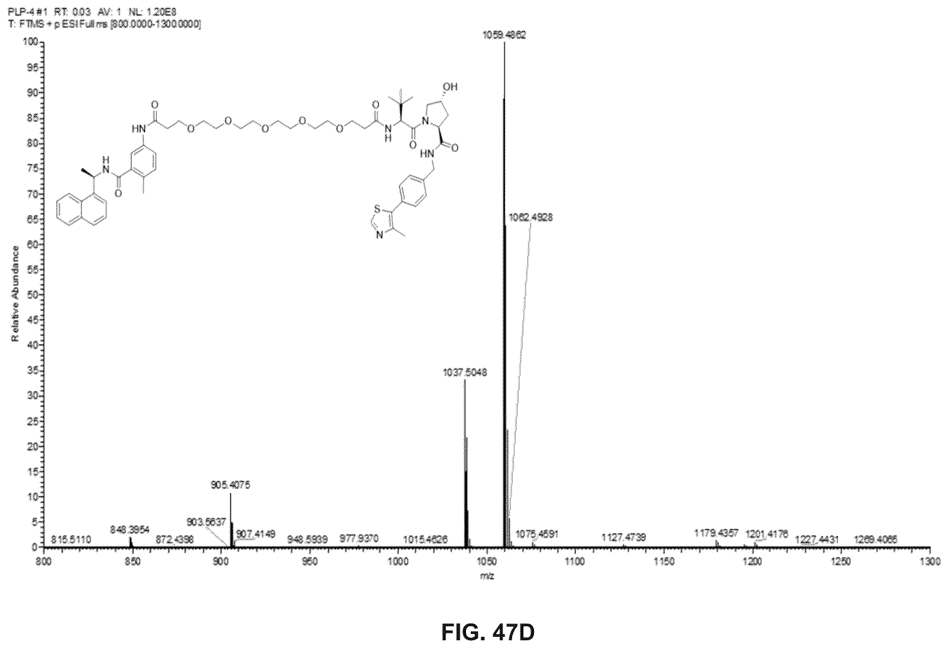

The synthesis of N¹-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N¹⁹-(4-methyl-3-(((R)-1-(naphthalen-1-yl)ethyl)carbamoyl)phenyl)-4,7,10,13,16-pentaoxanonadecanediamide (PLP-4) is shown in FIG. 47A. To a solution of GLR-06GLR-0617 (6.1 mg, 0.02 mmol) and (S,R,S)-AHPC-PEG₅-COOH (15.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (36% acetonitrile) to get product as light yellow solid (9.6 mg, 49%). ¹H NMR (methanol-d₄, 500 MHz): 9.02 (s, 1H), 8.15 (d, J=8.5 Hz, 1H), 7.80 (d, J=8.1 Hz, 1H), 7.71 (d, J=8.2 Hz, 1H), 7.54 (d, J=7.1 Hz, 1H), 7.49-7.44 (overlapped, 2H), 7.43-7.34 (overlapped, 4H), 7.34-7.29 (overlapped, 2H), 7.06 (d, J=8.4 Hz, OH), 5.95 (dt, J=6.9, 6.9 Hz, 1H), 4.48-4.42 (m, 2H), 4.38-4.36 (m, 1H), 4.24 (d, J=15.5 Hz, 1H), 3.78-3.67 (m, 3H), 3.63-3.55 (m, 2H), 3.52-3.40 (m, overlapped 14H), 2.46-2.32 (m, 2H), 2.38 (s, 3H), 2.12-1.94 (m, 2H), 1.60 (d, J=6.9 Hz, 3H), 0.93 (s, 9H), as shown in FIG. 47B. ¹³C-NMR (methanol-d₄, 125 MHz): 173.1, 172.3, 171.1, 170.7, 170.1, 152.3, 139.5, 138.9, 136.9, 136.1, 134.1, 131.0, 131.0, 130.7, 129.2, 129.0, 128.5, 127.7, 127.6, 125.9, 125.4, 125.1, 122.9, 122.3, 121.1, 118.6, 70.1, 70.1-70.0 (overlapped), 69.7, 66.9, 66.8, 59.4, 57.5, 56.6, 44.9, 42.3, 37.5, 37.1, 35.9, 35.4, 25.6, 22.8, 20.0, 17.7, 13.8, as shown in FIG. 47C. HR-ESI-MS (m/z) calculated for 1059.4846 (M+Na)⁺, found 1059.4862 (FIG. 47D).

### Synthesis of Boc-HoTyr-(Ac)Dap-G-OH

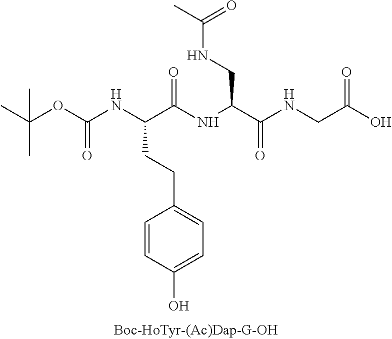 [SMILES: CC(=O)NC[C@H](NC(=O)[C@H](CCc1ccc(O)cc1)NC(=O)OC(C)(C)C)C(=O)NCC(=O)O]

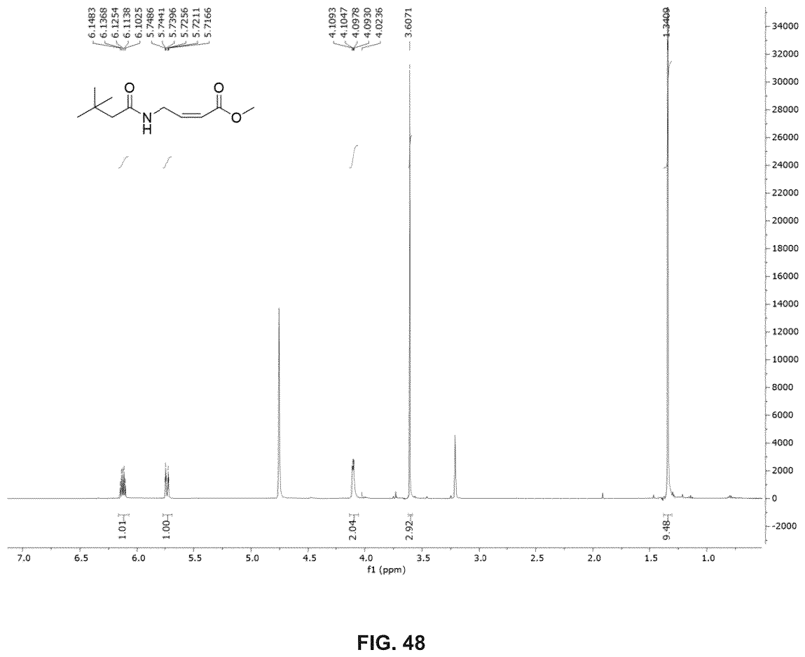

Boc-HoTyr-(Ac)Dap-G-OH was synthesized according to the solid phase peptide synthesis method. First, Fmoc-(Ac)Dap-OH (2.5 eq) was attached to H-Gly-Cl-Trt resin (0.790 mmol/g, 1.3 g) using HATU (2.5 eq), DIEA (5.0 eq), and HOAt (0.5 eq) in DMF as coupling reagents. Then, the Fmoc protecting group was removed using 20% piperidine in DMF (2 cycles: 5, and 15 min). Boc-HoTyr(Trt)-OH (2.5 eq) was coupled to the H₂N—(Ac)Dap-G-resin using HATU (2.5 eq), DIEA (5.0 eq), and HOAt (0.5 eq) in DMF. Then, the resin was washed five times with DMF, three times with DCM, and three times with MeOH, and dried under vacuum. Next, the peptide was removed from the resin with a mixture of 20% Trifluoroethanol in DCM, precipitated in Et₂O, purified on HPLC, and lyophilized. The purity of peptide was confirmed using HPLC. HR-ESI-MS (m/z) calculated for 503.2113 (M+Na)⁺, found 503.2159 (FIG. 48).

### Synthesis of PLP-3PY

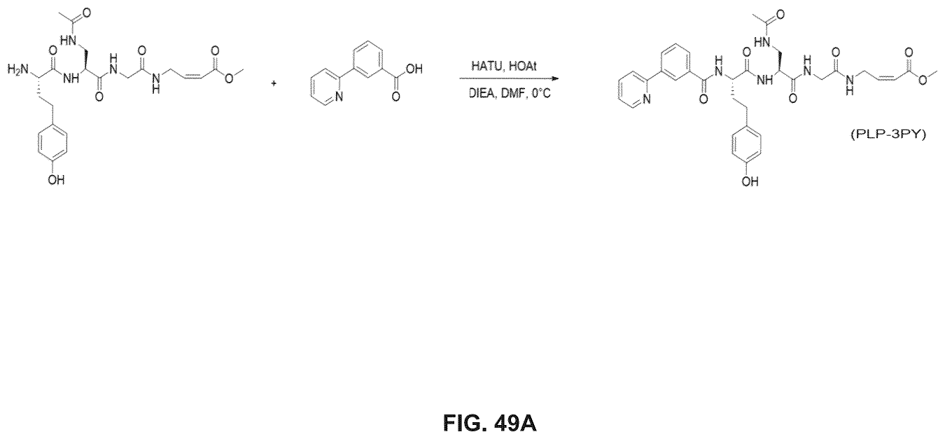

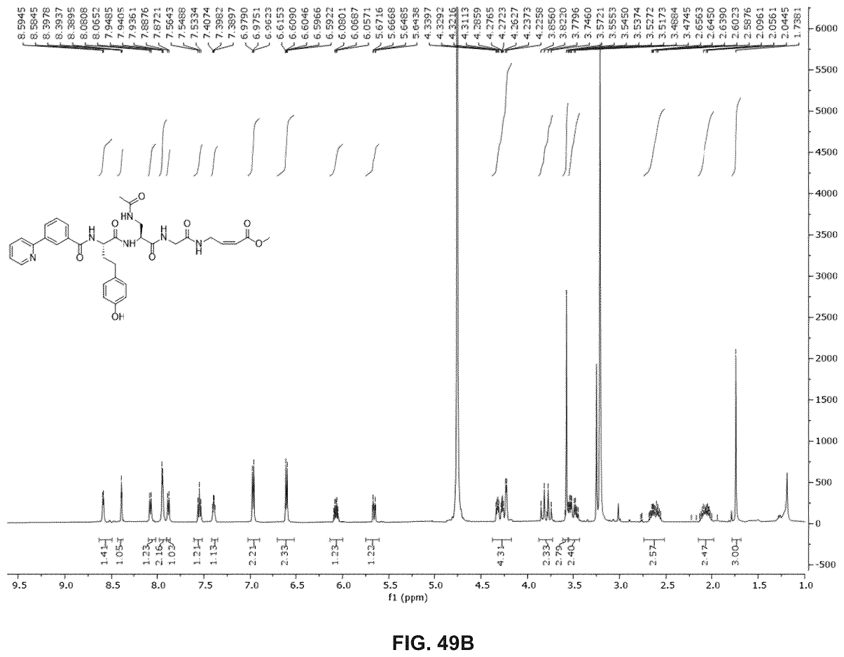

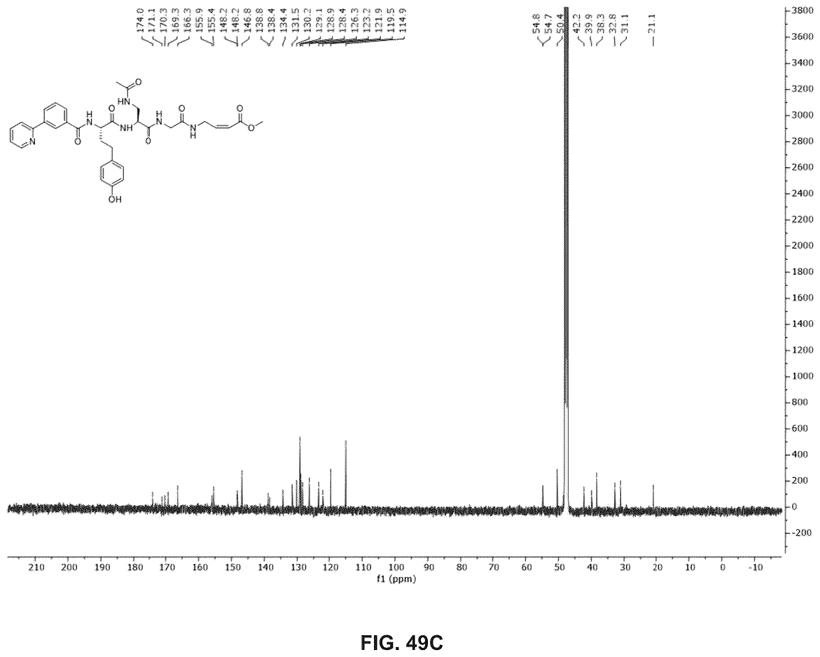

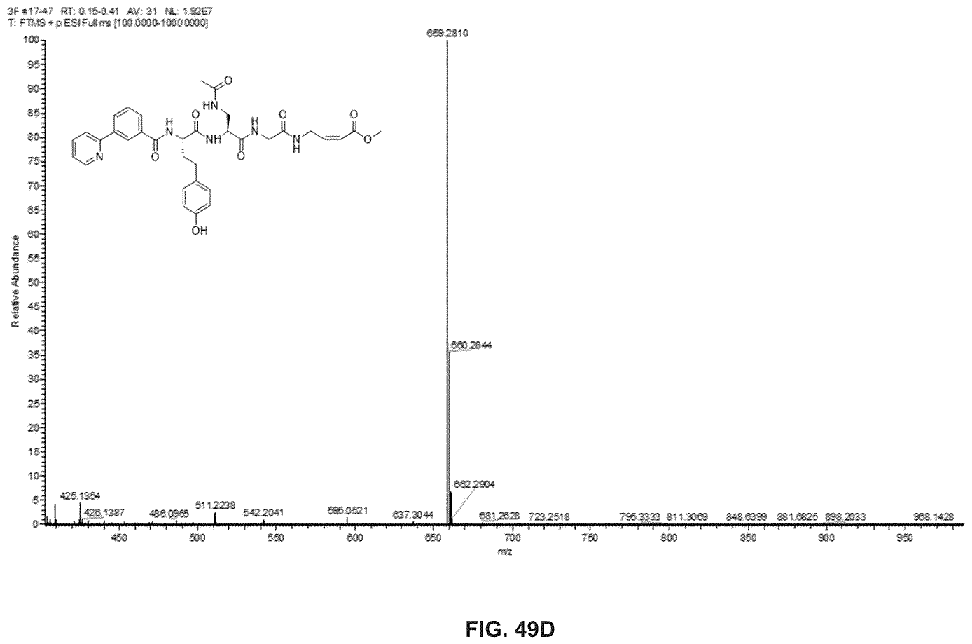

The synthesis of methyl (3S,6S,Z)-6-(acetamidomethyl)-3-(4-hydroxyphenethyl)-1,4,7,10-tetraoxo-1-(3-(pyridin-2-yl)phenyl)-2,5,8,11-tetraazapentadec-13-en-15-oate (PLP-3PY) is shown in FIG. 49A. To a solution of PLP-TA-2 (9.5 mg, 0.02 mmol) and 3-(pyridin-2-yl)benzoic acid (4.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (32% acetonitrile) to get product as light yellow solid (6.1 mg, 46%). ¹H NMR (methanol-d₄, 500 MHz): 8.59 (d, J=5.0 Hz, 1H), 8.39 (d, J=2.1 Hz, 1H), 8.07 (d, J=7.8 Hz, 11H), 8.00-7.86 (overlapped, 2H), 7.55 (t, J=7.7 Hz, 11H), 7.39 (q, J=4.7 Hz, 1H), 6.97 (d, J=8.4 Hz, 2H), 6.60 (d, J=8.4 Hz, 2H), 6.07 (dt, J=11.4, 5.7 Hz, 1H), 5.66 (dt, J=11.5, 2.4 Hz, 1H), 4.33 (dd, J=9.0, 5.2 Hz, 1H), 4.27 (dd, J=6.9, 4.7 Hz, 1H), 4.23 (d, J=5.8 Hz, 2H), 3.90-3.71 (m, 2H), 3.57 (s, 3H), 3.55-3.42 (m, 2H), 2.70-2.54 (m, 2H), 2.14-1.99 (m, 2H), 1.74 (s, 3H), as shown in FIG. 49B. ¹³C-NMR (methanol-d₄, 125 MHz): 174.0, 171.2, 170.3, 169.3, 166.3, 155.9, 155.4, 148.3, 148.2, 146.8, 138.8, 138.4, 134.4, 131.5, 130.2, 129.1, 128.9, 128.4, 126.3, 123.2, 121.9, 119.5, 114.9, 54.8, 54.7, 50.4, 42.2, 39.9, 38.3, 32.8, 31.1, 21.1, as shown in FIG. 49C. HR-ESI-MS (m/z) calculated for 659.2826 (M+H)⁺, found 659.2810 (FIG. 49D).

### Synthesis of PLP-4PY

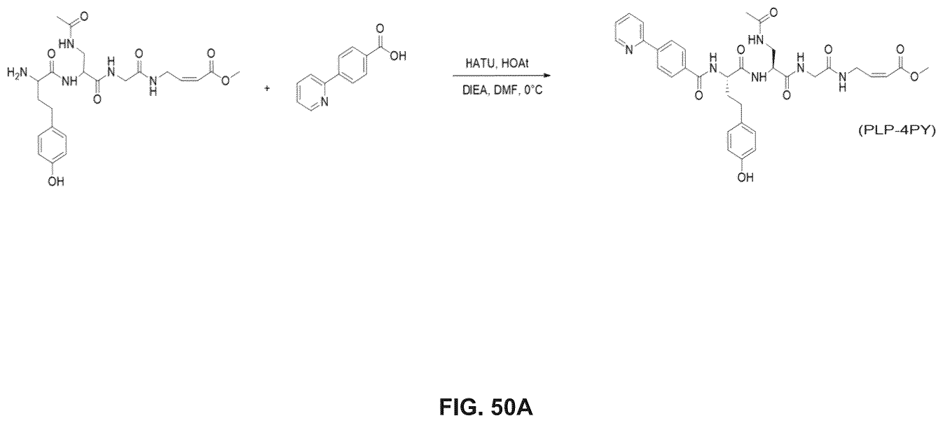

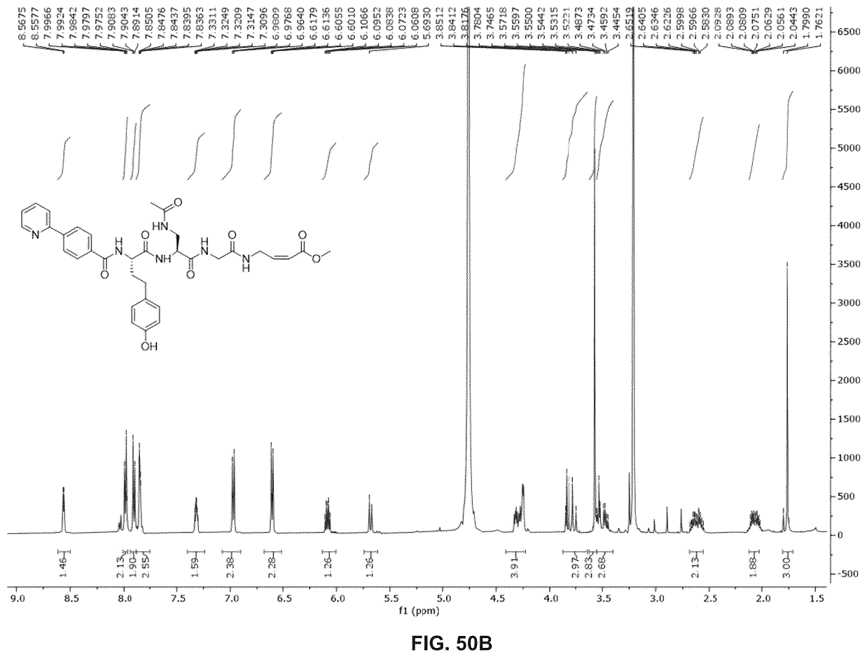

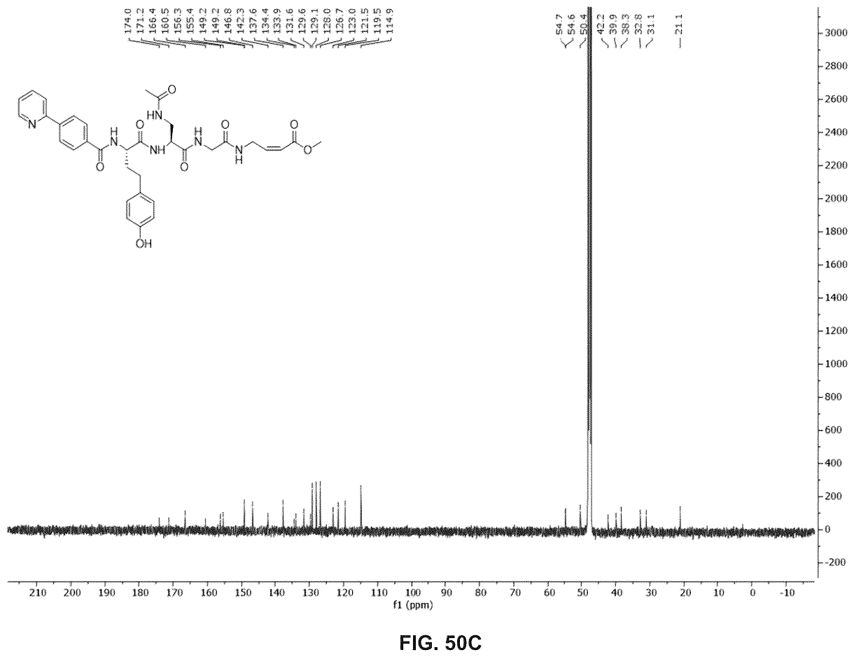

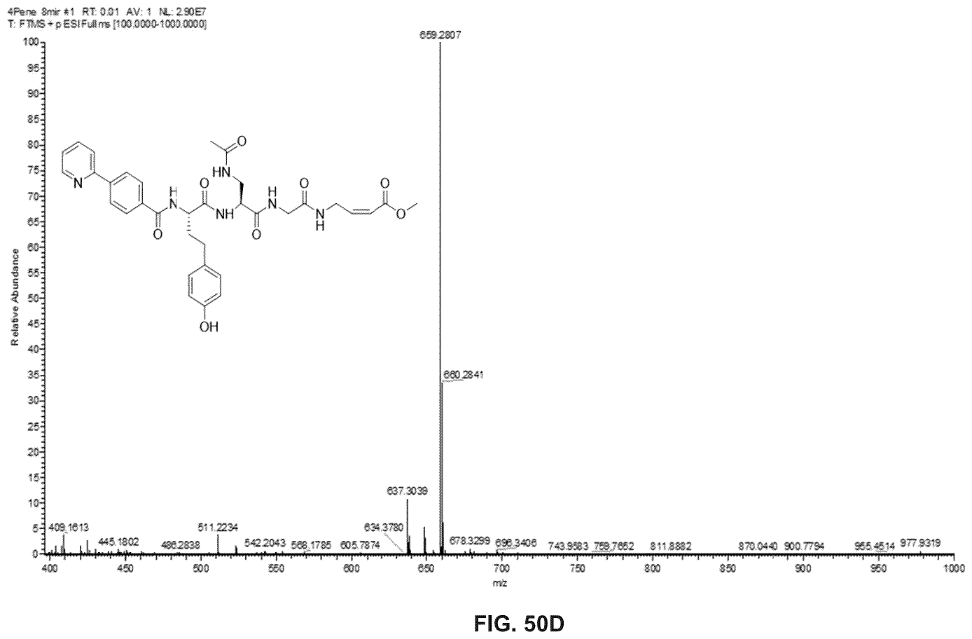

The synthesis of methyl (3S,6S,Z)-6-(acetamidomethyl)-3-(4-hydroxyphenethyl)-1,4,7,10-tetraoxo-1-(4-(pyridin-2-yl)phenyl)-2,5,8,11-tetraazapentadec-13-en-15-oate (PLP-4PY) is shown in FIG. 50A. To a solution of PLP-TA-2 (9.5 mg, 0.02 mmol) and 4-(pyridin-2-yl)benzoic acid (4.0 mg, 0.02 mmol) in 1 mL DMF, DIEA (17.5 μL, 0.1 mmol), HATU (15.2 mg, 0.04 mmol), and HOAt (0.6 mg, 0.004 mmol) were added under ice bath. The reaction was stirred at 0° C. for half hour, then the mixture was purified with HPLC (28% acetonitrile) to get product as light yellow solid (5.9 mg, 46%). ¹H NMR (methanol-d₄, 500 MHz): 8.56 (d, J=4.9 Hz, 1H), 7.99 (d, J=8.4 Hz, 2H), 7.90 (d, J=8.5 Hz, 2H), 7.85-7.84 (overlapped, 2H), 7.32 (dt, J=5.7, 2.8 Hz, 1H), 6.97 (d, J=8.5 Hz, 2H), 6.61 (d, J=8.4 Hz, 2H), 6.08 (dt, J=11.5, 5.7 Hz, 1H), 5.68 (dt, J=11.5, 2.3 Hz, 11H), 4.35-4.26 (m, 2H), 4.25 (dt, J=5.7, 2.1 Hz, 2H), 3.87-3.72 (m, 2H), 3.57 (s, 3H), 3.56-3.43 (m, 2H), 2.73-2.54 (m, 2H), 2.14-2.00 (m, 2H), 1.76 (s, 3H), as shown in FIG. 50B. ¹³C-NMR (methanol-d₄, 125 MHz): 173.98, 171.16, 166.37, 160.54, 156.26, 155.40, 149.24, 149.20, 146.81, 142.31, 137.63, 134.40, 133.90, 131.57, 129.60, 129.13, 127.96, 126.73, 123.01, 121.46, 119.52, 114.90, 54.70, 54.63, 50.37, 42.20, 39.90, 38.34, 32.82, 31.07, 21.12, as shown in FIG. 50C. HR-ESI-MS (m/z) calculated for 659.2826 (M+H)*, found 659.2807 (FIG. 50D).

### Activity

The following Table 1 shows the Mpro degradation activity of compounds MP-18, MP-19, MP-20, MP-21, and MP-22 at 500 nm.

TABLE 1
Degradation activity of compounds tested against the
Mpro target protein at 500 nM

| Compound | Control | MP-18 | MP-19 | MP-20 | MP-21 | MP-22 |
|---|---|---|---|---|---|---|
| Mpro | 100% | 45% | 32% | 28% | 52% | 75% |
| (relative | | | | | | |
| concentration) | | | | | | |

The following Table 2 shows the Mpro degradation activity of MP-28, MP-29, MP-30, MP-31, and MP-32 to be tested against the target protein at 500 nM.

TABLE 2
Degradation activity of compounds tested against the
Mpro target protein at 500 nM

| Compound | Control | MP-28 | MP-29 | MP-30 | MP-31 | MP-32 |
|---|---|---|---|---|---|---|
| Mpro | 100% | 33% | 40% | 52% | 65% | 80% |
| (relative | | | | | | |
| concentration) | | | | | | |

The following Table 3 shows the Mpro degradation activity of MP-38, MP-39, MP-40, MP-41, and MP-42.

TABLE 3
Degradation activity of compounds tested against the
Mpro target protein at 500 nM

| Compound | Control | MP-38 | MP-39 | MP-40 | MP-41 | MP-42 |
|---|---|---|---|---|---|---|
| Mpro | 100% | 50% | 40% | 25% | 45% | 70% |
| (relative | | | | | | |
| concentration) | | | | | | |

### Dual Targeting of PLpro and Mpro

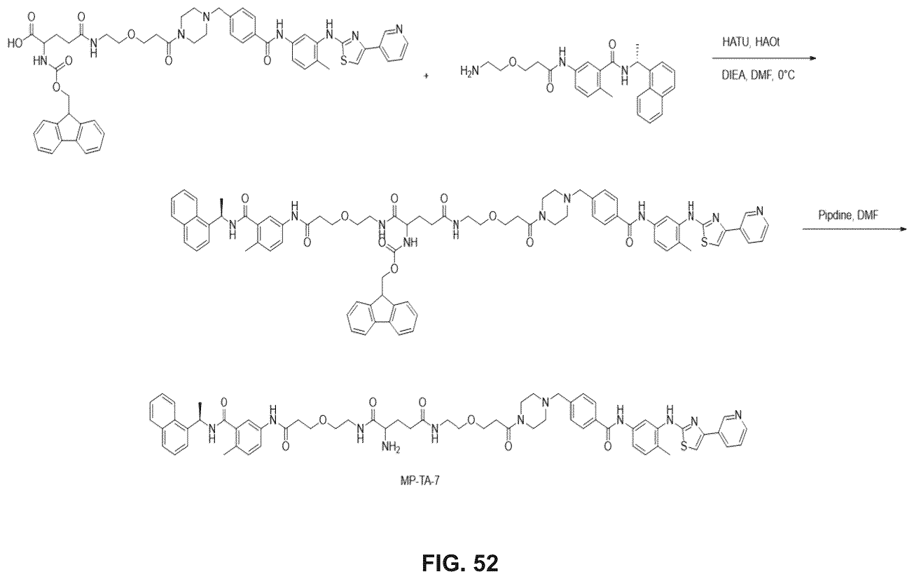

FIGS. 51B, 52 show the synthesis of dual targeting PROTAC compounds that include both a Mpro ligand and a PLpro inhibitor. FIG. 52 shows the synthesis of MP-TA-7:

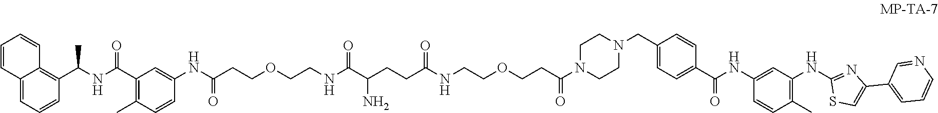 [SMILES: Cc1ccc(NC(=O)c2ccc(CN3CCN(C(=O)CCOCCNC(=O)CCC(N)C(=O)NCCOCCC(=O)Nc4ccc(C)c(C(=O)N[C@H](C)c5cccc6ccccc56)c4)CC3)cc2)cc1Nc1nc(-c2cccnc2)cs1]

A similar procedure was followed to obtain MP-TA-8:

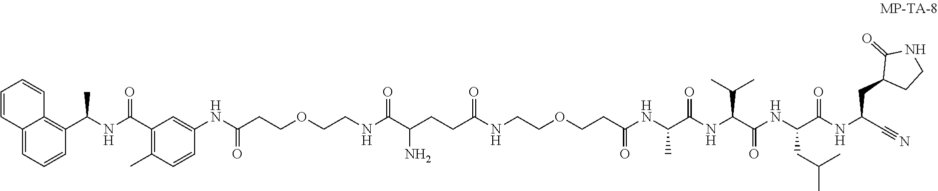 [SMILES: Cc1ccc(NC(=O)CCOCCNC(=O)C(N)CCC(=O)NCCOCCC(=O)N[C@@H](C)C(=O)N[C@H](C(=O)N[C@@H](CC(C)C)C(=O)N[C@H](C#N)C[C@@H]2CCNC2=O)C(C)C)cc1C(=O)N[C@H](C)c1cccc2ccccc12]

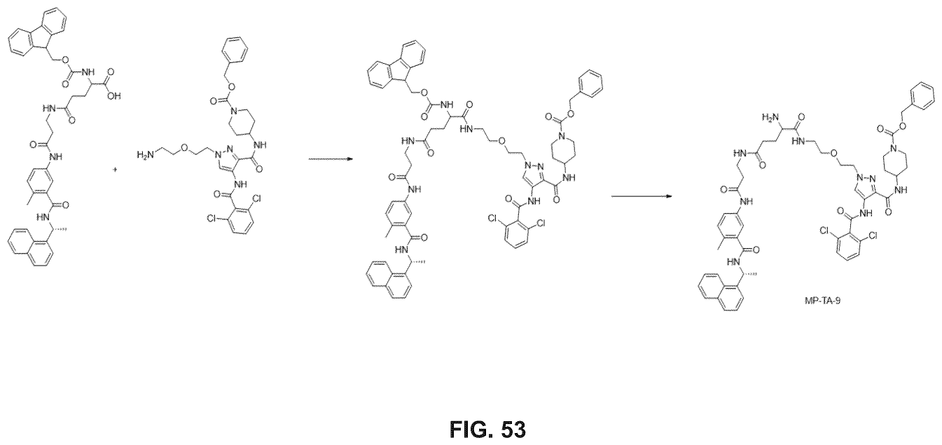

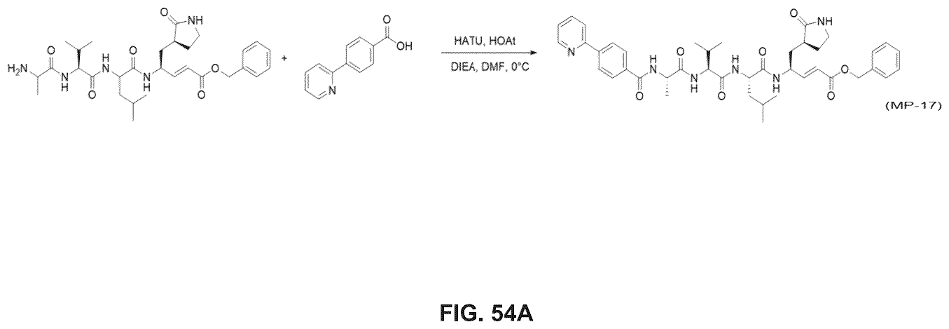

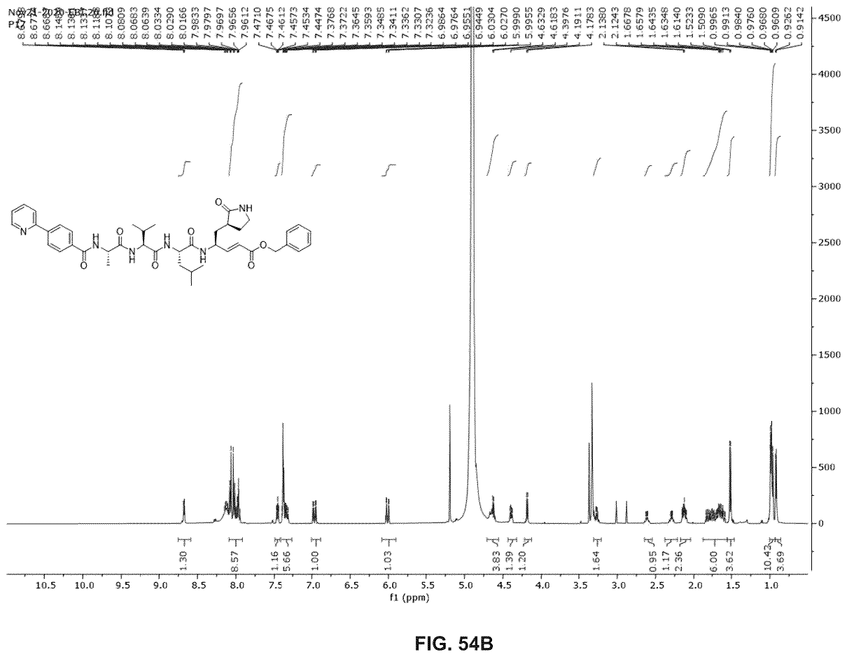

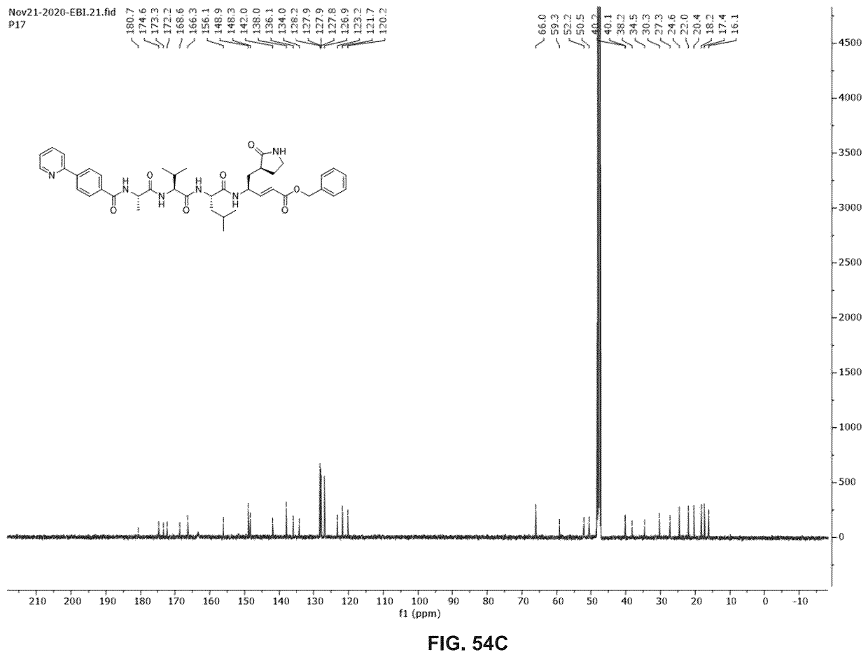

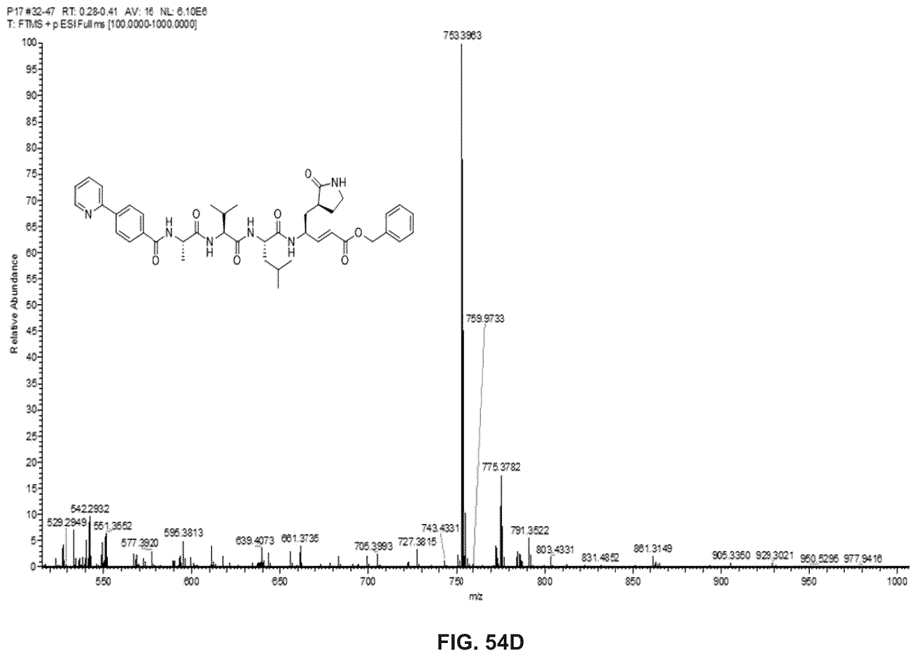

FIG. 53 shows the synthesis of MP-TA-9:

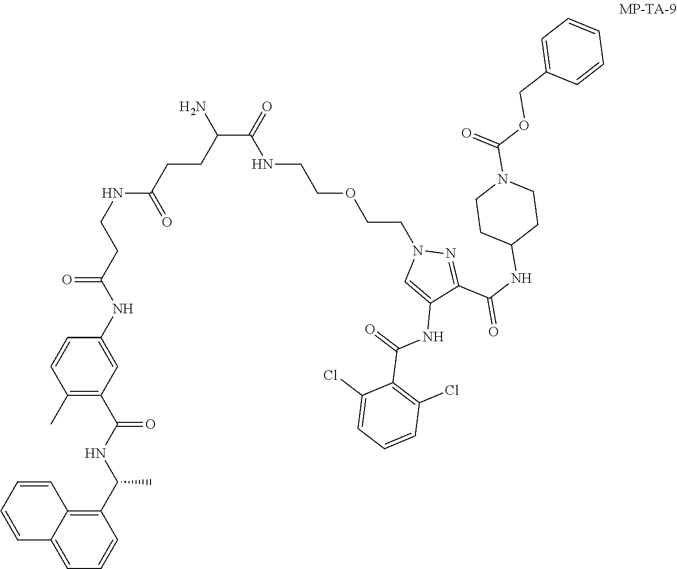 [SMILES: Cc1ccc(NC(=O)CCNC(=O)CCC(N)C(=O)NCCOCCn2cc(NC(=O)c3c(Cl)cccc3Cl)c(C(=O)NC3CCN(C(=O)OCc4ccccc4)CC3)n2)cc1C(=O)N[C@H](C)c1cccc2ccccc12]

FIGS. 9A-9F show the synthesis of compounds MP-48 through MP-71.

Compounds MP-48 through MP-51 share the following general structure DT-1:

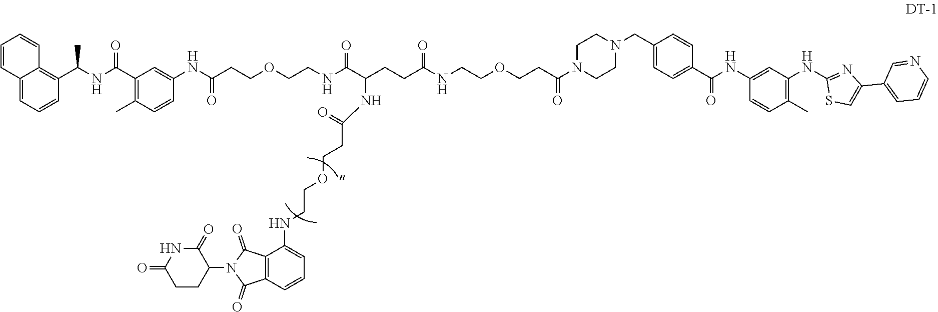

where n ranges from 1-4. When n is 1, the compound is MP-48. When n is 2, the compound is MP-49. When n is 3, the compound is MP-50. When n is 4, the compound is MP-51.

Compounds MP-52 through MP-55 share the following general structure DT-2:

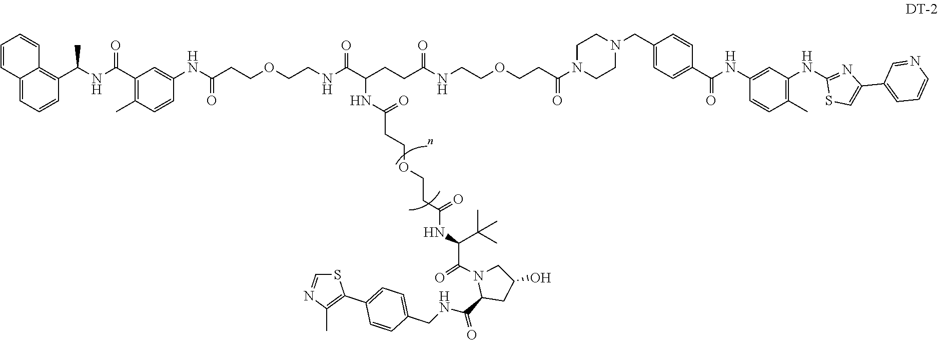

where n ranges from 1 to 4. When is 1, the compound is MP-52. When n is 2, the compound is MP-53. When n is 3, the compound is MP-54. When n is 4, the compound is MP-55.

Compounds MP-56 through MP-59 share the following general structure DT-3:

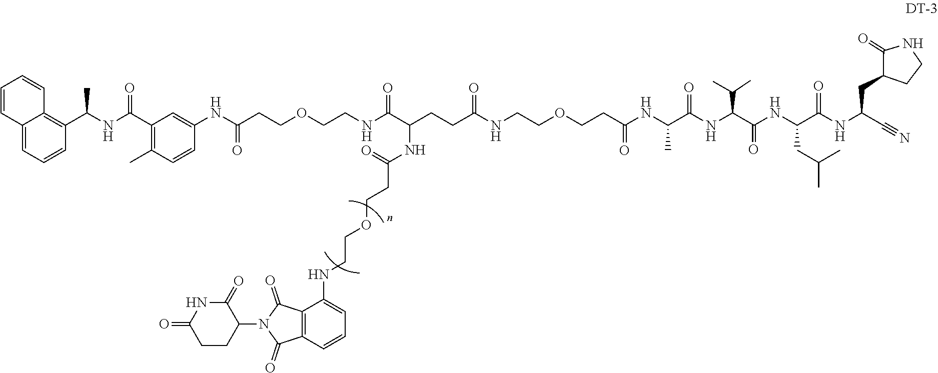

where n ranges from 1 to 4. When n is 1, the compound is MP-56. When n is 2, the compound is MP-57. When n is 3, the compound is MP-58. When n is 4, the compound is MP-59.

Compounds MP-60 through MP-63 share the following general structure DT-4:

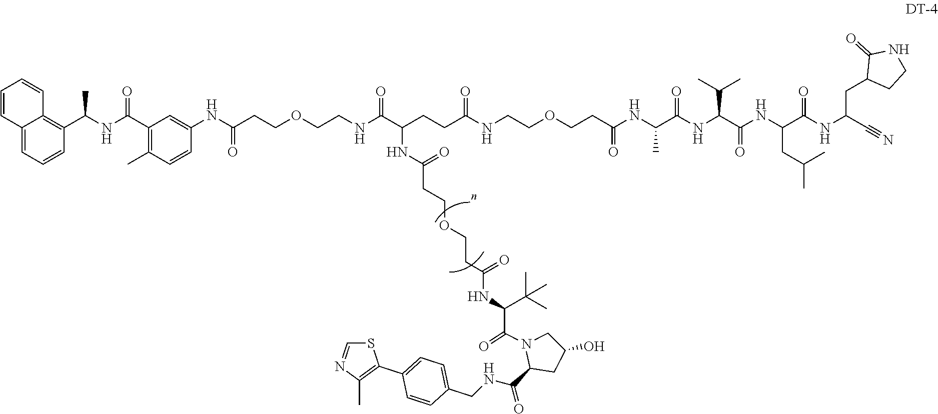

where n ranges from 1 to 4. When n is 1, the compound is MP-60. When n is 2, the compound is MP-61. When n is 3, the compound is MP-62. When n is 4, the compound MP-63.

Compounds MP-64 through MP-67 share the following general structure DT-5:

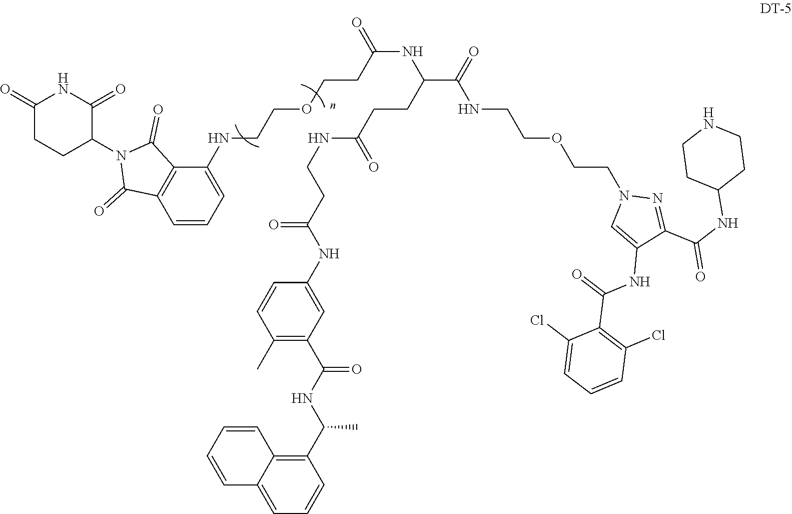

where n ranges from 1 to 4. When n is 1, the compound is MP-64. When n is 2, the compound is MP-65. When n is 3, the compound is MP-66. When n is 4, the compound MP-67.

Compounds MP-68 through MP-72 share the following general structure DT-6:

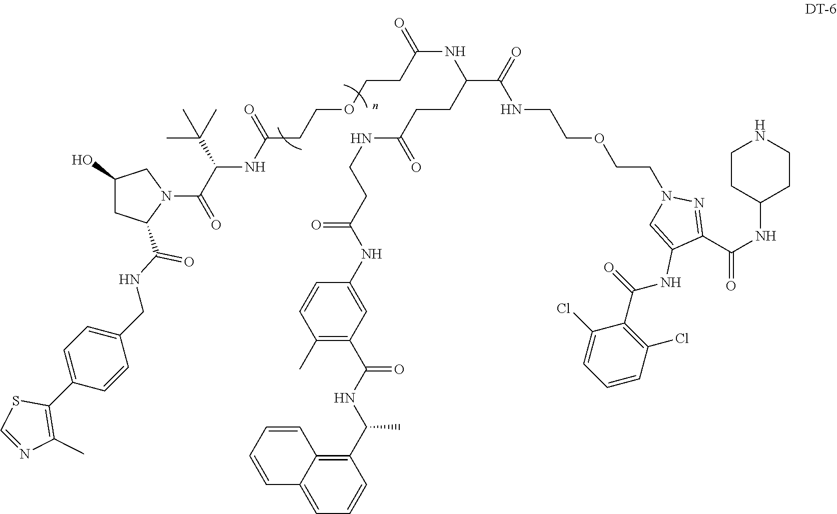

where n ranges from 1 to 4. When n is 1, the compound is MP-68. When n is 2, the compound is MP-69. When n is 3, the compound is MP-70. When n is 4, the compound MP-71.

Notably, in all of these compounds, the following group:

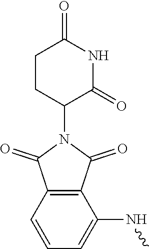

can be replaced by the following group:

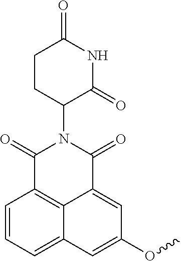

### Activity of Dual Targeting Compounds

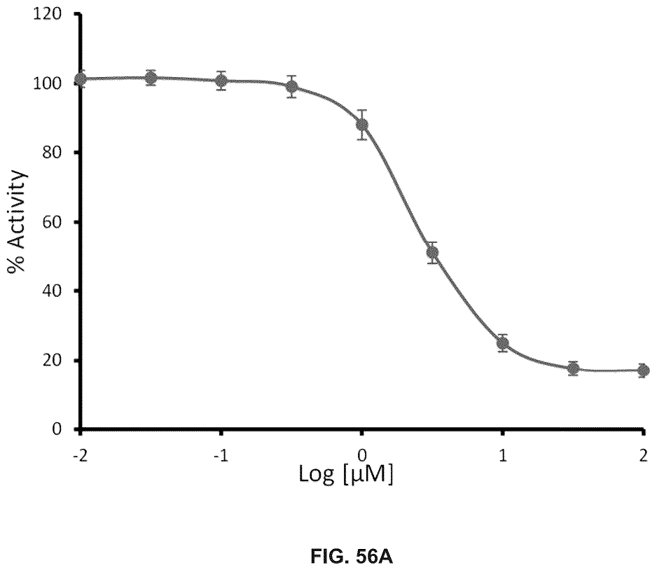

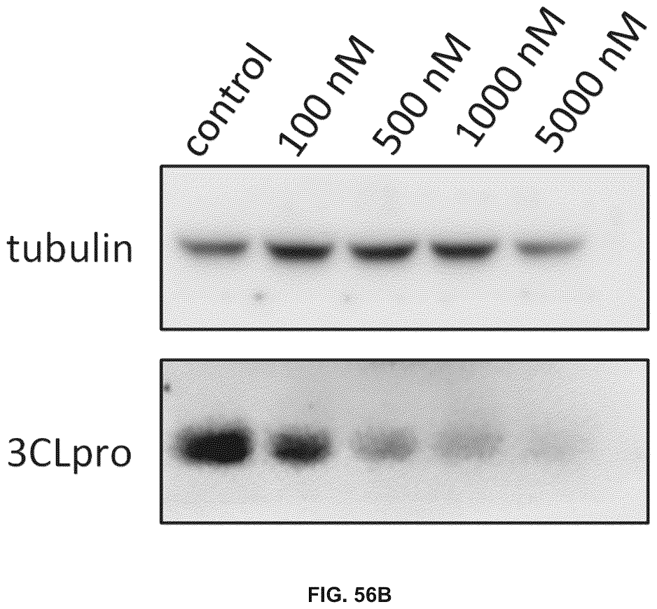

An all-in-one expression plasmid expressing both Mpro and PLpro was established. The activity of dual targeting PROTAC compounds was tested, and it was found that certain dual targeting PROTAC compounds could not only effectively degrade Mpro but also effectively inhibit deubiquitination activity of PLpro. FIGS. 56A-56B show the results of testing the dual targeting PROTAC compound referred to as DTP depicted in FIG. 51A. FIG. 56A shows a concentration-dependent inhibition of the dual targeting PROTAC compound DTP on PLpro. FIG. 56B shows the dose-dependent effects of the dual targeting PROTAC compound DTP on Mpro-T7 level.

The most active of the dual targeting PROTAC compounds tested were MP-48, MP-56, and MP-60. The following Table 4 shows the Mpro and PLpro degradation activity of compounds MP-48, MP-49, MP-50, and MP-51 against the target proteins at 500 nM.

TABLE 4
The degradation activity of dual targeting PROTAC compounds
tested against the target proteins at 500 nM

| Compound | Control | MP-48 | MP-49 | MP-50 | MP-51 |
|---|---|---|---|---|---|
| Mpro | 100% | 35% | 72% | 88% | 94% |
| (relative concentration) | | | | | |
| PLpro | 100% | 28% | 66% | 80% | 90% |
| (relative concentration) | | | | | |

The following Table 5 shows the Mpro and PLpro degradation activity of MP-56, MP-57, MP-58, and MP-59 against the target proteins at 500 nm.

TABLE 5
The degradation activity of dual targeting PROTAC compounds
tested against the target proteins at 500 nM

| Compound | Control | MP-56 | MP-57 | MP-58 | MP-59 |
|---|---|---|---|---|---|
| Mpro | 100% | 30% | 75% | 90% | 95% |
| (relative concentration) | | | | | |
| PLpro | 100% | 25% | 65% | 80% | 93% |
| (relative concentration) | | | | | |

The following Table 6 shows the Mpro and PLpro degradation activity of MP-60. MP-61, MP-62, and MP-63 against the target proteins at 500 nm.

TABLE 6
The degradation activity of dual targeting PROTAC compounds
tested against the target proteins at 500 nM

| Compound | Control | MP-60 | MP-61 | MP-62 | MP-63 |
|---|---|---|---|---|---|
| Mpro | 100% | 45% | 80% | 95% | 95% |
| (relative concentration) | | | | | |
| PLpro | 100% | 30% | 65% | 85% | 95% |
| (relative concentration) | | | | | |

### Activity Against Human Coronavirus OC43

To evaluate the anti-virus activity of the PROTACs, the low-virulence human coronavirus OC43 (HCoV-OC43) was used instead of SARS-CoV-2. Briefly, RD cells were infected with HCoV-OC43 and incubated at 33° C. for 1 h to allow virus adsorption. Then, the viral inoculum was removed. An overlay containing 0.2% Avicel supplemented with 2% FBS in DMEM containing serial concentrations of testing compounds was added and incubated in a 33° C. incubator for 4-5 days. The plaque formation was detected by staining the cell monolayer with crystal violet, and the plaque areas were quantified. EC₅₀ values were determined by plotting the percent CPE versus log₁₀ compound concentrations from best-fit dose-response curves with variable slope. The following tables give the results.

TABLE 7
EC₅₀ activity of compounds (nM)

| Compound | MP-18 | MP-19 | MP-20 | MP-21 | MP-22 |
|---|---|---|---|---|---|
| EC50 | 336 | 318 | 310 | 480 | 615 |

TABLE 8
EC₅₀ activity of compounds (nM)

| Compound | MP-28 | MP-29 | MP-30 | MP-31 | MP-32 |
|---|---|---|---|---|---|
| EC50 | 315 | 380 | 435 | 520 | 830 |

TABLE 9
EC₅₀ activity of compounds (nM)

| Compound | MP-38 | MP-39 | MP-40 | MP-41 | MP-42 |
|---|---|---|---|---|---|
| EC50 | 380 | 235 | 225 | 325 | 410 |

TABLE 10
EC₅₀ activity of compounds (nM)

| Compound | MP-48 | MP-49 | MP-50 | MP-51 |
|---|---|---|---|---|
| EC50 | 285 | 301 | 425 | 426 |

TABLE 11
EC₅₀ activity of compounds (nM)

| Compound | MP-56 | MP-57 | MP-58 | MP-59 |
|---|---|---|---|---|
| EC50 | 300 | 320 | 363 | 525 |

TABLE 12
EC₅₀ activity of compounds (nM)

| Compound | MP-48 | MP-49 | MP-50 | MP-51 |
|---|---|---|---|---|
| EC50 | 325 | 372 | 422 | 437 |

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising:
an E3 ligase ligand attached to a linker; and
a Mpro ligand attached to the linker.

2. The composition of claim 1, wherein the Mpro ligand comprises any of ML-1, ML-2, ML-3, ML-4, ML-4, ML-5, ML-6, ML-7, ML-8, ML-9, ML-10, ML-11, ML-12, ML-13, ML-14, ML-15, ML-16, ML-17, ML-18, ML-19, or ML-20:

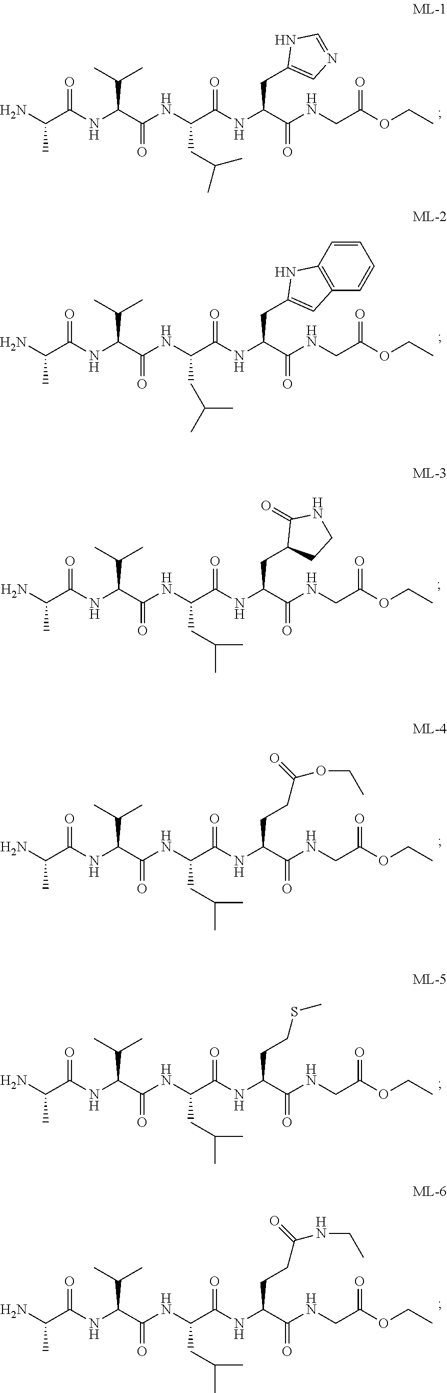

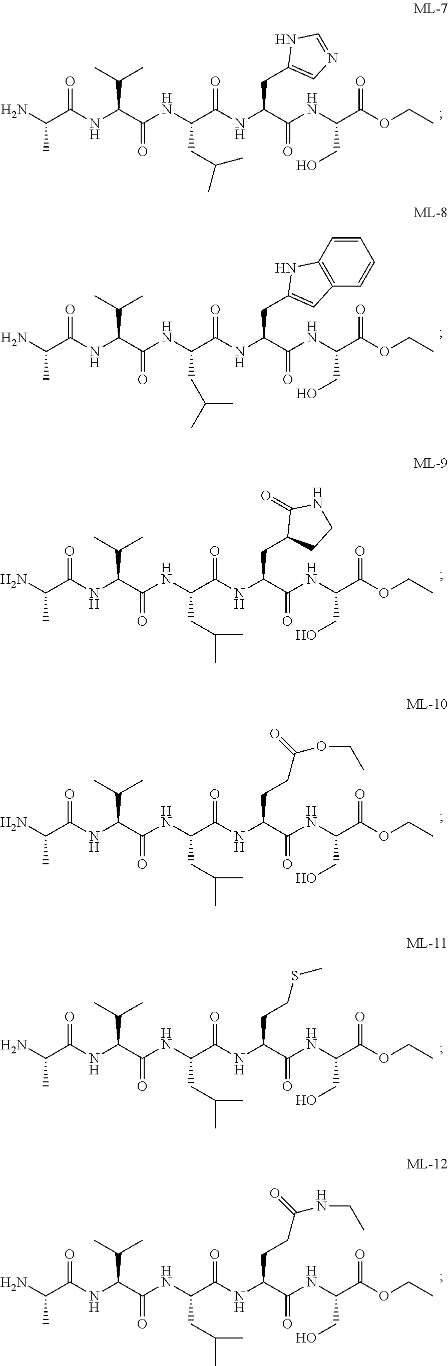

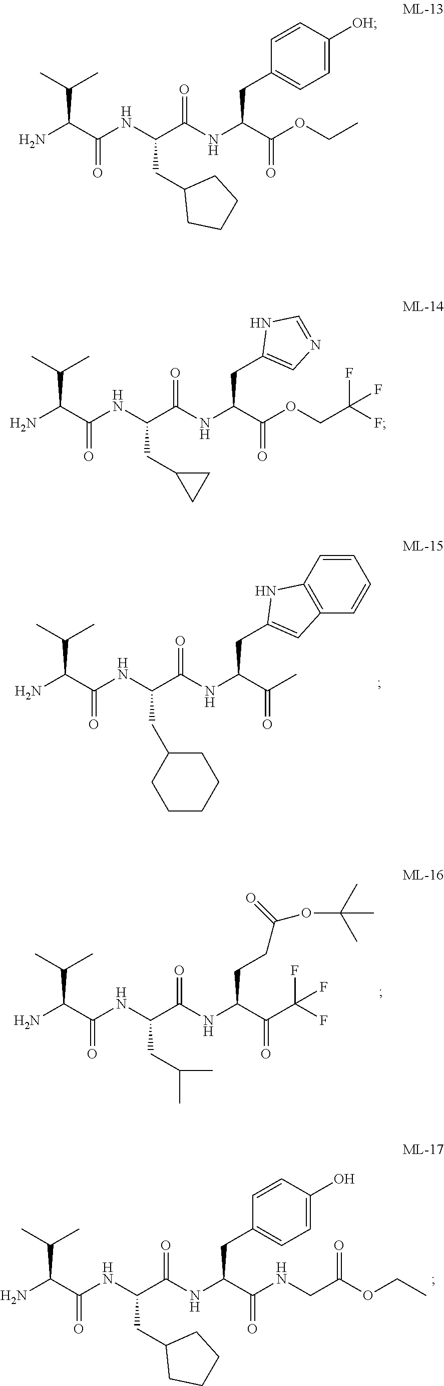 [SMILES: CCOC(=O)[C@H](Cc1ccc(O)cc1)NC(=O)[C@H](CC1CCCC1)NC(=O)[C@@H](N)C(C)C ; CC(C)[C@H](N)C(=O)N[C@@H](CC1CC1)C(=O)N[C@@H](Cc1cnc[nH]1)C(=O)OCC(F)(F)F ; CC(=O)[C@H](Cc1cc2ccccc2[nH]1)NC(=O)[C@H](CC1CCCCC1)NC(=O)[C@@H](N)C(C)C ; CC(C)C[C@H](NC(=O)[C@@H](N)C(C)C)C(=O)N[C@@H](CCC(=O)OC(C)(C)C)C(=O)C(F)(F)F ; CCOC(=O)CNC(=O)[C@H](Cc1ccc(O)cc1)NC(=O)[C@H](CC1CCCC1)NC(=O)[C@@H](N)C(C)C]

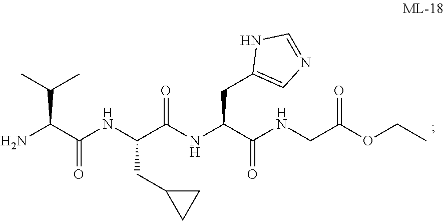 [SMILES: CCOC(=O)CNC(=O)[C@H](Cc1cnc[nH]1)NC(=O)[C@H](CC1CC1)NC(=O)[C@@H](N)C(C)C]

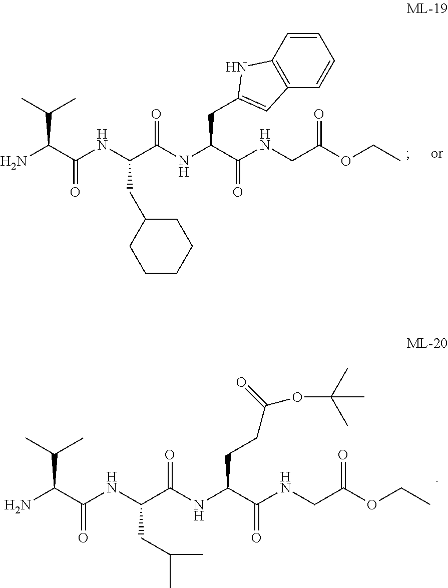 [SMILES: CCOC(=O)CNC(=O)[C@H](Cc1cc2ccccc2[nH]1)NC(=O)[C@H](CC1CCCCC1)NC(=O)[C@@H](N)C(C)C ; CCOC(=O)CNC(=O)[C@H](CCC(=O)OC(C)(C)C)NC(=O)[C@H](CC(C)C)NC(=O)[C@@H](N)C(C)C]

3. The composition of claim 1, wherein the E3 ligase ligand comprises pomalidomide or a von Hippel-Lindau (VHL) ligand.

4. The composition of claim 1, wherein the E3 ligase ligand comprises pomalidomide, an acetylated pomalidomide, an N-alkylated pomalidomide, a pomalidomide derivative with a PEG segment, 4-hydroxythalidomide, an alkyl-connected thalidomide derivative, lenalidomide, a 5-aminothalidomide derivative, thalidomide, methylbestatin, nutilin-3, 4-hydroxythalidomide, or an alkyl-connected thalidomide derivative.

5. The composition of claim 1, wherein the E3 ligase ligand comprises AHPC-PEGₙ-butyl COOH wherein n is 2-10; pomalidomide-PEGₙ-COOH, wherein n is 2-10; or pomalidomide-Cₙ-COOH wherein n is 3-10.

6. The composition of claim 1, wherein the E3 ligase ligand comprises an alkyl group, a PEG chain, an extended glycol chain, an alkyl group containing a PEG segment, an alkyelene group, a heterocyclic group, a von Hippel-Lindau (VHL) ligand, a primary amine, an alkyne, a triazole, a saturated heterocycle, or a combination thereof.

7. (canceled)

8. The composition of claim 1, wherein:
the E3 ligase ligand comprises pomalidomide, and
the linker comprises at least one of:
a polyethylene glycol (PEG) chain or segment, or
an alkyl group having from 1 to 6 carbons.

9. The composition of claim 1, wherein the E3 ligase ligand comprises the von Hippel-Lindau (VHL) ligand and the linker comprises a polyethylene glycol (PEG) chain or segment.

10. The composition of claim 1, wherein the composition comprises: Formula I, Formula II, or Formula III: 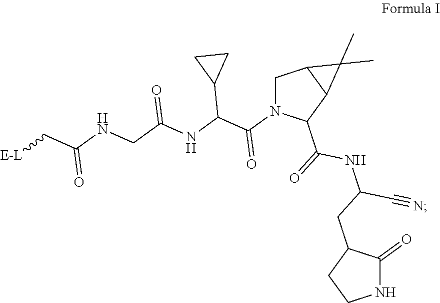 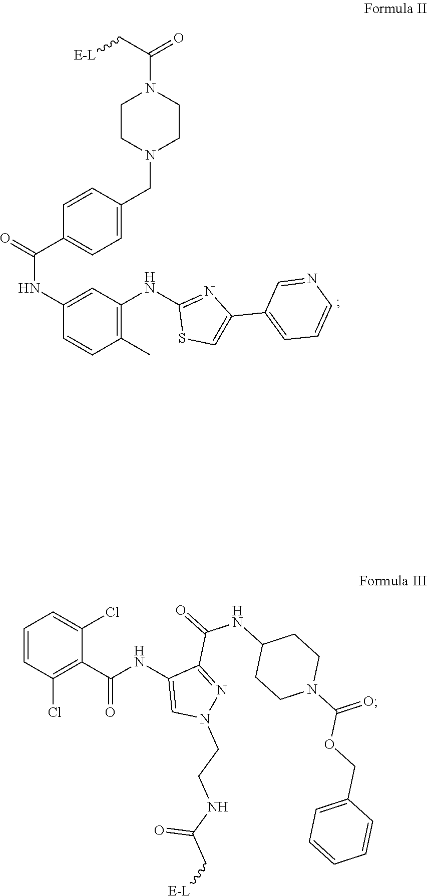
wherein E is the E3 ligase ligand, and L is the linker.

11. The composition of claim 1, wherein the composition comprises Formula II: 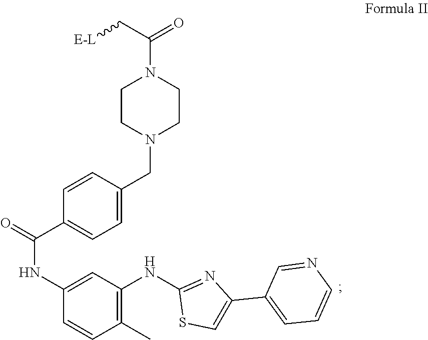
wherein E is the E3 ligase ligand, and L is the linker.

12. (canceled)

13. The composition of claim 1, comprising MP-18:

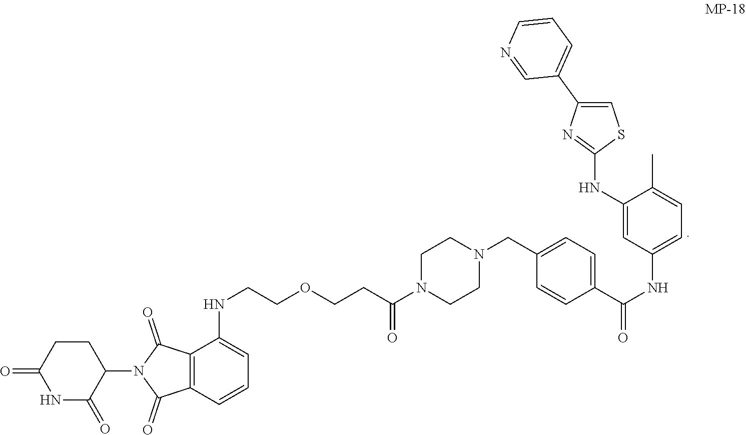 [SMILES: Cc1ccc(NC(=O)c2ccc(CN3CCN(C(=O)CCOCCNc4cccc5c4C(=O)N(C4CCC(=O)NC4=O)C5=O)CC3)cc2)cc1Nc1nc(-c2cccnc2)cs1]

14. The composition of claim 1, comprising: MP-18, MP-19, MP-20, MP-21, MP-22, MP-28, MP-29, MP-30, MP-31, MP-32, MP-38, MP-39, MP-40, MP-41, MP-42, or MP-C-4N:

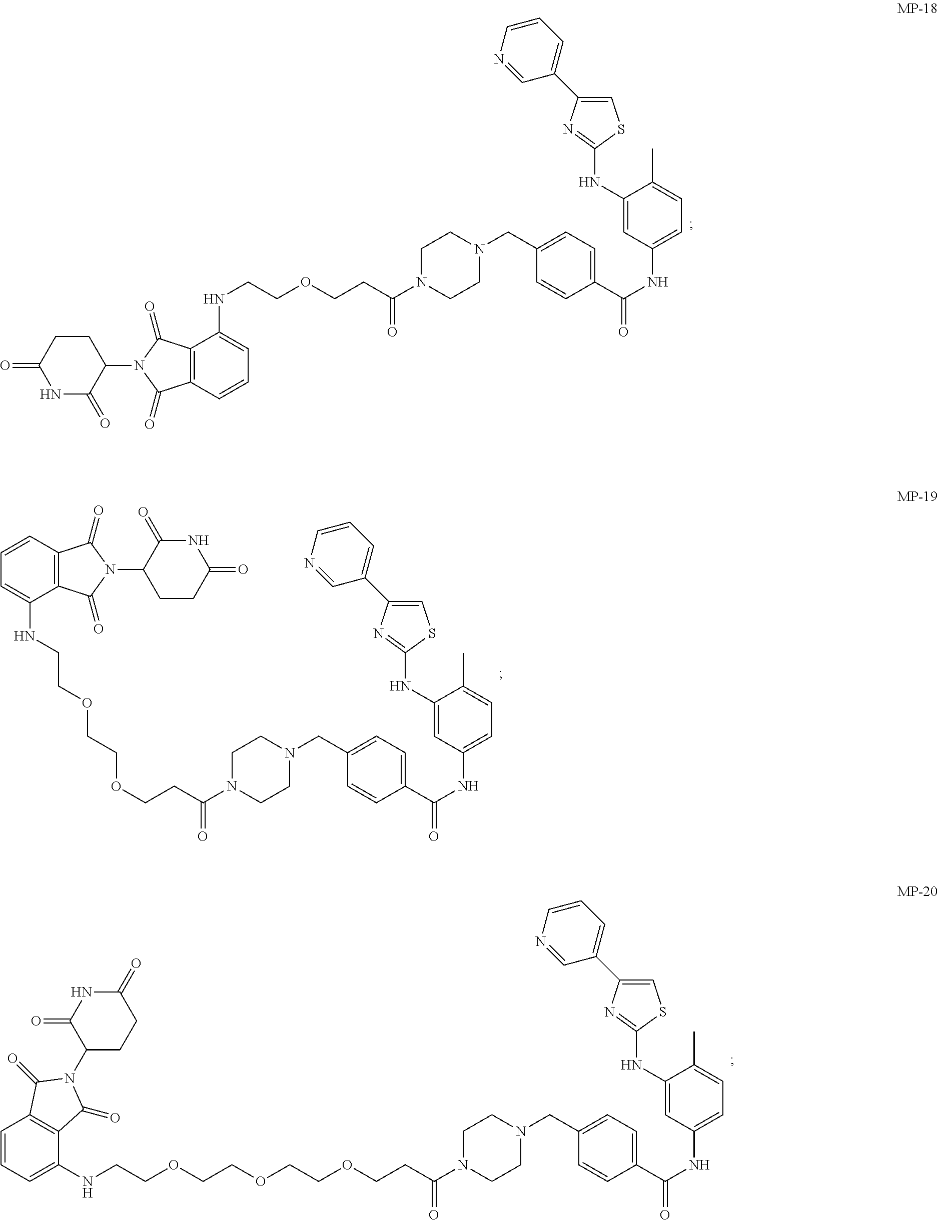 [SMILES: Cc1ccc(NC(=O)c2ccc(CN3CCN(C(=O)CCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCNc4cccc5c4C(=O)N(C4CCC(=O)NC4=O)C5=O)CC3)cc2)cc1Nc1nc(-c2cccnc2)cs1]

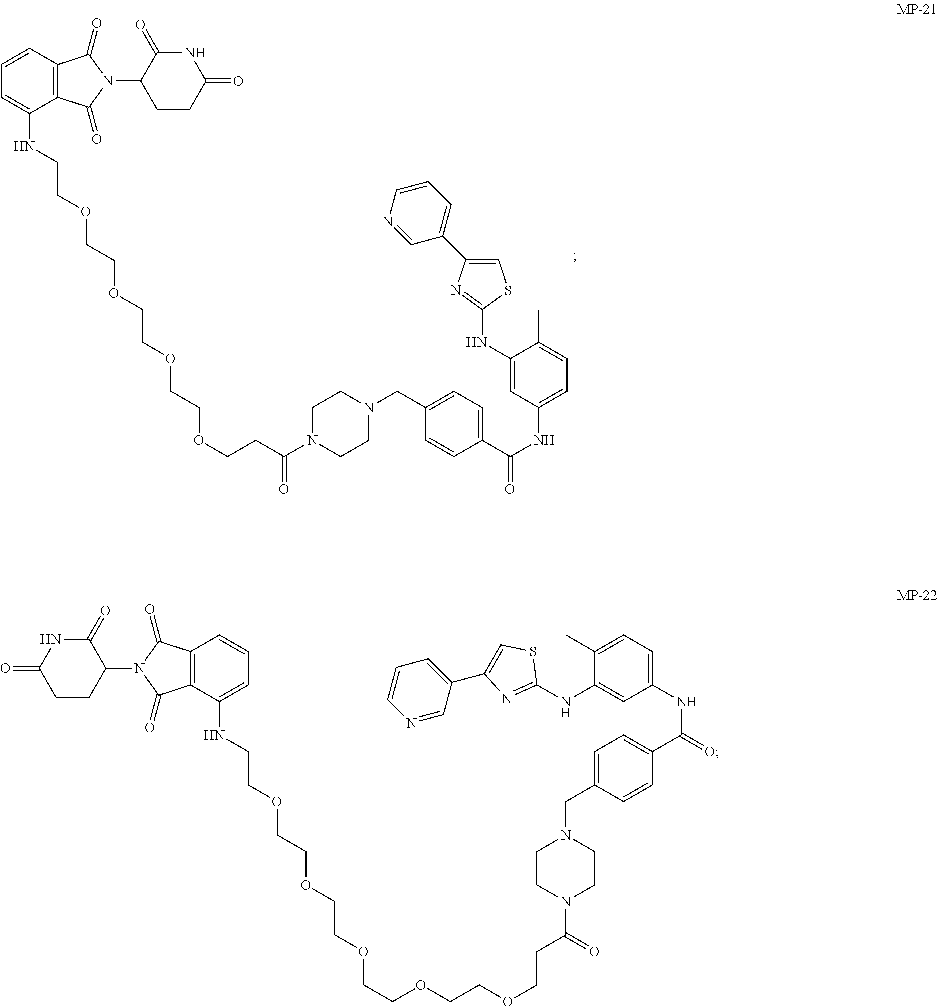 [SMILES: Cc1ccc(NC(=O)c2ccc(CN3CCN(C(=O)CCOCCOCCOCCOCCNc4cccc5c4C(=O)N(C4CCC(=O)NC4=O)C5=O)CC3)cc2)cc1Nc1nc(-c2cccnc2)cs1 ; Cc1ccc(NC(=O)c2ccc(CN3CCN(C(=O)CCOCCOCCOCCOCCOCCNc4cccc5c4C(=O)N(C4CCC(=O)NC4=O)C5=O)CC3)cc2)cc1Nc1nc(-c2cccnc2)cs1]

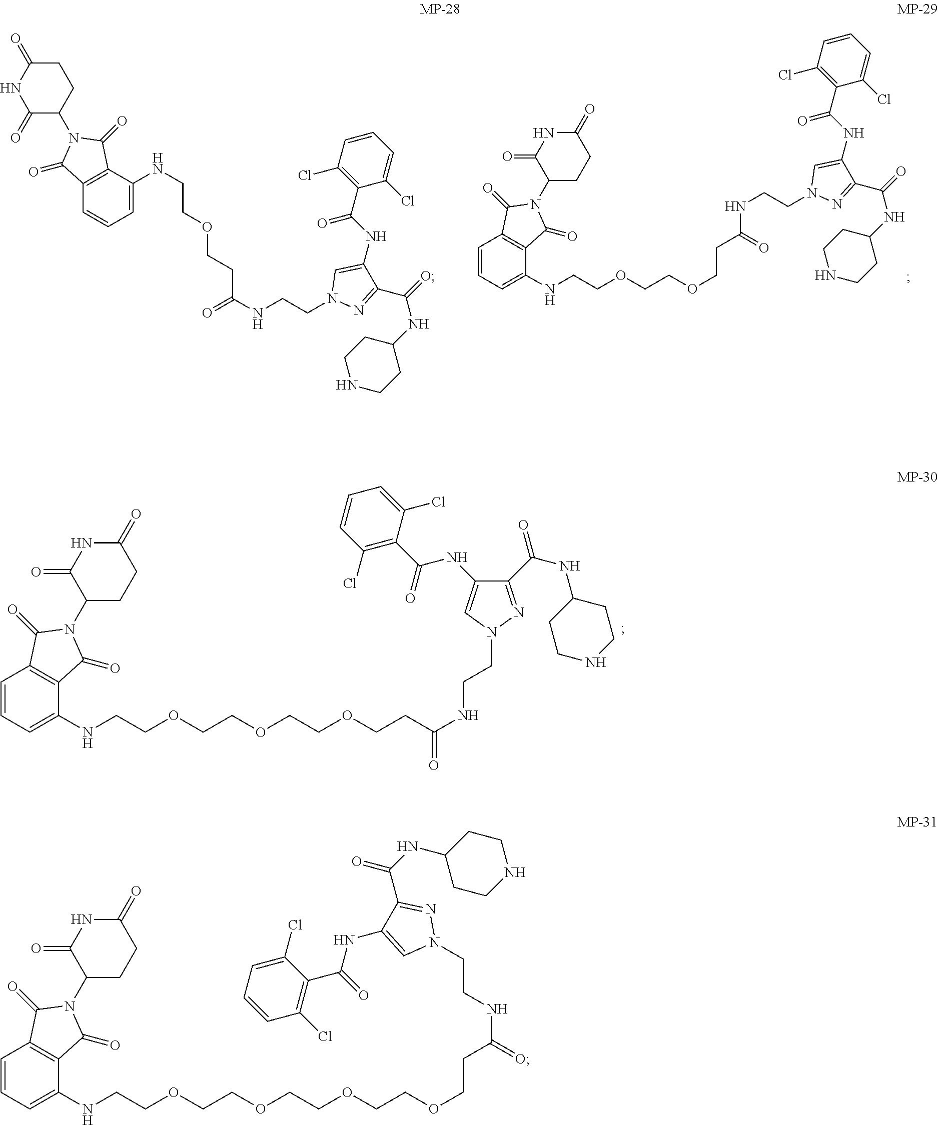 [SMILES: O=C(CCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; O=C(CCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; O=C(CCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; O=C(CCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1]

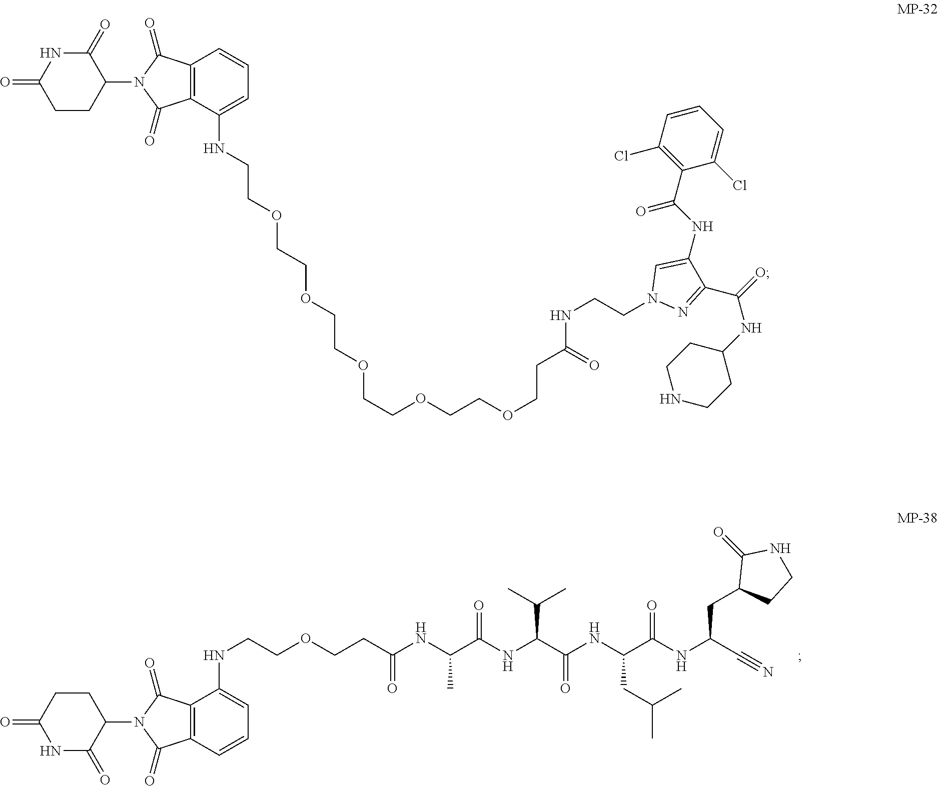 [SMILES: O=C(CCOCCOCCOCCOCCOCCNc1cccc2c1C(=O)N(C1CCC(=O)NC1=O)C2=O)NCCn1cc(NC(=O)c2c(Cl)cccc2Cl)c(C(=O)NC2CCNCC2)n1 ; CC(C)C[C@H](NC(=O)[C@@H](NC(=O)[C@H](C)NC(=O)CCOCCNc1cccc2c1C(=O)N(C1CCC(=O)NC1=O)C2=O)C(C)C)C(=O)N[C@H](C#N)C[C@@H]1CCNC1=O]

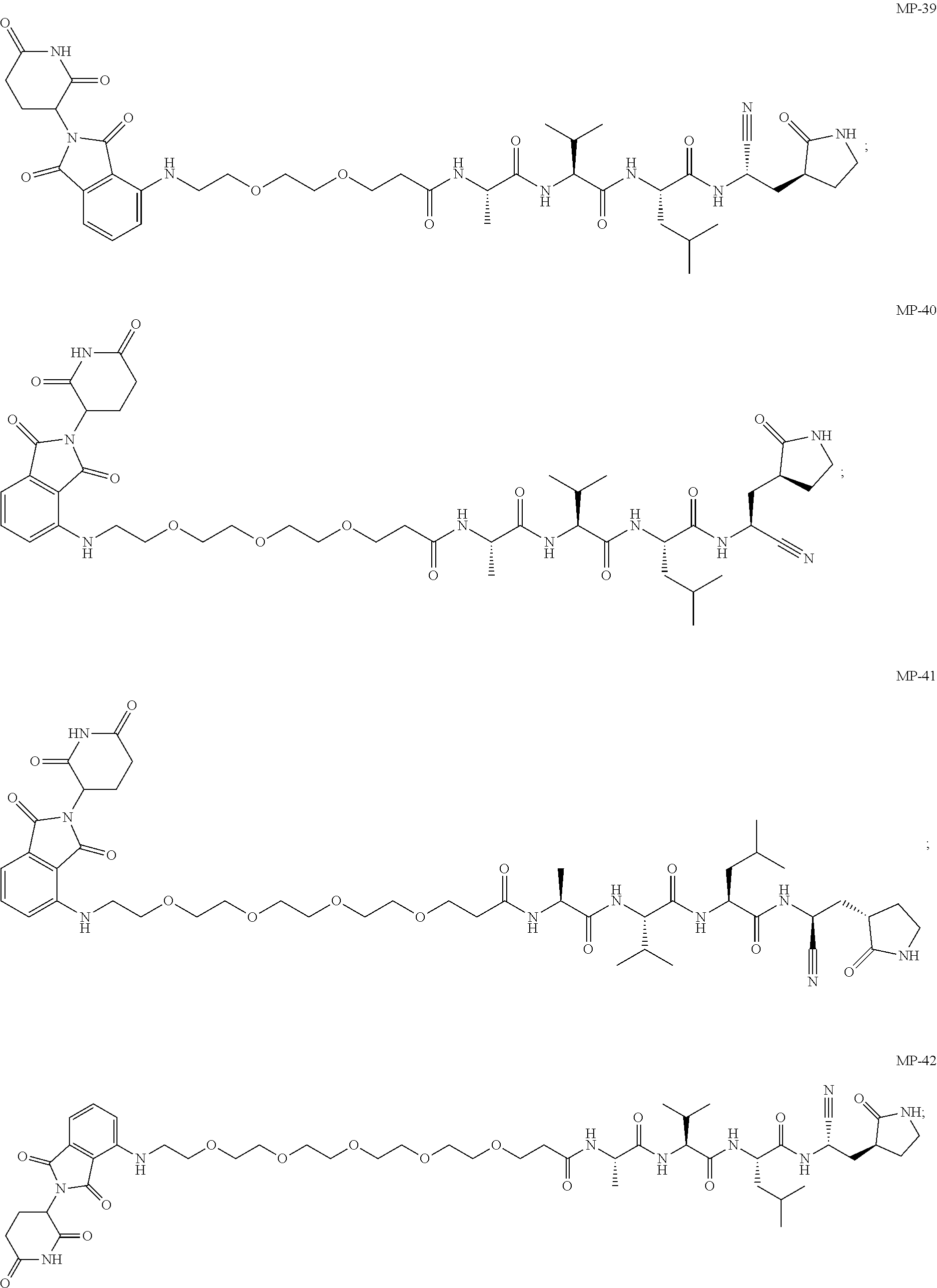

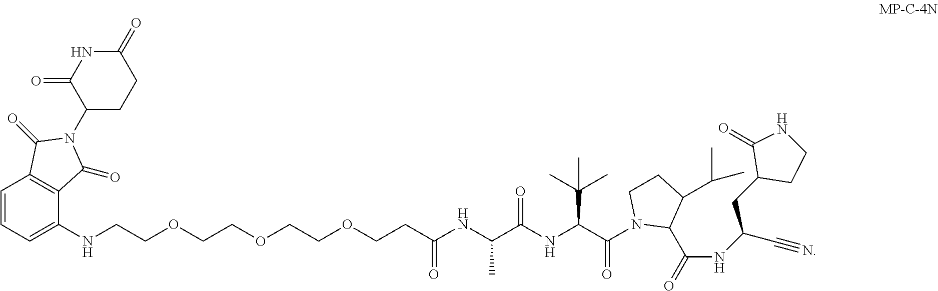 [SMILES: CC(C)C1CCN(C(=O)[C@@H](NC(=O)[C@H](C)NC(=O)CCOCCOCCOCCNc2cccc3c2C(=O)N(C2CCC(=O)NC2=O)C3=O)C(C)(C)C)C1C(=O)N[C@H](C#N)CC1CCNC1=O]

15-28. (canceled)

29. The composition of claim 1, further comprising a PLpro inhibitor attached to the linker.

30. The composition of claim 29, wherein the PLpro inhibitor comprises: PL-1, PL-2, PL-3, PL-4, PL-5, or PL-6:

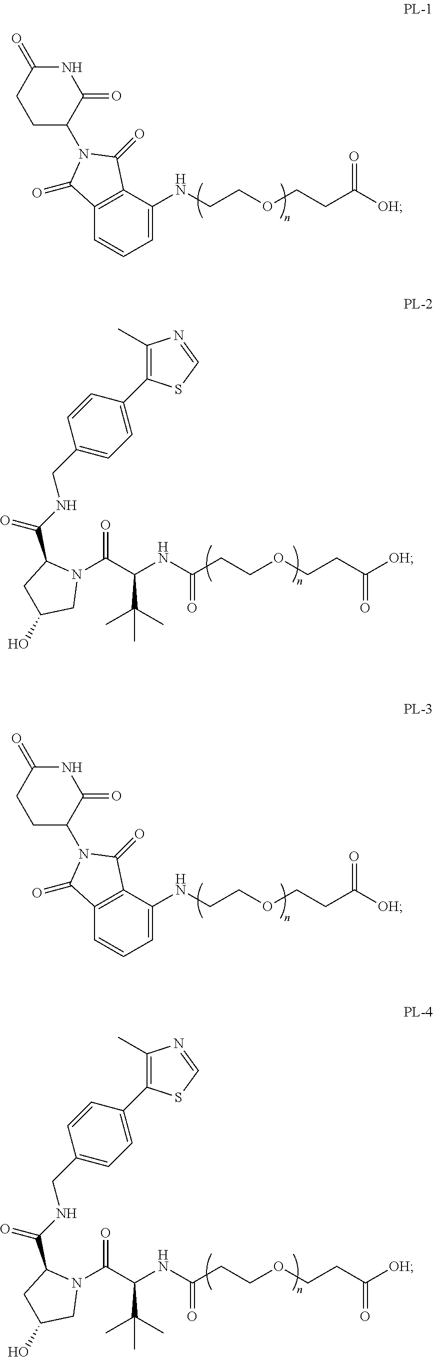

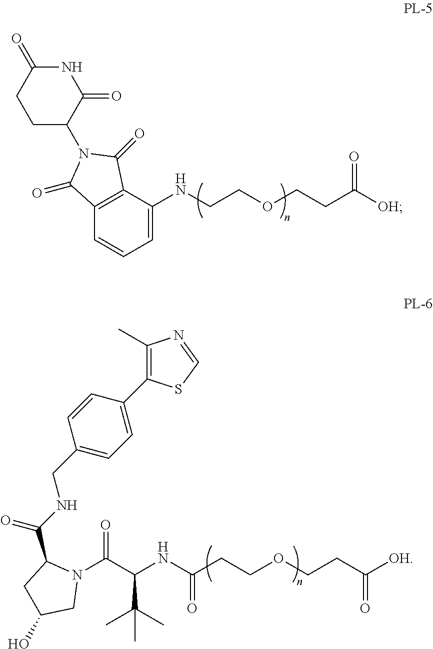

31-35. (canceled)

36. The composition of claim 29, comprising structure: DT-1, DT-2, DT-3, or DT-4: 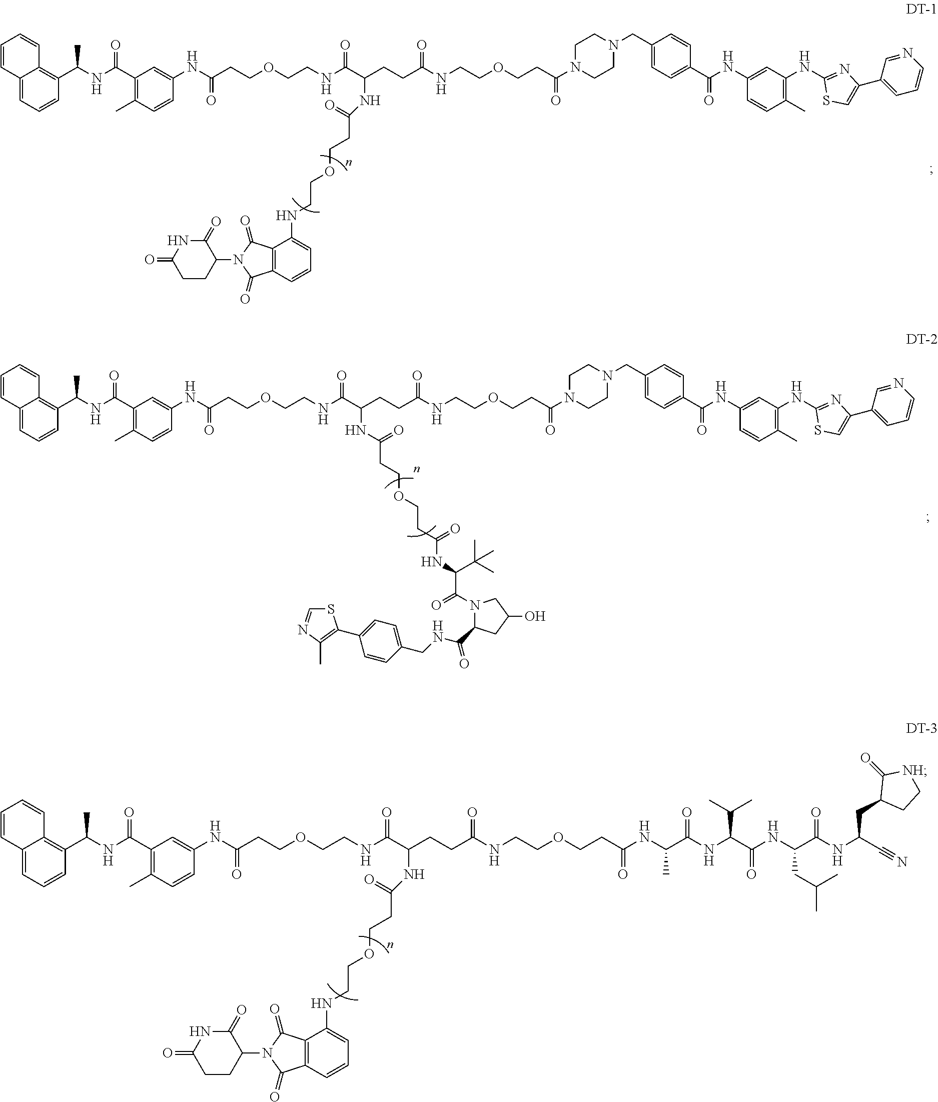 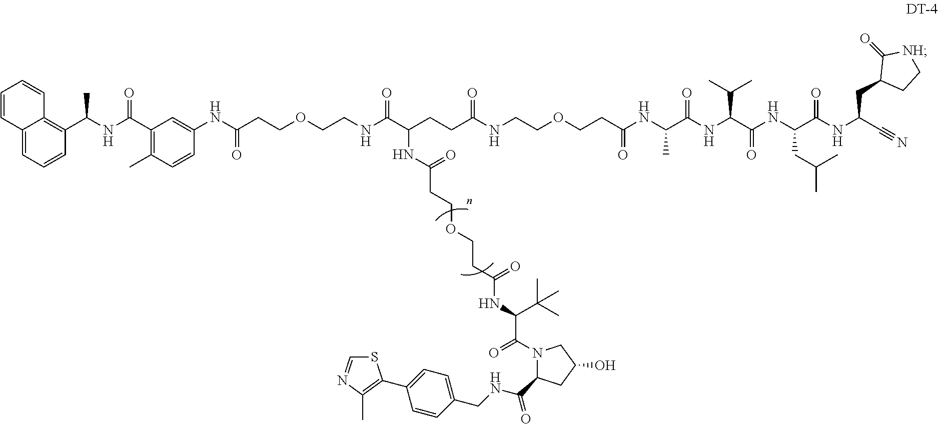
wherein n is 1-4.

37-51. (canceled)

52. The composition of claim 29, comprising structure DT-5: 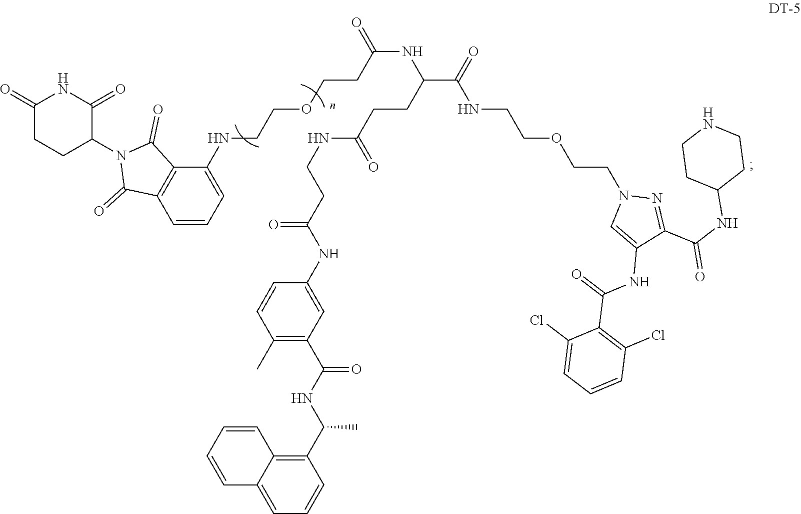
wherein n is 1-4.

53-55. (canceled)

56. The composition of claim 29, comprising structure DT-6: 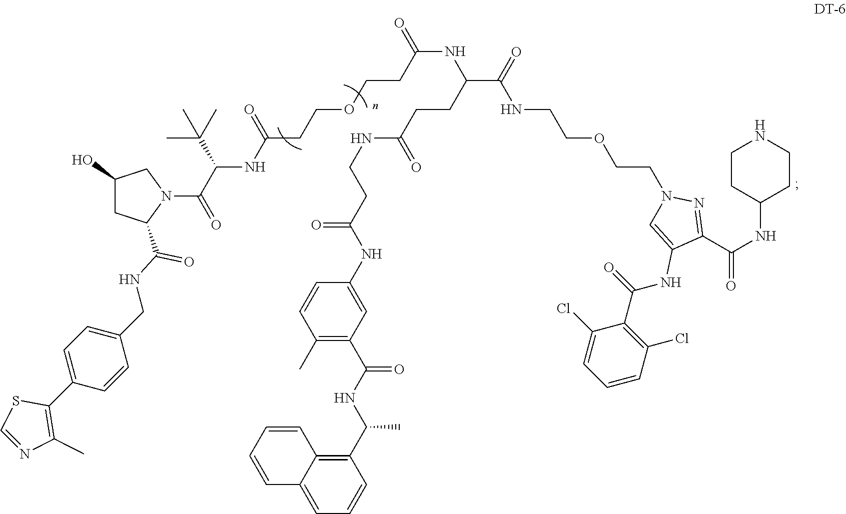
wherein n is 1-4.

57-59. (canceled)

60. A dual targeting PROTAC compound comprising:
an E3 ligase ligand;
a PLpro inhibitor;
a Mpro ligand; and
at least one linker connecting the E3 ligase ligand, the PLpro inhibitor, and the Mpro ligand.

61-64. (canceled)

65. A method of degrading Mpro activity in a cell infected with a coronavirus, the method comprising contacting the cell with an effective amount of a PROTAC compound capable of binding to a Mpro protein of the coronavirus, and degrading Mpro activity in the cell.

66. The method of claim 65, wherein the coronavirus is SARS-CoV-2.

67-71. (canceled)
